(19) United States
(12) Patent Application Publication
(10) Pub. No.: US 2020/0115741 A1
(43) Pub. Date: Apr. 16, 2020

(54) ANALYTICAL METHODS AND ARRAYS FOR USE IN THE SAME

(71) Applicant: SENZAGEN AB, Lund (SE)

(72) Inventors: Malin Marie Lindstedt, Södra Sandby (SE); Henrik Johansson, Malmö (SE); Kathrin Zeller Walther, Lund (SE)

(21) Appl. No.: 16/500,827

(22) PCT Filed: Apr. 23, 2018
(86) PCT No.: PCT/EP2018/060386

§ 371 Date: Oct. 4, 2019

(30) Foreign Application Priority Data
Apr. 24, 2017 (GB) 1706464.3

(51) Int. Cl. C12Q 1/6837; G01N 33/50; C12N 15/10

(52) U.S. Cl. CPC C12Q 1/6837; C12N 15/1086; C12N 15/1003; G01N 33/5047

(57) ABSTRACT

The present invention relates to a method for identifying proteins which are allergenic in a mammal, and arrays and analytical kits for use in such methods.

### FIELD OF THE INVENTION

The present invention relates to a method for identifying proteins which are allergenic in a mammal, and arrays and analytical kits for use in such methods.

### BACKGROUND

Allergy is a chronic disease with increasing prevalence and it is of outmost importance for the industry and authorities to identify potential allergens as early as possible to limit the exposure of workers and the general populations. Several hundreds of chemicals are known to be able to cause allergic contact dermatitis [1, 2], a type IV delayed hypersensitivity reaction, whereas less chemicals are known to sensitize the respiratory tract and to induce type I allergic responses [3]. Most substances causing respiratory allergy are proteins of environmental origin e.g. allergens from house dust mite feces, pollen, or fungi, while others are present in an occupational setting such as enzymes used in flavor, fragrance, detergents and pharmaceutical production [4, 5]. The risk of developing adverse reactions following occupational exposure exists; thus, a strict focus on occupational safety is mandatory. Sensitization has been observed for workers exposed to certain industrial enzymes such as α-amylase, proteases, pancreatinin, and trypsin [6, 7]. New enzymes are continuously developed for existing as well as for new applications, such as genetically modified enzymes used in food processing and flavor production and may also lead to occupational health risks [5, 7].

To date, no validated assay is available specifically for predicting the allergenicity of novel proteins, rendering a weight-of-evidence approach to be the most acceptable means of allergy safety assessment. There is, however, a growing consensus that the allergenic potential of compounds, including proteins, should be evaluated with regard to their biochemical characteristics and the protein's potential to induce a specific immune response (European COST Project impARAS [8]). A combination of physical traits of proteins, the molecular interaction between human cells and proteins, as well as their impact on cell-cell interactions play a role in understanding and eventually predicting protein allergenicity [9, 10].

The Genomic Allergen Rapid Detection (GARD) assay was initially developed to provide information about the capacity of chemicals to induce skin sensitization (accuracy: 89% [13, 17]). This in vitro assay utilizes a myeloid cell line resembling dendritic cells (DCs) as a model system. DCs are antigen-presenting cells and central for the induction and regulation of adaptive immune responses [14]. This assay was recognized by both the European Reference Laboratory—European Center for Validation of Alternative Methods (EURL-ECVAM) and the OECD as a valuable method for addressing key event 3 (Dendritic cell activation and maturation) of the AOP for skin sensitization [15]. Forreryd et al. [16] successfully demonstrated that a modified protocol of the assay is able to predict respiratory chemical sensitizers with an accuracy of 84% based on a biomarker signature consisting of 389 transcripts.

Hence, an in vitro assay specifically optimised for predicting the allergenicity of novel proteins remains desirable.

### DISCLOSURE OF THE INVENTION

The inventors have now shown that a genomic biomarker profile can be developed using the GARD platform for the allergenic assessment specifically of proteins.

Accordingly, a first aspect of the invention provides provides a method for identifying proteins which are allergenic in a mammal comprising or consisting of the steps of:
(a) providing a population of dendritic cells or a population of dendritic-like cells;
(b) exposing the cells provided in step (a) to a test protein; and
(c) measuring in the cells of step (b) the expression of two or more biomarkers selected from the group defined in Table A;

wherein the expression of the two or more biomarkers measured in step (c) is indicative of the allergenicity of the test protein of step (b).

In an additional or alternative embodiment one or more of the biomarkers measured in step (c) is selected from the group defined in Table A(i).

In an additional or alternative embodiment step (c) comprises or consists of measuring the expression of one or more biomarker listed in Table A(i), for example, 2, 3, 4, 5, 6, 7, 8, 9, 10, 11, 12, or 13 of the biomarkers listed in Table A(i). For example, step (c) may comprise or consist of measuring the expression of all of the biomarkers listed in Table A(i).

The method may include or exclude measuring the expression of TRIML2. The method may include or exclude measuring the expression of CYP1A2. The method may include or exclude measuring the expression of MAP9. The method may include or exclude measuring the expression of LOCI 00131971. The method may include or exclude measuring the expression of Transcript ID GENSCAN00000048751///ENST00000354794. The method may include or exclude measuring the expression of GRP. The method may include or exclude measuring the expression of Transcript ID GENSCAN00000015233///ENST00000358162. The method may include or exclude measuring the expression of MOBKL1B. The method may include or exclude measuring the expression of Transcript ID ENST00000411383///ENST00000386420. The method may include or exclude measuring the expression of BNC2. The method may include or exclude measuring the expression of SFTPA1///SFTPA1B///SFTPA2///SFTPA2B (Probe Set ID 7934708). The method may include or exclude measuring the expression of C21orf118. The method may include or exclude measuring the expression of Transcript ID ENST00000365169.

In an additional or alternative embodiment step (c) comprises or consists of measuring the expression of one or more biomarkers listed in Table A(ii), for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, or 378 of the biomarkers listed in Table A(ii). For example, step (c) may comprise or consist of measuring the expression of all of the biomarkers listed in Table A(ii).

The method may include or exclude measuring the expression of SFTPA1///SFTPA1B///SFTPA2///SFTPA2B (Probe Set ID 7934698). The method may include or exclude measuring the expression of RPE65. The method may include or exclude measuring the expression of FAM167A. The method may include or exclude measuring the expression of Transcript ID ENST00000387309. The method may include or exclude measuring the expression of FLJ44313. The method may include or exclude measuring the expression of NTN4. The method may include or exclude measuring the expression of STAT4. The method may include or exclude measuring the expression of SLC45A2. The method may include or exclude measuring the expression of LOC100133036///FAM95B1 (Probe Set ID 8161381). The method may include or exclude measuring the expression of GLT6D1. The method may include or exclude measuring the expression of Transcript ID AF119888. The method may include or exclude measuring the expression of SYCP2L. The method may include or exclude measuring the expression of KLK3. The method may include or exclude measuring the expression of Transcript ID GENSCAN00000042517. The method may include or exclude measuring the expression of RP11-191L9.1. The method may include or exclude measuring the expression of SLC17A8. The method may include or exclude measuring the expression of ST8SIA2. The method may include or exclude measuring the expression of Transcript ID ENST00000319817. The method may include or exclude measuring the expression of Transcript ID ENST00000387801///ENST00000387652///ENST00000387676///ENST00000387734///ENST00000386042. The method may include or exclude measuring the expression of Transcript ID ENST00000385544. The method may include or exclude measuring the expression of IRX6. The method may include or exclude measuring the expression of LOCI 00133036///FAM95B1 (Probe Set ID 8155627). The method may include or exclude measuring the expression of Transcript ID GENSCAN00000024384///ENST00000364863. The method may include or exclude measuring the expression of Transcript ID ENST00000362375. The method may include or exclude measuring the expression of C12orf36. The method may include or exclude measuring the expression of RPRM. The method may include or exclude measuring the expression of OR10AD1. The method may include or exclude measuring the expression of Transcript ID ENST00000326734///BC118644. The method may include or exclude measuring the expression of Transcript ID ENST00000411186. The method may include or exclude measuring the expression of Transcript ID ENST00000387641. The method may include or exclude measuring the expression of LRRC55. The method may include or exclude measuring the expression of FERMT2. The method may include or exclude measuring the expression of LOC100130815. The method may include or exclude measuring the expression of RTP2. The method may include or exclude measuring the expression of PLCZ1. The method may include or exclude measuring the expression of FLJ25328. The method may include or exclude measuring the expression of Transcript ID ENST00000411400///ENST00000385589. The method may include or exclude measuring the expression of Transcript ID GENSCAN00000027599///ENST00000286193. The method may include or exclude measuring the expression of Transcript ID GENSCAN00000026551///ENST00000329491. The method may include or exclude measuring the expression of CYP2A13///CYP2A7///CYP2A6. The method may include or exclude measuring the expression of PRY///PRY2 (Probe Set ID 8176935). The method may include or exclude measuring the expression of GPR45. The method may include or exclude measuring the expression of Probe Set ID 8070930. The method may include or exclude measuring the expression of Transcript ID AK095738. The method may include or exclude measuring the expression of Transcript ID ENST00000377462. The method may include or exclude measuring the expression of CITED1. The method may include or exclude measuring the expression of Transcript ID ENST00000385536. The method may include or exclude measuring the expression of PXK. The method may include or exclude measuring the expression of Transcript ID ENST00000365399. The method may include or exclude measuring the expression of Transcript ID ENST00000387878. The method may include or exclude measuring the expression of MGAT5B. The method may include or exclude measuring the expression of RP11-35F15.2. The method may include or exclude measuring the expression of MAP1LC3B. The method may include or exclude measuring the expression of PRY///PRY2 (Probe Set ID 8177395). The method may include or exclude measuring the expression of CYP2C19. The method may include or exclude measuring the expression of LOC646187. The method may include or exclude measuring the expression of LOC158381. The method may include or exclude measuring the expression of TERF1. The method may include or exclude measuring the expression of WBP5. The method may include or exclude measuring the expression of Transcript ID ENST00000340456///AK128036. The method may include or exclude measuring the expression of LOC153328. The method may include or exclude measuring the expression of RAB10. The method may include or exclude measuring the expression of DI0305. The method may include or exclude measuring the expression of PDE11A. The method may include or exclude measuring the expression of EGR1. The method may include or exclude measuring the expression of C9orf7. The method may include or exclude measuring the expression of Transcript ID ENST00000365097. The method may include or exclude measuring the expression of CHAC1. The method may include or exclude measuring the expression of Transcript ID ENST00000384640. The method may include or exclude measuring the expression of DAOA. The method may include or exclude measuring the expression of Transcript ID ENST00000356058///AK128129. The method may include or exclude measuring the expression of IFNA7///IFNA14. The method may include or exclude measuring the expression of POM121L1///DKFZp434K191///DKFZP434P211 (Probe Set ID 8071168). The method may include or exclude measuring the expression of Transcript ID AF304443. The method may include or exclude measuring the expression of Transcript ID ENST00000364415. The method may include or exclude measuring the expression of PSD. The method may include or exclude measuring the expression of IQCF2. The method may include or exclude measuring the expression of OR52A4. The method may include or exclude measuring the expression of FOS. The method may include or exclude measuring the expression of MSTP9///MST1. The method may include or exclude measuring the expression of MAF. The method may include or exclude measuring the expression of Transcript ID ENST00000388431///ENST00000388445 (Probe Set ID 7943954). The method may include or exclude measuring the expression of EMID2. The method may include or exclude measuring the expression of MDGA2. The method may include or exclude measuring the expression of Transcript ID hsa-mir-15a///hsa-mir-15a. The method may include or exclude measuring the expression of LOC93432. The method may include or exclude measuring the expression of NPC1L1. The method may include or exclude measuring the expression of NR4A2. The method may include or exclude measuring the expression of Transcript ID BC008359. The method may include or exclude measuring the expression of OPN5. The method may include or exclude measuring the expression of Transcript ID ENST00000385543. The method may include or exclude measuring the expression of Transcript ID ENST00000385921///ENST00000410743. The method may include or exclude measuring the expression of AADACL2. The method may include or exclude measuring the expression of C12orf54. The method may include or exclude measuring the expression of Transcript ID ENST00000387268. The method may include or exclude measuring the expression of Transcript ID ENST00000364509. The method may include or exclude measuring the expression of PRY///PRY2 (Probe Set ID 8176806). The method may include or exclude measuring the expression of WBP11P1. The method may include or exclude measuring the expression of SPRED3. The method may include or exclude measuring the expression of MAPT. The method may include or exclude measuring the expression of Probe Set ID 8058145. The method may include or exclude measuring the expression of Transcript ID ENST00000410136. The method may include or exclude measuring the expression of PGR. The method may include or exclude measuring the expression of SLC26A5. The method may include or exclude measuring the expression of LOC642538///LOC642521 (Probe Set ID 7934731). The method may include or exclude measuring the expression of CRP. The method may include or exclude measuring the expression of WDR38. The method may include or exclude measuring the expression of S100A5. The method may include or exclude measuring the expression of Transcript ID ENST00000411154///ENST00000387157. The method may include or exclude measuring the expression of CSN1S1. The method may include or exclude measuring the expression of Transcript ID ENST00000384294. The method may include or exclude measuring the expression of AP3S1. The method may include or exclude measuring the expression of ENPP5. The method may include or exclude measuring the expression of FXYD7. The method may include or exclude measuring the expression of CADPS. The method may include or exclude measuring the expression of RNF38. The method may include or exclude measuring the expression of Transcript ID ENST00000404200///ENST00000401594///ENST00000366307. The method may include or exclude measuring the expression of ASB16. The method may include or exclude measuring the expression of AVPR1A. The method may include or exclude measuring the expression of Transcript ID ENST00000387477. The method may include or exclude measuring the expression of CBLL1. The method may include or exclude measuring the expression of C15orf51. The method may include or exclude measuring the expression of FOSB. The method may include or exclude measuring the expression of Transcript ID ENST00000384559. The method may include or exclude measuring the expression of LOC642538///LOC642521 (Probe Set ID 7934729). The method may include or exclude measuring the expression of C10orf90. The method may include or exclude measuring the expression of BCAN. The method may include or exclude measuring the expression of PPBPL2. The method may include or exclude measuring the expression of Transcript ID GENSCAN00000020848///ENST00000409669///ENST00000410082///ENST00000409686. The method may include or exclude measuring the expression of IL1F10. The method may include or exclude measuring the expression of C1D (Probe Set ID 7932964). The method may include or exclude measuring the expression of PRAMEF7///PRAMEF8 (Probe Set ID 7912606). The method may include or exclude measuring the expression of Transcript ID ENST00000387283. The method may include or exclude measuring the expression of FKBP9L. The method may include or exclude measuring the expression of LOC728264. The method may include or exclude measuring the expression of HAPLN2. The method may include or exclude measuring the expression of PRAMEF7///PRAMEF8 (Probe Set ID 7912591). The method may include or exclude measuring the expression of UNQ9370. The method may include or exclude measuring the expression of MAP1LC3C. The method may include or exclude measuring the expression of PRAMEF7///PRAMEF8 (Probe Set ID 7897991). The method may include or exclude measuring the expression of SLC15A1. The method may include or exclude measuring the expression of Transcript ID ENST00000388545. The method may include or exclude measuring the expression of C10orf110. The method may include or exclude measuring the expression of Transcript ID hsa-mir-375///hsa-mir-375. The method may include or exclude measuring the expression of GP1BB. The method may include or exclude measuring the expression of LOC100129581. The method may include or exclude measuring the expression of BRS3. The method may include or exclude measuring the expression of CCDC63. The method may include or exclude measuring the expression of ONECUT2. The method may include or exclude measuring the expression of Transcript ID ENST00000387825. The method may include or exclude measuring the expression of Transcript ID ENST00000364793. The method may include or exclude measuring the expression of TBX18. The method may include or exclude measuring the expression of DKFZP686I15217. The method may include or exclude measuring the expression of C9orf98. The method may include or exclude measuring the expression of MYH1. The method may include or exclude measuring the expression of CASQ1. The method may include or exclude measuring the expression of DUSP1. The method may include or exclude measuring the expression of Transcript ID AK125575. The method may include or exclude measuring the expression of ZNF781. The method may include or exclude measuring the expression of Transcript ID ENST00000354690. The method may include or exclude measuring the expression of Transcript ID ENST00000363355. The method may include or exclude measuring the expression of C1D (Probe Set ID 8052698). The method may include or exclude measuring the expression of Transcript ID ENST00000388656. The method may include or exclude measuring the expression of LOH3CR2A. The method may include or exclude measuring the expression of MTNR1A. The method may include or exclude measuring the expression of Transcript ID ENST00000388431///ENST00000388445 (Probe Set ID 7951701). The method may include or exclude measuring the expression of TMEM38B. The method may include or exclude measuring the expression of ENST00000387816. The method may include or exclude measuring the expression of UROC1. The method may include or exclude measuring the expression of Transcript ID ENST00000363309. The method may include or exclude measuring the expression of Transcript ID ENST00000316807. The method may include or exclude measuring the expression of C13orf1. The method may include or exclude measuring the expression of UGCG. The method may include or exclude measuring the expression of POM121L1///DKFZp434K191///DKFZP434P211 (Probe Set ID 8074714). The method may include or exclude measuring the expression of FLJ38773. The method may include or exclude measuring the expression of LRRFIP1. The method may include or exclude measuring the expression of FCRL6. The method may include or exclude measuring the expression of FLJ38723. The method may include or exclude measuring the expression of HSP90AA6P. The method may include or exclude measuring the expression of CALR3. The method may include or exclude measuring the expression of ST18. The method may include or exclude measuring the expression of Transcript ID GENSCAN00000025928///ENST00000312946///ENST00000402897. The method may include or exclude measuring the expression of PTGS2. The method may include or exclude measuring the expression of NICN1///AMT. The method may include or exclude measuring the expression of TTC28. The method may include or exclude measuring the expression of MCL1. The method may include or exclude measuring the expression of SULT1A1. The method may include or exclude measuring the expression of Transcript ID ENST00000386719///ENST00000410999. The method may include or exclude measuring the expression of IGLL3. The method may include or exclude measuring the expression of FAM98B. The method may include or exclude measuring the expression of SLC26A4. The method may include or exclude measuring the expression of Probe Set ID 8180281. The method may include or exclude measuring the expression of PDE12. The method may include or exclude measuring the expression of SLC1A1. The method may include or exclude measuring the expression of PLSCR4. The method may include or exclude measuring the expression of TPT1. The method may include or exclude measuring the expression of SNRPN///SNORD116-25. The method may include or exclude measuring the expression of Transcript ID BC068044. The method may include or exclude measuring the expression of C1orf127. The method may include or exclude measuring the expression of FLJ11827. The method may include or exclude measuring the expression of CD2. The method may include or exclude measuring the expression of TMSB10. The method may include or exclude measuring the expression of PRY///PRY2 (Probe Set ID 8177323). The method may include or exclude measuring the expression of DPM3. The method may include or exclude measuring the expression of LOC442132. The method may include or exclude measuring the expression of NAALAD2. The method may include or exclude measuring the expression of ANO5. The method may include or exclude measuring the expression of GPR160. The method may include or exclude measuring the expression of SCN2A. The method may include or exclude measuring the expression of Transcript ID ENST00000364421. The method may include or exclude measuring the expression of TSPYL1. The method may include or exclude measuring the expression of DR1. The method may include or exclude measuring the expression of Transcript ID ENST00000410179. The method may include or exclude measuring the expression of ELOVL5. The method may include or exclude measuring the expression of FAM127C. The method may include or exclude measuring the expression of TNFSFI4. The method may include or exclude measuring the expression of FZD8. The method may include or exclude measuring the expression of ATPAF1. The method may include or exclude measuring the expression of Transcript ID ENST00000388700///ENST00000411294. The method may include or exclude measuring the expression of LOC100132357 (Probe Set ID 8161437). The method may include or exclude measuring the expression of C17orf82. The method may include or exclude measuring the expression of ACER2. The method may include or exclude measuring the expression of LOC100132357 (Probe Set ID 8161426). The method may include or exclude measuring the expression of C3orf58. The method may include or exclude measuring the expression of CBLN1. The method may include or exclude measuring the expression of PNPLA7. The method may include or exclude measuring the expression of ATP7B. The method may include or exclude measuring the expression of NCCRP1. The method may include or exclude measuring the expression of LOC100132357 (Probe Set ID 8155569). The method may include or exclude measuring the expression of ZNF835. The method may include or exclude measuring the expression of Transcript ID ENST00000322493. The method may include or exclude measuring the expression of Transcript ID ENST00000384168. The method may include or exclude measuring the expression of FOXJ3. The method may include or exclude measuring the expression of IKZF3. The method may include or exclude measuring the expression of Transcript ID ENST00000386866. The method may include or exclude measuring the expression of GABRR1. The method may include or exclude measuring the expression of MED31. The method may include or exclude measuring the expression of LRRC32. The method may include or exclude measuring the expression of MFSD6L. The method may include or exclude measuring the expression of CYP19A1. The method may include or exclude measuring the expression of ZNF565. The method may include or exclude measuring the expression of CSNK2A1P///CSNK2A1. The method may include or exclude measuring the expression of DNAH11. The method may include or exclude measuring the expression of Transcript ID ENST00000365299. The method may include or exclude measuring the expression of Probe Set ID 8180232. The method may include or exclude measuring the expression of OSMR. The method may include or exclude measuring the expression of Transcript ID ENST00000391137. The method may include or exclude measuring the expression of SUMO4. The method may include or exclude measuring the expression of SCGB1D1. The method may include or exclude measuring the expression of RPL39L. The method may include or exclude measuring the expression of Transcript ID ENST00000363408. The method may include or exclude measuring the expression of Transcript ID ENST00000384108. The method may include or exclude measuring the expression of MESP2. The method may include or exclude measuring the expression of EHF. The method may include or exclude measuring the expression of ERO1L. The method may include or exclude measuring the expression of EEF1E1. The method may include or exclude measuring the expression of SLC7A3. The method may include or exclude measuring the expression of SPATA19. The method may include or exclude measuring the expression of NCR1. The method may include or exclude measuring the expression of KLK4. The method may include or exclude measuring the expression of KLHDC7B. The method may include or exclude measuring the expression of MBTPS2. The method may include or exclude measuring the expression of PAH. The method may include or exclude measuring the expression of C4orf27. The method may include or exclude measuring the expression of HUS1. The method may include or exclude measuring the expression of DNAH9. The method may include or exclude measuring the expression of FLJ27255. The method may include or exclude measuring the expression of TMEM33. The method may include or exclude measuring the expression of SGCZ. The method may include or exclude measuring the expression of HLA-DQB2. The method may include or exclude measuring the expression of KRT24. The method may include or exclude measuring the expression of GTSF1L. The method may include or exclude measuring the expression of NETO2. The method may include or exclude measuring the expression of TTTY9A///TTTY9B (Probe Set ID 8177195). The method may include or exclude measuring the expression of MIR21. The method may include or exclude measuring the expression of TTTY9A///TTTY9B (Probe Set ID 8176692). The method may include or exclude measuring the expression of EVX2. The method may include or exclude measuring the expression of Transcript ID ENST00000363948. The method may include or exclude measuring the expression of TBXASI. The method may include or exclude measuring the expression of ADAM12. The method may include or exclude measuring the expression of CD7. The method may include or exclude measuring the expression of ATXN10. The method may include or exclude measuring the expression of ZNF826. The method may include or exclude measuring the expression of SLC35F2. The method may include or exclude measuring the expression of FGF1. The method may include or exclude measuring the expression of IL8. The method may include or exclude measuring the expression of Transcript ID ENST00000364677. The method may include or exclude measuring the expression of CLSTN2. The method may include or exclude measuring the expression of FLJ00049. The method may include or exclude measuring the expression of RCOR1. The method may include or exclude measuring the expression of VPS37A. The method may include or exclude measuring the expression of Transcript ID hsa-mir-221///hsa-mir-221. The method may include or exclude measuring the expression of Probe Set ID 8091186. The method may include or exclude measuring the expression of Transcript ID ENST00000386767. The method may include or exclude measuring the expression of UBE2K. The method may include or exclude measuring the expression of MYH15. The method may include or exclude measuring the expression of DUSP22. The method may include or exclude measuring the expression of HIAT1. The method may include or exclude measuring the expression of KLHL33. The method may include or exclude measuring the expression of Transcript ID ENST00000411322. The method may include or exclude measuring the expression of C7orf29. The method may include or exclude measuring the expression of Transcript ID ENST00000387115///ENST00000408541. The method may include or exclude measuring the expression of Transcript ID ENST00000386002. The method may include or exclude measuring the expression of Transcript ID hsa-mir-33a///hsa-mir-33a. The method may include or exclude measuring the expression of PTPLB. The method may include or exclude measuring the expression of TANC1. The method may include or exclude measuring the expression of DACT1. The method may include or exclude measuring the expression of PTPRD. The method may include or exclude measuring the expression of CDC26. The method may include or exclude measuring the expression of TSPYL3. The method may include or exclude measuring the expression of RXFP3. The method may include or exclude measuring the expression of Transcript ID ENST00000410526///ENST00000386460. The method may include or exclude measuring the expression of FLJ13224. The method may include or exclude measuring the expression of TAF1D///SNORA40. The method may include or exclude measuring the expression of HPRT1. The method may include or exclude measuring the expression of C10orf10. The method may include or exclude measuring the expression of Transcript ID ENST00000364910. The method may include or exclude measuring the expression of MGC16121. The method may include or exclude measuring the expression of Probe Set ID 8180344. The method may include or exclude measuring the expression of Transcript ID ENST00000387217. The method may include or exclude measuring the expression of GDA. The method may include or exclude measuring the expression of SNORD63. The method may include or exclude measuring the expression of YWHAG. The method may include or exclude measuring the expression of SNRPN///SNORD116-26. The method may include or exclude measuring the expression of ASNS. The method may include or exclude measuring the expression of Transcript ID ENST00000385636. The method may include or exclude measuring the expression of Transcript ID ENST00000389074. The method may include or exclude measuring the expression of Transcript ID ENST00000363891. The method may include or exclude measuring the expression of KLHL11. The method may include or exclude measuring the expression of KCNK6. The method may include or exclude measuring the expression of SVIP. The method may include or exclude measuring the expression of KLRA1. The method may include or exclude measuring the expression of CPSF6. The method may include or exclude measuring the expression of Transcript ID ENST00000384449. The method may include or exclude measuring the expression of Transcript ID ENST00000389758///ENST00000396517///ENST00000327506. The method may include or exclude measuring the expression of Transcript ID GENSCAN00000041083///ENST00000309074. The method may include or exclude measuring the expression of POGK. The method may include or exclude measuring the expression of TRPM6. The method may include or exclude measuring the expression of C9orf6. The method may include or exclude measuring the expression of Transcript ID ENST00000411285///ENST00000388598. The method may include or exclude measuring the expression of RAB2A. The method may include or exclude measuring the expression of NAV3. The method may include or exclude measuring the expression of Probe Set ID 8091118. The method may include or exclude measuring the expression of SLC22A4. The method may include or exclude measuring the expression of NAP1L5. The method may include or exclude measuring the expression of Transcript ID ENST00000363618. The method may include or exclude measuring the expression of TGFBRAP1. The method may include or exclude measuring the expression of RPL27A. The method may include or exclude measuring the expression of TP53TG3///LOC729355. The method may include or exclude measuring the expression of JSRP1. The method may include or exclude measuring the expression of Transcript ID ENST00000411174///ENST00000388411. The method may include or exclude measuring the expression of CCNA2. The method may include or exclude measuring the expression of AADACL3. The method may include or exclude measuring the expression of Transcript ID ENST00000363794. The method may include or exclude measuring the expression of TMEM184C. The method may include or exclude measuring the expression of POLR3G. The method may include or exclude measuring the expression of CLDN9///LOC100134406. The method may include or exclude measuring the expression of LRRC58. The method may include or exclude measuring the expression of Transcript ID GENSCAN00000001581. The method may include or exclude measuring the expression of Transcript ID hsa-mir-34b///hsa-mir-34b. The method may include or exclude measuring the expression of OR10K2. The method may include or exclude measuring the expression of Transcript ID GENSCAN00000048378///ENST00000404638. The method may include or exclude measuring the expression of KRAS. The method may include or exclude measuring the expression of ORC5L. The method may include or exclude measuring the expression of MYO15B. The method may include or exclude measuring the expression of TNFRSF10D. The method may include or exclude measuring the expression of NIPA2. The method may include or exclude measuring the expression of Transcript ID ENST00000386231///ENST00000411190. The method may include or exclude measuring the expression of C17orf88. The method may include or exclude measuring the expression of PPP1R15B. The method may include or exclude measuring the expression of CSNK2A2. The method may include or exclude measuring the expression of LOC100133036///FAM95B1 (Probe Set ID 8155418). The method may include or exclude measuring the expression of MPZL2. The method may include or exclude measuring the expression of LOC100134722///LOC100133005. The method may include or exclude measuring the expression of IER3IP1. The method may include or exclude measuring the expression of CCDC117. The method may include or exclude measuring the expression of DDIT4. The method may include or exclude measuring the expression of SEC24A.

In an additional or alternative embodiment step (c) comprises or consists of measuring the expression of three or more of the biomarkers listed in Table A, for example, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or 391 of the biomarkers listed in Table A. For example, step (c) may comprise or consist of measuring the expression of all of the biomarkers listed in Table A.

By “expression” we mean the presence, level and/or amount of the biomarker.

By “biomarker” we include any biological molecule, or component or fragment thereof, the measurement of which can provide information useful in determining the allergenicity of a protein. Thus, in the context of Table A, the biomarker may be a nucleic acid molecule, such as a mRNA or cDNA. Alternatively, the biomarker may be a protein encoded by the nucleic acid molecule or carbohydrate moiety, or an antigenic component or fragment thereof.

In an additional or alternative embodiment the method comprises the further steps of:
d) exposing a separate population of the dendritic cells or dendritic-like cells to one or more negative control agent that is not allergenic in a mammal; and
e) measuring in the cells of step (d) the expression of the two or more biomarkers measured in step (c)
wherein the test protein is identified as allergenic in the event that the expression of the two or more biomarkers measured in step (e) differs from the expression of the two or more biomarkers measured in step (c).

A vehicle control may be used as the negative control agent. The vehicle control may comprise DMSO.

In an additional or alternative embodiment unstimulated cells may be used as the negative control. By “unstimulated cells” we include or mean cells which have not been exposed to a specific test protein. In other words, the separate population of cells in step (d) is not exposed to a test protein. However, the separate population of cells may be exposed to cell media containing serum (e.g. at about 20% by volume) which comprises negative control proteins.

In an additional or alternative embodiment the expression of the two or more biomarkers measured in step (c) is measured in the cells provided in step (a) prior to and following exposure to the test protein, and wherein the difference in expression between the two or more biomarkers prior to and following exposure to the test protein is indicative of the allergenicity of the test protein of step (b). Hence, the cells provided in step (a) may provide both the negative control and the test result.

By “differs from the expression of the two or more biomarkers measured in step (c)” and “difference in expression” we include that the presence and or amount in a first sample (e.g., a test protein sample) differs from that of a second sample (e.g., a control agent sample).

For example, the presence and/or amount in the test sample may differ from that of the one or more negative control sample in a statistically significant manner. Preferably the expression of the two or more biomarkers in the cell population exposed to the test protein is:
less than or equal to 80% of that of the cell population exposed to the negative control agent, for example, no more than 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or 0% of that of the cell population exposed to the negative control or negative control agent; or
at least 120% of that of the cell population exposed to the negative control agent, for example, at least 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 225%, 250%, 275%, 300%, 325%, 350%, 375%, 400%, 425%, 450%, 475% or at least 500% of that of the cell population exposed to the negative control or negative control agent

By “differs from the expression of the two or more biomarkers measured in step (c)” we alternatively or additionally include that the test sample is classified as belonging to a different group as the one or more negative control sample. For example, where an SVM is used, the test sample is on the other side of the decision value threshold as the one or more negative control sample (e.g., if the test agent is classified as a protein allergen if one or more test (or replicate thereof) has an SVM decision value of 50, then the one or more positive control samples (or the majority thereof) should also have an SVM decision value of 50).

In an additional or alternative embodiment, the one or more negative control agent provided in step (d) is selected from the group consisting of: unstimulated cells; cell media; vehicle control; DMSO; LPS.

In an additional or alternative embodiment, the one or more negative control agent provided in step (d) is cell media. Preferably the media contains serum (preferably at about 20% by volume) which comprises negative control proteins.

In an additional or alternative embodiment, at least 2 negative controls and/or control non-allergenic agents are provided, for example, at least 3, 4, or at least 5 negative controls and/or control non-allergenic agents.

In an additional or alternative embodiment the method comprises the further steps of:
f) exposing a separate population of the dendritic cells or dendritic-like cells to one or more positive control agent that is allergenic in a mammal; and
g) measuring in the cells of step (f) the expression of the two or more biomarkers measured in step (c)
wherein the test protein is identified as allergenic in the event that the expression of the two or more biomarkers measured in step (f) corresponds to the expression of the two or more biomarkers measured in step (c).

By “corresponds to the expression of the two or more biomarkers measured in step (c)” we mean the expression of the two or more biomarkers in the cell population exposed to the test protein is identical to, or does not differ significantly from, that of the cell population exposed to the one more positive control agent. Preferably the expression of the two or more biomarkers in the cell population exposed to the test protein is between 81% and 119% of that of the cell population exposed to the one more positive control agent, for example, greater than or equal to 82%, 83%, 84%, 85%, 86%, 87%, 88%, 89%, 90%, 91%, 92%, 93%, 94%, 95%, 96%, 97%, 98% or 99% of that of the cell population exposed to the one more positive control agent, and less than or equal to 101%, 102%, 103%, 104%, 105%, 106%, 107%, 108%, 109%, 110%, 111%, 112%, 113%, 114%, 115%, 116%, 117%, 118% or 119% of that of the cell population exposed to the one more positive control agent.

By “corresponds to the expression of the two or more biomarkers measured in step (c)” we alternatively or additionally include that the test sample is classified as belonging to the same group as the one or more positive control sample. For example, where an SVM is used, the test sample is on the same side of the decision value threshold as the one or more positive control sample (e.g., if the test protein is classified as allergenic if one or more test (or replicate thereof) has an SVM decision value of >0, then the one or more positive control samples (or the majority thereof) should also have an SVM decision value of >0).

In an additional or alternative embodiment, the one or more positive control agent provided in step (f) comprises or consists of one or more agent selected from the group consisting of: Der p 1; and Der p 7.

In an additional or alternative embodiment, at least 2 control allergenic agents are provided.

In an additional or alternative embodiment, the control allergenic agents are allergenic proteins. In an additional or alternative embodiment, the control non-allergenic agents are non-allergenic proteins.

In an additional or alternative embodiment, the method is indicative of the allergenic potency of the protein to be tested. For example, the method may be used to predict the relative allergenic potency of a test protein compared to a positive control and/or compared to one or more additional test protein.

In an additional or alternative embodiment the method comprises the further step of:
(h) identifying the allergenicity of the test protein.

For example, step (h) may identify the test protein as being an allergen or a non-allergen. Alternatively or additionally, step (h) may identify the relative allergenicity or allergenic potency of the test protein compared to a positive control and/or one or more additional test proteins.

The identification may be performed using any suitable statistical method or machine learning algorithm known in the art, such as Random Forest (RF), Support Vector Machine (SVM), Principal Component Analysis (PCA), ordinary least squares (OLS), partial least squares regression (PLS), orthogonal partial least squares regression (O-PLS) and other multivariate statistical analyses (e.g., backward stepwise logistic regression model). For a review of multivariate statistical analysis see, for example, Schervish, Mark J. (November 1987). “A Review of Multivariate Analysis”. Statistical Science 2 (4): 396-413 which is incorporated herein by reference. Preferably, Support Vector Machine (SVM) is used.

Typically, allergenic proteins are identified using a support vector machine (SVM), such as those available from http://crans-project.org/web/packages/e1071/index.html (e.g. e1071 1.5-24). However, any other suitable means may also be used. SVMs may also be used to determine the ROC AUCs of biomarker signatures comprising or consisting of one or more Table A biomarkers as defined herein.

Support vector machines (SVMs) are a set of related supervised learning methods used for classification and regression. Given a set of training examples, each marked as belonging to one of two categories, an SVM training algorithm builds a model that predicts whether a new example falls into one category or the other. Intuitively, an SVM model is a representation of the examples as points in space, mapped so that the examples of the separate categories are divided by a clear gap that is as wide as possible. New examples are then mapped into that same space and predicted to belong to a category based on which side of the gap they fall on.

More formally, a support vector machine constructs a hyperplane or set of hyperplanes in a high or infinite dimensional space, which can be used for classification, regression or other tasks. Intuitively, a good separation is achieved by the hyperplane that has the largest distance to the nearest training datapoints of any class (so-called functional margin), since in general the larger the margin the lower the generalization error of the classifier. For more information on SVMs, see for example, Burges, 1998, Data Mining and Knowledge Discovery, 2:121-167.

In one embodiment of the invention, the SVM is ‘trained’ prior to performing the methods of the invention using biomarker profiles of known agents (namely, known allergenic or non-allergenic agents). By running such training samples, the SVM is able to learn what biomarker profiles are associated with proteins capable of inducing allergy. Once the training process is complete, the SVM is then able to predict whether or not the biomarker sample tested is from an allergenic or non-allergenic protein. Decision values for individual SVMs can be determined by the skilled person on a case-by-case basis. In one embodiment, the test protein is classified as allergenic if one or more test (or replicate thereof) have an SVM decision value of >0. In one embodiment, the test protein is classified as a non-allergenic protein if one or more test (or replicate thereof) have an SVM decision value of 50. This allows test proteins to be classified as allergenic or non-allergenic.

However, this training procedure can be by-passed by pre-programming the SVM with the necessary training parameters. For example, allergenic proteins can be identified according to the known SVM parameters using the SVM algorithm described in the Examples, based on the measurement of all the biomarkers listed in Table A.

It will be appreciated by skilled persons that suitable SVM parameters can be determined for any combination of the biomarkers listed Table A by training an SVM machine with the appropriate selection of data (i.e. biomarker measurements from cells exposed to known allergenic and/or non-allergenic agents). Alternatively, the Table A biomarkers may be used to identify allergenic proteins according to any other suitable statistical method known in the art.

Alternatively, the Table A data may be used to identify agents capable of inducing respiratory sensitization according to any other suitable statistical method known in the art (e.g., ANOVA, ANCOVA, MANOVA, MANCOVA, Multivariate regression analysis, Principal components analysis (PCA), Factor analysis, Canonical correlation analysis, Canonical correlation analysis, Redundancy analysis Correspondence analysis (CA; reciprocal averaging), Multidimensional scaling, Discriminant analysis, Linear discriminant analysis (LDA), Clustering systems, Recursive partitioning and Artificial neural networks).

Preferably, the methods of the invention have an accuracy of at least 60%, for example, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% accuracy. In a preferred embodiment, the methods of the invention have an accuracy of at least 93%

Preferably, the methods of the invention have a sensitivity of at least 60%, for example, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sensitivity. In a preferred embodiment, the methods of the invention have a sensitivity of at least 92%.

Preferably, the methods of the invention have a specificity of at least 60%, for example, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% specificity. In a preferred embodiment, the methods of the invention have a specificity of 100%.

By “accuracy” we mean the proportion of correct outcomes of a method, by “sensitivity” we mean the proportion of all positive proteins that are correctly classified as positives, and by “specificity” we mean the proportion of all negative proteins that are correctly classified as negatives.

In a preferred embodiment, step (c) comprises or consists of measuring the expression of a nucleic acid molecule of one or more of the biomarkers. The nucleic acid molecule may be a DNA molecule or a cDNA molecule or an mRNA molecule. Preferably, the nucleic acid molecule is an mRNA molecule. However, the nucleic acid molecule may be a cDNA molecule.

In one embodiment the measurement of the expression of one or more of the biomarkers in step (c) is performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation. Preferably, the expression of one or more biomarker(s) is measured using a DNA microarray.

In an additional or alternative embodiment the one or more biomarkers measured in step (c) is measured using an array (e.g., a DNA array). In an additional or alternative embodiment the one or more biomarkers measured in step (c) is measured using a whole genome array (e.g., the Affymetrix Human Gene 1.0 ST array or Affymetrix Human Gene 2.0 ST array). In an alternative or additional embodiment, the Nanostring nCounter system is used (e.g., custom Nanostring nCounter code sets based on selection from a whole genome array (e.g., Affymetrix Human Gene 1.0 ST array or Affymetrix Human Gene 2.0 ST array).

The method may comprise measuring the expression of one or more biomarkers in step (c) using one or more binding moieties, each capable of binding selectively to a nucleic acid molecule encoding one of the biomarkers identified in Table A. Preferably, the method comprises measuring the expression of two or more biomarkers in step (c) using two or more binding moieties, each capable of binding selectively to a nucleic acid molecule encoding one of the biomarkers identified in Table A. For example, the expression of any particular combination of biomarkers described above may be measured using an equivalent combination of binding moieties capable of binding selectively to each of those biomarkers.

In one embodiment the one or more binding moieties each comprise or consist of a nucleic acid molecule. In a further embodiment the one or more binding moieties each comprise or consist of DNA, RNA, PNA, LNA, GNA, TNA or PMO. Preferably, the one or more binding moieties each comprise or consist of DNA. In one embodiment, the one or more binding moieties are 5 to 100 nucleotides in length. However, in an alternative embodiment, they are 15 to 35 nucleotides in length.

The one or more binding moieties may comprise or consist of one or more probe from the Human Gene 1.0 ST Array (Affymetrix, Santa Clara, Calif., USA). Probe identification numbers are provided in Table A herein.

Suitable binding agents (also referred to as binding molecules or binding moieties) may be selected or screened from a library based on their ability to bind a given nucleic acid, protein or amino acid motif, as discussed below.

In a preferred embodiment, the binding moiety comprises a detectable moiety.

By a “detectable moiety” we include a moiety which permits its presence and/or relative amount and/or location (for example, the location on an array) to be determined, either directly or indirectly.

Suitable detectable moieties are well known in the art.

For example, the detectable moiety may be a fluorescent and/or luminescent and/or chemiluminescent moiety which, when exposed to specific conditions, may be detected. Such a fluorescent moiety may need to be exposed to radiation (i.e. light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

The detectable moiety may be a radioactive moiety and comprise or consists of a radioactive atom. The radioactive atom may be selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

Hence, the detectable moiety may be selected from the group consisting of: a fluorescent moiety; a luminescent moiety; a chemiluminescent moiety; a radioactive moiety (for example, a radioactive atom); or an enzymatic moiety.

Clearly, the agent to be detected (such as, for example, the one or more biomarkers in the test sample and/or control sample described herein and/or an antibody molecule for use in detecting a selected protein) must have sufficient of the appropriate atomic isotopes in order for the detectable moiety to be readily detectable.

In an alternative preferred embodiment, the detectable moiety of the binding moiety is a fluorescent moiety.

The radio- or other labels may be incorporated into the biomarkers present in the samples of the methods of the invention and/or the binding moieties of the invention in known ways. For example, if the binding agent is a polypeptide it may be biosynthesised or may be synthesised by chemical amino acid synthesis using suitable amino acid precursors involving, for example, fluorine-19 in place of hydrogen. Labels such as ^{99m}Tc, ¹²³I, ¹⁸⁶Rh, ¹⁸⁸Rh and ¹¹¹In can, for example, be attached via cysteine residues in the binding moiety. Yttrium-90 can be attached via a lysine residue. The IODOGEN method (Fraker et al (1978) Biochem. Biophys. Res. Comm. 80, 49-57) can be used to incorporate ¹²³I. Reference (“Monoclonal Antibodies in Immunoscintigraphy”, J-F Chatal, CRC Press, 1989) describes other methods in detail. Methods for conjugating other detectable moieties (such as enzymatic, fluorescent, luminescent, chemiluminescent or radioactive moieties) to proteins are well known in the art.

It will be appreciated by persons skilled in the art that biomarkers in the sample(s) to be tested may be labelled with a moiety which indirectly assists with determining the presence, amount and/or location of said proteins. Thus, the moiety may constitute one component of a multicomponent detectable moiety. For example, the biomarkers in the sample(s) to be tested may be labelled with biotin, which allows their subsequent detection using streptavidin fused or otherwise joined to a detectable label.

The method provided in the first aspect of the present invention may comprise or consist of, in step (c), determining the expression of the protein of one or more biomarker defined in Table A. The method may comprise measuring the expression of one or more biomarkers in step (c) using one or more binding moieties each capable of binding selectively to one of the biomarkers identified in Table A. The one or more binding moieties may comprise or consist of an antibody or an antigen-binding fragment thereof such as a monoclonal antibody or fragment thereof.

The term “antibody” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecules capable of binding to an antigen in an immunoassay format that is known to those skilled in the art.

We also include the use of antibody-like binding agents, such as affibodies and aptamers.

A general review of the techniques involved in the synthesis of antibody fragments which retain their specific binding sites is to be found in Winter & Milstein (1991) Nature 349, 293-299.

Additionally, or alternatively, one or more of the first binding molecules may be an aptamer (see Collett et al., 2005, Methods 37:4-15).

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

The molecular libraries may be expressed in vivo in prokaryotic cells (Clackson et al, 1991, op. cit.; Marks et al, 1991, op. cit.) or eukaryotic cells (Kieke et al, 1999, Proc Natl Acad Sci USA, 96(10):5651-6) or may be expressed in vitro without involvement of cells (Hanes & Pluckthun, 1997, Proc Natl Acad Sci USA 94(10):4937-42; He & Taussig, 1997, Nucleic Acids Res 25(24):5132-4; Nemoto et al, 1997, FEBS Lett, 414(2):405-8).

In cases when protein based libraries are used, the genes encoding the libraries of potential binding molecules are often packaged in viruses and the potential binding molecule displayed at the surface of the virus (Clackson et al, 1991, supra; Marks et al, 1991, supra; Smith, 1985, supra).

Perhaps the most commonly used display system is filamentous bacteriophage displaying antibody fragments at their surfaces, the antibody fragments being expressed as a fusion to the minor coat protein of the bacteriophage (Clackson et al, 1991, supra; Marks et al, 1991, supra). However, other suitable systems for display include using other viruses (EP 39578), bacteria (Gunneriusson et al, 1999, supra; Daugherty et al, 1998, Protein Eng 11(9):825-32; Daugherty et al, 1999, Protein Eng 12(7):613-21), and yeast (Shusta et al, 1999, J Mol Biol 292(5):949-56).

In addition, display systems have been developed utilising linkage of the polypeptide product to its encoding mRNA in so-called ribosome display systems (Hanes & Pluckthun, 1997, supra; He & Taussig, 1997, supra; Nemoto et al, 1997, supra), or alternatively linkage of the polypeptide product to the encoding DNA (see U.S. Pat. No. 5,856,090 and WO 98/37186).

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

The antibody or antigen-binding fragment may be selected from the group consisting of intact antibodies, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]). Preferably, the antibody or antigen-binding fragment is a single chain Fv (scFv).

The one or more binding moieties may alternatively comprise or consist of an antibody-like binding agent, for example an affibody or aptamer.

By “scFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

The antibodies may be monoclonal or polyclonal. Suitable monoclonal antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J G R Hurrell (CRC Press, 1982), both of which are incorporated herein by reference.

When potential binding molecules are selected from libraries, one or more selector peptides having defined motifs are usually employed. Amino acid residues that provide structure, decreasing flexibility in the peptide or charged, polar or hydrophobic side chains allowing interaction with the binding molecule may be used in the design of motifs for selector peptides. For example:
(i) Proline may stabilise a peptide structure as its side chain is bound both to the alpha carbon as well as the nitrogen;
(ii) Phenylalanine, tyrosine and tryptophan have aromatic side chains and are highly hydrophobic, whereas leucine and isoleucine have aliphatic side chains and are also hydrophobic;
(iii) Lysine, arginine and histidine have basic side chains and will be positively charged at neutral pH, whereas aspartate and glutamate have acidic side chains and will be negatively charged at neutral pH;
(iv) Asparagine and glutamine are neutral at neutral pH but contain a amide group which may participate in hydrogen bonds;
(v) Serine, threonine and tyrosine side chains contain hydroxyl groups, which may participate in hydrogen bonds.

Typically, selection of binding molecules may involve the use of array technologies and systems to analyse binding to spots corresponding to types of binding molecules.

The one or more protein-binding moieties may comprise a detectable moiety. The detectable moiety may be selected from the group consisting of a fluorescent moiety, a luminescent moiety, a chemiluminescent moiety, a radioactive moiety and an enzymatic moiety.

In a further embodiment of the methods of the invention, step (c) may be performed using an assay comprising a second binding agent capable of binding to the one or more proteins, the second binding agent also comprising a detectable moiety. Suitable second binding agents are described in detail above in relation to the first binding agents.

Thus, the proteins of interest in the sample to be tested may first be isolated and/or immobilised using the first binding agent, after which the presence and/or relative amount of said biomarkers may be determined using a second binding agent.

In one embodiment, the second binding agent is an antibody or antigen-binding fragment thereof; typically a recombinant antibody or fragment thereof. Conveniently, the antibody or fragment thereof is selected from the group consisting of: scFv; Fab; a binding domain of an immunoglobulin molecule. Suitable antibodies and fragments, and methods for making the same, are described in detail above.

Alternatively, the second binding agent may be an antibody-like binding agent, such as an affibody or aptamer.

Alternatively, where the detectable moiety on the protein in the sample to be tested comprises or consists of a member of a specific binding pair (e.g. biotin), the second binding agent may comprise or consist of the complimentary member of the specific binding pair (e.g. streptavidin).

Where a detection assay is used, it is preferred that the detectable moiety is selected from the group consisting of: a fluorescent moiety; a luminescent moiety; a chemiluminescent moiety; a radioactive moiety; an enzymatic moiety. Examples of suitable detectable moieties for use in the methods of the invention are described above.

Preferred assays for detecting serum or plasma proteins include enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies. Exemplary sandwich assays are described by David et al in U.S. Pat. Nos. 4,376,110 and 4,486,530, hereby incorporated by reference. Antibody staining of cells on slides may be used in methods well known in cytology laboratory diagnostic tests, as well known to those skilled in the art.

Thus, in one embodiment the assay is an ELISA (Enzyme Linked Immunosorbent Assay) which typically involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemiluminescent systems based on enzymes such as luciferase can also be used.

Conjugation with the vitamin biotin is frequently used since this can readily be detected by its reaction with enzyme-linked avidin or streptavidin to which it binds with great specificity and affinity.

In an alternative embodiment, the assay used for protein detection is conveniently a fluorometric assay. Thus, the detectable moiety of the second binding agent may be a fluorescent moiety, such as an Alexa fluorophore (for example Alexa-647).

Preferably, steps (c), (e), and/or (g) of the methods described in the first aspect are performed using an array. The array may be a bead-based array or a surface-based array. The array may be selected from the group consisting of: macroarray; microarray; nanoarray.

Arrays per se are well known in the art. Typically they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising proteins, polynucleotides and other suitable molecules and/or conducting an immunoassay. The binding processes are well known in the art and generally consist of cross-linking covalently binding or physically adsorbing a protein molecule, polynucleotide or the like to the solid support. Alternatively, affinity coupling of the probes via affinity-tags or similar constructs may be employed. By using well-known techniques, such as contact or non-contact printing, masking or photolithography, the location of each spot can be defined. For reviews see Jenkins, R. E., Pennington, S. R. (2001, Proteomics, 2, 13-29) and Lal et al (2002, Drug Discov Today 15; 7(18 Suppl):S143-9).

Typically the array is a microarray. By “microarray” we include the meaning of an array of regions having a density of discrete regions of at least about 100/cm², and preferably at least about 1000/cm². The regions in a microarray have typical dimensions, e.g. diameter, in the range of between about 10-250 μm, and are separated from other regions in the array by about the same distance. The array may alternatively be a macroarray or a nanoarray.

Once suitable binding molecules (discussed above) have been identified and isolated, the skilled person can manufacture an array using methods well known in the art of molecular biology.

In an additional or alternative embodiment one or more biomarkers measured in step (c) comprise or consist of one or more homologous gene product expressed by human cells. In an additional or alternative embodiment one or more biomarkers measured in step (c) is a protein or polypeptide. In an additional or alternative embodiment one or more biomarker measured in step (c) is a nucleic acid (e.g., DNA, mRNA or cDNA etc).

In an additional or alternative embodiment method is performed in vitro, in vivo, ex vivo or in silico. For example, the method may in particular be performed in vitro.

By “test protein” we include any protein or proteinaceous entity (or mixture of proteins or proteinaceous entities) for which allergenic or sensitization status is to be determined.

By “allergenic” we include or mean a protein (or mixture of proteins) which is an allergen, and/or which is capable of inducing an allergic response, in a mammal.

In an additional or alternative embodiment the allergenicity comprises a hypersensitivity response (e.g., a cell-mediated hypersensitivity response). In an additional or alternative embodiment the hypersensitivity response is a type I hypersensitivity response. In an additional or alternative embodiment the hypersensitivity response is respiratory allergy.

In an additional or alternative embodiment, the method is for identifying the sensitization status of a protein in a mammal. For example, the expression of the two or more biomarkers measured in step (c) may be indicative of the sensitization status of the test protein.

By “sensitization status” we include or mean whether or not a test protein (or mixture of test proteins) is a sensitizer or not (e.g., a skin sensitizer and/or a respiratory sensitizer).

In an additional or alternative embodiment, the method is for identifying proteins which are capable of inducing respiratory sensitization in a mammal. For example, the expression of the two or more biomarkers measured in step (c) may be indicative of the respiratory sensitizing effect of the test protein.

In one embodiment, the method is for identifying proteins capable of inducing a respiratory hypersensitivity response. Preferably, the hypersensitivity response is a humoral hypersensitivity response, for example, a type I hypersensitivity response. In one embodiment, the method is for identifying agents capable of inducing respiratory allergy.

By “indicative of the respiratory sensitizing effect of the test protein” we include determining whether or not the test protein is a respiratory sensitizer and/or determining the potency of the test protein as a respiratory sensitizer.

By proteins “capable of inducing respiratory sensitization” we mean any protein capable of inducing and triggering a Type I immediate hypersensitivity reaction in the respiratory tract of a mammal. Preferably the mammal is a human. Preferably, the Type I immediate hypersensitivity reaction is DC-mediated and/or involves the differentiation of T cells into Th2 cells. Preferably the Type I immediate hypersensitivity reaction results in humoral immunity and/or respiratory allergy.

The conducting zone of the mammalian lung contains the trachea, the bronchi, the bronchioles, and the terminal bronchioles. The respiratory zone contains the respiratory bronchioles, the alveolar ducts, and the alveoli. The conducting zone is made up of airways, has no gas exchange with the blood, and is reinforced with cartilage in order to hold open the airways. The conducting zone humidifies inhaled air and warms it to 37° C. (99° F.). It also cleanses the air by removing particles via cilia located on the walls of all the passageways. The respiratory zone is the site of gas exchange with blood.

In one embodiment, the protein “capable of inducing respiratory sensitization” is a protein capable of inducing and triggering a Type I immediate hypersensitivity reaction at a site of lung epithelium in a mammal. Preferably, the site of lung epithelium is in the respiratory zone of the lung, but may alternatively or additionally be in the conductive zone of the lung.

In an additional or alternative embodiment, the method is for identifying food proteins which are allergenic in a mammal. For example, the expression of the two or more biomarkers measured in step (c) may be indicative of the allergenicity of the food protein. Preferably, the allergenicity of the food protein is due to a Type 1 hypersensitivity response.

The mammal may be any domestic or farm animal. Preferably, the mammal is a rat, mouse, guinea pig, cat, dog, horse or a primate. Most preferably, the mammal is human.

In an additional or alternative embodiment the population of dendritic cells or population of dendritic-like cells comprises or consists of immortal cells. By “immortal” we mean cells that are not limited by a point at which they can no longer continue to divide, which might otherwise be due to DNA damage or shortened telomeres.

In an additional or alternative embodiment the population of dendritic cells or population of dendritic-like cells comprises or consists of non-naturally occurring cells. By “non-naturally occurring” cells, we mean that the cells are different to, modified from, or variants of, those which would be found in nature; in other words, they are not cells which would normally occur in nature.

In an additional or alternative embodiment the population of dendritic cells or population of dendritic-like cells is a population of dendritic-like cells. In an additional or alternative embodiment the dendritic-like cells are myeloid dendritic-like cells. In an additional or alternative embodiment the myeloid dendritic-like cells are derived from myeloid dendritic cells.

In an additional or alternative embodiment the cells derived from myeloid dendritic cells are myeloid leukaemia-derived cells. In an additional or alternative embodiment the myeloid leukaemia-derived cells are selected from the group consisting of KG-1, THP-1, U-937, HL-60, Monomac-6, AML-193 and MUTZ-3. In an additional or alternative embodiment the dendritic-like cells are MUTZ-3 cells. MUTZ-3 cells are human acute myelomonocytic leukemia cells that are available from Deutsche Sammlung für Mikroorganismen and Zellkulturen GmbH (DSMZ), Braunschweig, Germany (www.dsmz.de; DMSZ No. ACC 295).

By “dendritic-like cells” we mean non-dendritic cells that exhibit functional and phenotypic characteristics specific to dendritic cells such as morphological characteristics, expression of costimulatory molecules and MHC class II molecules, and the ability to pinocytose macromolecules and to activate resting T cells.

In one embodiment, the dendritic-like cells, after stimulation with cytokine, present antigens through CD1d, MHC class I and II and/or induce specific T-cell proliferation.

In one embodiment, the dendritic-like cells are CD34⁺ dendritic cell progenitors. Optionally, the CD34⁺ dendritic cell progenitors can acquire, upon cytokine stimulation, the phenotypes of presenting antigens through CD1d, MHC class I and II, induce specific T-cell proliferation, and/or displaying a mature transcriptional and phenotypic profile upon stimulation with inflammatory mediators (i.e. similar phenotypes to immature dendritic cells or Langerhans-like dendritic cells).

In one embodiment, the population of dendritic cells or population of dendritic-like cells is a population of dendritic cells. Preferably, the dendritic cells are primary dendritic cells. Preferably, the dendritic cells are myeloid dendritic cells.

Dendritic cells may be recognized by function, by phenotype and/or by gene expression pattern, particularly by cell surface phenotype. These cells are characterized by their distinctive morphology, high levels of surface MHC-class II expression and ability to present antigen to CD4+ and/or CD8+ T cells, particularly to naïve T cells (Steinman et al. (1991) Ann. Rev. Immunol. 9: 271).

The cell surface of dendritic cells is unusual, with characteristic veil-like projections, and is characterized by expression of the cell surface markers CD11c and MHC class II. Most DCs are negative for markers of other leukocyte lineages, including T cells, B cells, monocytes/macrophages, and granulocytes. Subpopulations of dendritic cells may also express additional markers including 33D1, CCR1, CCR2, CCR4, CCR5, CCR6, CCR7, CD1a-d, CD4, CD5, CD8alpha, CD9, CD11b, CD24, CD40, CD48, CD54, CD58, CD80, CD83, CD86, CD91, CD117, CD123 (IL3Ra), CD134, CD137, CD150, CD153, CD162, CXCR1, CXCR2, CXCR4, DCIR, DC-LAMP, DC-SIGN, DEC205, E-cadherin, Langerin, Mannose receptor, MARCO, TLR2, TLR3 TLR4, TLR5, TLR6, TLR9, and several lectins.

The patterns of expression of these cell surface markers may vary along with the maturity of the dendritic cells, their tissue of origin, and/or their species of origin. Immature dendritic cells express low levels of MHC class II, but are capable of endocytosing antigenic proteins and processing them for presentation in a complex with MHC class II molecules. Activated dendritic cells express high levels of MHC class 11, ICAM-1 and CD86, and are capable of stimulating the proliferation of naive allogeneic T cells, e. g. in a mixed leukocyte reaction (MLR).

Functionally, dendritic cells or dendritic-like cells may be identified by any convenient assay for determination of antigen presentation. Such assays may include testing the ability to stimulate antigen-primed and/or naive T cells by presentation of a test antigen, followed by determination of T cell proliferation, release of IL-2, and the like.

Methods of detecting and/or measuring the concentration of protein and/or nucleic acid are well known to those skilled in the art, see for example Sambrook and Russell, 2001, Cold Spring Harbor Laboratory Press.

Preferred methods for detection and/or measurement of protein include Western blot, North-Western blot, immunosorbent assays (ELISA), antibody microarray, tissue microarray (TMA), immunoprecipitation, in situ hybridisation and other immunohistochemistry techniques, radioimmunoassay (RIA), immunoradiometric assays (IRMA) and immunoenzymatic assays (IEMA), including sandwich assays using monoclonal and/or polyclonal antibodies. Exemplary sandwich assays are described by David et aL, in U.S. Pat. Nos. 4,376,110 and 4,486,530, hereby incorporated by reference. Antibody staining of cells on slides may be used in methods well known in cytology laboratory diagnostic tests, as well known to those skilled in the art.

Typically, ELISA involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

Conjugation with the vitamin biotin is frequently used since this can readily be detected by its reaction with enzyme-linked avidin or streptavidin to which it binds with great specificity and affinity.

In an additional or alternative embodiment, the method comprises one or more of the following steps:

(i) cultivating dendritic or dendritic-like cells;

(ii) seeding cells of (i) in one or more wells, preferably at steady state growth phase, e.g. wells of one or more multi-well assay plate;

(iii) adding to one or more well(s) of (ii) the protein(s) to be tested;

(iv) adding to one or more separate well(s) of (ii) positive control(s), e.g. Der p 1 and/or Der p 7;

(v) adding to one or more separate well(s) of (ii) negative control(s), e.g. DMSO; and/or leaving one or more separate well(s) of (ii) unstimulated to obtain a medium control; (vi) incubating cells in wells of (iii)-(v), preferably for about 24 hours; and, optionally, harvesting cells from wells of (iii)-(v); and, further optionally, removing supernatant and storing in TRIzol reagent;

(vii) isolating purified total RNA from the cells of (vi) and, optionally, converting mRNA into cDNA;

(viii) quantifying expression levels of individual mRNA transcripts from (vii), e.g. using an array, such as an Affymetrix Human Gene 1.0 ST array;

(ix) exporting and normalizing data from (viii), e.g. using appropriate algorithms;

(x) isolating data from (ix) originating from biomarkers of the GARD Protein Allergen Prediction Signature (i.e. the biomarkers of Table A);

(xi) applying a prediction model to the data of (x), e.g. a frozen SVM model previously established and trained on historical data, e.g. data obtained in Example 1, to predict the allergenicity (e.g. classify as allergen/non-allergen), of tested protein(s) and negative/positive control(s).

A second aspect of the invention provides an array for use in the method according to the first aspect of the invention, the array comprising one or more binding moiety as defined in the first aspect of the invention.

In an additional or alternative embodiment the array comprises one or more binding moiety for each of the biomarkers as defined in the first aspect of the invention.

In an additional or alternative embodiment the one or more binding moiety is immobilised.

In an additional or alternative embodiment the array is a bead-based array. In an additional or alternative embodiment the array is a surface-based array.

In an additional or alternative embodiment the array is selected from the group consisting of: macroarray; microarray; nanoarray.

The array of the second aspect of the invention may comprise one or more, preferably two or more, binding moieties, wherein the binding moieties are each capable of binding selectively to a biomarker as defined in the first aspect. Therefore, the array may comprise or consist of a particular selection of biomarker-specific binding moieties which correlates to any particular selection of biomarkers as defined in the first aspect.

For example, in an additional or alternative embodiment, the array comprises 2, 3, 4, 5, 6, 7, 8, 9, 10, 11, 12, or 13 different binding moieties, wherein the different binding moieties are each capable of binding selectively to a different biomarker listed in Table A(i). For example, the array may comprise or consist of 13 different binding moieties, each capable of binding selectively to a different biomarker listed in Table A(i).

A third aspect of the invention provides the use of two or more biomarkers as defined in the first aspect of the invention for determining the allergenicity of a protein.

In an additional or alternative embodiment there is provided the use of two or more biomarkers selected from the group defined in Table A for determining the determining the allergenicity of a protein, preferably wherein one or more of the biomarkers is selected from the group defined in Table A(i).

In an additional or alternative embodiment there is provided the use of two or more binding moieties each with specificity for a biomarker selected from the group defined in Table A for determining the allergenicity of a protein, preferably wherein one or more of the binding moieties has specificity for a biomarker selected from the group defined in Table A(i).

A fourth aspect of the invention provides an analytical kit for use in a method according the first aspect of the invention comprising:
(a) an array according to the second aspect of the invention; and
(b) instructions for performing the method as defined in the first aspect of the invention (optional).

In an additional or alternative embodiment the analytical kit further comprising one or more control agents as defined in the first aspect of the invention.

A fifth aspect of the invention provides a method of treating or preventing an allergic reaction (for example a type I hypersensitivity reaction, such as respiratory asthma) in a patient comprising the steps of:
(a) providing one or more test protein that the patient is or has been exposed to;
(b) determining whether the one or more test protein provided in step (a) is allergenic using a method of the present invention; and
(c) where one or more test protein is identified as allergenic, reducing or preventing exposure of the patient to the one or more test proteins and/or providing appropriate treatment for the symptoms of allergenicity.

Preferably, the one or more test protein that the patient is or has been exposed to is a protein that the patient is presently exposed to at least once a month, for example, at least once every two weeks, at least once every week, or at least once every day.

Treatments of the symptoms of allergy may include, for example: short-acting beta2-adrenoceptor agonists (SABA), such as salbutamol; anticholinergic medications, such as ipratropium bromide; other adrenergic agonists, such as inhaled epinephrine; corticosteroids such as beclomethasone; long-acting beta-adrenoceptor agonists (LABA) such as salmeterol and formoterol; leukotriene antagonists such as montelukast and zafirlukast; mast cell stabilizers (such as cromolyn sodium) are another non-preferred alternative to corticosteroids; antihistamines; desensitization therapies via prolonged low concentration exposure; epinephrine shots (for acute anaphylactic shock symptoms); and/or avoidance of the sensitizing agent.

Preferably, the method of treatment is consistent with the method described in the first aspect of the invention, and one or more of the embodiments described therein.

A sixth aspect of the invention provides a computer program for operating the methods the invention, for example, for interpreting the expression data of step (c) (and subsequent expression measurement steps) and thereby determining whether one or more test protein is allergenic. The computer program may be a programmed SVM. The computer program may be recorded on a suitable computer-readable carrier known to persons skilled in the art. Suitable computer-readable-carriers may include compact discs (including CD-ROMs, DVDs, Blue Rays and the like), floppy discs, flash memory drives, ROM or hard disc drives. The computer program may be installed on a computer suitable for executing the computer program.

The skilled person will appreciate that all non-conflicting embodiments may be used in combination. Hence, embodiments from one aspect of the invention may equally be applied to a second aspect of the invention.

Preferred, non-limiting examples which embody certain aspects of the invention will now be described, with reference to the following figures:

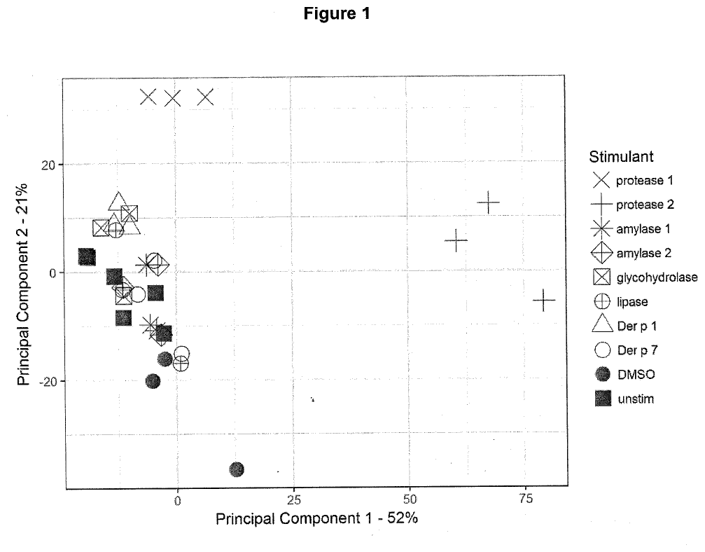

FIG. 1. Principal component analysis plot visualizing differences in transcript expression levels based on at least biological triplicate samples treated with the indicated substances (multigroup ANOVA comparing treatments; FDR=0.01, 1037 genes. Unstim=control cells “treated” only with (vehicle) cell culture medium).

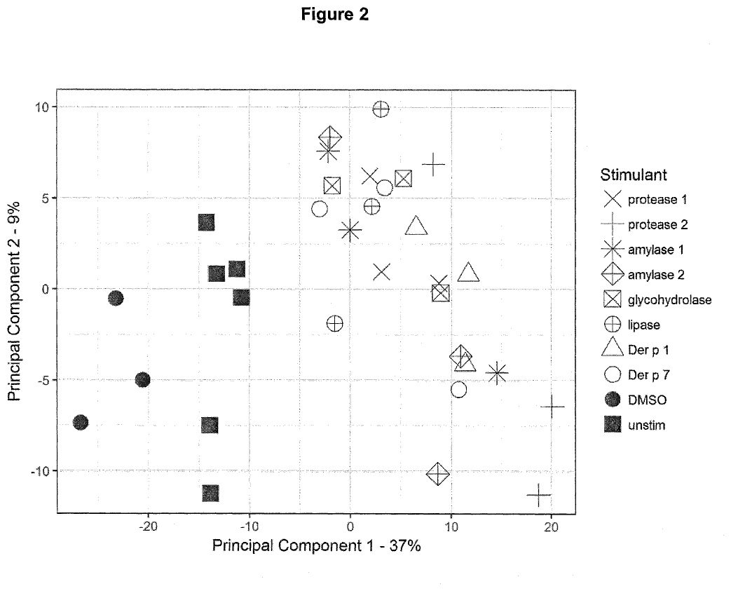

FIG. 2. Principal component analysis plot visualizing differences in transcript expression levels of all 33 samples (at least biological triplicates treated with the indicated substances) based on an input of the identified 391 potential biomarkers. Unstim=control cells “treated” only with (vehicle) cell culture medium).

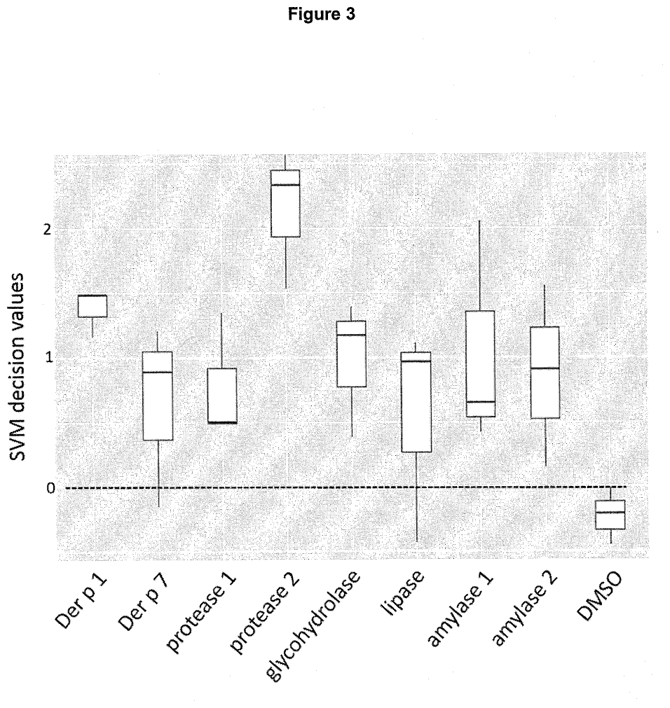

FIG. 3. A series of leave-one-out cross-validations was performed. For each cross-validation exercise, all replicates of the indicated treatment were removed and the backward elimination procedure was performed as described in Materials and Methods. Resulting predictions for the respective treatment are presented as boxplots depicting Support vector machine (SVM) decision values for each replicate. Negative DVs correspond to non-allergens, positive DVs to allergens.

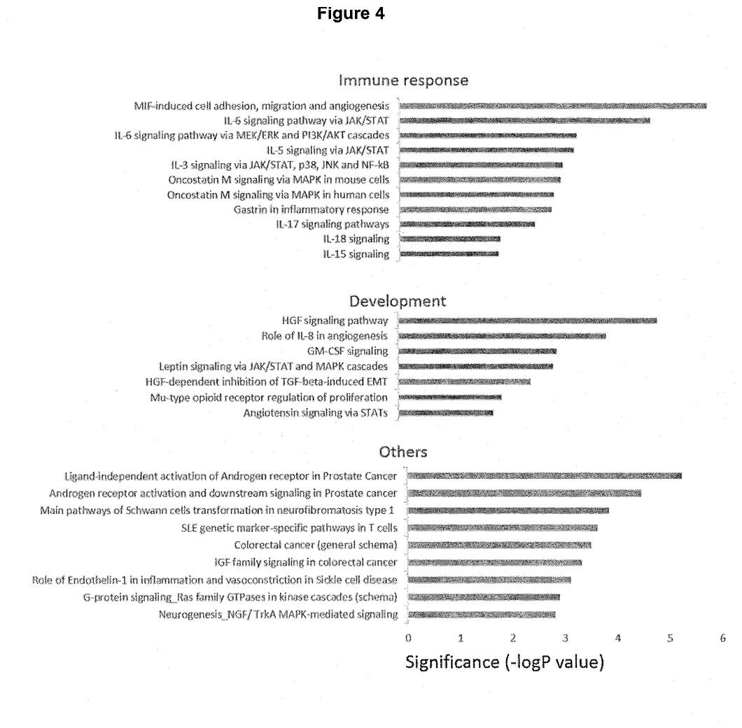

FIG. 4. Key Pathway Advisor analysis results in 27 significantly regulated pathways using the 391 transcripts as input, which were identified by a two group comparison between six protein allergens and controls followed by the application of a backward elimination algorithm.

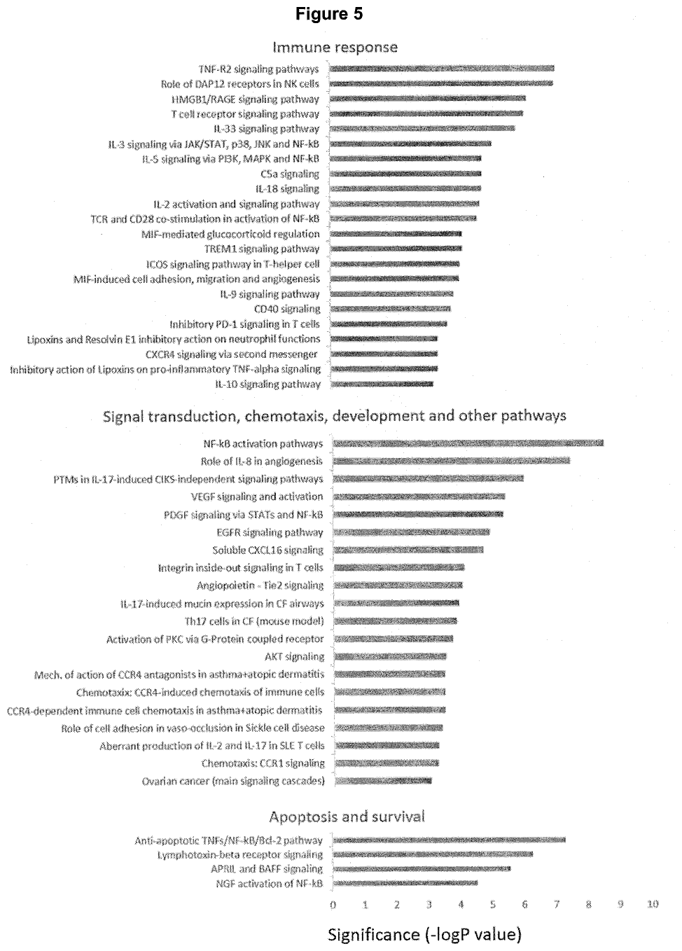

FIG. 5. Key Pathway Advisor analysis based on an input of the most significant 272 transcripts resulting from a two-group comparison of unstimulated control samples and protease-treated samples. The most significant pathways are presented (Union p-values≤0.001, corresponding to a −log p-value of 3).

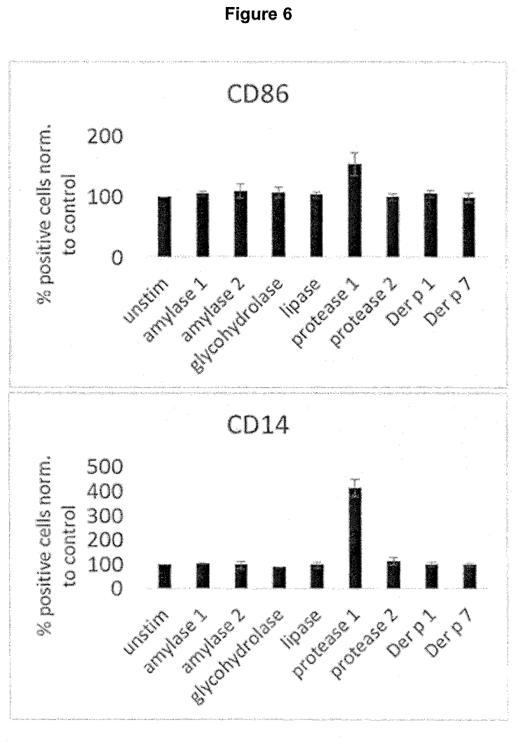

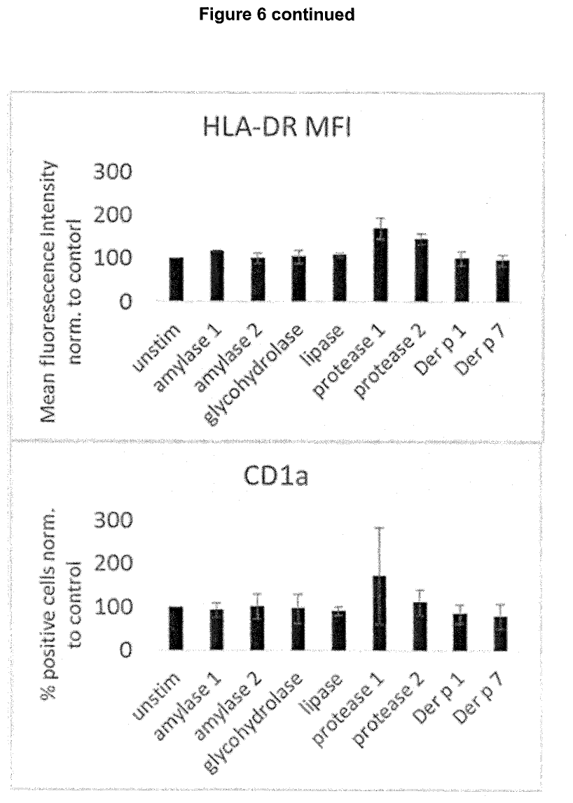

FIG. 6. Changes in cell surface expression levels of CD86, CD14, CD1a, and HLA-DR in MUTZ-3 cells stimulated with the indicated substances for 24 hours as determined by flow cytometry (n=3, error bars show standard deviations). Data are normalized to respective control (unstimulated) sample (control=100%). MFI=mean fluorescence intensity.

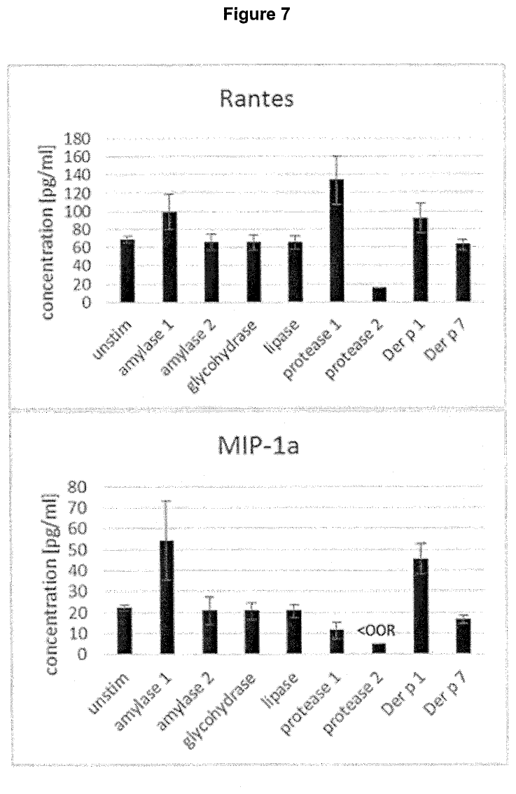

FIG. 7. Changes in Rantes and MIP-1α protein levels in MUTZ-3 supernatants after stimulation with the indicated substances for 24 hours, as determined by Bio-Plex® technology. (n=3, error bars show standard deviations).

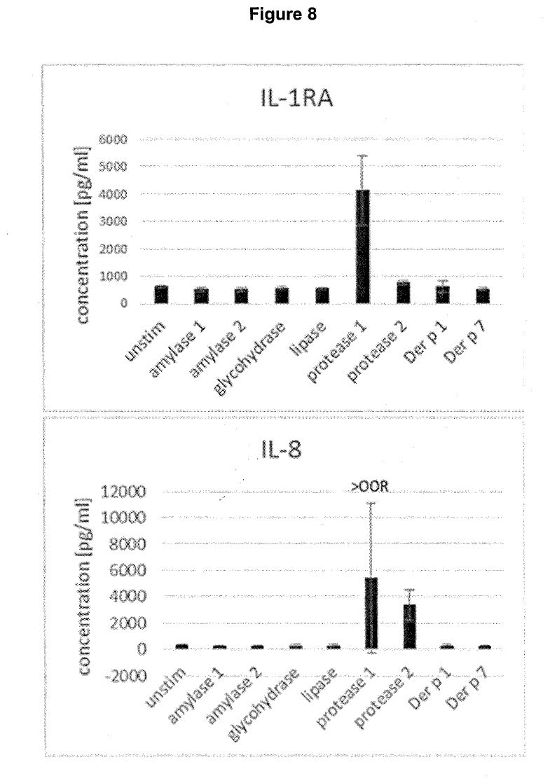

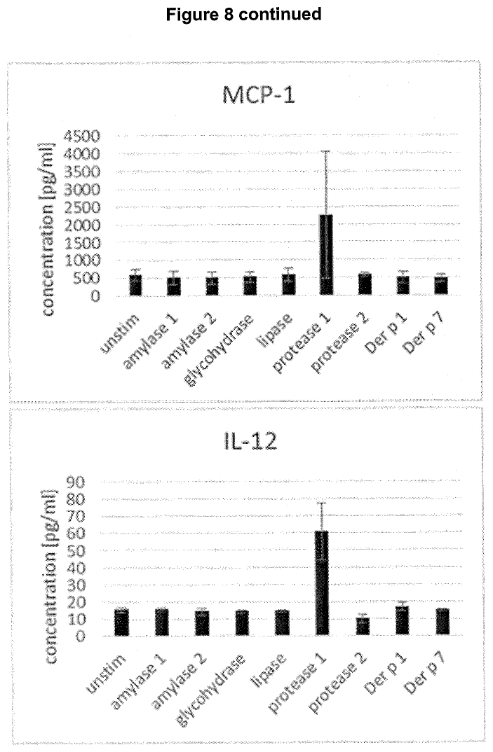

FIG. 8: Changes in protein levels of the indicated cytokines in MUTZ-3 supernatants after stimulation with the indicated substances for 24 hours, as determined by Bio-Plex®. (n=3, error bars show standard deviations).

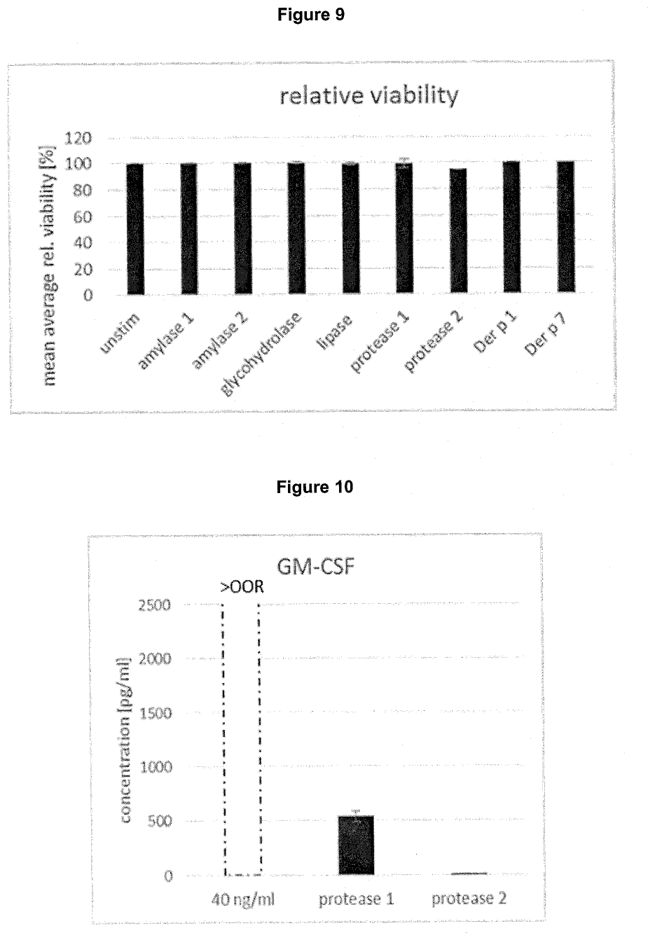

FIG. 9: Viability assessed with propidium iodide staining and flow cytometry; cells were treated with the indicated substances for 24 hours (n=3, error bars show standard deviations).

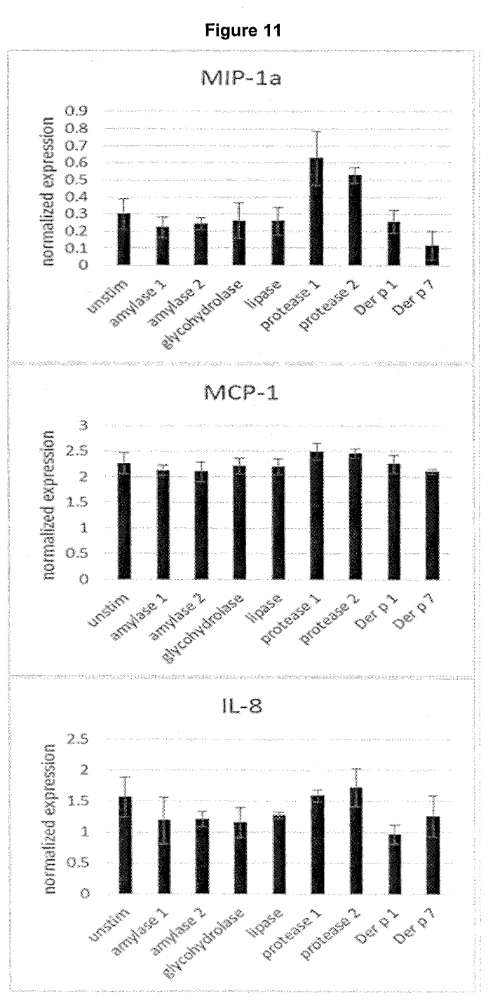

FIG. 10: Changes in GM-CSF protein levels in MUTZ-3 supernatants after stimulation with the indicated substances for 24 hours, as determined by Bio-Plex® technology (n=3, error bars show standard deviations). GM-CSF was part of the complete cell culture medium (added to 40 ng/ml) in all samples, which is reflected by values out of range (oor) of the upper part of the standard curve (left bar), proteases seem to degrade GM-CSF (middle and right bar).

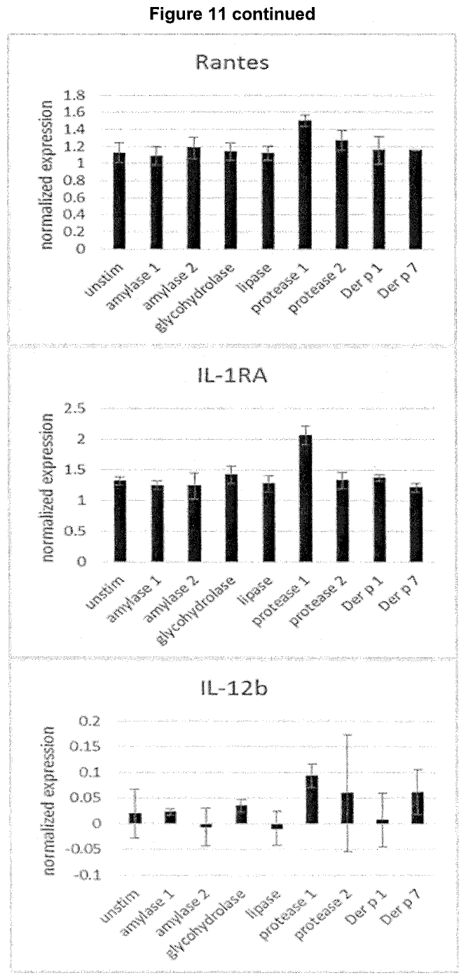

FIG. 11: RNA microarray expression data of the indicated cytokines. (n=3, error bars show standard deviations. Microarray data is normalized using the scan algorithm [48, 49], and should thus not be interpreted in a linear fashion).

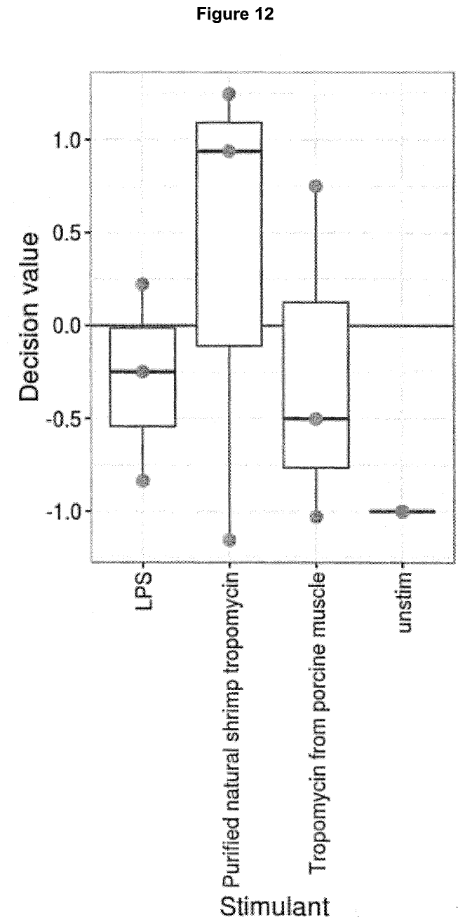

FIG. 12: GARD Protein Allergen Prediction Signature (PAPS) Decision Values for biological triplicate samples of test substances.

### Example 1

### Introduction

Allergy is a chronic disease with increasing prevalence and it is of outmost importance for the industry and authorities to identify potential allergens as early as possible to limit the exposure of workers and the general populations. Several hundreds of chemicals are known to be able to cause allergic contact dermatitis [1, 2], a type IV delayed hypersensitivity reaction, whereas less chemicals are known to sensitize the respiratory tract and to induce type I allergic responses [3]. Most substances causing respiratory allergy are proteins of environmental origin e.g. allergens from house dust mite feces, pollen, or fungi, while others are present in an occupational setting such as enzymes used in flavor, fragrance, detergents and pharmaceutical production [4, 5]. The risk of developing adverse reactions following occupational exposure exists; thus, a strict focus on occupational safety is mandatory. Sensitization has been observed for workers exposed to certain industrial enzymes such as α-amylase, proteases, pancreatinin, and trypsin [6, 7]. New enzymes are continuously developed for existing as well as for new applications, such as genetically modified enzymes used in food processing and flavor production and may also lead to occupational health risks [5, 7].

To date, no validated assay is available specifically for predicting the sensitizing potential of novel proteins, rendering a weight-of-evidence approach to be the most acceptable means of allergy safety assessment. There is, however, a growing consensus that the allergenic potential of compounds, including proteins, should be evaluated with regard to their biochemical characteristics and the protein's potential to induce a specific immune response (European COST Project impARAS [8]). A combination of physical traits of proteins, the molecular interaction between human cells and proteins, as well as their impact on cell-cell interactions play a role in understanding and eventually predicting protein allergenicity [9, 10].

The Adverse Outcome Pathway (AOP) concept is a framework for collecting and organizing information relevant to an adverse outcome at different levels of biological organization [11]. These AOPs are based on available information on substance-response and response-response relationships and allow the development of relevant predictive animal-free test methods and approaches, as well as the contextualization of the results across a diverse range of biological mechanisms and toxicity endpoints. The mechanisms driving respiratory sensitization are not fully disclosed yet, but emerging data suggest that events driving respiratory and skin sensitization can be structured in the same adverse outcome pathway (AOP). In contrast to skin sensitization, properly evaluated test methods addressing the key events of respiratory sensitization induction are not yet available [12].

The Genomic Allergen Rapid Detection (GARD) assay was initially developed to provide information about the capacity of chemicals to induce skin sensitization (accuracy: 89% [13, 17]). This in vitro assay utilizes a myeloid cell line resembling dendritic cells (DCs) as a model system. DCs are antigen-presenting cells and central for the induction and regulation of adaptive immune responses [14]. This assay was recognized by both the European Reference Laboratory—European Center for Validation of Alternative Methods (EURL-ECVAM) and the OECD as a valuable method for addressing key event 3 (Dendritic cell activation and maturation) of the AOP for skin sensitization [15]. Forreryd et al. [16] successfully demonstrated that a modified protocol of the assay is able to predict respiratory chemical sensitizers with an accuracy of 84% based on a biomarker signature consisting of 389 transcripts.

In this example, this protocol is assessed for its capacity to provide a mechanistic understanding of the protein sensitization at DC level. A potential gene profile for the classifications of proteinaceous allergens was identified. For this purpose, the effect of eight respiratory protein allergens on the gene expression in the myeloid cell line was investigated using Affymetrix RNA expression array technology. A panel of potential biomarkers distinguishing the allergens from control samples was identified using data-driven approaches including machine learning, and cross-validation exercises indicated that the identified transcripts are indeed useful for classification. These biomarkers were further investigated with regard to associated biological pathways.

Materials and Methods

Respiratory Allergens

All allergens contain low levels of endotoxin as summarized in Table 1. LoTox™ recombinant Der p 7 (# LTR-DP7-1) and LoTox™ natural Der p 1 (# LTN-DP1-1) were obtained from Indoor biotechnologies, Charlottesville, USA. The other allergens were provided by Novozymes A/S, Bagsvaerd, Denmark, and tested for endotoxin content by Sahlgrenska Universitetssjukhuset, Bakteriologiska laboratoriet, Göteborg.

TABLE 1
Identity and concentrations of allergens and controls.

| | Test compound | Endotoxin in well |
|---|---|---|
| Substance | concentration | conc. [EU/mL] |
| amylase 1 | 25 μg/mL | 0.04 |
| amylase 2 | 25 μg/mL | 0.059 |
| protease 1 | 25 μg/mL | <0.0012 |
| protease 2 | 25 μg/mL | <0.0006 |
| glycohydrolase | 25 μg/mL | 0.033 |
| lipase | 25 μg/mL | 0.022 |
| Der p 1 | 25 μg/mL | <0.75 |
| Der p 7 | 25 μg/mL | <0.25 |
| DMSO | 0.1% | NA |
| unstimulated | | NA |

Cell Culture and Stimulations

The maintenance of the cell line was performed as described in [17]. In short, the MUTZ-3 derivative cells were cultured in MEM alpha modification (Nordic Biolabs/GE Healthcare Bio-Sciences, Taby, Sweden) supplemented with 20% fetal bovine serum (Thermo Fisher Scientific/Life Technologies, Stockholm, Sweden) and 40 ng/mL recombinant human GM-CSF (Miltenyi Biotec Norden AB, Lund, Sweden). A dose finding experiment was performed based on earlier protocols optimized for this cell line to identify the highest enzyme concentration resulting in a relative cell viability of 90% after 24 hours of incubation. Longer exposures (48 hours) to certain enzymes resulted in substantially decreased cell viability (data not shown). A phenotypic control analysis of cells prior to each experiment was carried out by flow cytometry (see below) in order to assure an immature state. In short, three batches of cells were exposed to allergens and control substances dissolved in complete cell culture medium for 24 hours in at least three independent experiments (Table 1). Proteins present in serum served as non-allergen protein controls. Phenotypic controls and viability of the cells were assessed after the stimulation period by analyzing cell surface expression using flow cytometry as described below. Cells aimed for RNA extraction were lysed in TRIzol® (Life Technologies/Thermo Fisher Scientific, Waltham, USA) and stored until further use in minus 20° C.

Flow Cytometry

As a quality control both before and during the experiments, the following monoclonal antibodies were used during phenotypic analysis as described previously [18]: CD1a (DakoCytomation, Glostrup, Denmark), CD34, CD86, HLA-DR (BD Biosciences, Franklin Lakes, N.J.), all FITC-conjugated; CD14 (DakoCytomation), CD54, CD80 (BD Biosciences), all PE-conjugated. FITC- and PE-conjugated mouse IgG1 (BD Biosciences) were used as isotype controls and Propidium iodide was included as a marker for non-viable cells (BD Biosciences). After 24 hours incubation with the allergens and control samples, viability, CD14, CD1a, HLA-DR and CD86 expression were assessed. FACS samples were analyzed on a FACSCanto II instrument with FACS Diva software for data acquisition. 10 000 events were acquired and further analysis was performed in FCS Express V4 (De Novo Software, Los Angeles, Calif.). An endotoxin stimulation was included as internal control for cell quality.

RNA Extraction, cDNA and Array Hybridization

RNA isolation from cells lysed in TRIzol® was performed according to manufacturer's instructions. Labeled sense DNA is synthesized according to Affymetrix' protocols using the recommended kits and controls. The cDNA was hybridized to Human Gene 1.0 ST arrays and further processed and scanned as recommended by the supplier (Affymetrix, Cleveland, USA).

Data Acquisition and Analysis

The dataset, comprising 33 samples in total, was quality-controlled and then normalized using Single Channel Array Normalization [19, 20]. Statistical analysis was performed primarily by Analysis of Variation (ANOVA). ANOVAs and PCA visualization of results were performed with Qlucore 3.2 (Qlucore AB, Lund, Sweden). Significance was evaluated with the multiple-hypothesis corrected p-value (q-value, in this article referred to as false discovery rate (FDR) [21]). An FDR≤105 was considered as statistically significant. Decision values were calculated in a support vector machine (SVM) model [22], constructed in R (R Development Core Team, 2008) and using the package e1071 (R package e1071). The same R package was used to program the backward elimination algorithm [23]. The backward elimination algorithm was applied to a dataset consisting of nine non-allergen and 24 allergen samples and the 1052 most significant genes based on a two group comparison between allergens and controls (p=0.007, FDR=0.19212). Cross-validations were performed in a similar manner based on an input where one treatment at a time had been removed. All additional statistical computing and handling of data was performed in Excel (Microsoft Office 2013) and R (www.R-project.org).

Bio-Plex 200 Cytokine Analysis

Cell supernatants were analyzed using a BioPlex 200 system (Bio-Rad, Hercules, USA) with the Novex® Human Cytokine Magnetic 30-Plex Panel kit (# LHC6003M, Invitrogen/Thermo Fisher Scientific, Waltham, USA) according to the manufacturer's recommendations. Biological triplicates, based on three batches of cells, were analysed in technical duplicates (maximum CV accepted was 20%) and concentrations in range were accepted according to the Bio-Plex 200 Manager 6.1 software (Bio-Rad, Hercules, USA) unless stated otherwise. Data are presented as mean values; error bars represent standard deviations. Due to a limited number of samples (biological triplicates, technical duplicates) and non-normally distributed data, p-values as a measure of statistical significance are not appropriate to provide. An endotoxin stimulation was included as internal assay control.

Key Pathway Analysis

The Key Pathway Advisor (KPA) tool [24] versions 16.4 and 16.6, were used to investigate the identified 391 genes (shown in Table A) comprising the protein prediction signature in a biological context. KPA associates e.g. differentially expressed genes derived from RNA microarray data with both upstream and downstream processes in order to allow biological interpretation. The investigated data set consisted of 33 samples, comprising allergen and control samples in at least three replicates. Both p values and fold changes were used as input based on a two group comparison between allergens and non-allergens (Key Hubs Calculation Algorithm: causal reasoning analysis; Key Hubs p-value threshold=0.01; Key Processes p-value threshold=0.05; unrecognized IDs: 105). An analysis using the same parameters was performed with 272 genes as input, which resulted as the most significant genes from a p-value filtering of proteases versus unstimulated samples (p=0.001335, FDR=0.05) based on a variance-prefiltered data set containing 10054 variables. 254 key hubs were predicted and nine IDs were not recognized.

Scripts

Listed below are details of the script, written in R code, used to perform the method:

# Required files:
# - GARD_PAPS.R
# - raw affymetrix files of test samples in subdir: raw_affy/
# - Annotation of the new data describing the unstimulated samples
raw_affy/annotation.rds
# - Historical data stored in trainingset.rds
# Load required dependencies
source(‘~/GARD_PAPS.R’)
# Read Training Data
train = readRDS(‘~/trainingset.rds’)
# Read new data and annotations
new_data = read_raw_affy(‘~/raw_affy/*.CEL’)
new_data_ref = readRDS(‘~/raw_affy/annotation.rds’)
# Normalize the new data
normalized_data = normalize_train_test(train = train, test = new_data,
test_reference = new_data_ref)
# Train model on historical data
model = train_svm(normalized_data$train)
# Predict New Samples
predictions = predict_test_samples(model = model,
data=normalized_data$test)

Results

Statistical Analysis of Expression Array Data

The Data Set Consisted of 33 Samples in Total, Nine Non-Allergen Controls and 24 Allergen samples (Table 1). When comparing these two groups (i.e. controls, “unstimulated” and “DMSO”, versus allergens), no significantly regulated genes could be detected based on a false discovery rate (FDR) of 0.05. With a chosen cut-off of p=0.0001 (FDR=0.0847), 31 variables were left. This indicates that only smaller differences exist between the group of protein allergens and non-allergen controls. When comparing all treatments to each other (multigroup ANOVA, FDR=0.01), 1037 significantly regulated genes were identified. All samples cluster rather tightly except the proteases as presented in a Principal Component Analysis (PCA) plot in FIG. 1, indicating that the RNA expression patterns induced by each protease differ somewhat from all the other samples.

Identification of a Protein Allergen Prediction Signature

In order to develop a classification approach, a so-called wrapper method was used to identify the most relevant genes for distinguishing allergens from non-allergens after initial p-value filtering. The used backward elimination algorithm [23] based on support vector machine (SVM) predictions [25] results in an optimized list of genes. Each gene or feature in this list has been evaluated not only for its importance for the classification itself, but also how it performs together with the other features. Using an input of the most significant 1052 genes, based on a two-group comparison (allergen versus non-allergen, p=0.007, FDR=0.19), the algorithms identified a signature consisting of 391 genes (see Table A). When the dataset is visualized by PCA using these potential biomarkers as an input, a clear separation between allergens and non-allergens can be seen (FIG. 2).

In order to estimate the accuracy of the model and to ensure that the presented results were not achieved by chance, a series of leave-one-out cross-validations was performed. For each cross-validation exercise, all replicates of one stimulation were removed and the backward elimination procedure was repeated as described above. The obtained gene signature was then used to predict the left out samples using an SVM model based on a training set consisting of the remaining samples in the dataset. All stimulations were removed once except “unstimulated”; without the “unstimulated” samples, the dataset would be too unbalanced to be used for training of a model. The resulting SVM predictions are visualized as boxplots in FIG. 3. All allergens were correctly predicted as such by majority voting, even though one replicate each for lipase and Der p 7 was predicted as non-allergen (negative decision value). SVM predictions were also performed after a similar exercise based on a dataset where DMSO and Der p 1 samples were left out and subsequently used as a test set. Also here, DMSO was correctly classified as non-allergen and Der p 1 as an allergen (data not shown).

Biological Pathway Analysis Reveals Association of Gene Lists with Immunological Pathway Regulation

The association of the 391 identified biomarkers with biological pathways was investigated using the Key Pathway Advisor tool, and the gene list together with associated p-values and fold changes based on a two-group comparison (allergen yes/no) were used as input. Of in total 27 significant pathways identified (FIG. 4), roughly one third was allocated to immune system-related pathways, e.g. IL-3, IL-5, IL-6, IL-15, IL-17 and IL-18 signalling pathways. Examples of suggested key hubs, all with increased activity, were MHC class I, NFκB variants p50 and p50/p65, IL-10, IL-6R, IL-10R, FcγRIIα, glutaredoxin, and integrins α6β4 and α2B.

When investigating biological pathways based on a comparison between proteases and unstimulated samples and the resulting 272 most significant genes, 59 significant pathways were identified and over 40% are related to immune responses as judged by their name (FIG. 5), e.g. IL-2, IL-10, IL-33 and CD40 signalling. Further, “CCR4-induced chemotaxis of immune cells”, “CCR4-dependent immune cell chemotaxis in asthma and atopic dermatitis” and NFκB-related pathways are associated to inflammatory responses. NFκB also appeared as a predicted key hub, along with other molecules such as MyD88, an adaptor protein used by almost all TLRs to activate NFκB. Toll-like receptor 4 and high-mobility group box-1 (HMGB1), a transcriptional regulator involved in inflammation and cell differentiation, were two more examples of predicted key hubs

Protein Allergens Affect Both Surface Marker Expression and Secreted Cytokines

As a complement to the transcriptional analysis, effects on the protein level were investigated by FACS, focusing on a set of differentiation and activation markers as described in FIG. 6. All cells were positive for HLA-DR in the unstimulated control as well as after exposure to all substances (data not shown). There was no protein allergen that reduced the presence of any investigated marker; however, protease 1 seemed to increase expression of e.g. CD86, a known activation marker. Notably, the relative viability after all treatments was % (FIG. 9). When screening 30 soluble cytokines using Bio-Plex® technology, Der p 1 and amylase 1 treatment increased the levels of MIP-la and Rantes in supernatants (FIG. 7). The proteases reduced the levels of several cytokines; likely at least for some by degradation as supported by the results for GM-CSF. GM-CSF is added to the cell medium at 40 ng/mL, but after incubation with the proteases, the values were lowered to a few pg/mL (protease 2) and to approximately 500 pg/mL (protease 1), respectively (FIG. 10), whereas the values of all the other allergens and controls were as expected out of range at the upper limit. However, the proteases increased the levels of e.g. IL-8 (FIG. 8), and protease 1 treatment induced higher levels of Rantes (FIG. 7A), IL-12, MCP-1 and IL-1RA levels (FIG. 8). The data on cytokine protein expression were in part correlated with the changes on RNA level (FIG. 11).

### DISCUSSION

In this example, the development of an in vitro method for the prediction of protein allergens using eight known respiratory allergens as model substances is described. Using data-driven approaches based on machine learning, a list of biomarkers was identified (Table A), which was further investigated with regard to associated biological pathways.

Comparing the RNA expression pattern of cells exposed to controls and those exposed to the respiratory protein allergens, no significantly regulated transcripts based on an FDR of 0.05 could be observed. This could be due to several factors, e.g. relatively small differences between the groups, but also high variation in the transcriptional changes induced by the structurally and functionally different enzymes. Unexpectedly, even when comparing the intended positive controls, Der p 1 and Der p 7, separately to the unstimulated control samples, no significantly regulated genes could be identified. On the protein level, however, Der p 1 seemed to increase Rantes and MIP-1α cytokine levels (FIG. 7), which would be consistent with a pro-inflammatory response induced by Der p 1 [26, 27]. As endotoxins even at low concentrations may activate cells it is important to consider their effects as they may obliterate allergen signals. Der p 1 and Der p 7 have therefore been obtained as “low endotoxin” preparations; nevertheless they contain the highest amounts of endotoxin of all samples considering final concentrations (Table 1, 0.75 EU/mL and 0.25 EU/mL, respectively). As comparison, standard endotoxin levels in fetal bovine serum usually account to 5.1 ng/mL or 510 EU/mL, respectively, according to the industrial standard [28]. However, endotoxins are likely also an intrinsic part of allergic reactions in “real life”, as endotoxins are more or less ubiquitously present. Furthermore, Der p 1 preparations, including the product used here, commonly do not exhibit cysteine protease activity due to the production process. For Der p 1, cysteine protease activity has been described to enhance synthesis of Der p 1-specific IgE and allergenicity in a mouse inhalation model [29, 30]. On the other hand, data presented by Porter et al. [31] indicate that Der p 1 in house dust samples does not have measurable protease activity. In the here presented study, Der p 1's cysteine protease activity was not restored by reducing treatment, as we could not detect any difference in activation of monocyte-derived dendritic cells when comparing cells stimulated with Der p 1 treated with DTT in order to activate its cysteine activity with non-treated Der p 1 (data not shown). Nevertheless, the identified biomarker signature consisting of 391 transcripts was able to separate the dataset into two clearly distinct groups, i.e. controls and allergens (FIG. 2). Though separate statistical analyses could not identify significantly regulated transcripts between these two groups, this together with the cross-validation results (FIG. 3) indicates that several transcripts together in a signature may still be useful for the prediction of the protein allergens, for example those capable of sensitizing the respiratory tract.

Biological pathway analysis using the KPA tool and the potential biomarker signature as input, revealed an overrepresentation of numerous pathways linked to immune responses. Further evidence supporting the relevance of the identified pathways for respiratory sensitization is provided by the current understanding of the NF-κB signaling pathway, which was a suggested upstream regulator for the identified transcriptional changes. NF-κB activation is causally related to increased release of various signal molecules such as IL-33, IL-25 [32, 33], endogenous danger factors (e.g. HMGB-1, uric acid and ATP by epithelial cells and DCs); further to DC activation and migration [34], and the induction of ovalbumin [35] and Der p 2 sensitization [36]. The release of ATP and uric acid drives the activation of the NLRP3 inflammasome complex resulting in cleavage of pro-IL-1p to mature IL-16 through caspase 1 [37]. Uric acid may play an important role in Th2 skewing [38].

Although human and animal data indicate that the here investigated protein allergens do not differ significantly in terms of allergenicity [39], in our in vitro system, protease 1 and 2 induced a particular RNA expression pattern and also influenced the protein levels of certain cell surface markers and cytokines. This may not necessarily be associated with their allergenicity, as it could also be related to cytotoxic and adjuvant properties as described for several cysteine and serine proteases and certain proteases used in detergents [40-43]. When the cells were exposed to the proteases for 48 hours, relative cell viability decreased (data not shown), however, the relative viability never dropped below 90% after 24 hours (FIG. 10). Although it is expected that proteases are able to degrade certain proteins such as cytokines as shown for GM-CSF (FIG. 11), based on the here presented investigations, it is not possible to make conclusions about which mechanisms are involved. It seems likely that both cytokines and cell surface proteins can be cleaved and thus direct and indirect effects on e.g. cell differentiation and activation status can be exerted. Importantly, in order to be able to test these and similar substances in vitro, it is important to carefully choose concentrations not exceeding a certain cytotoxicity. The responses may otherwise be dominated by events reflecting cytotoxicity; comparable to how the irritant properties of certain contact allergens can mask the effects linked to sensitization [44]. On the other hand, irritant or cytotoxic effects may also allow or facilitate sensitization by creating a pro-inflammatory environment as shown for several skin sensitizing chemicals [45-47]. For proteases, impairment of barrier functions and thus higher permeability may play a role as well [41, 42].

Taken together, our results indicate that the identified 391 biomarkers can be useful in order to predict the allergenicity of proteins including proteases, and particularly the sensitizing ability of respiratory protein allergens, including proteases. The results of a series of cross-validations support a valid model (FIG. 3). The biomarker signature also seems to reflect relevant biological pathways.

### Example 2

This example describes allergenic predictions of two samples of Tropomyosin with different species origin, along with appropriate controls. It provides a proof of concept of the applicability of the GARD Protein Allergen Prediction Signature of Table A (herein referred to as “GARD PAPS”) to perform allergenic predictions on protein samples.

The readout of GARD is a set of genomic predictors, referred to as the GARD Prediction Signature (GPS).

In this example, the functionality of GARD PAPS was further demonstrated by assessing the allergenicity two Tropomyosin samples, with different species origin.

The genetic material of the cells are isolated from cell samples stimulated with the test substances. The transcriptional levels of the GARD PAPS are quantified and compared to a reference data set by the use of multivariate statistical prediction models. Each sample is assigned a decision value based on its transcriptional levels of the GARD PAPS, as measured by Affymetrix microarray technology. Final predictions are based on the mean value from biological triplicate samples.

In this example, results from 2 proteins, an LPS control and unstimulated cells are presented.

For a results summary, see Table 2.

TABLE 2
Summary Results.

| Test substance ID | GARD PAPS Prediction |
|---|---|
| Purified natural shrimp tropomyosin | Sensitizer |
| Tropomyosin from pork muscle | Non-sensitizer |
| LPS | Non-sensitizer |
| Unstimulated cells | Non-sensitizer |

Results

Test Substance Handling and GARD Input Concentrations

All proteins and test substances were stored and prepared according to instructions provided by the supplier. The proteins were screened for cytotoxic effects and the GARD input concentration was established for each protein. Results from this screening and resulting GARD PAPS input concentrations are presented in Table 3.

TABLE 3
Test substance(s) details.

| | | | | GARD input |
|---|---|---|---|---|
| Test substance ID | Vehicle | Max. screen^{I} | Rv90^{II} | concentration^{III} |
| Tropomyosin shrimp | PBS | 25 | — | 25 |
| Tropomyosin pork | PBS | 25 | — | 25 |
| LPS | PBS | — | — | 10⁻⁴ |
| Unstimulated cells | — | — | — | — |

^{I}The highest concentration used in screening titration range. Concentration is given in μg/ml.
^{II}Concentration yielding 90% relative viability. Concentration is given in μg/ml.
^{III}Based on Max. screen and Rv90. Concentration is given in μg/ml.

LPS is here used as a negative control, ensuring that observed signals generated by either of the two Tropomyosin samples are not due to endotoxin contaminants. Endotoxin contents of the Tropomyosin samples were quantified using a LAL test. The LPS concentration used as a negative controls was set to correspond to the highest endotoxin concentration present in either Tropomyosin sample.

All test proteins and substances were assayed in biological triplicates.

GARD PAPS Classifications

All replicates of test substances were assigned decision values using the GARD PAPS prediction model, as described (see materials and methods below). Decision values from test substances are presented in FIG. 12.

Materials & Methods

The comprehensive materials and methods for the GARD testing strategy, used to generate data described in this example, is included below.

Deviations from Standard Protocols

The cytotoxic effects of the test proteins were monitored in the concentration range 1-25 μg/ml.

No cytotoxicity could be detected and 25 μg/ml was used as the GARD input concentration, based on findings in scientific literature on in vitro methods for protein allergen predictions.

When stimulating the human myeloid cell line with the test substances, the proteins were first dissolved in PBS to a concentration of 1000 μg/ml. 50 μl of the dissolved proteins were added to 1.95 ml of seeded cells. LPS was diluted in PBS to a final concentration of 0.1 μg/ml and 2 μl were added to 1.998 ml of cell suspension.

The cells were stimulated for 24 h after which they were lysed in TRIzol reagent. RNA was purified, labeled and hybridized to arrays by the SCIBLU core facility for Affymetrix technology, Lund, Sweden.

The quantified transcription levels were single chain array normalized (SCAN) and the GARD PAPS was extracted from the data set. Unstimulated samples, from the test samples and the reference samples used to build the prediction model, were used to remove batch effects between the two data sets.

Final classifications were made using a support vector machine (SVM) which had been trained on the reference samples used to establish the GARD PAPS.

Cell Line Maintenance and Seeding of Cells for Stimulation

The human myeloid leukemia-derived cell line is maintained in α-MEM (Thermo Scientific Hyclone, Logan, Utah) supplemented with 20% (volume/volume) fetal calf serum (Life Technologies, Carlsbad, Calif.) and 40 ng/ml rhGM-CSF (Bayer HealthCare Pharmaceuticals, Seattle, Wash.), as described (Johansson et al., 2011). A media change during expansion is performed every 3-4 days, or when cell-density exceeds 5-600.000 cells/ml. Proliferating progenitor cells are used for the assay, with no further differentiation steps applied. During media exchange, cells are counted and suspended to 200.000 cells/ml. Working stocks of cultures are grown for a maximum of 20 passages or two months after thawing. For chemical stimulation of cells, 1.8 ml is seeded in 24-well plates at a concentration of 222.000 cells/ml. The compound to be used for stimulation is added in a volume of 200 μl, diluting the cell density to 200.000 cells/ml during incubation.

Phenotypic Analysis

Prior to any chemical stimulation, a qualitative phenotypic analysis is performed to ensure that proliferating cells are in an immature stage. All cell surface staining and washing steps are performed in PBS containing 1% BSA (w/v). Cells are incubated with specific mouse monoclonal antibodies (mAbs) for 15 min at 4° C. The following mAbs are used for flow cytometry: FITC-conjugated CD1a (DakoCytomation, Glostrup, Denmark), CD34, CD86, and HLA-DR (BD Biosciences, San Diego, Calif.), PE-conjugated CD14 (DakoCytomation), CD54 and CD80 (BD Biosciences). Mouse IgG1, conjugated to FITC or PE are used as isotype controls (BD Biosciences) and propidium iodide (PI) (BD Biosciences) is used to assess cell viability. FACSDiva software is used for data acquisition with FACSCanto II instrument (BD Bioscience). 10,000 events are acquired, gates are set based on light scatter properties to exclude debris and non-viable cells, and quadrants are set according to the signals from isotype controls. Further data analysis is performed, using FCS Express V3 (De Novo Software, Los Angeles, Calif.). For a reference phenotype of unstimulated cells, see Johansson et al., 2011. Accepted ranges of listed phenotypic markers are listed in Table 4.

TABLE 4
Accepted ranges of proportion of positive cells
for acceptance criteria I: Phenotypic analysis.

| Phenotypic biomarker | Accepted range of positive cells (%) |
|---|---|
| CD86 | 10-40 |
| CD54 | >95 |
| HLA-DR | >60 |
| CD80 | <10 |
| CD34 | 35-70 |
| CD14 | 5-50 |
| CD1a | 10-60 |
| Propidium Iodide (Cell viability) | <15 |

Chemical Handling and Assessment of Cytotoxicity

All chemicals are stored according to instructions from the supplier, in order to ensure stability of compounds. Chemicals are dissolved in water when possible or DMSO for hydrophobic compounds. As many chemicals will have a toxic effect on the cells, cytotoxic effects of test substances are monitored. Some chemicals are poorly dissolved in cell media; therefore the maximum soluble concentration is assessed as well. The chemical that is to be tested is titrated to concentrations ranging from 1 μM to the maximum soluble concentration in cell media. For freely soluble compounds, 500 μM is set as the upper end of the titration range. For cell stimulations, chemicals are dissolved in its appropriate solvent as 1000× stocks of target in-well concentration, called stock A. A 10× stock, called stock B, is prepared by taking 10 μl of stock A to 990 μl of cell media. 200 μl of stock B is then added to the wells containing 1.8 ml seeded cells. For the samples dissolved in DMSO, the in-well concentration of DMSO will thus be 0.1%. Following incubation for 24 h at 37° C. and 5% CO2, harvested cells are stained with PI and analyzed with a flow cytometer. PI-negative cells are defined as viable, and the relative viability of cells stimulated with each concentration in the titration range is calculated as

$$Relative viability = \frac{fraction of viable stimulated cells}{fraction of viable unstimulated cells} \cdot 100$$

For toxic compounds, the concentration yielding 90% relative viability (Rv90) is used for the GARD assay, the reason being that this concentration demonstrates bioavailability of the compound used for stimulation, while not impairing immunological responses. For non-toxic compounds, a concentration of 500 μM is used if possible. For non-toxic compounds that are insoluble at 500 μM in cell media, the highest soluble concentration is used. Whichever of these three criteria is met, only one concentration will be used for the genomic assay. The concentration to be used for any given chemical is termed the ‘GARD input concentration’.

Chemical Exposure of Cells for GARD

Once the GARD input concentration for chemicals to be assayed is established, the cells are stimulated again as described above, this time only using the GARD input concentration. All assessments of test substances are assayed in biological triplicates, performed at different time-points and using different cell cultures. Following incubation for 24 h at 37° C. and 5% CO2, cells from one well are lysed in 0.5 ml TRIzol reagent (Life Technologies) and stored at −20° C. until RNA is extracted. In parallel, a small sample of stimulated cells is taken for PI staining and analysis with flow cytometry, to ensure the expected relative viability of stimulated cells is reached.

Preparation of Benchmark Controls

In addition to any test substance(s) to be assayed within a campaign, a set of benchmark controls are performed, for the purpose of prediction model calibration and estimation of prediction performance. For details regarding benchmark controls used in each specific campaign, see the main document to which this appendix is attached.

Isolation of RNA and GPS Quantification Using Nanostring nCounter System

RNA isolation from lysed cells is performed using commercially available kits (Direct-Zol RNA MiniPrep, Zymo Research, Irvine, Calif.). Total RNA is quantified and quality controlled using BioAnalyzer equipment (Agilent, Santa Clara, Calif.). A total of 100 ng of RNA is used as sample input in a hybridization assay with GPS specific reporter probe CodeSet (Nanostring, Seattle, Wash.). The hybridized RNA-CodeSet sample is prepared on chip using nCounter Prepstation and individual transcripts of the GPS is quantified using Nanostring Digital Analyzer (Nanostring).

Data Acquisition and Normalization

Raw data is exported from the Digital Analyzer and counts of individual transcripts of the GPS are single-chip normalized with a count per total counts algorithm. Normalized data consists of a S by V matrix, where S denotes the number of samples in the GARD campaign, and V denotes the number of quantified transcripts of the GPS.

Data Analysis—Generation of Calibrated Support Vector Machine Decision Values

All further downstream analysis is performed using application-based software, developed in the open source statistical environment R. A support vector machine (SVM) is trained using historical data used for GPS establishment (Johansson et al., 2011). All samples from test substances and benchmark controls from the specific GARD campaign are predicted using the trained SVM, assigning each sample with a SVM decision value. The predictor performance is estimated by identification of the area under the receiver operating characteristic (ROC AUC) of the predicted class of benchmark controls. The full details of this methodology are the same as for Example 1.

GARD Classifications of Test Substance(s)

The GARD prediction model is defined as follows:

If the mean decision value of all available biological replicates of a test substance is greater than zero, the test substance is classified as a sensitizer.

TABLE A

| | | | | |
|---|---|---|---|---|
| Probe | | | | Gene |
| Set ID | Transcript ID | Transcript Description | Gene Title | Symbol |

Table A(i)

| | | | | | |
|---|---|---|---|---|---|
| 8104107 | ENST00000326754 /// | Tripartite motif family-like | tripartite motif family- | TRIML2 | 1 |
| | BC111959 /// | protein 2 | like 2 | | |
| | NM_173553 | gene:ENSG00000179046 /// | | | |
| | | Homo sapiens tripartite motif | | | |
| | | family-like 2, mRNA (cDNA | | | |
| | | clone MGC:138164 | | | |
| | | IMAGE:8327427), complete | | | |
| | | cds. /// Homo sapiens tripartite | | | |
| | | motif family-like 2 (TRIML2), | | | |
| | | mRNA. | | | |
| 7984862 | ENST00000343932 /// | Isoform 2 of Cytochrome P450 | cytochrome P450, | CYP1A2 | 2 |
| | M55053 /// | 1A2 gene:ENSG00000140505 /// | family 1, subfamily A, | | |
| | NM_000761 | Human cytochrome P-3-450 | polypeptide 2 | | |
| | | mRNA, complete cds. /// Homo sapiens | | | |
| | | cytochrome P450, | | | |
| | | family 1, subfamily A, | | | |
| | | polypeptide 2 (CYP1A2), | | | |
| | | mRNA. | | | |
| 8103341 | ENST00000311277 /// | Isoform 1 of Microtubule- | microtubule- | MAP9 | 3 |
| | ENST00000393836 /// | associated protein 9 | associated protein 9 | | |
| | ENST00000379248 /// | gene:ENSG00000164114 /// | | | |
| | ENST00000393834 /// | Isoform 2 of Microtubule- | | | |
| | AY690636 /// | associated protein 9 | | | |
| | NM_001039580 | gene:ENSG00000164114 /// | | | |
| | | Putative uncharacterized | | | |
| | | protein MAP9 | | | |
| | | gene:ENSG00000164114 /// | | | |
| | | Putative uncharacterized | | | |
| | | protein MAP9 | | | |
| | | gene:ENSG00000164114 /// | | | |
| | | Homo sapiens ASAP mRNA, | | | |
| | | complete cds. /// Homo sapiens | | | |
| | | microtubule-associated protein | | | |
| | | 9 (MAP9), mRNA. | | | |
| 8021468 | GENSCAN00000031245 /// | cdna:Genscan | similar to 40S | LOC100131971 | 4 |
| | XM_001722472 | chromosome:NCBI36:18:55579770:55587249:1 /// | ribosomal protein S26 | | |
| | | PREDICTED: Homo sapiens | | | |
| | | similar to 40S ribosomal protein | | | |
| | | S26 (LOC100131971), mRNA. | | | |
| 8166945 | GENSCAN00000048751 /// | cdna:Genscan | — | — | 5 |
| | ENST00000354794 | chromosome:NCBI36:X:44028478:44030436:1 /// | | | |
| | | cdna:pseudogene | | | |
| | | chromosome:NCBI36:X:44028257:44030436:1 | | | |
| | | gene:ENSG00000198414 | | | |
| 8021461 | ENST00000256857 /// | gastrin-releasing peptide | gastrin-releasing | GRP | 6 |
| | BC004488 /// | isoform 1 preproprotein | peptide | | |
| | NM_001012512 /// | gene:ENSG00000134443 /// | | | |
| | NM_001012513 /// | Homo sapiens gastrin-releasing | | | |
| | NM_002091 | peptide, mRNA (cDNA clone | | | |
| | | MGC:10712 IMAGE:3936083), | | | |
| | | complete cds. /// Homo sapiens | | | |
| | | gastrin-releasing peptide | | | |
| | | (GRP), transcript variant 2, | | | |
| | | mRNA. /// Homo sapiens | | | |
| | | gastrin-releasing peptide | | | |
| | | (GRP), transcript variant 3, | | | |
| | | mRNA. /// Homo sapiens | | | |
| | | gastrin-releasing peptide | | | |
| | | (GRP), transcript variant 1, | | | |
| | | mRNA. | | | |
| 8088903 | GENSCAN00000015233 /// | cdna:Genscan | — | — | 7 |
| | ENST00000358162 | chromosome:NCBI36:3:75557484:75756994:−1 /// | | | |
| | | cdna:pseudogene | | | |
| | | chromosome:NCBI36:3:75730726:75731229:−1 | | | |
| | | gene:ENSG00000196454 | | | |
| 8053064 | ENST00000409969 /// | cdna:known | MOB1, Mps One | MOBKL1B | 8 |
| | ENST00000264089 /// | chromosome:NCBI36:2:74235673:74259503:−1 | Binder kinase | | |
| | ENST00000377668 /// | gene:ENSG00000114978 /// | activator-like 1B | | |
| | AK001650 /// | Isoform 1 of Mps one binder | (yeast) | | |
| | NM_018221 | kinase activator-like 1B | | | |
| | | gene:ENSG00000114978 /// | | | |
| | | Putative uncharacterized | | | |
| | | protein MOBKL1B (Fragment) | | | |
| | | gene:ENSG00000114978 /// | | | |
| | | Homo sapiens cDNA FLJ10788 | | | |
| | | fis, clone NT2RP4000498, | | | |
| | | moderately similar to MOB1 | | | |
| | | PROTEIN. /// Homo sapiens | | | |
| | | MOB1, Mps One Binder kinase | | | |
| | | activator-like 1B (yeast) | | | |
| | | (MOBKL1B), mRNA. | | | |
| 7945130 | ENST00000411383 /// | ncrna:misc_RNA | — | — | 9 |
| | ENST00000386420 | chromosome:NCBI36:11:126782956:126783275:1 | | | |
| | | gene:ENSG00000223315 /// | | | |
| | | ncrna:snRNA_pseudogene | | | |
| | | chromosome:NCBI36:11:126782956:126783196:1 | | | |
| | | gene:ENSG00000209155 | | | |
| 8160260 | ENST00000380672 /// | Isoform 1 of Zinc finger protein | basonuclin 2 | BNC2 | 10 |
| | ENST00000380667 /// | basonuclin-2 | | | |
| | ENST00000380666 /// | gene:ENSG00000173068 /// | | | |
| | AY438376 /// | Basonuclin 2 | | | |
| | NM_017637 | gene:ENSG00000173068 /// | | | |
| | | Isoform 2 of Zinc finger protein | | | |
| | | basonuclin-2 | | | |
| | | gene:ENSG00000173068 /// | | | |
| | | Homo sapiens basonuclin2 | | | |
| | | mRNA, complete cds. /// Homo sapiens | | | |
| | | basonuclin 2 (BNC2), | | | |
| | | mRNA. | | | |
| 7934708 | ENST00000372329 /// | Pulmonary surfactant- | surfactant protein A1 /// | SFTPA1 /// | 11 |
| | ENST00000372327 /// | associated protein A2 | surfactant protein | SFTPA1B /// | |
| | ENST00000372325 /// | gene:ENSG00000182314 /// | A1B /// surfactant | SFTPA2 /// | |
| | ENST00000398636 /// | cDNA FLJ54288, moderately | protein A2 /// | SFTPA2B | |
| | ENST00000372316 /// | similar to Pulmonary surfactant- | surfactant protein | | |
| | ENST00000372313 /// | associated protein A1 | A2B | | |
| | ENST00000372308 /// | gene:ENSG00000185303 /// | | | |
| | ENST00000394569 /// | Pulmonary surfactant- | | | |
| | AK298029 /// | associated protein A1 | | | |
| | AK298034 /// | gene:ENSG00000185303 /// | | | |
| | BC157866 /// | Pulmonary surfactant- | | | |
| | BC157890 /// | associated protein A1 | | | |
| | NM_006926 /// | gene:ENSG00000185303 /// | | | |
| | NM_001098668 /// | Pulmonary surfactant- | | | |
| | NM_001093770 /// | associated protein A2 | | | |
| | NM_005411 | gene:ENSG00000122854 /// | | | |
| | | cDNA FLJ54288, moderately | | | |
| | | similar to Pulmonary surfactant- | | | |
| | | associated protein A1 | | | |
| | | gene:ENSG00000122852 /// | | | |
| | | Pulmonary surfactant- | | | |
| | | associated protein A1 | | | |
| | | gene:ENSG00000122852 /// | | | |
| | | Pulmonary surfactant- | | | |
| | | associated protein A1 | | | |
| | | gene:ENSG00000122852 /// | | | |
| | | Homo sapiens cDNA FLJ51913 | | | |
| | | complete cds, highly similar to | | | |
| | | Pulmonary surfactant- | | | |
| | | associated protein A1 | | | |
| | | precursor. /// Homo sapiens | | | |
| | | cDNA FLJ50593 complete cds, | | | |
| | | moderately similar to | | | |
| | | Pulmonary surfactant- | | | |
| | | associated protein A1 | | | |
| | | precursor. /// Homo sapiens | | | |
| | | surfactant protein A2B, mRNA | | | |
| | | (cDNA clone MGC:189761 | | | |
| | | IMAGE:9057085), complete | | | |
| | | cds. /// Homo sapiens | | | |
| | | surfactant protein A2B, mRNA | | | |
| | | (cDNA clone MGC:189714 | | | |
| | | IMAGE:8862711), complete | | | |
| | | cds. /// Homo sapiens | | | |
| | | surfactant protein A2B | | | |
| | | (SFTPA2B), mRNA. /// Homo sapiens | | | |
| | | surfactant protein A2 | | | |
| | | (SFTPA2), mRNA. /// Homo sapiens | | | |
| | | surfactant protein A1 | | | |
| | | (SFTPA1), mRNA. /// Homo sapiens | | | |
| | | surfactant protein A1B | | | |
| | | (SFTPA1B), mRNA. | | | |
| 8068046 | ENST00000407713 /// | B lymphocyte activation-related | chromosome 21 open | C21orf118 | 12 |
| | AF304442 | protein BC-1514 | reading frame 118 | | |
| | | gene:ENSG00000219130 /// | | | |
| | | Homo sapiens B lymphocyte | | | |
| | | activation-related protein BC- | | | |
| | | 1514 mRNA, complete cds. | | | |
| 8131301 | ENST00000365169 | ncrna:snRNA | — | — | 13 |
| | | chromosome:NCBI36:7:5186218:5186319:1 | | | |
| | | gene:ENSG00000202039 | | | |

Table A(ii)

| | | | | | |
|---|---|---|---|---|---|
| 7934698 | ENST00000372329 /// | Pulmonary surfactant- | surfactant protein A1 /// | SFTPA1 /// | 14 |
| | ENST00000372327 /// | associated protein A2 | surfactant protein | SFTPA1B /// | |
| | ENST00000372325 /// | gene:ENSG00000182314 /// | A1B /// surfactant | SFTPA2 /// | |
| | ENST00000398636 /// | cDNA FLJ54288, moderately | protein A2 /// | SFTPA2B | |
| | ENST00000372316 /// | similar to Pulmonary surfactant- | surfactant protein | | |
| | ENST00000372313 /// | associated protein A1 | A2B | | |
| | ENST00000372308 /// | gene:ENSG00000185303 /// | | | |
| | ENST00000394569 /// | Pulmonary surfactant- | | | |
| | AK298029 /// | associated protein A1 | | | |
| | AK298034 /// | gene:ENSG00000185303 /// | | | |
| | BC157866 /// | Pulmonary surfactant- | | | |
| | BC157890 /// | associated protein A1 | | | |
| | NM_006926 /// | gene:ENSG00000185303 /// | | | |
| | NM_001098668 /// | Pulmonary surfactant- | | | |
| | NM_001093770 /// | associated protein A2 | | | |
| | NM_005411 | gene:ENSG00000122854 /// | | | |
| | | cDNA FLJ54288, moderately | | | |
| | | similar to Pulmonary surfactant- | | | |
| | | associated protein A1 | | | |
| | | gene:ENSG00000122852 /// | | | |
| | | Pulmonary surfactant- | | | |
| | | associated protein A1 | | | |
| | | gene:ENSG00000122852 /// | | | |
| | | Pulmonary surfactant- | | | |
| | | associated protein A1 | | | |
| | | gene:ENSG00000122852 /// | | | |
| | | Homo sapiens cDNA FLJ51913 | | | |
| | | complete cds, highly similar to | | | |
| | | Pulmonary surfactant- | | | |
| | | associated protein A1 | | | |
| | | precursor. /// Homo sapiens | | | |
| | | cDNA FLJ50593 complete cds, | | | |
| | | moderately similar to | | | |
| | | Pulmonary surfactant- | | | |
| | | associated protein A1 | | | |
| | | precursor. /// Homo sapiens | | | |
| | | surfactant protein A2B, mRNA | | | |
| | | (cDNA clone MGC:189761 | | | |
| | | IMAGE:9057085), complete | | | |
| | | cds. /// Homo sapiens | | | |
| | | surfactant protein A2B, mRNA | | | |
| | | (cDNA clone MGC:189714 | | | |
| | | IMAGE:8862711), complete | | | |
| | | cds. /// Homo sapiens | | | |
| | | surfactant protein A2B | | | |
| | | (SFTPA2B), mRNA. /// Homo sapiens | | | |
| | | surfactant protein A2 | | | |
| | | (SFTPA2), mRNA. /// Homo sapiens | | | |
| | | surfactant protein A1 | | | |
| | | (SFTPA1), mRNA. /// Homo sapiens | | | |
| | | surfactant protein A1B | | | |
| | | (SFTPA1B), mRNA. | | | |
| 7916882 | ENST00000262340 /// | Retinal pigment epithelium- | retinal pigment | RPE65 | 15 |
| | U18991 /// | specific 65 kDa protein | epithelium-specific | | |
| | NM_000329 | gene:ENSG00000116745 /// | protein 65 kDa | | |
| | | Human retinal pigment | | | |
| | | epithelium-specific 61 kDa | | | |
| | | protein (RPE65) mRNA, | | | |
| | | complete cds. /// Homo sapiens | | | |
| | | retinal pigment epithelium- | | | |
| | | specific protein 65 kDa | | | |
| | | (RPE65), mRNA. | | | |
| 8149324 | ENST00000284486 /// | UPF0484 protein FAM167A | family with sequence | FAM167A | 16 |
| | ENST00000398342 /// | gene:ENSG00000154319 /// | similarity 167, | | |
| | AL834122 /// | FAM167A protein | member A | | |
| | NM_053279 | gene:ENSG00000154319 /// | | | |
| | | Homo sapiens mRNA; cDNA | | | |
| | | DKFZp761F1821 (from clone | | | |
| | | DKFZp761F1821). /// Homo sapiens | | | |
| | | family with sequence | | | |
| | | similarity 167, member A | | | |
| | | (FAM167A), mRNA. | | | |
| 7914992 | ENST00000387309 | ncrna:rRNA_pseudogene | — | — | 17 |
| | | chromosome:NCBI36:1:37502886:37502974:−1 | | | |
| | | gene:ENSG00000210044 | | | |
| 8021774 | ENST00000405150 /// | FLJ44313 protein | FLJ44313 protein | FLJ44313 | 18 |
| | AK126293 | gene:ENSG00000220032 /// | | | |
| | | Homo sapiens cDNA FLJ44313 | | | |
| | | fis, clone TRACH2025911. | | | |
| 7965573 | ENST00000344911 /// | Isoform 2 of Netrin-4 | netrin 4 | NTN4 | 19 |
| | ENST00000343702 /// | gene:ENSG00000074527 /// | | | |
| | AF278532 /// | Isoform 1 of Netrin-4 | | | |
| | NM_021229 | gene:ENSG00000074527 /// | | | |
| | | Homo sapiens beta-netrin | | | |
| | | mRNA, complete cds. /// Homo sapiens | | | |
| | | netrin 4 (NTN4), | | | |
| | | mRNA. | | | |
| 8057771 | ENST00000358470 /// | Signal transducer and activator | signal transducer and | STAT4 | 20 |
| | ENST00000392320 /// | of transcription 4 | activator of | | |
| | BC031212 /// | gene:ENSG00000138378 /// | transcription 4 | | |
| | NM_003151 | Signal transducer and activator | | | |
| | | of transcription 4 | | | |
| | | gene:ENSG00000138378 /// | | | |
| | | Homo sapiens signal | | | |
| | | transducer and activator of | | | |
| | | transcription 4, mRNA (cDNA | | | |
| | | clone MGC:39492 | | | |
| | | IMAGE:4830583), complete | | | |
| | | cds. /// Homo sapiens signal | | | |
| | | transducer and activator of | | | |
| | | transcription 4 (STAT4), mRNA. | | | |
| 8111417 | ENST00000342059 /// | Isoform AIM-1c of Membrane- | solute carrier family | SLC45A2 | 21 |
| | ENST00000296589 /// | associated transporter protein | 45, member 2 | | |
| | ENST00000382102 /// | gene:ENSG00000164175 /// | | | |
| | ENST00000345083 /// | Isoform AIM-1a of Membrane- | | | |
| | AF172849 /// | associated transporter protein | | | |
| | NM_016180 /// | gene:ENSG00000164175 /// | | | |
| | NM_001012509 | membrane-associated | | | |
| | | transporter protein isoform b | | | |
| | | gene:ENSG00000164175 /// | | | |
| | | Isoform AIM-1b of Membrane- | | | |
| | | associated transporter protein | | | |
| | | gene:ENSG00000164175 /// | | | |
| | | Homo sapiens AIM-1 protein | | | |
| | | mRNA, complete cds. /// Homo sapiens | | | |
| | | solute carrier family 45, | | | |
| | | member 2 (SLC45A2), | | | |
| | | transcript variant 1, mRNA. /// | | | |
| | | Homo sapiens solute carrier | | | |
| | | family 45, member 2 | | | |
| | | (SLC45A2), transcript variant 2, | | | |
| | | mRNA. | | | |
| 8161381 | ENST00000316269 /// | hypothetical protein | hypothetical protein | LOC100133036 /// | 22 |
| | AK125850 /// | gene:ENSG00000204831 /// | LOC100133036 /// | FAM95B1 | |
| | AL833349 | Homo sapiens cDNA FLJ43862 | family with sequence | | |
| | | fis, clone TESTI4007775. /// | similarity 95, member | | |
| | | Homo sapiens mRNA; cDNA | B1 | | |
| | | DKFZp686P0734 (from clone | | | |
| | | DKFZp686P0734). | | | |
| 8165032 | ENST00000371763 /// | Glycosyltransferase 6 domain- | glycosyltransferase 6 | GLT6D1 | 23 |
| | AY336054 /// | containing protein 1 | domain containing 1 | | |
| | NM_182974 | gene:ENSG00000204007 /// | | | |
| | | Homo sapiens | | | |
| | | gycosyltransferase family 6 like- | | | |
| | | protein mRNA, complete cds. /// | | | |
| | | Homo sapiens | | | |
| | | glycosyltransferase 6 domain | | | |
| | | containing 1 (GLT6D1), mRNA. | | | |
| 8113276 | AF119888 | Homo sapiens PRO2613 | — | — | 24 |
| | | mRNA, complete cds. | | | |
| 8116874 | ENST00000283141 /// | Isoform 1 of Synaptonemal | synaptonemal | SYCP2L | 25 |
| | ENST00000341041 /// | complex protein 2-like | complex protein 2-like | | |
| | AK128130 /// | gene:ENSG00000153157 /// | | | |
| | NM_001040274 | Isoform 1 of Synaptonemal | | | |
| | | complex protein 2-like | | | |
| | | gene:ENSG00000153157 /// | | | |
| | | Homo sapiens cDNA FLJ46251 | | | |
| | | fis, clone TESTI4021713, | | | |
| | | weakly similar to Homo sapiens | | | |
| | | synaptonemal complex | | | |
| | | protein 2 (SYCP2). /// Homo sapiens | | | |
| | | synaptonemal complex | | | |
| | | protein 2-like (SYCP2L), | | | |
| | | mRNA. | | | |
| 8030753 | ENST00000326003 /// | Prostate-specific antigen | kallikrein-related | KLK3 | 26 |
| | ENST00000326052 /// | gene:ENSG00000142515 /// | peptidase 3 | | |
| | ENST00000360617 /// | prostate specific antigen | | | |
| | BC005307 /// | isoform 5 preproprotein | | | |
| | NM_001648 /// | gene:ENSG00000142515 /// | | | |
| | NM_001030047 /// | prostate specific antigen | | | |
| | NM_001030048 /// | isoform 3 preproprotein | | | |
| | NM_001030049 /// | gene:ENSG00000142515 /// | | | |
| | NM_001030050 | Homo sapiens kallikrein-related | | | |
| | | peptidase 3, mRNA (cDNA | | | |
| | | clone MGC:12378 | | | |
| | | IMAGE:3950475), complete | | | |
| | | cds. /// Homo sapiens kallikrein- | | | |
| | | related peptidase 3 (KLK3), | | | |
| | | transcript variant 1, mRNA. /// | | | |
| | | Homo sapiens kallikrein-related | | | |
| | | peptidase 3 (KLK3), transcript | | | |
| | | variant 3, mRNA. /// Homo sapiens | | | |
| | | kallikrein-related | | | |
| | | peptidase 3 (KLK3), transcript | | | |
| | | variant 4, mRNA. /// Homo sapiens | | | |
| | | kallikrein-related | | | |
| | | peptidase 3 (KLK3), transcript | | | |
| | | variant 5, mRNA. /// Homo sapiens | | | |
| | | kallikrein-related | | | |
| | | peptidase 3 (KLK3), transcript | | | |
| | | variant 6, mRNA. | | | |
| 8098439 | GENSCAN00000042517 | cdna:Genscan | — | — | 27 |
| | | chromosome:NCBI36:4:182680810:182745578:1 | | | |
| 8076819 | ENST00000380990 /// | Conserved hypothetical protein | FLJ46257 protein | RP11- | 28 |
| | AK128136 | gene:ENSG00000205634 /// | | 191L9.1 | |
| | | Homo sapiens cDNA FLJ46257 | | | |
| | | fis, clone TESTI4024240. | | | |
| 7957819 | ENST00000392989 /// | Isoform 2 of Vesicular | solute carrier family | SLC17A8 | 29 |
| | ENST00000323346 /// | glutamate transporter 3 | 17 (sodium- | | |
| | AK128319 /// | gene:ENSG00000179520 /// | dependent inorganic | | |
| | NM_139319 | Isoform 1 of Vesicular | phosphate | | |
| | | glutamate transporter 3 | cotransporter), | | |
| | | gene:ENSG00000179520 /// | member 8 | | |
| | | Homo sapiens cDNA FLJ46460 | | | |
| | | fis, clone THYMU3021404, | | | |
| | | highly similar to Homo sapiens | | | |
| | | solute carrier family 17 | | | |
| | | (sodium-dependent inorganic | | | |
| | | phosphate cotransporter), | | | |
| | | member 8 (SLC17A8), mRNA. /// | | | |
| | | Homo sapiens solute carrier | | | |
| | | family 17 (sodium-dependent | | | |
| | | inorganic phosphate | | | |
| | | cotransporter), member 8 | | | |
| | | (SLC17A8), mRNA. | | | |
| 7986229 | ENST00000268164 /// | Alpha-2,8-sialyltransferase 8B | ST8 alpha-N-acetyl- | ST8SIA2 | 30 |
| | ENST00000378973 /// | gene:ENSG00000140557 /// | neuraminide alpha- | | |
| | BC096202 /// | ST8SIA2 protein | 2,8-sialyltransferase 2 | | |
| | NM_006011 | gene:ENSG00000140557 /// | | | |
| | | Homo sapiens ST8 alpha-N- | | | |
| | | acetyl-neuraminide alpha-2,8- | | | |
| | | sialyltransferase 2, mRNA | | | |
| | | (cDNA clone MGC:116854 | | | |
| | | IMAGE:40004644), complete | | | |
| | | cds. /// Homo sapiens ST8 | | | |
| | | alpha-N-acetyl-neuraminide | | | |
| | | alpha-2,8-sialyltransferase 2 | | | |
| | | (ST8SIA2), mRNA. | | | |
| 7995310 | ENST00000319817 | Putative uncharacterized | — | — | 31 |
| | | protein MGC3480 | | | |
| | | gene:ENSG00000179755 | | | |
| 8138920 | ENST00000387801 /// | ncrna:snRNA_pseudogene | — | — | 32 |
| | ENST00000387652 /// | chromosome:NCBI36:12:62305744:62305830:−1 | | | |
| | ENST00000387676 /// | gene:ENSG00000210536 /// | | | |
| | ENST00000387734 /// | ncrna:snRNA_pseudogene | | | |
| | ENST00000386042 | chromosome:NCBI36:7:29701450:29701536:1 | | | |
| | | gene:ENSG00000210387 /// | | | |
| | | ncrna:snRNA_pseudogene | | | |
| | | chromosome:NCBI36:7:32724496:32724582:−1 | | | |
| | | gene:ENSG00000210411 /// | | | |
| | | ncrna:snRNA_pseudogene | | | |
| | | chromosome:NCBI36:7:35155590:35155669:−1 | | | |
| | | gene:ENSG00000210469 /// | | | |
| | | ncrna:snRNA_pseudogene | | | |
| | | chromosome:NCBI36:7:102669899:102669985:−1 | | | |
| | | gene:ENSG00000208777 | | | |
| 8055492 | ENST00000385544 | ncrna:Mt_tRNA_pseudogene | — | — | 33 |
| | | chromosome:NCBI36:2:140697147:140697215:−1 | | | |
| | | gene:ENSG00000208279 | | | |
| 7995674 | ENST00000290552 /// | Iroquois-class homeodomain | iroquois homeobox 6 | IRX6 | 34 |
| | AK125053 /// | protein IRX-6 | | | |
| | NM_024335 | gene:ENSG00000159387 /// | | | |
| | | Homo sapiens cDNA FLJ43063 | | | |
| | | fis, clone BRTHA3008310, | | | |
| | | moderately similar to Mus musculus | | | |
| | | mRNA for iroquois | | | |
| | | homeobox protein 6. /// Homo sapiens | | | |
| | | iroquois homeobox 6 | | | |
| | | (IRX6), mRNA. | | | |
| 8155627 | ENST00000316269 /// | hypothetical protein | hypothetical protein | LOC100133036 /// | 35 |
| | AK125850 /// | gene:ENSG00000204831 /// | LOC100133036 /// | FAM95B1 | |
| | AL833349 | Homo sapiens cDNA FLJ43862 | family with sequence | | |
| | | fis, clone TESTI4007775. /// | similarity 95, member | | |
| | | Homo sapiens mRNA; cDNA | B1 | | |
| | | DKFZp686P0734 (from clone | | | |
| | | DKFZp686P0734). | | | |
| 7941608 | GENSCAN00000024384 /// | cdna:Genscan | — | — | 36 |
| | ENST00000364863 | chromosome:NCBI36:11:65945227:65956926:1 /// | | | |
| | | ncrna:snoRNA | | | |
| | | chromosome:NCBI36:11:65956813:65956949:1 | | | |
| | | gene:ENSG00000201733 | | | |
| 7957495 | ENST00000362375 | ncrna:misc_RNA | — | — | 37 |
| | | chromosome:NCBI36:12:87348350:87348458:1 | | | |
| | | gene:ENSG00000199245 | | | |
| 7961413 | ENST00000318426 /// | Putative uncharacterized | chromosome 12 open | C12orf36 | 38 |
| | BC101220 /// | protein C12orf36 | reading frame 36 | | |
| | NM_182558 | gene:ENSG00000180861 /// | | | |
| | | Homo sapiens chromosome 12 | | | |
| | | open reading frame 36, mRNA | | | |
| | | (cDNA clone MGC:120138 | | | |
| | | IMAGE:40022214), complete | | | |
| | | cds. /// Homo sapiens | | | |
| | | chromosome 12 open reading | | | |
| | | frame 36 (C12orf36), mRNA. | | | |
| 8055941 | ENST00000325926 /// | Protein reprimo | reprimo, TP53 | RPRM | 39 |
| | BC002908 /// | gene:ENSG00000177519 /// | dependent G2 arrest | | |
| | NM_019845 | Homo sapiens reprimo, TP53 | mediator candidate | | |
| | | dependent G2 arrest mediator | | | |
| | | candidate, mRNA (cDNA clone | | | |
| | | MGC:11260 IMAGE:3942270), | | | |
| | | complete cds. /// Homo sapiens | | | |
| | | reprimo, TP53 dependent G2 | | | |
| | | arrest mediator candidate | | | |
| | | (RPRM), mRNA. | | | |
| 7962792 | ENST00000310248 /// | Olfactory receptor 10AD1 | olfactory receptor, | OR10AD1 | 40 |
| | NM_001004134 | gene:ENSG00000172640 /// | family 10, subfamily | | |
| | | Homo sapiens olfactory | AD, member 1 | | |
| | | receptor, family 10, subfamily | | | |
| | | AD, member 1 (OR10AD1), | | | |
| | | mRNA. | | | |
| 7896756 | ENST00000326734 /// | similar to hCG1735895 | — | — | 41 |
| | BC118644 | gene:ENSG00000177757 /// | | | |
| | | Homo sapiens cDNA clone | | | |
| | | IMAGE:40030978. | | | |
| 8054939 | ENST00000411186 | ncrna:misc_RNA | — | — | 42 |
| | | chromosome:NCBI36:2:124343303:124343617:−1 | | | |
| | | gene:ENSG00000223118 | | | |
| 7976057 | ENST00000387641 | ncrna:snoRNA_pseudogene | — | — | 43 |
| | | chromosome:NCBI36:14:81998185:81998284:1 | | | |
| | | gene:ENSG00000210376 | | | |
| 7940002 | ENST00000332362 /// | Leucine rich repeat containing | leucine rich repeat | LRRC55 | 44 |
| | BC136737 /// | 55 gene:ENSG00000183908 /// | containing 55 | | |
| | NM_001005210 | Homo sapiens leucine rich | | | |
| | | repeat containing 55, mRNA | | | |
| | | (cDNA clone MGC:168350 | | | |
| | | IMAGE:9020727), complete | | | |
| | | cds. /// Homo sapiens leucine | | | |
| | | rich repeat containing 55 | | | |
| | | (LRRC55), mRNA. | | | |
| 7979204 | ENST00000395631 /// | Isoform 1 of Fermitin family | fermitin family | FERMT2 | 45 |
| | ENST00000343279 /// | homolog 2 | homolog 2 | | |
| | ENST00000341590 /// | gene:ENSG00000073712 /// | (Drosophila) | | |
| | Z24725 /// | fermitin family homolog 2 | | | |
| | NM_006832 /// | isoform 2 | | | |
| | NM_001134999 /// | gene:ENSG00000073712 /// | | | |
| | NM_001135000 | Isoform 1 of Fermitin family | | | |
| | | homolog 2 | | | |
| | | gene:ENSG00000073712 /// | | | |
| | | H. sapiens mitogen inducible | | | |
| | | gene mig-2, complete CDS. /// | | | |
| | | Homo sapiens fermitin family | | | |
| | | homolog 2 (Drosophila) | | | |
| | | (FERMT2), transcript variant 1, | | | |
| | | mRNA. /// Homo sapiens | | | |
| | | fermitin family homolog 2 | | | |
| | | (Drosophila) (FERMT2), | | | |
| | | transcript variant 2, mRNA. /// | | | |
| | | Homo sapiens fermitin family | | | |
| | | homolog 2 (Drosophila) | | | |
| | | (FERMT2), transcript variant 3, | | | |
| | | mRNA. | | | |
| 7981190 | AL834311 | Homo sapiens mRNA; cDNA | hypothetical | LOC100130815 | 46 |
| | | DKFZp434O1614 (from clone | LOC100130815 | | |
| | | DKFZp434O1614). | | | |
| 8092686 | ENST00000358241 /// | Receptor-transporting protein 2 | receptor | RTP2 | 47 |
| | BC068081 /// | gene:ENSG00000198471 /// | (chemosensory) | | |
| | NM_001004312 | Homo sapiens receptor | transporter protein 2 | | |
| | | (chemosensory) transporter | | | |
| | | protein 2, mRNA (cDNA clone | | | |
| | | MGC:78665 IMAGE:6212901), | | | |
| | | complete cds. /// Homo sapiens | | | |
| | | receptor (chemosensory) | | | |
| | | transporter protein 2 (RTP2), | | | |
| | | mRNA. | | | |
| 7961604 | ENST00000266505 /// | Isoform 1 of 1- | phospholipase C, | PLCZ1 | 48 |
| | ENST00000318197 /// | phosphatidylinositol-4,5- | zeta 1 | | |
| | AY035866 /// | bisphosphate | | | |
| | NM_033123 | phosphodiesterase zeta-1 | | | |
| | | gene:ENSG00000139151 /// | | | |
| | | cDNA FLJ40236 fis, clone | | | |
| | | TESTI2023214, weakly similar | | | |
| | | to 1- | | | |
| | | PHOSPHATIDYLINOSITOL- | | | |
| | | 4,5-BISPHOSPHATE | | | |
| | | PHOSPHODIESTERASE | | | |
| | | DELTA 1 | | | |
| | | gene:ENSG00000139151 /// | | | |
| | | Homo sapiens testis- | | | |
| | | development related NYD- | | | |
| | | SP27 mRNA, complete cds. /// | | | |
| | | Homo sapiens phospholipase | | | |
| | | C, zeta 1 (PLCZ1), mRNA. | | | |
| 8026503 | ENST00000397365 /// | Putative uncharacterized | hypothetical | FLJ25328 | 49 |
| | ENST00000343017 /// | protein FLJ25328 | LOC148231 | | |
| | NR_024335 /// | gene:ENSG00000167459 /// | | | |
| | NR_024336 | FLJ25328 protein (Fragment) | | | |
| | | gene:ENSG00000167459 /// | | | |
| | | Homo sapiens hypothetical | | | |
| | | LOC148231 (FLJ25328), | | | |
| | | transcript variant 1, non-coding | | | |
| | | RNA. /// Homo sapiens | | | |
| | | hypothetical LOC148231 | | | |
| | | (FLJ25328), transcript variant 2, | | | |
| | | non-coding RNA. | | | |
| 8084887 | ENST00000411400 /// | ncrna:misc_RNA | — | — | 50 |
| | ENST00000385589 | chromosome:NCBI36:3:195346160:195346450:1 | | | |
| | | gene:ENSG00000223332 /// | | | |
| | | ncrna:scRNA_pseudogene | | | |
| | | chromosome:NCBI36:3:195346161:195346454:1 | | | |
| | | gene:ENSG00000208324 | | | |
| 7904417 | GENSCAN00000027599 /// | cdna:Genscan | — | — | 51 |
| | ENST00000286193 | chromosome:NCBI36:1:119806478:119858791:1 /// | | | |
| | | cdna:pseudogene | | | |
| | | chromosome:NCBI36:1:119811367:119817360:1 | | | |
| | | gene:ENSG00000187481 | | | |
| 8112666 | GENSCAN00000026551 /// | cdna:Genscan | — | — | 52 |
| | ENST00000329491 | chromosome:NCBI36:5:74319149:74349847:−1 /// | | | |
| | | cdna:pseudogene | | | |
| | | chromosome:NCBI36:5:74321774:74322218:−1 | | | |
| | | gene:ENSG00000182383 | | | |
| 8036969 | ENST00000301141 /// | Cytochrome P450 2A6 | cytochrome P450, | CYP2A13 /// | 53 |
| | ENST00000301146 /// | gene:ENSG00000213052 /// | family 2, subfamily A, | CYP2A7 /// | |
| | ENST00000291764 /// | Cytochrome P450 2A7 | polypeptide 13 /// | CYP2A6 | |
| | ENST00000359667 /// | gene:ENSG00000198077 /// | cytochrome P450, | | |
| | ENST00000330436 /// | cytochrome P450, family 2, | family 2, subfamily A, | | |
| | AF209774 /// | subfamily A, polypeptide 7 | polypeptide 7 /// | | |
| | M33317 /// | isoform 2 | cytochrome P450, | | |
| | M33318 /// | gene:ENSG00000198077 /// | family 2, subfamily A, | | |
| | NM_000764 /// | cdna:known | polypeptide 6 | | |
| | NM_000762 /// | chromosome:NCBI36:19:46107659:46108594:−1 | | | |
| | NM_000766 /// | gene:ENSG00000198251 /// | | | |
| | NM_030589 | Cytochrome P450 2A13 | | | |
| | | gene:ENSG00000197838 /// | | | |
| | | Homo sapiens cytochrome | | | |
| | | P450 2A13 (CYP2A13)mRNA, | | | |
| | | complete cds. /// Human | | | |
| | | cytochrome P450IIA4 | | | |
| | | (CYP2A4) mRNA, complete | | | |
| | | cds. /// Human cytochrome | | | |
| | | P450IIA3 (CYP2A3) mRNA, | | | |
| | | complete cds. /// Homo sapiens | | | |
| | | cytochrome P450, family 2, | | | |
| | | subfamily A, polypeptide 7 | | | |
| | | (CYP2A7), transcript variant 1, | | | |
| | | mRNA. /// Homo sapiens | | | |
| | | cytochrome P450, family 2, | | | |
| | | subfamily A, polypeptide 6 | | | |
| | | (CYP2A6), mRNA. /// Homo sapiens | | | |
| | | cytochrome P450, | | | |
| | | family 2, subfamily A, | | | |
| | | polypeptide 13 (CYP2A13), | | | |
| | | mRNA. /// Homo sapiens | | | |
| | | cytochrome P450, family 2, | | | |
| | | subfamily A, polypeptide 7 | | | |
| | | (CYP2A7), transcript variant 2, | | | |
| | | mRNA. | | | |
| 8176935 | ENST00000303804 /// | Isoform 1 of PTPN13-like | PTPN13-like, Y-linked /// | PRY /// | 54 |
| | ENST00000303728 /// | protein, Y-linked | PTPN13-like, Y- | PRY2 | |
| | ENST00000343584 /// | gene:ENSG00000169807 /// | linked 2 | | |
| | ENST00000303593 /// | Isoform 1 of PTPN13-like | | | |
| | ENST00000306589 /// | protein, Y-linked | | | |
| | ENST00000338673 /// | gene:ENSG00000169789 /// | | | |
| | AF000988 /// | Isoform 1 of PTPN13-like | | | |
| | NM_001002758 /// | protein, Y-linked | | | |
| | NM_004676 | gene:ENSG00000169763 /// | | | |
| | | Isoform 2 of PTPN13-like | | | |
| | | protein, Y-linked | | | |
| | | gene:ENSG00000169763 /// | | | |
| | | Isoform 1 of PTPN13-like | | | |
| | | protein, Y-linked | | | |
| | | gene:ENSG00000172283 /// | | | |
| | | Isoform 2 of PTPN13-like | | | |
| | | protein, Y-linked | | | |
| | | gene:ENSG00000172283 /// | | | |
| | | Homo sapiens testis-specific | | | |
| | | PTP-BL Related Y protein | | | |
| | | (PRY) mRNA, complete cds. /// | | | |
| | | Homo sapiens PTPN13-like, Y- | | | |
| | | linked 2 (PRY2), mRNA. /// | | | |
| | | Homo sapiens PTPN13-like, Y- | | | |
| | | linked (PRY), mRNA. | | | |
| 8044124 | ENST00000258456 /// | High-affinity lysophosphatidic | G protein-coupled | GPR45 | 55 |
| | U92642 /// | acid receptor homolog | receptor 45 | | |
| | NM_007227 | gene:ENSG00000135973 /// | | | |
| | | Human high-affinity | | | |
| | | lysophosphatidic acid receptor | | | |
| | | homolog mRNA, complete cds. /// | | | |
| | | Homo sapiens G protein- | | | |
| | | coupled receptor 45 (GPR45), | | | |
| | | mRNA. | | | |
| 8070930 | — | — | — | — | 56 |
| 8015037 | AK095738 | Homo sapiens cDNA FLJ38419 | — | — | 57 |
| | | fis, clone FEBRA2009846. | | | |
| 7969482 | ENST00000377462 | similar to hCG30005 | — | — | 58 |
| | | gene:ENSG00000102794 | | | |
| 8173524 | ENST00000373619 /// | Cbp/p300-interacting | Cbp/p300-interacting | CITED1 | 59 |
| | ENST00000246139 /// | transactivator 1 | transactivator, with | | |
| | U65092 /// | gene:ENSG00000125931 /// | Glu/Asp-rich carboxy- | | |
| | NM_004143 | Cbp/p300-interacting | terminal domain, 1 | | |
| | | transactivator 1 | | | |
| | | gene:ENSG00000125931 /// | | | |
| | | Human melanocyte-specific | | | |
| | | gene 1 (msg1) mRNA, | | | |
| | | complete cds. /// Homo sapiens | | | |
| | | Cbp/p300-interacting | | | |
| | | transactivator, with Glu/Asp-rich | | | |
| | | carboxy-terminal domain, 1 | | | |
| | | (CITED1), mRNA. | | | |
| 8143747 | ENST00000385536 | ncrna:tRNA_pseudogene | — | — | 60 |
| | | chromosome:NCBI36:7:148966447:148966513:−1 | | | |
| | | gene:ENSG00000208271 | | | |
| 8080781 | ENST00000356151 /// | Isoform 1 of PX domain- | PX domain containing | PXK | 61 |
| | ENST00000302779 /// | containing protein kinase-like | serine/threonine | | |
| | ENST00000383715 /// | protein | kinase | | |
| | ENST00000383716 /// | gene:ENSG00000168297 /// | | | |
| | AY274811 /// | Isoform 4 of PX domain- | | | |
| | NM_017771 | containing protein kinase-like | | | |
| | | protein | | | |
| | | gene:ENSG00000168297 /// | | | |
| | | Isoform 2 of PX domain- | | | |
| | | containing protein kinase-like | | | |
| | | protein | | | |
| | | gene:ENSG00000168297 /// | | | |
| | | Isoform 6 of PX domain- | | | |
| | | containing protein kinase-like | | | |
| | | protein | | | |
| | | gene:ENSG00000168297 /// | | | |
| | | Homo sapiens PX | | | |
| | | serine/threonine kinase mRNA, | | | |
| | | complete cds. /// Homo sapiens | | | |
| | | PX domain containing | | | |
| | | serine/threonine kinase (PXK), | | | |
| | | mRNA. | | | |
| 8111118 | ENST00000365399 | ncrna:snoRNA | — | — | 62 |
| | | chromosome:NCBI36:5:15163895:15164029:−1 | | | |
| | | gene:ENSG00000202269 | | | |
| 7934883 | ENST00000387878 | ncrna:scRNA_pseudogene | — | — | 63 |
| | | chromosome:NCBI36:10:89582555:89582827:−1 | | | |
| | | gene:ENSG00000210613 | | | |
| 8010113 | ENST00000374998 /// | Isoform 3 of Alpha-1,6- | mannosyl (alpha-1,6-)- | MGAT5B | 64 |
| | ENST00000374999 /// | mannosylglycoprotein 6-beta-N- | glycoprotein beta- | | |
| | ENST00000301618 /// | acetylglucosaminyltransferase | 1,6-N-acetyl- | | |
| | AB114297 /// | B gene:ENSG00000167889 /// | glucosaminyltransferase, | | |
| | NM_144677 /// | Isoform 1 of Alpha-1,6- | isozyme B | | |
| | NM_198955 | mannosylglycoprotein 6-beta-N- | | | |
| | | acetylglucosaminyltransferase | | | |
| | | B gene:ENSG00000167889 /// | | | |
| | | beta(1,6)-N- | | | |
| | | acetylglucosaminyltransferase | | | |
| | | V isoform 1 | | | |
| | | gene:ENSG00000167889 /// | | | |
| | | Homo sapiens hGnTVb mRNA | | | |
| | | for UDP-N-acetylglucosamine:alpha1,6- | | | |
| | | D-mannoside beta1,6-N- | | | |
| | | acetylglucosaminyltransferase | | | |
| | | b, complete cds. /// Homo sapiens | | | |
| | | mannosyl (alpha-1,6)- | | | |
| | | glycoprotein beta-1,6-N-acetyl- | | | |
| | | glucosaminyltransferase, | | | |
| | | isozyme B (MGAT5B), | | | |
| | | transcript variant 1, mRNA. /// | | | |
| | | Homo sapiens mannosyl | | | |
| | | (alpha-1,6-)-glycoprotein beta- | | | |
| | | 1,6-N-acetyl- | | | |
| | | glucosaminyltransferase, | | | |
| | | isozyme B (MGAT5B), | | | |
| | | transcript variant 2, mRNA. | | | |
| 8175524 | ENST00000370540 /// | Uncharacterized protein | hypothetical | RP11- | 65 |
| | NM_001013403 | LOC347487 | LOC347487 | 35F15.2 | |
| | | gene:ENSG00000203933 /// | | | |
| | | Homo sapiens hypothetical | | | |
| | | LOC347487 (LOC347487), | | | |
| | | mRNA. | | | |
| 7997740 | ENST00000268607 /// | Microtubule-associated proteins | microtubule- | MAP1LC3B | 66 |
| | BC045759 /// | 1A/1B light chain 3B | associated protein 1 | | |
| | NM_022818 | gene:ENSG00000140941 /// | light chain 3 beta | | |
| | | Homo sapiens microtubule- | | | |
| | | associated protein 1 light chain | | | |
| | | 3 beta, mRNA (cDNA clone | | | |
| | | MGC:48651 IMAGE:4828857), | | | |
| | | complete cds. /// Homo sapiens | | | |
| | | microtubule-associated protein | | | |
| | | 1 light chain 3 beta | | | |
| | | (MAP1LC3B), mRNA. | | | |
| 8177395 | ENST00000303804 /// | isoform 1 of PTPN13-like | PTPN13-like, Y-linked /// | PRY /// | 67 |
| | ENST00000303728 /// | protein, Y-linked | PTPN13-like, Y- | PRY2 | |
| | ENST00000343584 /// | gene:ENSG00000169807 /// | linked 2 | | |
| | ENST00000303593 /// | Isoform 1 of PTPN13-like | | | |
| | ENST00000306589 /// | protein, Y-linked | | | |
| | ENST00000338673 /// | gene:ENSG00000169789 /// | | | |
| | AF000988 /// | Isoform 1 of PTPN13-like | | | |
| | NM_001002758 /// | protein, Y-linked | | | |
| | NM_004676 | gene:ENSG00000169763 /// | | | |
| | | Isoform 2 of PTPN13-like | | | |
| | | protein, Y-linked | | | |
| | | gene:ENSG00000169763 /// | | | |
| | | Isoform 1 of PTPN13-like | | | |
| | | protein, Y-linked | | | |
| | | gene:ENSG00000172283 /// | | | |
| | | Isoform 2 of PTPN13-like | | | |
| | | protein, Y-linked | | | |
| | | gene:ENSG00000172283 /// | | | |
| | | Homo sapiens testis-specific | | | |
| | | PTP-BL Related Y protein | | | |
| | | (PRY) mRNA, complete cds. /// | | | |
| | | Homo sapiens PTPN13-like, Y- | | | |
| | | linked 2 (PRY2), mRNA. /// | | | |
| | | Homo sapiens PTPN13-like, Y- | | | |
| | | linked (PRY), mRNA. | | | |
| 7929478 | ENST00000371321 /// | Cytochrome P450 2C19 | cytochrome P450, | CYP2C19 | 68 |
| | M61854 /// | gene:ENSG00000165841 /// | family 2, subfamily C, | | |
| | NM_000769 | Human cytochrome P4502C19 | polypeptide 19 | | |
| | | (CYP2C19) mRNA, clone 11a. /// | | | |
| | | Homo sapiens cytochrome | | | |
| | | P450, family 2, subfamily C, | | | |
| | | polypeptide 19 (CYP2C19), | | | |
| | | mRNA. | | | |
| 8102781 | GENSCAN00000015129 /// | cdna:Genscan | similar to | LOC646187 | 69 |
| | XR_016991 | chromosome:NCBI36:4:132863736:133029672:−1 /// | hCG2026352 | | |
| | | PREDICTED: Homo sapiens | | | |
| | | similar to hCG2026352 | | | |
| | | (LOC646187), mRNA. | | | |
| 8155026 | ENST00000329395 /// | Putative FetA-like protein | ATPase, Class I, type | LOC158381 | 70 |
| | BC031276 /// | gene:ENSG00000179766 /// | 8B family | | |
| | NR_003581 /// | Homo sapiens ATPase, Class I, | pseudogene | | |
| | NR_003582 | type 8B family pseudogene, | | | |
| | | mRNA (cDNA clone | | | |
| | | MGC:39768 IMAGE:5295199), | | | |
| | | complete cds. /// Homo sapiens | | | |
| | | ATPase, Class I, type 8B family | | | |
| | | pseudogene (LOC158381), | | | |
| | | transcript variant 1, non-coding | | | |
| | | RNA. /// Homo sapiens | | | |
| | | ATPase, Class I, type 8B family | | | |
| | | pseudogene (LOC158381), | | | |
| | | transcript variant 2, non-coding | | | |
| | | RNA. | | | |
| 8146914 | ENST00000276603 /// | Isoform 1 of Telomeric repeat- | telomeric repeat | TERF1 | 71 |
| | ENST00000276602 /// | binding factor 1 | binding factor (NIMA- | | |
| | U74382 /// | gene:ENSG00000147601 /// | interacting) 1 | | |
| | NM_003218 /// | Isoform 2 of Telomeric repeat- | | | |
| | NM_017489 | binding factor 1 | | | |
| | | gene:ENSG00000147601 /// | | | |
| | | Human telomeric repeat DNA- | | | |
| | | binding protein (PIN2) mRNA, | | | |
| | | complete cds. /// Homo sapiens | | | |
| | | telomeric repeat binding factor | | | |
| | | (NIMA-interacting) 1 (TERF1), | | | |
| | | transcript variant 2, mRNA. /// | | | |
| | | Homo sapiens telomeric repeat | | | |
| | | binding factor (NIMA- | | | |
| | | interacting) 1 (TERF1), | | | |
| | | transcript variant 1, mRNA. | | | |
| 8169022 | ENST00000372661 /// | WW domain-binding protein 5 | WW domain binding | WBP5 | 72 |
| | ENST00000372656 /// | gene:ENSG00000185222 /// | protein 5 | | |
| | BC023544 /// | WW domain-binding protein 5 | | | |
| | NM_001006612 /// | gene:ENSG00000185222 /// | | | |
| | NM_016303 /// | Homo sapiens WW domain | | | |
| | NM_001006614 /// | binding protein 5, mRNA (cDNA | | | |
| | NM_001006613 | clone MGC:15211 | | | |
| | | IMAGE:4122244), complete | | | |
| | | cds. /// Homo sapiens WW | | | |
| | | domain binding protein 5 | | | |
| | | (WBP5), transcript variant 2, | | | |
| | | mRNA. /// Homo sapiens WW | | | |
| | | domain binding protein 5 | | | |
| | | (WBP5), transcript variant 1, | | | |
| | | mRNA. /// Homo sapiens WW | | | |
| | | domain binding protein 5 | | | |
| | | (WBP5), transcript variant 4, | | | |
| | | mRNA. /// Homo sapiens WW | | | |
| | | domain binding protein 5 | | | |
| | | (WBP5), transcript variant 3, | | | |
| | | mRNA. | | | |
| 7944952 | ENST00000340456 /// | cdna:known | — | — | 73 |
| | AK128036 | chromosome:NCBI36:11:124488267:124501887:1 | | | |
| | | gene:ENSG00000187686 /// | | | |
| | | Homo sapiens cDNA FLJ46155 | | | |
| | | fis, clone TESTI4001517. | | | |
| 8108199 | BC025747 | Homo sapiens similar to | similar to CG4995 | LOC153328 | 74 |
| | | CG4995 gene product, mRNA | gene product | | |
| | | (cDNA clone MGC:35539 | | | |
| | | IMAGE:5200129), complete | | | |
| | | cds. | | | |
| 8040618 | ENST00000264710 /// | Ras-related protein Rab-10 | RAB10, member RAS | RAB10 | 75 |
| | AK023223 /// | gene:ENSG00000084733 /// | oncogene family | | |
| | NM_016131 | Homo sapiens cDNA FLJ13161 | | | |
| | | fis, clone NT2RP3003589, | | | |
| | | highly similar to Homo sapiens | | | |
| | | ras-related GTP-binding protein | | | |
| | | mRNA. /// Homo sapiens | | | |
| | | RAB10, member RAS | | | |
| | | oncogene family (RAB10), | | | |
| | | mRNA. | | | |
| 7981326 | hsa-mir-1247 /// | MI0006382 Homo sapiens miR- | DIO3 opposite strand | DIO3OS | 76 |
| | hsa-mir-1247 /// | 1247 stem-loop /// MI0006382 | (non-protein coding) | | |
| | AF305836 /// | Homo sapiens miR-1247 stem- | | | |
| | AF469206 /// | loop /// Homo sapiens uterine- | | | |
| | BC065701 | derived 14 kDa protein mRNA, | | | |
| | | complete cds. /// Homo sapiens | | | |
| | | clone 8 DIO3AS mRNA, partial | | | |
| | | sequence; alternatively spliced. /// | | | |
| | | Homo sapiens deiodinase, | | | |
| | | iodothyronine, type /// opposite | | | |
| | | strand, mRNA (cDNA clone | | | |
| | | IMAGE:6205020). | | | |
| 8057004 | ENST00000358450 /// | Isoform 2 of Dual 3′,5′-cyclic- | phosphodiesterase | PDE11A | 77 |
| | ENST00000286063 /// | AMP and -GMP | 11A | | |
| | ENST00000389683 /// | phosphodiesterase 11A | | | |
| | ENST00000409504 /// | gene:ENSG00000128655 /// | | | |
| | AB036704 /// | Isoform 1 of Dual 3′,5′-cyclic- | | | |
| | NM_001077358 /// | AMP and -GMP | | | |
| | NM_016953 /// | phosphodiesterase 11A | | | |
| | NM_001077196 /// | gene:ENSG00000128655 /// | | | |
| | NM_001077197 | Isoform 4 of Dual 3′,5′-cyclic- | | | |
| | | AMP and -GMP | | | |
| | | phosphodiesterase 11A | | | |
| | | gene:ENSG00000128655 /// | | | |
| | | cdna:known | | | |
| | | chromosome:NCBI36:2:178202021:178495768:−1 | | | |
| | | gene:ENSG00000128655 /// | | | |
| | | Homo sapiens HSPDE11A | | | |
| | | mRNA for phosphodiesterase | | | |
| | | 11A, complete cds. /// Homo sapiens | | | |
| | | phosphodiesterase | | | |
| | | 11A (PDE11A), transcript | | | |
| | | variant 2, mRNA. /// Homo sapiens | | | |
| | | phosphodiesterase | | | |
| | | 11A (PDE11A), transcript | | | |
| | | variant 4, mRNA. /// Homo sapiens | | | |
| | | phosphodiesterase | | | |
| | | 11A (PDE11A), transcript | | | |
| | | variant 1, mRNA. /// Homo sapiens | | | |
| | | phosphodiesterase | | | |
| | | 11A (PDE11A), transcript | | | |
| | | variant 3, mRNA. | | | |
| 8108370 | ENST00000239938 /// | Early growth response protein 1 | early growth response | EGR1 | 78 |
| | M62829 /// | gene:ENSG00000120738 /// | 1 | | |
| | NM_001964 | Human transcription factor | | | |
| | | ETR103 mRNA, complete cds. /// | | | |
| | | Homo sapiens early growth | | | |
| | | response 1 (EGR1), mRNA. | | | |
| 8159078 | ENST00000316948 /// | Isoform 1 of Transmembrane | chromosome 9 open | C9orf7 | 79 |
| | ENST00000291722 /// | protein C9orf7 | reading frame 7 | | |
| | AK074852 /// | gene:ENSG00000160325 /// | | | |
| | NM_017586 /// | Isoform 2 of Transmembrane | | | |
| | NM_001135775 | protein C9orf7 | | | |
| | | gene:ENSG00000160325 /// | | | |
| | | Homo sapiens cDNA FLJ90371 | | | |
| | | fis, clone NT2RP2004524. /// | | | |
| | | Homo sapiens chromosome 9 | | | |
| | | open reading frame 7 (C9orf7), | | | |
| | | transcript variant 1, mRNA. /// | | | |
| | | Homo sapiens chromosome 9 | | | |
| | | open reading frame 7 (C9orf7), | | | |
| | | transcript variant 2, mRNA. | | | |
| 8035956 | ENST00000365097 | ncrna:misc_RNA | — | — | 80 |
| | | chromosome:NCBI36:19:38045533:38045838:−1 | | | |
| | | gene:ENSG00000201967 | | | |
| 7982868 | ENST00000249798 /// | Cation transport regulator-like | ChaC, cation | CHAC1 | 81 |
| | ENST00000397434 /// | protein 1 | transport regulator | | |
| | BC019625 /// | gene:ENSG00000128965 /// | homolog 1 (E. coli) | | |
| | NM_024111 | Cation transport protein-like | | | |
| | | protein | | | |
| | | gene:ENSG00000128965 /// | | | |
| | | Homo sapiens ChaC, cation | | | |
| | | transport regulator homolog 1 | | | |
| | | (E. coli), mRNA (cDNA clone | | | |
| | | MGC:24988 IMAGE:4473135), | | | |
| | | complete cds. /// Homo sapiens | | | |
| | | ChaC, cation transport | | | |
| | | regulator homolog 1 (E. coli) | | | |
| | | (CHAC1), mRNA. | | | |
| 8084878 | ENST00000384640 | ncrna:misc_RNA | — | — | 82 |
| | | chromosome:NCBI36:3:194824672:194824784:1 | | | |
| | | gene:ENSG00000207370 | | | |
| 7969959 | ENST00000375936 /// | Isoform 1 of D-amino acid | D-amino acid oxidase | DAOA | 83 |
| | ENST00000329625 /// | oxidase activator | activator | | |
| | DQ343761 /// | gene:ENSG00000182346 /// | | | |
| | NM_172370 | Putative uncharacterized | | | |
| | | protein DAOA | | | |
| | | gene:ENSG00000182346 /// | | | |
| | | Homo sapiens schizophrenia | | | |
| | | and bipolar disorder associated | | | |
| | | protein G72 form A mRNA, | | | |
| | | complete cds, alternatively | | | |
| | | spliced. /// Homo sapiens D- | | | |
| | | amino acid oxidase activator | | | |
| | | (DAOA), mRNA. | | | |
| 8137330 | ENST00000356058 /// | cDNA FLJ46250 fis, clone | — | — | 84 |
| | AK128129 | TESTI4021569, moderately | | | |
| | | similar to ATP-binding cassette, | | | |
| | | sub-family B, member 8, | | | |
| | | mitochondrial | | | |
| | | gene:ENSG00000197150 /// | | | |
| | | Homo sapiens cDNA FLJ46250 | | | |
| | | fis, clone TESTI4021569, | | | |
| | | moderately similar to ATP- | | | |
| | | binding cassette, sub-family B, | | | |
| | | member 8, mitochondrial | | | |
| | | precursor. | | | |
| 8160383 | ENST00000259555 /// | Interferon alpha-14 | interferon, alpha 7 /// | IFNA7 /// | 85 |
| | ENST00000380220 /// | gene:ENSG00000186809 /// | interferon, alpha 14 | IFNA14 | |
| | ENST00000239347 /// | Interferon alpha-14 | | | |
| | M34913 /// | gene:ENSG00000186809 /// | | | |
| | V00542 /// | Interferon alpha-7 | | | |
| | NM_021057 /// | gene:ENSG00000214042 /// | | | |
| | NM_002172 | Human interferon-alpha-J1 | | | |
| | | (IFN-alpha-J1) mRNA, | | | |
| | | complete cds. /// Messenger | | | |
| | | RNA for human leukocyte | | | |
| | | (alpha) interferon. /// Homo sapiens | | | |
| | | interferon, alpha 7 | | | |
| | | (IFNA7), mRNA. /// Homo sapiens | | | |
| | | interferon, alpha 14 | | | |
| | | (IFNA14), mRNA. | | | |
| 8071168 | ENST00000342005 /// | cDNA FLJ60978, weakly similar | POM121 membrane | POM121L1 /// | 86 |
| | ENST00000329949 /// | to Nuclear envelope pore | glycoprotein-like 1 | DKFZp434K191 /// | |
| | ENST00000402027 /// | membrane protein POM 121 | (rat) /// POM121 | DKFZP434P211 | |
| | ENST00000248992 /// | gene:ENSG00000182356 /// | membrane | | |
| | AK292412 /// | Putative uncharacterized | glycoprotein-like 1 | | |
| | AK302597 /// | protein ENSP00000383394 | pseudogene /// | | |
| | AY358961 /// | gene:ENSG00000217261 /// | POM121 membrane | | |
| | NR_003714 | POM121-like 1 protein | glycoprotein-like 1 | | |
| | | gene:ENSG00000183169 /// | pseudogene | | |
| | | POM121-like | | | |
| | | gene:ENSG00000128262 /// | | | |
| | | Homo sapiens cDNA FLJ76724 | | | |
| | | complete cds. /// Homo sapiens | | | |
| | | cDNA FLJ60978 complete cds, | | | |
| | | weakly similar to Nuclear | | | |
| | | envelope pore membrane | | | |
| | | protein POM 121. /// Homo sapiens | | | |
| | | clone DNA107786 | | | |
| | | POM121-like (UNQ2565) | | | |
| | | mRNA, complete cds. /// Homo sapiens | | | |
| | | POM121-like protein | | | |
| | | (DKFZP434P211), non-coding | | | |
| | | RNA. | | | |
| 8067965 | AF304443 | Homo sapiens B lymphocyte | — | — | 87 |
| | | activation-related protein BC- | | | |
| | | 2048 mRNA, complete cds. | | | |
| 8175589 | ENST00000364415 | ncrna:rRNA | — | — | 88 |
| | | chromosome:NCBI36:X:146897312:146897427:−1 | | | |
| | | gene:ENSG00000201285 | | | |
| 7935990 | ENST00000406432 /// | Isoform 1 of PH and SEC7 | pleckstrin and Sec7 | PSD | 89 |
| | ENST00000020673 /// | domain-containing protein 1 | domain containing | | |
| | BC142643 /// | gene:ENSG00000059915 /// | | | |
| | NM_002779 | Isoform 1 of PH and SEC7 | | | |
| | | domain-containing protein 1 | | | |
| | | gene:ENSG00000059915 /// | | | |
| | | Homo sapiens pleckstrin and | | | |
| | | Sec7 domain containing, mRNA | | | |
| | | (cDNA clone MGC:164849 | | | |
| | | IMAGE:40147927), complete | | | |
| | | cds. /// Homo sapiens pleckstrin | | | |
| | | and Sec7 domain containing | | | |
| | | (PSD), mRNA. | | | |
| 8080138 | ENST00000333127 /// | IQ domain-containing protein | IQ motif containing F2 | IQCF2 | 90 |
| | AK128883 /// | F2 gene:ENSG00000184345 /// | | | |
| | NM_203424 | Homo sapiens cDNA FLJ46915 | | | |
| | | fis, clone TESTI2014474. /// | | | |
| | | Homo sapiens IQ motif | | | |
| | | containing F2 (IQCF2), mRNA. | | | |
| 7946021 | ENST00000380369 /// | Putative olfactory receptor | olfactory receptor, | OR52A4 | 91 |
| | NM_001005222 | 52A4 gene:ENSG00000205494 /// | family 52, subfamily | | |
| | | Homo sapiens olfactory | A, member 4 | | |
| | | receptor, family 52, subfamily | | | |
| | | A, member 4 (OR52A4), | | | |
| | | mRNA. | | | |
| 7975779 | ENST00000303562 /// | Proto-oncogene protein c-fos | v-fos FBJ murine | FOS | 92 |
| | BX647104 /// | gene:ENSG00000170345 /// | osteosarcoma viral | | |
| | NM_005252 | Homo sapiens mRNA; cDNA | oncogene homolog | | |
| | | DKFZp686J04124 (from clone | | | |
| | | DKFZp686J04124). /// Homo sapiens | | | |
| | | v-fos FBJ murine | | | |
| | | osteosarcoma viral oncogene | | | |
| | | homolog (FOS), mRNA. | | | |
| 7912863 | ENST00000383728 /// | Hepatocyte growth factor-like | macrophage | MSTP9 /// | 93 |
| | ENST00000308124 /// | protein homolog | stimulating, | MST1 | |
| | ENST00000389184 /// | gene:ENSG00000173531 /// | pseudogene 9 /// | | |
| | ENST00000334998 /// | macrophage stimulating 1 | macrophage | | |
| | AY192149 /// | gene:ENSG00000173531 /// 64 | stimulating 1 | | |
| | L11924 /// | kDa protein | (hepatocyte growth | | |
| | NR_002729 /// | gene:ENSG00000186715 /// | factor-like) | | |
| | NM_020998 | Isoform 2 of Putative | | | |
| | | macrophage-stimulating protein | | | |
| | | MSTP9 | | | |
| | | gene:ENSG00000186715 /// | | | |
| | | Homo sapiens brain-rescue- | | | |
| | | factor-1 mRNA, complete cds. /// | | | |
| | | Homo sapiens macrophage- | | | |
| | | stimulating protein (MST1) | | | |
| | | mRNA, complete cds. /// Homo sapiens | | | |
| | | macrophage | | | |
| | | stimulating, pseudogene 9 | | | |
| | | (MSTP9), non-coding RNA. /// | | | |
| | | Homo sapiens macrophage | | | |
| | | stimulating 1 (hepatocyte | | | |
| | | growth factor-like) (MST1), | | | |
| | | mRNA. | | | |
| 8002969 | ENST00000393350 /// | Isoform Short of Transcription | v-maf | MAF | 94 |
| | ENST00000326043 /// | factor Maf | musculoaponeurotic | | |
| | BC081542 /// | gene:ENSG00000178573 /// | fibrosarcoma | | |
| | NM_005360 /// | Isoform Long of Transcription | oncogene homolog | | |
| | NM_001031804 | factor Maf | (avian) | | |
| | | gene:ENSG00000178573 /// | | | |
| | | Homo sapiens v-maf | | | |
| | | musculoaponeurotic | | | |
| | | fibrosarcoma oncogene | | | |
| | | homolog (avian), mRNA (cDNA | | | |
| | | clone MGC:71685 | | | |
| | | IMAGE:30347784), complete | | | |
| | | cds. /// Homo sapiens v-maf | | | |
| | | musculoaponeurotic | | | |
| | | fibrosarcoma oncogene | | | |
| | | homolog (avian) (MAF), | | | |
| | | transcript variant 1, mRNA. /// | | | |
| | | Homo sapiens v-maf | | | |
| | | musculoaponeurotic | | | |
| | | fibrosarcoma oncogene | | | |
| | | homolog (avian) (MAF), | | | |
| | | transcript variant 2, mRNA. | | | |
| 7943954 | ENST00000388431 /// | ncrna:scRNA_pseudogene | — | — | 95 |
| | ENST00000388445 | chromosome:NCBI36:11:112053855:112053924:−1 | | | |
| | | gene:ENSG00000211166 /// | | | |
| | | ncrna:scRNA_pseudogene | | | |
| | | chromosome:NCBI36:11:112986610:112986679:1 | | | |
| | | gene:ENSG00000211180 | | | |
| 8135099 | ENST00000397927 /// | Isoform 1 of Collagen alpha- | EMI domain | EMID2 | 96 |
| | ENST00000313669 /// | 1(XXVI) chain | containing 2 | | |
| | BC110393 /// | gene:ENSG00000160963 /// | | | |
| | NM_133457 | Isoform 2 of Collagen alpha- | | | |
| | | 1(XXVI) chain | | | |
| | | gene:ENSG00000160963 /// | | | |
| | | Homo sapiens EMI domain | | | |
| | | containing 2, mRNA (cDNA | | | |
| | | clone MGC:117329 | | | |
| | | IMAGE:5195867), complete | | | |
| | | cds. /// Homo sapiens EMI | | | |
| | | domain containing 2 (EMID2), | | | |
| | | mRNA. | | | |
| 7978801 | ENST00000399232 /// | MAM domain-containing | MAM domain | MDGA2 | 97 |
| | ENST00000399222 /// | glycosylphosphatidylinositol | containing | | |
| | ENST00000357362 /// | anchor protein 2 | glycosylphosphatidylinositol | | |
| | AY369208 /// | gene:ENSG00000139915 /// | anchor 2 | | |
| | NM_182830 /// | MAM domain containing 1 | | | |
| | NM_001113498 | isoform 2 | | | |
| | | gene:ENSG00000139915 /// | | | |
| | | MAM domain containing 1 | | | |
| | | isoform 2 | | | |
| | | gene:ENSG00000139915 /// | | | |
| | | Homo sapiens MAM domain- | | | |
| | | containing | | | |
| | | glycosylphosphatidylinositol | | | |
| | | anchor 2 (MDGA2) mRNA, | | | |
| | | complete cds. /// Homo sapiens | | | |
| | | MAM domain containing | | | |
| | | glycosylphosphatidylinositol | | | |
| | | anchor 2 (MDGA2), transcript | | | |
| | | variant 2, mRNA. /// Homo sapiens | | | |
| | | MAM domain containing | | | |
| | | glycosylphosphatidylinositol | | | |
| | | anchor 2 (MDGA2), transcript | | | |
| | | variant 1, mRNA. | | | |
| 7971661 | hsa-mir-15a /// | MI0000069 Homo sapiens miR- | — | — | 98 |
| | hsa-mir-15a | 15a stem-loop /// MI0000069 | | | |
| | | Homo sapiens miR-15a stem- | | | |
| | | loop | | | |
| 8136709 | ENST00000397504 /// | Putative maltase-glucoamylase- | maltase- | LOC93432 | 99 |
| | BC111973 /// | like protein LOC93432 | glucoamylase-like | | |
| | NR_003715 | gene:ENSG00000214088 /// | pseudogene | | |
| | | Homo sapiens maltase- | | | |
| | | glucoamylase-like pseudogene, | | | |
| | | mRNA (cDNA clone | | | |
| | | IMAGE:8327441), complete | | | |
| | | cds. /// Homo sapiens maltase- | | | |
| | | glucoamylase-like pseudogene | | | |
| | | (LOC93432), non-coding RNA. | | | |
| 8139367 | ENST00000289547 /// | Niemann-Pick C1-like protein 1 | NPC1 (Niemann-Pick | NPC1L1 | 100 |
| | ENST00000381160 /// | isoform 1 | disease, type C1, | | |
| | ENST00000381159 /// | gene:ENSG00000015520 /// | gene)-like 1 | | |
| | AF192522 /// | Isoform 2 of Niemann-Pick C1- | | | |
| | NM_013389 /// | like protein 1 | | | |
| | NM_001101648 | gene:ENSG00000015520 /// | | | |
| | | Isoform 3 of Niemann-Pick C1- | | | |
| | | like protein 1 | | | |
| | | gene:ENSG00000015520 /// | | | |
| | | Homo sapiens Niemann-Pick | | | |
| | | C1-like protein 1 (NPC1L1) | | | |
| | | mRNA, complete cds. /// Homo sapiens | | | |
| | | NPC1 (Niemann-Pick | | | |
| | | disease, type C1, gene)-like 1 | | | |
| | | (NPC1L1), transcript variant 1, | | | |
| | | mRNA. /// Homo sapiens NPC1 | | | |
| | | (Niemann-Pick disease, type | | | |
| | | C1, gene)-like 1 (NPC1L1), | | | |
| | | transcript variant 2, mRNA. | | | |
| 8055952 | ENST00000339562 /// | Nuclear receptor subfamily 4 | nuclear receptor | NR4A2 | 101 |
| | ENST00000409572 /// | group A member 2 | subfamily 4, group A, | | |
| | ENST00000409108 /// | gene:ENSG00000153234 /// | member 2 | | |
| | BC009288 /// | cdna:known | | | |
| | NM_006186 | chromosome:NCBI36:2:156889856:156907106:−1 | | | |
| | | gene:ENSG00000153234 /// | | | |
| | | cdna:known | | | |
| | | chromosome:NCBI36:2:156890502:156895465:−1 | | | |
| | | gene:ENSG00000153234 /// | | | |
| | | Homo sapiens nuclear receptor | | | |
| | | subfamily 4, group A, member | | | |
| | | 2, mRNA (cDNA clone | | | |
| | | MGC:14354 IMAGE:4298967), | | | |
| | | complete cds. /// Homo sapiens | | | |
| | | nuclear receptor subfamily 4, | | | |
| | | group A, member 2 (NR4A2), | | | |
| | | mRNA. | | | |
| 7942809 | BC008359 | Homo sapiens cDNA clone | — | — | 102 |
| | | IMAGE:3606756. | | | |
| 8120151 | ENST00000244799 /// | Opsin-5 | opsin 5 | OPN5 | 103 |
| | ENST00000393699 /// | gene:ENSG00000124818 /// | | | |
| | ENST00000371211 /// | Opsin-5 | | | |
| | ENST00000393696 /// | gene:ENSG00000124818 /// | | | |
| | BX647224 /// | opsin 5 isoform 2 | | | |
| | NM_181744 /// | gene:ENSG00000124818 /// | | | |
| | NM_001030051 | opsin 5 isoform 2 | | | |
| | | gene:ENSG00000124818 /// | | | |
| | | Homo sapiens mRNA; cDNA | | | |
| | | DKFZp686D0636 (from clone | | | |
| | | DKFZp686D0636). /// Homo sapiens | | | |
| | | opsin 5 (OPN5), | | | |
| | | transcript variant 1, mRNA. /// | | | |
| | | Homo sapiens opsin 5 (OPN5), | | | |
| | | transcript variant 2, mRNA. | | | |
| 8143710 | ENST00000385543 | ncrna:tRNA_pseudogene | — | — | 104 |
| | | chromosome:NCBI36:7:148684678:148684753:−1 | | | |
| | | gene:ENSG00000208278 | | | |
| 8091676 | ENST00000385921 /// | ncrna:rRNA_pseudogene | — | — | 105 |
| | ENST00000410743 | chromosome:NCBI36:3:158374399:158374523:−1 | | | |
| | | gene:ENSG00000208656 /// | | | |
| | | ncrna:rRNA | | | |
| | | chromosome:NCBI36:3:158374401:158374523:−1 | | | |
| | | gene:ENSG00000222675 | | | |
| 8083409 | ENST00000356517 /// | Isoform 1 of Arylacetamide | arylacetamide | AADACL2 | 106 |
| | BC065724 /// | deacetylase-like 2 | deacetylase-like 2 | | |
| | NM_207365 | gene:ENSG00000197953 /// | | | |
| | | Homo sapiens arylacetamide | | | |
| | | deacetylase-like 2, mRNA | | | |
| | | (cDNA clone MGC:72001 | | | |
| | | IMAGE:6663150), complete | | | |
| | | cds. /// Homo sapiens | | | |
| | | arylacetamide deacetylase-like | | | |
| | | 2 (AADACL2), mRNA. | | | |
| 7955119 | ENST00000380491 /// | Isoform 2 of Uncharacterized | chromosome 12 open | C12orf54 | 107 |
| | ENST00000314014 /// | protein C12orf54 | reading frame 54 | | |
| | BC031670 | gene:ENSG00000177627 /// | | | |
| | | Isoform 1 of Uncharacterized | | | |
| | | protein C12orf54 | | | |
| | | gene:ENSG00000177627 /// | | | |
| | | Homo sapiens chromosome 12 | | | |
| | | open reading frame 54, mRNA | | | |
| | | (cDNA clone MGC:35033 | | | |
| | | IMAGE:5165130), complete | | | |
| | | cds. | | | |
| 8005757 | ENST00000387268 | ncrna:Mt_tRNA_pseudogene | — | — | 108 |
| | | chromosome:NCBI36:17:21952378:21952445:1 | | | |
| | | gene:ENSG00000210003 | | | |
| 8129313 | ENST00000364509 | ncrna:snRNA | — | — | 109 |
| | | chromosome:NCBI36:6:121905331:121905471:−1 | | | |
| | | gene:ENSG00000201379 | | | |
| 8176806 | ENST00000303804 /// | Isoform 1 of PTPN13-like | PTPN13-like, Y-linked /// | PRY /// | 110 |
| | ENST00000341740 /// | protein, Y-linked | PTPN13-like, Y- | PRY2 | |
| | ENST00000338793 /// | gene:ENSG00000169807 /// | linked 2 | | |
| | ENST00000303728 /// | Isoform 2 of PTPN13-like | | | |
| | ENST00000343584 /// | protein, Y-linked | | | |
| | ENST00000303593 /// | gene:ENSG00000169807 /// | | | |
| | ENST00000306589 /// | Isoform 2 of PTPN13-like | | | |
| | ENST00000338673 /// | protein, Y-linked | | | |
| | AF517635 /// | gene:ENSG00000169789 /// | | | |
| | NM_001002758 /// | Isoform 1 of PTPN13-like | | | |
| | NM_004676 | protein, Y-linked | | | |
| | | gene:ENSG00000169789 /// | | | |
| | | Isoform 1 of PTPN13-like | | | |
| | | protein, Y-linked | | | |
| | | gene:ENSG00000169763 /// | | | |
| | | Isoform 2 of PTPN13-like | | | |
| | | protein, Y-linked | | | |
| | | gene:ENSG00000169763 /// | | | |
| | | Isoform 1 of PTPN13-like | | | |
| | | protein, Y-linked | | | |
| | | gene:ENSG00000172283 /// | | | |
| | | Isoform 2 of PTPN13-like | | | |
| | | protein, Y-linked | | | |
| | | gene:ENSG00000172283 /// | | | |
| | | Homo sapiens testis-specific | | | |
| | | PTP-BL related Y protein | | | |
| | | mRNA, complete cds, | | | |
| | | alternatively spliced. /// Homo sapiens | | | |
| | | PTPN13-like, Y-linked | | | |
| | | 2 (PRY2), mRNA. /// Homo sapiens | | | |
| | | PTPN13-like, Y-linked | | | |
| | | (PRY), mRNA. | | | |
| 8020842 | NR_003558 | Homo sapiens WW domain | WW domain binding | WBP11P1 | 111 |
| | | binding protein 11 pseudogene | protein 11 | | |
| | | 1 (WBP11P1), non-coding | pseudogene 1 | | |
| | | RNA. | | | |
| 8028389 | ENST00000396877 /// | sprouty-related, EVH1 domain | sprouty-related, EVH1 | SPRED3 | 112 |
| | ENST00000338502 /// | containing 3 isoform b | domain containing 3 | | |
| | DQ323928 /// | gene:ENSG00000188766 /// | | | |
| | NM_001039616 /// | Sprouty-related, EVH1 domain- | | | |
| | NM_001042522 | containing protein 3 | | | |
| | | gene:ENSG00000188766 /// | | | |
| | | Homo sapiens Spred3 mRNA, | | | |
| | | complete cds. /// Homo sapiens | | | |
| | | sprouty-related, EVH1 domain | | | |
| | | containing 3 (SPRED3), | | | |
| | | transcript variant 2, mRNA. /// | | | |
| | | Homo sapiens sprouty-related, | | | |
| | | EVH1 domain containing 3 | | | |
| | | (SPRED3), transcript variant 1, | | | |
| | | mRNA. | | | |
| 8007828 | ENST00000344290 /// | Isoform Tau-G of Microtubule- | microtubule- | MAPT | 113 |
| | ENST00000262410 /// | associated protein tau | associated protein tau | | |
| | ENST00000351559 /// | gene:ENSG00000186868 /// | | | |
| | ENST00000340799 /// | Isoform PNS-tau of | | | |
| | ENST00000354326 /// | Microtubule-associated protein | | | |
| | ENST00000347967 /// | tau gene:ENSG00000186868 /// | | | |
| | ENST00000334239 /// | Isoform Tau-F of | | | |
| | BC114948 /// | Microtubule-associated protein | | | |
| | NM_001123067 /// | tau gene:ENSG00000186868 /// | | | |
| | NM_016835 /// | Isoform Tau-E of | | | |
| | NM_016834 /// | Microtubule-associated protein | | | |
| | NM_016841 /// | tau gene:ENSG00000186868 /// | | | |
| | NM_005910 /// | Isoform Tau-C of | | | |
| | NM_001123066 | Microtubule-associated protein | | | |
| | | tau gene:ENSG00000186868 /// | | | |
| | | Isoform Tau-D of | | | |
| | | Microtubule-associated protein | | | |
| | | tau gene:ENSG00000186868 /// | | | |
| | | Isoform Tau-A of | | | |
| | | Microtubule-associated protein | | | |
| | | tau gene:ENSG00000186868 /// | | | |
| | | Homo sapiens microtubule- | | | |
| | | associated protein tau, mRNA | | | |
| | | (cDNA clone | | | |
| | | IMAGE:40007445), complete | | | |
| | | cds. /// Homo sapiens | | | |
| | | microtubule-associated protein | | | |
| | | tau (MAPT), transcript variant 5, | | | |
| | | mRNA. /// Homo sapiens | | | |
| | | microtubule-associated protein | | | |
| | | tau (MAPT), transcript variant 1, | | | |
| | | mRNA. /// Homo sapiens | | | |
| | | microtubule-associated protein | | | |
| | | tau (MAPT), transcript variant 3, | | | |
| | | mRNA. /// Homo sapiens | | | |
| | | microtubule-associated protein | | | |
| | | tau (MAPT), transcript variant 4, | | | |
| | | mRNA. /// Homo sapiens | | | |
| | | microtubule-associated protein | | | |
| | | tau (MAPT), transcript variant 2, | | | |
| | | mRNA. /// Homo sapiens | | | |
| | | microtubule-associated protein | | | |
| | | tau (MAPT), transcript variant 6, | | | |
| | | mRNA. | | | |
| 8058145 | — | — | — | — | 114 |
| 8055348 | ENST00000410136 | ncrna:misc_RNA | — | — | 115 |
| | | chromosome:NCBI36:2:133865128:133865440:−1 | | | |
| | | gene:ENSG00000222068 | | | |
| 7951165 | ENST00000263463 /// | Progesterone receptor, isoform | progesterone receptor | PGR | 116 |
| | ENST00000325455 /// | CRA_c | | | |
| | X51730 /// | gene:ENSG00000082175 /// | | | |
| | NM_000926 | Isoform B of Progesterone | | | |
| | | receptor | | | |
| | | gene:ENSG00000082175 /// | | | |
| | | Human mRNA and promoter | | | |
| | | DNA for progesterone receptor. /// | | | |
| | | Homo sapiens progesterone | | | |
| | | receptor (PGR), mRNA. | | | |
| 8141922 | ENST00000339444 /// | Isoform 2 of Prestin | solute carrier family | SLC26A5 | 117 |
| | ENST00000393735 /// | gene:ENSG00000170615 /// | 26, member 5 | | |
| | ENST00000356767 /// | Isoform 3 of Prestin | (prestin) | | |
| | ENST00000393730 /// | gene:ENSG00000170615 /// | | | |
| | ENST00000354356 /// | Isoform 4 of Prestin | | | |
| | ENST00000306312 /// | gene:ENSG00000170615 /// | | | |
| | ENST00000393732 /// | SLC26A5 protein | | | |
| | ENST00000393729 /// | gene:ENSG00000170615 /// | | | |
| | ENST00000393723 /// | Prestin | | | |
| | ENST00000393727 /// | gene:ENSG00000170615 /// | | | |
| | ENST00000393722 /// | Isoform 1 of Prestin | | | |
| | AF523354 /// | gene:ENSG00000170615 /// | | | |
| | NM_206884 /// | SLC26A5 protein | | | |
| | NM_198999 /// | gene:ENSG00000170615 /// | | | |
| | NM_206883 /// | SLC26A5 protein | | | |
| | NM_206885 | gene:ENSG00000170615 /// | | | |
| | | Prestin isoform SLC26A5e | | | |
| | | gene:ENSG00000170615 /// | | | |
| | | Prestin | | | |
| | | gene:ENSG00000170615 /// | | | |
| | | Putative uncharacterized | | | |
| | | protein SLC26A5 | | | |
| | | gene:ENSG00000170615 /// | | | |
| | | Homo sapiens prestin (PRES) | | | |
| | | mRNA, complete cds. /// Homo sapiens | | | |
| | | solute carrier family 26, | | | |
| | | member 5 (prestin) (SLC26A5), | | | |
| | | transcript variant c, mRNA. /// | | | |
| | | Homo sapiens solute carrier | | | |
| | | family 26, member 5 (prestin) | | | |
| | | (SLC26A5), transcript variant a, | | | |
| | | mRNA. /// Homo sapiens solute | | | |
| | | carrier family 26, member 5 | | | |
| | | (prestin) (SLC26A5), transcript | | | |
| | | variant b, mRNA. /// Homo sapiens | | | |
| | | solute carrier family 26, | | | |
| | | member 5 (prestin) (SLC26A5), | | | |
| | | transcript variant d, mRNA. | | | |
| 7934731 | ENST00000318965 /// | cdna:pseudogene | similar to | LOC642538 /// | 118 |
| | ENST00000372288 /// | chromosome:NCBI36:10:32840249:32840671:−1 | hCG1791993 /// | LOC642521 | |
| | ENST00000372287 /// | gene:ENSG00000181993 /// | similar to | | |
| | ENST00000405868 /// | cdna:pseudogene | hCG1791993 | | |
| | XM_001723653 /// | chromosome:NCBI36:10:81774473:81774898:−1 | | | |
| | XM_926017 | gene:ENSG00000204042 /// | | | |
| | | cdna:pseudogene | | | |
| | | chromosome:NCBI36:10:81781704:81782129:−1 | | | |
| | | gene:ENSG00000204041 /// | | | |
| | | cdna:pseudogene | | | |
| | | chromosome:NCBI36:10:81790363:81790779:−1 | | | |
| | | gene:ENSG00000217279 /// | | | |
| | | PREDICTED: Homo sapiens | | | |
| | | similar to hCG1791993 | | | |
| | | (LOC642538), mRNA. /// | | | |
| | | PREDICTED: Homo sapiens | | | |
| | | similar to hCG1791993 | | | |
| | | (LOC642521), mRNA. | | | |
| 7921449 | ENST00000255030 /// | Isoform 1 of C-reactive protein | C-reactive protein, | CRP | 119 |
| | ENST00000368112 /// | gene:ENSG00000132693 /// | pentraxin-related | | |
| | ENST00000368111 /// | Isoform 2 of C-reactive protein | | | |
| | ENST00000368110 /// | gene:ENSG00000132693 /// C- | | | |
| | ENST00000343919 /// | reactive protein, pentraxin- | | | |
| | AK289443 /// | related | | | |
| | NM_000567 | gene:ENSG00000132693 /// C- | | | |
| | | reactive protein, pentraxin- | | | |
| | | related | | | |
| | | gene:ENSG00000132693 /// C- | | | |
| | | reactive protein, pentraxin- | | | |
| | | related | | | |
| | | gene:ENSG00000132693 /// | | | |
| | | Homo sapiens cDNA FLJ78115 | | | |
| | | complete cds, highly similar to | | | |
| | | Homo sapiens C-reactive | | | |
| | | protein, pentraxin-related | | | |
| | | (CRP), mRNA. /// Homo sapiens | | | |
| | | C-reactive protein, | | | |
| | | pentraxin-related (CRP), | | | |
| | | mRNA. | | | |
| 8157818 | ENST00000373574 /// | WD repeat-containing protein | WD repeat domain 38 | WDR38 | 120 |
| | BC127949 /// | 38 gene:ENSG00000136918 /// | | | |
| | NM_001045476 | Homo sapiens WD repeat | | | |
| | | domain 38, mRNA (cDNA clone | | | |
| | | MGC:158102 | | | |
| | | IMAGE:40132852), complete | | | |
| | | cds. /// Homo sapiens WD | | | |
| | | repeat domain 38 (WDR38), | | | |
| | | mRNA. | | | |
| 7920264 | ENST00000359215 /// | S100 calcium binding protein | S100 calcium binding | S100A5 | 121 |
| | ENST00000368718 /// | A5 gene:ENSG00000196420 /// | protein A5 | | |
| | ENST00000368717 /// | S100 calcium binding protein | | | |
| | BC093955 /// | A5 gene:ENSG00000196420 /// | | | |
| | NM_002962 | S100 calcium binding protein | | | |
| | | A5 gene:ENSG00000196420 /// | | | |
| | | Homo sapiens S100 calcium | | | |
| | | binding protein A5, mRNA | | | |
| | | (cDNA clone MGC:120990 | | | |
| | | IMAGE:7939800), complete | | | |
| | | cds. /// Homo sapiens S100 | | | |
| | | calcium binding protein A5 | | | |
| | | (S100A5), mRNA. | | | |
| 8099362 | ENST00000411154 /// | ncrna:rRNA | — | — | 122 |
| | ENST00000387157 | chromosome:NCBI36:4:9726478:9726606:−1 | | | |
| | | gene:ENSG00000223086 /// | | | |
| | | ncrna:rRNA_pseudogene | | | |
| | | chromosome:NCBI36:4:9726517:9726606:−1 | | | |
| | | gene:ENSG00000209892 | | | |
| 8095412 | ENST00000381066 /// | Casein | casein alpha s1 | CSN1S1 | 123 |
| | ENST00000354865 /// | gene:ENSG00000126545 /// | | | |
| | ENST00000246891 /// | Isoform 3 of Alpha-S1-casein | | | |
| | BC128227 /// | gene:ENSG00000126545 /// | | | |
| | NM_001025104 /// | Isoform 1 of Alpha-S1-casein | | | |
| | NM_001890 | gene:ENSG00000126545 /// | | | |
| | | Homo sapiens casein alpha s1, | | | |
| | | mRNA (cDNA clone | | | |
| | | MGC:149367 | | | |
| | | IMAGE:40114618), complete | | | |
| | | cds. /// Homo sapiens casein | | | |
| | | alpha s1 (CSN1S1), transcript | | | |
| | | variant 2, mRNA. /// Homo sapiens | | | |
| | | casein alpha s1 | | | |
| | | (CSN1S1), transcript variant 1, | | | |
| | | mRNA. | | | |
| 8010622 | ENST00000384294 | ncrna:misc_RNA | — | — | 124 |
| | | chromosome:NCBI36:17:77150953:77151065:1 | | | |
| | | gene:ENSG00000207021 | | | |
| 8107421 | ENST00000316788 /// | AP-3 complex subunit sigma-1 | adaptor-related | AP3S1 | 125 |
| | BC012614 /// | gene:ENSG00000177879 /// | protein complex 3, | | |
| | NM_001284 | Homo sapiens adaptor-related | sigma 1 subunit | | |
| | | protein complex 3, sigma 1 | | | |
| | | subunit, mRNA (cDNA clone | | | |
| | | IMAGE:4281620). /// Homo sapiens | | | |
| | | adaptor-related protein | | | |
| | | complex 3, sigma 1 subunit | | | |
| | | (AP3S1), mRNA. | | | |
| 8126750 | ENST00000230565 /// | Ectonucleotide | ectonucleotide | ENPP5 | 126 |
| | ENST00000371383 /// | pyrophosphatase/phosphodiesterase | pyrophosphatase/ | | |
| | BX647968 /// | family member 5 | phosphodiesterase 5 | | |
| | NM_021572 | gene:ENSG00000112796 /// | (putative function) | | |
| | | Ectonucleotide | | | |
| | | pyrophosphatase/phosphodiesterase | | | |
| | | family member 5 | | | |
| | | gene:ENSG00000112796 /// | | | |
| | | Homo sapiens mRNA; cDNA | | | |
| | | DKFZp686E1552 (from clone | | | |
| | | DKFZp686E1552). /// Homo sapiens | | | |
| | | ectonucleotide | | | |
| | | pyrophosphatase/phosphodiesterase | | | |
| | | 5 (putative function) | | | |
| | | (ENPP5), mRNA. | | | |
| 8027770 | ENST00000270310 /// | FXYD domain-containing ion | FXYD domain | FXYD7 | 127 |
| | BC018619 /// | transport regulator 7 | containing ion | | |
| | NM_022006 | gene:ENSG00000126258 /// | transport regulator 7 | | |
| | | Homo sapiens FXYD domain | | | |
| | | containing ion transport | | | |
| | | regulator 7, mRNA (cDNA clone | | | |
| | | MGC:31815 IMAGE:3626060), | | | |
| | | complete cds. /// Homo sapiens | | | |
| | | FXYD domain containing ion | | | |
| | | transport regulator 7 (FXYD7), | | | |
| | | mRNA. | | | |
| 8088491 | ENST00000383710 /// | Isoform 1 of Calcium- | Ca++-dependent | CADPS | 128 |
| | ENST00000383709 /// | dependent secretion activator 1 | secretion activator | | |
| | ENST00000283269 /// | gene:ENSG00000163618 /// | | | |
| | ENST00000357948 /// | Isoform 4 of Calcium- | | | |
| | ENST00000360097 /// | dependent secretion activator 1 | | | |
| | AF458662 /// | gene:ENSG00000163618 /// | | | |
| | NM_183393 /// | Isoform 3 of Calcium- | | | |
| | NM_003716 /// | dependent secretion activator 1 | | | |
| | NM_183394 | gene:ENSG00000163618 /// | | | |
| | | Isoform 2 of Calcium- | | | |
| | | dependent secretion activator 1 | | | |
| | | gene:ENSG00000163618 /// | | | |
| | | Isoform 5 of Calcium- | | | |
| | | dependent secretion activator 1 | | | |
| | | gene:ENSG00000163618 /// | | | |
| | | Homo sapiens calcium- | | | |
| | | dependent activator protein for | | | |
| | | secretion protein mRNA, | | | |
| | | complete cds. /// Homo sapiens | | | |
| | | Ca++-dependent secretion | | | |
| | | activator (CADPS), transcript | | | |
| | | variant 3, mRNA. /// Homo sapiens | | | |
| | | Ca++-dependent | | | |
| | | secretion activator (CADPS), | | | |
| | | transcript variant 1, mRNA. /// | | | |
| | | Homo sapiens Ca++-dependent | | | |
| | | secretion activator (CADPS), | | | |
| | | transcript variant 2, mRNA. | | | |
| 8161192 | ENST00000377877 /// | Ring finger protein 38 | ring finger protein 38 | RNF38 | 129 |
| | ENST00000357058 /// | gene:ENSG00000137075 /// | | | |
| | ENST00000350199 /// | ring finger protein 38 isoform 2 | | | |
| | ENST00000377885 /// | gene:ENSG00000137075 /// | | | |
| | ENST00000377876 /// | ring finger protein 38 isoform 2 | | | |
| | ENST00000353739 /// | gene:ENSG00000137075 /// | | | |
| | ENST00000259605 /// | ring finger protein 38 isoform 2 | | | |
| | ENST00000377870 /// | gene:ENSG00000137075 /// | | | |
| | AF394047 /// | ring finger protein 38 isoform 2 | | | |
| | NM_022781 /// | gene:ENSG00000137075 /// | | | |
| | NM_194328 /// | Isoform 2 of RING finger protein | | | |
| | NM_194329 /// | 38 gene:ENSG00000137075 /// | | | |
| | NM_194330 /// | Isoform 1 of RING finger protein | | | |
| | NM_194332 | 38 gene:ENSG00000137075 /// | | | |
| | | Ring finger protein 38 | | | |
| | | gene:ENSG00000137075 /// | | | |
| | | Homo sapiens RING finger | | | |
| | | protein 38 (RNF38) mRNA, | | | |
| | | complete cds. /// Homo sapiens | | | |
| | | ring finger protein 38 (RNF38), | | | |
| | | transcript variant 1, mRNA. /// | | | |
| | | Homo sapiens ring finger | | | |
| | | protein 38 (RNF38), transcript | | | |
| | | variant 2, mRNA. /// Homo sapiens | | | |
| | | ring finger protein 38 | | | |
| | | (RNF38), transcript variant 3, | | | |
| | | mRNA. /// Homo sapiens ring | | | |
| | | finger protein 38 (RNF38), | | | |
| | | transcript variant 5, mRNA. /// | | | |
| | | Homo sapiens ring finger | | | |
| | | protein 38 (RNF38), transcript | | | |
| | | variant 6, mRNA. | | | |
| 8124634 | ENST00000404200 /// | cdna:pseudogene | — | — | 130 |
| | ENST00000401594 /// | chromosome:NCBI36:c6_COX:29249580:29250516:−1 | | | |
| | ENST00000366307 | gene:ENSG00000219452 /// | | | |
| | | cdna:pseudogene | | | |
| | | chromosome:NCBI36:c6_QBL:29248513:29249449:−1 | | | |
| | | gene:ENSG00000216296 /// | | | |
| | | cdna:pseudogene | | | |
| | | chromosome:NCBI36:6:29213626:29214572:−1 | | | |
| | | gene:ENSG00000203492 | | | |
| 8007584 | ENST00000293414 /// | Ankyrin repeat and SOCS box | ankyrin repeat and | ASB16 | 131 |
| | BC075088 /// | protein 16 | SOCS box-containing | | |
| | NM_080863 | gene:ENSG00000161664 /// | 16 | | |
| | | Homo sapiens ankyrin repeat | | | |
| | | and SOCS box-containing 16, | | | |
| | | mRNA (cDNA clone | | | |
| | | MGC:103981 | | | |
| | | IMAGE:30915388), complete | | | |
| | | cds. /// Homo sapiens ankyrin | | | |
| | | repeat and SOCS box- | | | |
| | | containing 16 (ASB16), mRNA. | | | |
| 7964660 | ENST00000299178 /// | Vasopressin V1a receptor | arginine vasopressin | AVPR1A | 132 |
| | AY322550 /// | gene:ENSG00000166148 /// | receptor 1A | | |
| | NM_000706 | Homo sapiens arginine | | | |
| | | vasopressin receptor 1A | | | |
| | | mRNA, complete cds. /// Homo sapiens | | | |
| | | arginine vasopressin | | | |
| | | receptor 1A (AVPR1 A), mRNA. | | | |
| 7980003 | ENST00000387477 | ncrna:snRNA_pseudogene | — | — | 133 |
| | | chromosome:NCBI36:14:72783233:72783340:−1 | | | |
| | | gene:ENSG00000210212 | | | |
| 8135458 | ENST00000222597 /// | E3 ubiquitin-protein ligase | Cas-Br-M (murine) | CBLL1 | 134 |
| | AK026762 /// | Hakai | ecotropic retroviral | | |
| | NM_024814 /// | gene:ENSG00000105879 /// | transforming | | |
| | NR_024199 | Homo sapiens cDNA: | sequence-like 1 | | |
| | | FLJ23109 fis, clone LNG07754. /// | | | |
| | | Homo sapiens Cas-Br-M | | | |
| | | (murine) ecotropic retroviral | | | |
| | | transforming sequence-like 1 | | | |
| | | (CBLL1), transcript variant 1, | | | |
| | | mRNA. /// Homo sapiens Cas- | | | |
| | | Br-M (murine) ecotropic | | | |
| | | retroviral transforming | | | |
| | | sequence-like 1 (CBLL1), | | | |
| | | transcript variant 2, transcribed | | | |
| | | RNA. | | | |
| 7991512 | ENST00000352519 /// | Uncharacterized protein | chromosome 15 open | C15orf51 | 135 |
| | ENST00000341853 /// | C15orf51 (Fragment) | reading frame 51 | | |
| | AK302717 /// | gene:ENSG00000182397 /// | | | |
| | NR_003260 | UPF0621 protein C15orf51 | | | |
| | | gene:ENSG00000182397 /// | | | |
| | | Homo sapiens cDNA FLJ54911 | | | |
| | | complete cds. /// Homo sapiens | | | |
| | | chromosome 15 open reading | | | |
| | | frame 51 (C15orf51), non- | | | |
| | | coding RNA. | | | |
| 8029693 | ENST00000353609 /// | Protein fosB | FBJ murine | FOSB | 136 |
| | BC036724 /// | gene:ENSG00000125740 /// | osteosarcoma viral | | |
| | NM_006732 /// | Homo sapiens FBJ murine | oncogene homolog B | | |
| | NM_001114171 | osteosarcoma viral oncogene | | | |
| | | homolog B, mRNA (cDNA clone | | | |
| | | MGC:39968 IMAGE:5212854), | | | |
| | | complete cds. /// Homo sapiens | | | |
| | | FBJ murine osteosarcoma viral | | | |
| | | oncogene homolog B (FOSB), | | | |
| | | transcript variant 1, mRNA. /// | | | |
| | | Homo sapiens FBJ murine | | | |
| | | osteosarcoma viral oncogene | | | |
| | | homolog B (FOSB), transcript | | | |
| | | variant 2, mRNA. | | | |
| 7981773 | ENST00000384559 | ncrna:snRNA | — | — | 137 |
| | | chromosome:NCBI36:15:19204036:19204142:1 | | | |
| | | gene:ENSG00000207289 | | | |
| 7934729 | GENSCAN00000036525 /// | cdna:Genscan | similar to | LOC642538 /// | 138 |
| | ENST00000318965 /// | chromosome:NCBI36:10:81732033:81798683:−1 /// | hCG1791993 /// | LOC642521 | |
| | ENST00000372288 /// | cdna:pseudogene | similar to | | |
| | ENST00000372287 /// | chromosome:NCBI36:10:32840249:32840671:−1 | hCG1791993 | | |
| | ENST00000405868 /// | gene:ENSG00000181993 /// | | | |
| | XM_001723653 /// | cdna:pseudogene | | | |
| | XM_926017 | chromosome:NCBI36:10:81774473:81774898:−1 | | | |
| | | gene:ENSG00000204042 /// | | | |
| | | cdna:pseudogene | | | |
| | | chromosome:NCBI36:10:81781704:81782129:−1 | | | |
| | | gene:ENSG00000204041 /// | | | |
| | | cdna:pseudogene | | | |
| | | chromosome:NCBI36:10:81790363:81790779:−1 | | | |
| | | gene:ENSG00000217279 /// | | | |
| | | PREDICTED: Homo sapiens | | | |
| | | similar to hCG1791993 | | | |
| | | (LOC642538), mRNA. /// | | | |
| | | PREDICTED: Homo sapiens | | | |
| | | similar to hCG1791993 | | | |
| | | (LOC642521), mRNA. | | | |
| 7936996 | ENST00000356858 /// | Novel protein | chromosome 10 open | C10orf90 | 139 |
| | ENST00000284694 /// | gene:ENSG00000154493 /// | reading frame 90 | | |
| | ENST00000368674 /// | cDNA FLJ60307 | | | |
| | ENST00000392694 /// | gene:ENSG00000154493 /// | | | |
| | AK297577 /// | Novel protein | | | |
| | NM_001004298 | gene:ENSG00000154493 /// | | | |
| | | Novel protein | | | |
| | | gene:ENSG00000154493 /// | | | |
| | | Homo sapiens cDNA FLJ60307 | | | |
| | | complete cds. /// Homo sapiens | | | |
| | | chromosome 10 open reading | | | |
| | | frame 90 (C10orf90), mRNA. | | | |
| 7906205 | ENST00000329117 /// | Isoform 1 of Brevican core | brevican | BCAN | 140 |
| | ENST00000361588 /// | protein | | | |
| | ENST00000255029 /// | gene:ENSG00000132692 /// | | | |
| | AY358372 /// | Isoform 2 of Brevican core | | | |
| | NM_021948 /// | protein | | | |
| | NM_198427 | gene:ENSG00000132692 /// | | | |
| | | Hyaluronan binding protein | | | |
| | | (Fragment) | | | |
| | | gene:ENSG00000132692 /// | | | |
| | | Homo sapiens clone | | | |
| | | DNA98565 Brevican Core Pro | | | |
| | | (UNQ2525) mRNA, complete | | | |
| | | cds. /// Homo sapiens brevican | | | |
| | | (BCAN), transcript variant 1, | | | |
| | | mRNA. /// Homo sapiens | | | |
| | | brevican (BCAN), transcript | | | |
| | | variant 2, mRNA. | | | |
| 8100990 | BC132938 /// | Homo sapiens pro-platelet | pro-platelet basic | PPBPL2 | 141 |
| | L10403 | basic protein-like 2, mRNA | protein-like 2 | | |
| | | (cDNA clone MGC:164569 | | | |
| | | IMAGE:40146960), complete | | | |
| | | cds. /// Homo sapiens DNA | | | |
| | | binding protein for surfactant | | | |
| | | protein B mRNA, complete cds. | | | |
| 8156846 | GENSCAN00000020848 /// | cdna:Genscan | — | — | 142 |
| | ENST00000409669 /// | chromosome:NCBI36:9:101107215:101108714:1 /// | | | |
| | ENST00000410082 /// | cdna:pseudogene | | | |
| | ENST00000409686 | chromosome:NCBI36:9:101107215:101108714:1 | | | |
| | | gene:ENSG00000222026 /// | | | |
| | | cdna:pseudogene | | | |
| | | chromosome:NCBI36:9:101107215:101108714:1 | | | |
| | | gene:ENSG00000222034 /// | | | |
| | | cdna:pseudogene | | | |
| | | chromosome:NCBI36:9:101107215:101108714:1 | | | |
| | | gene:ENSG00000222039 | | | |
| 8044563 | ENST00000341010 /// | Isoform 1 of Interleukin-1 family | interleukin 1 family, | IL1F10 | 143 |
| | ENST00000337569 /// | member 10 | member 10 (theta) | | |
| | ENST00000393197 /// | gene:ENSG00000136697 /// | | | |
| | AY029413 /// | Isoform 2 of Interleukin-1 family | | | |
| | NM_032556 /// | member 10 | | | |
| | NM_173161 | gene:ENSG00000136697 /// | | | |
| | | Isoform 1 of Interleukin-1 family | | | |
| | | member 10 | | | |
| | | gene:ENSG00000136697 /// | | | |
| | | Homo sapiens interleukin-1 | | | |
| | | receptor antagonist-like FIL1 | | | |
| | | theta (FIL1-theta) mRNA, | | | |
| | | complete cds. /// Homo sapiens | | | |
| | | interleukin 1 family, member 10 | | | |
| | | (theta) (IL1F10), transcript | | | |
| | | variant 1, mRNA. /// Homo sapiens | | | |
| | | interleukin 1 family, | | | |
| | | member 10 (theta) (IL1F10), | | | |
| | | transcript variant 2, mRNA. | | | |
| 7932964 | ENST00000355848 /// | Nuclear nucleic acid-binding | nuclear DNA-binding | C1D | 144 |
| | ENST00000410067 /// | protein C1D | protein | | |
| | BC005235 /// | gene:ENSG00000197223 /// | | | |
| | NM_006333 /// | cdna:known | | | |
| | NM_173177 | chromosome:NCBI36:2:68123292:68143661:−1 | | | |
| | | gene:ENSG00000197223 /// | | | |
| | | Homo sapiens nuclear DNA- | | | |
| | | binding protein, mRNA (cDNA | | | |
| | | clone MGC:12261 | | | |
| | | IMAGE:3930648), complete | | | |
| | | cds. /// Homo sapiens nuclear | | | |
| | | DNA-binding protein (C1D), | | | |
| | | transcript variant 1, mRNA. /// | | | |
| | | Homo sapiens nuclear DNA- | | | |
| | | binding protein (C1D), transcript | | | |
| | | variant 2, mRNA. | | | |
| 7912606 | ENST00000361079 /// | PRAME family member 7 | PRAME family | PRAMEF7 /// | 145 |
| | ENST00000330881 /// | gene:ENSG00000204510 /// | member 7 /// PRAME | PRAMEF8 | |
| | ENST00000357367 /// | PRAME family member 7 | family member 8 | | |
| | NM_001012277 /// | gene:ENSG00000204510 /// | | | |
| | NM_001012276 | PRAME family member 8 | | | |
| | | gene:ENSG00000182330 /// | | | |
| | | Homo sapiens PRAME family | | | |
| | | member 7 (PRAMEF7), mRNA. /// | | | |
| | | Homo sapiens PRAME | | | |
| | | family member 8 (PRAMEF8), | | | |
| | | mRNA. | | | |
| 8099713 | ENST00000387283 | ncrna:Mt_tRNA_pseudogene | — | — | 146 |
| | | chromosome:NCBI36:4:25328670:25328727:−1 | | | |
| | | gene:ENSG00000210018 | | | |
| 8139723 | ENST00000324256 /// | Putative FK506-binding protein | FK506 binding protein | FKBP9L | 147 |
| | BC011872 /// | 9-like protein | 9-like | | |
| | NR_003949 | gene:ENSG00000176826 /// | | | |
| | | Homo sapiens FK506 binding | | | |
| | | protein 9-like, mRNA (cDNA | | | |
| | | clone MGC:20531 | | | |
| | | IMAGE:3028515), complete | | | |
| | | cds. /// Homo sapiens FK506 | | | |
| | | binding protein 9-like (FKBP9L), | | | |
| | | non-coding RNA. | | | |
| 8109159 | hsa-mir-145 /// | MI0000461 Homo sapiens miR- | hypothetical protein | LOC728264 | 148 |
| | hsa-mir-145 /// | 145 stem-loop /// M10000461 | LOC728264 | | |
| | AK093957 | Homo sapiens miR-145 stem- | | | |
| | | loop /// Homo sapiens cDNA | | | |
| | | FLJ36638 fis, clone | | | |
| | | TRACH2018950. | | | |
| 7906197 | ENST00000255039 /// | Hyaluronan and proteoglycan | hyaluronan and | HAPLN2 | 149 |
| | AB049054 /// | link protein 2 | proteoglycan link | | |
| | NM_021817 | gene:ENSG00000132702 /// | protein 2 | | |
| | | Homo sapiens BRAL1 mRNA | | | |
| | | for brain link protein-1, | | | |
| | | complete cds. /// Homo sapiens | | | |
| | | hyaluronan and proteoglycan | | | |
| | | link protein 2 (HAPLN2), | | | |
| | | mRNA. | | | |
| 7912591 | ENST00000361079 /// | PRAME family member 7 | PRAME family | PRAMEF7 /// | 150 |
| | ENST00000330881 /// | gene:ENSG00000204510 /// | member 7 /// PRAME | PRAMEF8 | |
| | ENST00000357367 /// | PRAME family member 7 | family member 8 | | |
| | NM_001012277 /// | gene:ENSG00000204510 /// | | | |
| | NM_001012276 | PRAME family member 8 | | | |
| | | gene:ENSG00000182330 /// | | | |
| | | Homo sapiens PRAME family | | | |
| | | member 7 (PRAMEF7), mRNA. /// | | | |
| | | Homo sapiens PRAME | | | |
| | | family member 8 (PRAMEF8), | | | |
| | | mRNA. | | | |
| 7986291 | AY358254 | Homo sapiens clone | IFMQ9370 | UNQ9370 | 151 |
| | | DNA172197 IFMQ9370 | | | |
| | | (UNQ9370) mRNA, complete | | | |
| | | cds. | | | |
| 7925504 | ENST00000357246 /// | Microtubule-associated proteins | microtubule- | MAP1LC3C | 152 |
| | BC132986 /// | 1A/1B light chain 3C | associated protein 1 | | |
| | NM_001004343 | gene:ENSG00000197769 /// | light chain 3 gamma | | |
| | | Homo sapiens microtubule- | | | |
| | | associated protein 1 light chain | | | |
| | | 3 gamma, mRNA (cDNA clone | | | |
| | | MGC:164617 | | | |
| | | IMAGE:40147008), complete | | | |
| | | cds. /// Homo sapiens | | | |
| | | microtubule-associated protein | | | |
| | | 1 light chain 3 gamma | | | |
| | | (MAP1LC3C), mRNA. | | | |
| 7897991 | ENST00000361079 /// | PRAME family member 7 | PRAME family | PRAMEF7 /// | 153 |
| | ENST00000330881 /// | gene:ENSG00000204510 /// | member 7 /// PRAME | PRAMEF8 | |
| | ENST00000357367 /// | PRAME family member 7 | family member 8 | | |
| | NM_001012277 /// | gene:ENSG00000204510 /// | | | |
| | NM_001012276 | PRAME family member 8 | | | |
| | | gene:ENSG00000182330 /// | | | |
| | | Homo sapiens PRAME family | | | |
| | | member 7 (PRAMEF7), mRNA. /// | | | |
| | | Homo sapiens PRAME | | | |
| | | family member 8 (PRAMEF8), | | | |
| | | mRNA. | | | |
| 7972461 | ENST00000376503 /// | Solute carrier family 15 member | solute carrier family | SLC15A1 | 154 |
| | ENST00000313260 /// | 1 gene:ENSG00000088386 /// | 15 (oligopeptide | | |
| | ENST00000376494 /// | pH-sensing regulatory factor of | transporter), member | | |
| | U21936 /// | peptide transporter | 1 | | |
| | NM_005073 | gene:ENSG00000088386 /// | | | |
| | | Solute carrier family 15 | | | |
| | | (Oligopeptide transporter), | | | |
| | | member 1 | | | |
| | | gene:ENSG00000088386 /// | | | |
| | | Human peptide transporter | | | |
| | | (HPEPT1) mRNA, complete | | | |
| | | cds. /// Homo sapiens solute | | | |
| | | carrier family 15 (oligopeptide | | | |
| | | transporter), member 1 | | | |
| | | (SLC15A1), mRNA. | | | |
| 8146857 | ENST00000388545 | ncrna:rRNA_pseudogene | — | — | 155 |
| | | chromosome:NCBI36:8:69770442:69770521:1 | | | |
| | | gene:ENSG00000211280 | | | |
| 7925846 | AF220183 /// | Homo sapiens uncharacterized | chromosome 10 open | C10orf110 | 156 |
| | BC104155 | hypothalamus protein HT009 | reading frame 110 | | |
| | | mRNA, complete cds. /// Homo sapiens | | | |
| | | chromosome 10 open | | | |
| | | reading frame 110, mRNA | | | |
| | | (cDNA clone | | | |
| | | IMAGE:40029412), complete | | | |
| | | cds. | | | |
| 8059026 | hsa-mir-375 /// | MI0000783 Homo sapiens miR- | — | — | 157 |
| | hsa-mir-375 | 375 stem-loop /// MI0000783 | | | |
| | | Homo sapiens miR-375 stem- | | | |
| | | loop | | | |
| 8071274 | L20860 | Human glycoprotein Ib beta | glycoprotein Ib | GP1BB | 158 |
| | | mRNA, complete cds. | (platelet), beta | | |
| | | | polypeptide | | |
| 8050113 | AK125905 | Homo sapiens cDNA FLJ43917 | hypothetical | LOC100129581 | 159 |
| | | fis, clone TESTI4011505. | LOC100129581 | | |
| 8170159 | ENST00000370648 /// | Bombesin receptor subtype-3 | bombesin-like | BRS3 | 160 |
| | L08893 /// | gene:ENSG00000102239 /// | receptor 3 | | |
| | NM_001727 | Human bombesin receptor | | | |
| | | subtype-3 mRNA, complete | | | |
| | | cds. /// Homo sapiens | | | |
| | | bombesin-like receptor 3 | | | |
| | | (BRS3), mRNA. | | | |
| 7958711 | ENST00000308208 /// | Coiled-coil domain-containing | coiled-coil domain | CCDC63 | 161 |
| | BC044815 /// | protein 63 | containing 63 | | |
| | NM_152591 | gene:ENSG00000173093 /// | | | |
| | | Homo sapiens coiled-coil | | | |
| | | domain containing 63, mRNA | | | |
| | | (cDNA clone IMAGE:5166270), | | | |
| | | complete cds. /// Homo sapiens | | | |
| | | coiled-coil domain containing | | | |
| | | 63 (CCDC63), mRNA. | | | |
| 8021357 | ENST00000262095 /// | one cut domain, family member | one cut homeobox 2 | ONECUT2 | 162 |
| | NM_004852 | 2 gene:ENSG00000119547 /// | | | |
| | | Homo sapiens one cut | | | |
| | | homeobox 2 (ONECUT2), | | | |
| | | mRNA. | | | |
| 8095161 | ENST00000387825 | ncrna:snRNA_pseudogene | — | — | 163 |
| | | chromosome:NCBI36:4:56359561:56359653:1 | | | |
| | | gene:ENSG00000210560 | | | |
| 7960861 | ENST00000364793 | ncrna:misc_RNA | — | — | 164 |
| | | chromosome:NCBI36:12:7894580:7894681:−1 | | | |
| | | gene:ENSG00000201663 | | | |
| 8127932 | ENST00000330469 /// | TBX18 protein (Fragment) | T-box 18 | TBX18 | 165 |
| | ENST00000369663 /// | gene:ENSG00000112837 /// T- | | | |
| | BC132715 /// | box transcription factor TBX18 | | | |
| | NM_001080508 | gene:ENSG00000112837 /// | | | |
| | | Homo sapiens T-box 18, mRNA | | | |
| | | (cDNA clone MGC:164346 | | | |
| | | IMAGE:40146737), complete | | | |
| | | cds. /// Homo sapiens T-box 18 | | | |
| | | (TBX18), mRNA. | | | |
| 8116607 | ENST00000399551 /// | hypothetical protein | hypothetical | DKFZP686I15217 | 166 |
| | AK123663 /// | gene:ENSG00000215076 /// | LOC401232 | | |
| | BC108683 | Homo sapiens cDNA FLJ41669 | | | |
| | | fis, clone FEBRA2028618. /// | | | |
| | | Homo sapiens cDNA clone | | | |
| | | IMAGE:5212284. | | | |
| 8164766 | ENST00000298545 /// | Isoform 1 of Putative adenylate | chromosome 9 open | C9orf98 | 167 |
| | BC050576 /// | kinase-like protein C9orf98 | reading frame 98 | | |
| | NM_152572 | gene:ENSG00000165695 /// | | | |
| | | Homo sapiens chromosome 9 | | | |
| | | open reading frame 98, mRNA | | | |
| | | (cDNA clone MGC:57797 | | | |
| | | IMAGE:5744517), complete | | | |
| | | cds. /// Homo sapiens | | | |
| | | chromosome 9 open reading | | | |
| | | frame 98 (C9orf98), mRNA. | | | |
| 8012726 | ENST00000379814 /// | MYH1 protein (Fragment) | myosin, heavy chain | MYH1 | 168 |
| | ENST00000226207 /// | gene:ENSG00000109061 /// | 1, skeletal muscle, | | |
| | AF111785 /// | Myosin-1 | adult | | |
| | NM_005963 | gene:ENSG00000109061 /// | | | |
| | | Homo sapiens myosin heavy | | | |
| | | chain IIx/d mRNA, complete | | | |
| | | cds. /// Homo sapiens myosin, | | | |
| | | heavy chain 1, skeletal muscle, | | | |
| | | adult (MYH1), mRNA. | | | |
| 7906552 | ENST00000368078 /// | Calsequestrin | calsequestrin 1 (fast- | CASQ1 | 169 |
| | ENST00000368079 /// | gene:ENSG00000143318 /// | twitch, skeletal | | |
| | BC022289 /// | Calsequestrin-1 | muscle) | | |
| | NM_001231 | gene:ENSG00000143318 /// | | | |
| | | Homo sapiens calsequestrin 1 | | | |
| | | (fast-twitch, skeletal muscle), | | | |
| | | mRNA (cDNA clone | | | |
| | | MGC:22462 IMAGE:4338020), | | | |
| | | complete cds. /// Homo sapiens | | | |
| | | calsequestrin 1 (fast-twitch, | | | |
| | | skeletal muscle) (CASQ1), | | | |
| | | nuclear gene encoding | | | |
| | | mitochondrial protein, mRNA. | | | |
| 8115831 | ENST00000239223 /// | Dual specificity protein | dual specificity | DUSP1 | 170 |
| | BC022463 /// | phosphatase 1 | phosphatase 1 | | |
| | NM_004417 | gene:ENSG00000120129 /// | | | |
| | | Homo sapiens dual specificity | | | |
| | | phosphatase 1, mRNA (cDNA | | | |
| | | clone MGC:26153 | | | |
| | | IMAGE:4794895), complete | | | |
| | | cds. /// Homo sapiens dual | | | |
| | | specificity phosphatase 1 | | | |
| | | (DUSP1), mRNA. | | | |
| 8143708 | AK125575 | Homo sapiens cDNA FLJ43587 | — | — | 171 |
| | | fis, clone SKNMC2009450. | | | |
| 8036430 | ENST00000358582 /// | Isoform 2 of Zinc finger protein | zinc finger protein 781 | ZNF781 | 172 |
| | BC108687 /// | 781 gene:ENSG00000196381 /// | | | |
| | NM_152605 | Homo sapiens zinc finger | | | |
| | | protein 781, mRNA (cDNA | | | |
| | | clone MGC:131783 | | | |
| | | IMAGE:6148649), complete | | | |
| | | cds. /// Homo sapiens zinc | | | |
| | | finger protein 781 (ZNF781), | | | |
| | | mRNA. | | | |
| 7937975 | ENST00000354690 | cdna:pseudogene | — | — | 173 |
| | | chromosome:NCBI36:11:4809975:4810860:1 | | | |
| | | gene:ENSG00000197984 | | | |
| 7973054 | ENST00000363355 | ncrna:rRNA | — | — | 174 |
| | | chromosome:NCBI36:14:19952986:19953103:1 | | | |
| | | gene:ENSG00000200225 | | | |
| 8052698 | ENST00000355848 /// | Nuclear nucleic acid-binding | nuclear DNA-binding | C1D | 175 |
| | ENST00000407324 /// | protein C1D | protein | | |
| | ENST00000410067 /// | gene:ENSG00000197223 /// 20 | | | |
| | ENST00000409302 /// | kDa protein | | | |
| | BC005235 /// | gene:ENSG00000197223 /// | | | |
| | NM_006333 /// | cdna:known | | | |
| | NM_173177 | chromosome:NCBI36:2:68123292:68143661:−1 | | | |
| | | gene:ENSG00000197223 /// | | | |
| | | cdna:known | | | |
| | | chromosome:NCBI36:2:68123313:68143645:−1 | | | |
| | | gene:ENSG00000197223 /// | | | |
| | | Homo sapiens nuclear DNA- | | | |
| | | binding protein, mRNA (cDNA | | | |
| | | clone MGC:12261 | | | |
| | | IMAGE:3930648), complete | | | |
| | | cds. /// Homo sapiens nuclear | | | |
| | | DNA-binding protein (C1D), | | | |
| | | transcript variant 1, mRNA. /// | | | |
| | | Homo sapiens nuclear DNA- | | | |
| | | binding protein (C1D), transcript | | | |
| | | variant 2, mRNA. | | | |
| 8113352 | ENST00000388656 | ncrna:Mt_tRNA_pseudogene | — | — | 176 |
| | | chromosome:NCBI36:5:99414667:99414734:−1 | | | |
| | | gene:ENSG00000211391 | | | |
| 8077499 | AF086709 /// | Homo sapiens NAG-7 protein | loss of | LOH3CR2A | 177 |
| | AK054898 /// | (NAG-7) mRNA, complete cds. /// | heterozygosity, 3, | | |
| | BC016278 | Homo sapiens cDNA | chromosomal region | | |
| | | FLJ30336 fis, clone | 2, gene A | | |
| | | BRACE2007358, moderately | | | |
| | | similar to Homo sapiens NAG-7 | | | |
| | | protein (NAG-7) mRNA. /// | | | |
| | | Homo sapiens loss of | | | |
| | | heterozygosity, 3, chromosomal | | | |
| | | region 2, gene A, mRNA (cDNA | | | |
| | | clone MGC:8781 | | | |
| | | IMAGE:3915957), complete | | | |
| | | cds. | | | |
| 8104074 | ENST00000307161 /// | Melatonin receptor type 1A | melatonin receptor 1A | MTNR1A | 178 |
| | BC126297 /// | gene:ENSG00000168412 /// | | | |
| | NM_005958 | Homo sapiens melatonin | | | |
| | | receptor 1A, mRNA (cDNA | | | |
| | | clone MGC:161575 | | | |
| | | IMAGE:8992013), complete | | | |
| | | cds. /// Homo sapiens | | | |
| | | melatonin receptor 1A | | | |
| | | (MTNR1A), mRNA. | | | |
| 7951701 | ENST00000388431 /// | ncrna:scRNA_pseudogene | — | — | 179 |
| | ENST00000388445 | chromosome:NCBI36:11:112053855:112053924:−1 | | | |
| | | gene:ENSG00000211166 /// | | | |
| | | ncrna:scRNA_pseudogene | | | |
| | | chromosome:NCBI36:11:112986610:112986679:1 | | | |
| | | gene:ENSG00000211180 | | | |
| 8157092 | ENST00000374692 /// | Trimeric intracellular cation | transmembrane | TMEM38B | 180 |
| | ENST00000374689 /// | channel type B | protein 38B | | |
| | ENST00000374688 /// | gene:ENSG00000095209 /// | | | |
| | BC000049 /// | Putative uncharacterized | | | |
| | NM_018112 | protein TMEM38B | | | |
| | | gene:ENSG00000095209 /// 26 | | | |
| | | kDa protein | | | |
| | | gene:ENSG00000095209 /// | | | |
| | | Homo sapiens transmembrane | | | |
| | | protein 38B, mRNA (cDNA | | | |
| | | clone MGC:960 | | | |
| | | IMAGE:3506969), complete | | | |
| | | cds. /// Homo sapiens | | | |
| | | transmembrane protein 38B | | | |
| | | (TMEM38B), mRNA. | | | |
| 7942814 | ENST00000387816 | ncrna:Mt_tRNA_pseudogene | — | — | 181 |
| | | chromosome:NCBI36:11:80940435:80940502:1 | | | |
| | | gene:ENSG00000210551 | | | |
| 8090366 | ENST00000290868 /// | Probable urocanate hydratase | urocanase domain | UROC1 | 182 |
| | ENST00000383579 /// | gene:ENSG00000159650 /// | containing 1 | | |
| | BC115405 /// | Urocanase family protein | | | |
| | NM_144639 | gene:ENSG00000159650 /// | | | |
| | | Homo sapiens urocanase | | | |
| | | domain containing 1, mRNA | | | |
| | | (cDNA clone MGC:135007 | | | |
| | | IMAGE:40076084), complete | | | |
| | | cds. /// Homo sapiens | | | |
| | | urocanase domain containing 1 | | | |
| | | (UROC1), mRNA. | | | |
| 8036300 | ENST00000363309 | ncrna:misc_RNA | — | — | 183 |
| | | chromosome:NCBI36:19:41386089:41386201:−1 | | | |
| | | gene:ENSG00000200179 | | | |
| 8036403 | ENST00000316807 | Putative uncharacterized | — | — | 184 |
| | | protein LOC400692 | | | |
| | | gene:ENSG00000180458 | | | |
| 7971644 | ENST00000378195 /// | Isoform 2 of Chronic | chromosome 13 open | C13orf1 | 185 |
| | ENST00000361840 /// | lymphocytic leukemia deletion | reading frame 1 | | |
| | AF334405 /// | region gene 6 protein | | | |
| | NM_020456 /// | gene:ENSG00000123178 /// | | | |
| | NM_001127482 /// | Isoform 1 of Chronic | | | |
| | NR_023351 | lymphocytic leukemia deletion | | | |
| | | region gene 6 protein | | | |
| | | gene:ENSG00000123178 /// | | | |
| | | Homo sapiens CLLL6 protein | | | |
| | | (CLLD6) mRNA, complete cds. /// | | | |
| | | Homo sapiens chromosome | | | |
| | | 13 open reading frame 1 | | | |
| | | (C13orf1), transcript variant 1, | | | |
| | | mRNA. /// Homo sapiens | | | |
| | | chromosome 13 open reading | | | |
| | | frame 1 (C13orf1), transcript | | | |
| | | variant 2, mRNA. /// Homo sapiens | | | |
| | | chromosome 13 open | | | |
| | | reading frame 1 (C13orf1), | | | |
| | | transcript variant 3, transcribed | | | |
| | | RNA. | | | |
| 8157216 | ENST00000374279 /// | Ceramide glucosyltransferase | UDP-glucose | UGCG | 186 |
| | BC038711 /// | gene:ENSG00000148154 /// | ceramide | | |
| | NM_003358 | Homo sapiens UDP-glucose | glucosyltransferase | | |
| | | ceramide glucosyltransferase, | | | |
| | | mRNA (cDNA clone | | | |
| | | MGC:33797 IMAGE:5295561), | | | |
| | | complete cds. /// Homo sapiens | | | |
| | | UDP-glucose ceramide | | | |
| | | glucosyltransferase (UGCG), | | | |
| | | mRNA. | | | |
| 8074714 | ENST00000342005 /// | cDNA FLJ60978, weakly similar | POM121 membrane | POM121L1 /// | 187 |
| | ENST00000329949 /// | to Nuclear envelope pore | glycoprotein-like 1 | DKFZp434K191 /// | |
| | ENST00000402027 /// | membrane protein POM 121 | (rat) /// POM 121 | DKFZP434P211 | |
| | ENST00000248992 /// | gene:ENSG00000182356 /// | membrane | | |
| | AK292412 /// | Putative uncharacterized | glycoprotein-like 1 | | |
| | AK302597 /// | protein ENSP00000383394 | pseudogene /// | | |
| | AY358961 /// | gene:ENSG00000217261 /// | POM121 membrane | | |
| | NR_003714 | POM121-like 1 protein | glycoprotein-like 1 | | |
| | | gene:ENSG00000183169 /// | pseudogene | | |
| | | POM121-like | | | |
| | | gene:ENSG00000128262 /// | | | |
| | | Homo sapiens cDNA FLJ76724 | | | |
| | | complete cds. /// Homo sapiens | | | |
| | | cDNA FLJ60978 complete cds, | | | |
| | | weakly similar to Nuclear | | | |
| | | envelope pore membrane | | | |
| | | protein POM 121. /// Homo sapiens | | | |
| | | clone DNA107786 | | | |
| | | POM121-like (UNQ2565) | | | |
| | | mRNA, complete cds. /// Homo sapiens | | | |
| | | POM121-like protein | | | |
| | | (DKFZP434P211), non-coding | | | |
| | | RNA. | | | |
| 8065758 | AK096092 | Homo sapiens cDNA FLJ38773 | hypothetical protein | FLJ38773 | 188 |
| | | fis, clone KIDNE2018071. | FLJ38773 | | |
| 8049530 | ENST00000308482 /// | leucine rich repeat (in FLII) | leucine rich repeat (in | LRRFIP1 | 189 |
| | NM_001137550 | interacting protein 1 isoform 1 | FLII) interacting | | |
| | | gene:ENSG00000124831 /// | protein 1 | | |
| | | Homo sapiens leucine rich | | | |
| | | repeat (in FLII) interacting | | | |
| | | protein 1 (LRRFIP1), transcript | | | |
| | | variant 1, mRNA. | | | |
| 7906475 | ENST00000321935 /// | Isoform 2 of Fc receptor-like | Fc receptor-like 6 | FCRL6 | 190 |
| | ENST00000368106 /// | protein 6 | | | |
| | ENST00000339348 /// | gene:ENSG00000181036 /// | | | |
| | ENST00000392235 /// | Isoform 1 of Fc receptor-like | | | |
| | AK131201 /// | protein 6 | | | |
| | NM_001004310 | gene:ENSG00000181036 /// | | | |
| | | Isoform 3 of Fc receptor-like | | | |
| | | protein 6 | | | |
| | | gene:ENSG00000181036 /// | | | |
| | | Isoform 4 of Fc receptor-like | | | |
| | | protein 6 | | | |
| | | gene:ENSG00000181036 /// | | | |
| | | Homo sapiens cDNA FLJ16056 | | | |
| | | fis, clone SPLEN2010588, | | | |
| | | weakly similar to CELL | | | |
| | | SURFACE GLYCOPROTEIN | | | |
| | | GP42 PRECURSOR. /// Homo sapiens | | | |
| | | Fc receptor-like 6 | | | |
| | | (FCRL6), mRNA. | | | |
| 7989476 | ENST00000304813 /// | cdna:known | hypothetical | FLJ38723 | 191 |
| | ENST00000299125 /// | chromosome:NCBI36:15:60322074:60323649:−1 | FLJ38723 | | |
| | AK096042 | gene:ENSG00000220356 /// | | | |
| | | Isoform 1 of Golgin subfamily A | | | |
| | | member 2-like protein 4 | | | |
| | | gene:ENSG00000166104 /// | | | |
| | | Homo sapiens cDNA FLJ38723 | | | |
| | | fis, clone KIDNE2010137, | | | |
| | | weakly similar to GOLGIN-95. | | | |
| 8103722 | ENST00000325407 /// | Heat shock protein 90Af | heat shock protein | HSP90AA6P | 192 |
| | AY956762 | gene:ENSG00000181359 /// | 90 kDa alpha | | |
| | | Homo sapiens heat shock | (cytosolic), class A | | |
| | | protein 90Af (HSP90Af) mRNA, | member 6 | | |
| | | complete cds. | (pseudogene) | | |
| 8035146 | ENST00000409035 /// | cdna:known | calreticulin 3 | CALR3 | 193 |
| | ENST00000269881 /// | chromosome:NCBI36:19:16450878:16600015:−1 | | | |
| | BC014595 /// | gene:ENSG00000141979 /// | | | |
| | NM_145046 | Calreticulin-3 | | | |
| | | gene:ENSG00000141979 /// | | | |
| | | Homo sapiens calreticulin 3, | | | |
| | | mRNA (cDNA clone | | | |
| | | MGC:26577 IMAGE:4822010), | | | |
| | | complete cds. /// Homo sapiens | | | |
| | | calreticulin 3 (CALR3), mRNA. | | | |
| 8150722 | ENST00000276480 /// | Suppression of tumorigenicity | suppression of | ST18 | 194 |
| | AB011107 /// | protein 18 | tumorigenicity 18 | | |
| | NM_014682 | gene:ENSG00000147488 /// | (breast carcinoma) | | |
| | | Homo sapiens mRNA for | (zinc finger protein) | | |
| | | KIAA0535 protein, complete | | | |
| | | cds. /// Homo sapiens | | | |
| | | suppression of tumorigenicity | | | |
| | | 18 (breast carcinoma) (zinc | | | |
| | | finger protein) (ST18), mRNA. | | | |
| 8053715 | GENSCAN00000025928 /// | cdna:Genscan | — | — | 195 |
| | ENST00000312946 /// | chromosome:NCBI36:2:89209283:89223706:−1 /// | | | |
| | ENST00000402897 | cdna:pseudogene | | | |
| | | chromosome:NCBI36:2:89209304:89210080:−1 | | | |
| | | gene:ENSG00000204732 /// | | | |
| | | cdna:pseudogene | | | |
| | | chromosome:NCBI36:2:89728732:89729523:1 | | | |
| | | gene:ENSG00000218041 | | | |
| 7922976 | ENST00000367468 /// | Prostaglandin G/H synthase 2 | prostaglandin- | PTGS2 | 196 |
| | AY151286 /// | gene:ENSG00000073756 /// | endoperoxide | | |
| | NM_000963 | Homo sapiens cyclooxygenase | synthase 2 | | |
| | | 2b mRNA, complete cds; | (prostaglandin G/H | | |
| | | alternatively spliced. /// Homo sapiens | synthase and | | |
| | | prostaglandin- | cyclooxygenase) | | |
| | | endoperoxide synthase 2 | | | |
| | | (prostaglandin G/H synthase | | | |
| | | and cyclooxygenase) (PTGS2), | | | |
| | | mRNA. | | | |
| 8087433 | ENST00000273588 /// | Aminomethyltransferase, | nicolin 1 /// | NICN1 /// | 197 |
| | ENST00000395338 /// | mitochondrial | aminomethyltransferase | AMT | |
| | ENST00000273598 /// | gene:ENSG00000145020 /// | | | |
| | AF538150 /// | Aminomethyltransferase | | | |
| | D13811 /// | gene:ENSG00000145020 /// | | | |
| | NM_000481 /// | Isoform 1 of Nicolin-1 | | | |
| | NM_032316 | gene:ENSG00000145029 /// | | | |
| | | Homo sapiens NPCEDRGP | | | |
| | | (NPCEDRG) mRNA, | | | |
| | | NPCEDRG-s allele, complete | | | |
| | | cds. /// Homo sapiens mRNA | | | |
| | | for glycine cleavage system T- | | | |
| | | protein, complete cds. /// Homo sapiens | | | |
| | | aminomethyltransferase (AMT), | | | |
| | | mRNA. /// Homo sapiens nicolin | | | |
| | | 1 (NICN1), mRNA. | | | |
| 8075142 | ENST00000397906 /// | Tetratricopeptide repeat protein | tetratricopeptide | TTC28 | 198 |
| | ENST00000266082 /// | 28 gene:ENSG00000100154 /// | repeat domain 28 | | |
| | AB028966 /// | Tetratricopeptide repeat protein | | | |
| | BC016465 | 28 gene:ENSG00000100154 /// | | | |
| | | Homo sapiens mRNA for | | | |
| | | KIAA1043 protein, partial cds. /// | | | |
| | | Homo sapiens | | | |
| | | tetratricopeptide repeat domain | | | |
| | | 28, mRNA (cDNA clone | | | |
| | | MGC:18145 IMAGE:4154050), | | | |
| | | complete cds. | | | |
| 7919751 | ENST00000307940 /// | Isoform 2 of Induced myeloid | myeloid cell leukemia | MCL1 | 199 |
| | ENST00000369026 /// | leukemia cell differentiation | sequence 1 (BCL2- | | |
| | BC017197 /// | protein Mcl-1 | related) | | |
| | NM_182763 /// | gene:ENSG00000143384 /// | | | |
| | NM_021960 | Isoform 1 of Induced myeloid | | | |
| | | leukemia cell differentiation | | | |
| | | protein Mcl-1 | | | |
| | | gene:ENSG00000143384 /// | | | |
| | | Homo sapiens myeloid cell | | | |
| | | leukemia sequence 1 (BCL2- | | | |
| | | related), mRNA (cDNA clone | | | |
| | | MGC:1839 IMAGE:3138465), | | | |
| | | complete cds. /// Homo sapiens | | | |
| | | myeloid cell leukemia sequence | | | |
| | | 1 (BCL2-related) (MCL1), | | | |
| | | transcript variant 2, mRNA. /// | | | |
| | | Homo sapiens myeloid cell | | | |
| | | leukemia sequence 1 (BCL2- | | | |
| | | related) (MCL1), transcript | | | |
| | | variant 1, mRNA. | | | |
| 8000590 | ENST00000314752 /// | Sulfotransferase 1A1 | sulfotransferase | SULT1A1 | 200 |
| | ENST00000395609 /// | gene:ENSG00000196502 /// | family, cytosolic, 1A, | | |
| | ENST00000395607 /// | Sulfotransferase 1A1 | phenol-preferring, | | |
| | ENST00000350842 /// | gene:ENSG00000196502 /// | member 1 | | |
| | AB209149 /// | Sulfotransferase 1A1 | | | |
| | NM_177529 /// | gene:ENSG00000196502 /// | | | |
| | NM_177530 /// | sulfotransferase family, | | | |
| | NM_177536 /// | cytosolic, 1A, phenol-preferring, | | | |
| | NM_001055 /// | member 1 isoform b | | | |
| | NM_177534 | gene:ENSG00000196502 /// | | | |
| | | Homo sapiens mRNA for | | | |
| | | Phenol-sulfating phenol | | | |
| | | sulfotransferase 1 variant | | | |
| | | protein. /// Homo sapiens | | | |
| | | sulfotransferase family, | | | |
| | | cytosolic, 1A, phenol-preferring, | | | |
| | | member 1 (SULT1A1), | | | |
| | | transcript variant 2, mRNA. /// | | | |
| | | Homo sapiens sulfotransferase | | | |
| | | family, cytosolic, 1A, phenol- | | | |
| | | preferring, member 1 | | | |
| | | (SULT1A1), transcript variant 3, | | | |
| | | mRNA. /// Homo sapiens | | | |
| | | sulfotransferase family, | | | |
| | | cytosolic, 1A, phenol-preferring, | | | |
| | | member 1 (SULT1A1), | | | |
| | | transcript variant 5, mRNA. /// | | | |
| | | Homo sapiens sulfotransferase | | | |
| | | family, cytosolic, 1A, phenol- | | | |
| | | preferring, member 1 | | | |
| | | (SULT1A1), transcript variant 1, | | | |
| | | mRNA. /// Homo sapiens | | | |
| | | sulfotransferase family, | | | |
| | | cytosolic, 1A, phenol-preferring, | | | |
| | | member 1 (SULT1A1), | | | |
| | | transcript variant 4, mRNA. | | | |
| 7973444 | ENST00000386719 /// | ncrna:snRNA_pseudogene | — | — | 201 |
| | ENST00000410999 | chromosome:NCBI36:14:23330971:23331182:1 | | | |
| | | gene:ENSG00000209454 /// | | | |
| | | ncrna:misc_RNA | | | |
| | | chromosome:NCBI36:14:23330979:23331267:1 | | | |
| | | gene:ENSG00000222931 | | | |
| 8072004 | ENST00000382738 /// | immunoglobulin lambda-like | immunoglobulin | IGLL3 | 202 |
| | NM_001013618 | polypeptide 3 | lambda-like | | |
| | | gene:ENSG00000206066 /// | polypeptide 3 | | |
| | | Homo sapiens immunoglobulin | | | |
| | | lambda-like polypeptide 3 | | | |
| | | (IGLL3), mRNA. | | | |
| 7982574 | ENST00000397609 /// | family with sequence similarity | family with sequence | FAM98B | 203 |
| | ENST00000305752 /// | 98, member B isoform 1 | similarity 98, member | | |
| | AK095745 /// | gene:ENSG00000171262 /// | B | | |
| | NM_173611 /// | Protein FAM98B | | | |
| | NM_001042429 | gene:ENSG00000171262 /// | | | |
| | | Homo sapiens cDNA FLJ38426 | | | |
| | | fis, clone FEBRA2012507. /// | | | |
| | | Homo sapiens family with | | | |
| | | sequence similarity 98, member | | | |
| | | B (FAM98B), transcript variant | | | |
| | | 1, mRNA. /// Homo sapiens | | | |
| | | family with sequence similarity | | | |
| | | 98, member B (FAM98B), | | | |
| | | transcript variant 2, mRNA. | | | |
| 8135436 | ENST00000265715 /// | Pendrin | solute carrier family | SLC26A4 | 204 |
| | AF030880 /// | gene:ENSG00000091137 /// | 26, member 4 | | |
| | NM_000441 | Homo sapiens pendrin (PDS) | | | |
| | | mRNA, complete cds. /// Homo sapiens | | | |
| | | solute carrier family 26, | | | |
| | | member 4 (SLC26A4), mRNA. | | | |
| 8180281 | — | — | — | — | 205 |
| 8080676 | ENST00000311180 /// | Isoform 1 of 2′,5′- | phosphodiesterase | PDE12 | 206 |
| | AK300374 /// | phosphodiesterase 12 | 12 | | |
| | NM_177966 | gene:ENSG00000174840 /// | | | |
| | | Homo sapiens cDNA FLJ54489 | | | |
| | | complete cds, highly similar to | | | |
| | | Homo sapiens 2′- | | | |
| | | phosphodiesterase (2-PDE), | | | |
| | | mRNA. /// Homo sapiens | | | |
| | | phosphodiesterase 12 | | | |
| | | (PDE12), mRNA. | | | |
| 8154135 | ENST00000262352 /// | Excitatory amino acid | solute carrier family 1 | SLC1A1 | 207 |
| | ENST00000381910 /// | transporter 3 | (neuronal/epithelial | | |
| | BC033040 /// | gene:ENSG00000106688 /// | high affinity glutamate | | |
| | NM_004170 | Solute carrier family 1 | transporter, system | | |
| | | (Neuronal/epithelial high affinity | Xag), member 1 | | |
| | | glutamate transporter, system | | | |
| | | Xag), member 1 | | | |
| | | gene:ENSG00000106688 /// | | | |
| | | Homo sapiens solute carrier | | | |
| | | family 1 (neuronal/epithelial | | | |
| | | high affinity glutamate | | | |
| | | transporter, system Xag), | | | |
| | | member 1, mRNA (cDNA clone | | | |
| | | MGC:33786 IMAGE:5261168), | | | |
| | | complete cds. /// Homo sapiens | | | |
| | | solute carrier family 1 | | | |
| | | (neuronal/epithelial high affinity | | | |
| | | glutamate transporter, system | | | |
| | | Xag), member 1 (SLC1A1), | | | |
| | | mRNA. | | | |
| 8091306 | ENST00000354952 /// | Phospholipid scramblase 4 | phospholipid | PLSCR4 | 208 |
| | ENST00000383083 /// | gene:ENSG00000114698 /// | scramblase 4 | | |
| | AF199023 /// | phospholipid scramblase 4 | | | |
| | NM_001128305 /// | isoform b | | | |
| | NM_020353 /// | gene:ENSG00000114698 /// | | | |
| | NM_001128304 /// | Homo sapiens phospholipid | | | |
| | NM_001128306 | scramblase 4 mRNA, complete | | | |
| | | cds. /// Homo sapiens | | | |
| | | phospholipid scramblase 4 | | | |
| | | (PLSCR4), transcript variant 3, | | | |
| | | mRNA. /// Homo sapiens | | | |
| | | phospholipid scramblase 4 | | | |
| | | (PLSCR4), transcript variant 2, | | | |
| | | mRNA. /// Homo sapiens | | | |
| | | phospholipid scramblase 4 | | | |
| | | (PLSCR4), transcript variant 1, | | | |
| | | mRNA. /// Homo sapiens | | | |
| | | phospholipid scramblase 4 | | | |
| | | (PLSCR4), transcript variant 4, | | | |
| | | mRNA. | | | |
| 7971375 | ENST00000379056 /// | Tumor protein, translationally- | tumor protein, | TPT1 | 209 |
| | ENST00000379060 /// | controlled 1, isoform CRA_a | translationally- | | |
| | ENST00000379055 /// | gene:ENSG00000133112 /// | controlled 1 | | |
| | ENST00000309246 /// | Translationally-controlled tumor | | | |
| | BC040008 /// | protein | | | |
| | NM_003295 | gene:ENSG00000133112 /// | | | |
| | | Tumor protein, translationally- | | | |
| | | controlled 1, isoform CRA_a | | | |
| | | gene:ENSG00000133112 /// | | | |
| | | Tumor protein, translationally- | | | |
| | | controlled 1 | | | |
| | | gene:ENSG00000133112 /// | | | |
| | | Homo sapiens tumor protein, | | | |
| | | translationally-controlled 1, | | | |
| | | mRNA (cDNA clone | | | |
| | | IMAGE:5219529), complete | | | |
| | | cds. /// Homo sapiens tumor | | | |
| | | protein, translationally- | | | |
| | | controlled 1 (TPT1), mRNA. | | | |
| 7981998 | NR_003339 | Homo sapiens small nucleolar | small nuclear | SNRPN /// | 210 |
| | | RNA, C/D box 116-25 | ribonucleoprotein | SNORD116-25 | |
| | | (SNORD116-25), non-coding | polypeptide N /// small | | |
| | | RNA. | nucleolar RNA, C/D | | |
| | | | box 116-25 | | |
| 7905077 | BC068044 | Homo sapiens cDNA clone | — | — | 211 |
| | | IMAGE:6380649, containing | | | |
| | | frame-shift errors. | | | |
| 7912349 | ENST00000377008 /// | Isoform 1 of Uncharacterized | chromosome 1 open | C1orf127 | 212 |
| | ENST00000377004 /// | protein C1orf127 | reading frame 127 | | |
| | AK094437 /// | gene:ENSG00000175262 /// | | | |
| | AK095152 /// | Isoform 2 of Uncharacterized | | | |
| | BC126349 | protein C1orf127 | | | |
| | | gene:ENSG00000175262 /// | | | |
| | | Homo sapiens cDNA FLJ37118 | | | |
| | | fis, clone BRACE2022328. /// | | | |
| | | Homo sapiens cDNA FLJ37833 | | | |
| | | fis, clone BRSSN2009702. /// | | | |
| | | Homo sapiens chromosome 1 | | | |
| | | open reading frame 127, mRNA | | | |
| | | (cDNA clone MGC:161627 | | | |
| | | IMAGE:8992065), complete | | | |
| | | cds. | | | |
| 8091097 | XR_040865 | PREDICTED: Homo sapiens | hypothetical protein | FLJ11827 | 213 |
| | | misc_RNA (FLJ11827), | FLJ11827 | | |
| | | miscRNA. | | | |
| 7904287 | ENST000003694787 /// | T-cell surface antigen CD2 | CD2 molecule | CD2 | 214 |
| | ENST00000369477 /// | gene:ENSG00000116824 /// | | | |
| | BC033583 /// | CD2 molecule | | | |
| | NM_001767 | gene:ENSG00000116824 /// | | | |
| | | Homo sapiens CD2 molecule, | | | |
| | | mRNA (cDNA clone | | | |
| | | MGC:34621 IMAGE:5227138), | | | |
| | | complete cds. /// Homo sapiens | | | |
| | | CD2 molecule (CD2), mRNA. | | | |
| 8043100 | ENST00000233143 /// | Thymosin beta-10 | thymosin beta 10 | TMSB10 | 215 |
| | BC107889 /// | gene:ENSG00000034510 /// | | | |
| | NM_021103 | Homo sapiens cDNA clone | | | |
| | | IMAGE:6651898. /// Homo sapiens | | | |
| | | thymosin beta 10 | | | |
| | | (TMSB10), mRNA. | | | |
| 8177323 | ENST00000303804 /// | Isoform 1 of PTPN13-like | PTPN13-like, Y-linked /// | PRY /// | 216 |
| | ENST00000341740 /// | protein, Y-linked | PTPN13-like, Y- | PRY2 | |
| | ENST00000338793 /// | gene:ENSG00000169807 /// | linked 2 | | |
| | ENST00000303728 /// | Isoform 2 of PTPN13-like | | | |
| | ENST00000343584 /// | protein, Y-linked | | | |
| | ENST00000303593 /// | gene:ENSG00000169807 /// | | | |
| | ENST00000306589 /// | Isoform 2 of PTPN13-like | | | |
| | ENST00000338673 /// | protein, Y-linked | | | |
| | AF517635 /// | gene:ENSG00000169789 /// | | | |
| | NM_001002758 /// | Isoform 1 of PTPN13-like | | | |
| | NM_004676 | protein, Y-linked | | | |
| | | gene:ENSG00000169789 /// | | | |
| | | Isoform 1 of PTPN13-like | | | |
| | | protein, Y-linked | | | |
| | | gene:ENSG00000169763 /// | | | |
| | | Isoform 2 of PTPN13-like | | | |
| | | protein, Y-linked | | | |
| | | gene:ENSG00000169763 /// | | | |
| | | Isoform 1 of PTPN13-like | | | |
| | | protein, Y-linked | | | |
| | | gene:ENSG00000172283 /// | | | |
| | | Isoform 2 of PTPN13-like | | | |
| | | protein, Y-linked | | | |
| | | gene:ENSG00000172283 /// | | | |
| | | Homo sapiens testis-specific | | | |
| | | PTP-BL related Y protein | | | |
| | | mRNA, complete cds, | | | |
| | | alternatively spliced. /// Homo sapiens | | | |
| | | PTPN13-like, Y-linked | | | |
| | | 2 (PRY2), mRNA. /// Homo sapiens | | | |
| | | PTPN13-like, Y-linked | | | |
| | | (PRY), mRNA. | | | |
| 7920633 | ENST00000368399 /// | cDNA FLJ60436, highly similar | dolichyl-phosphate | DPM3 | 217 |
| | ENST00000341298 /// | to Homo sapiens dolichyl- | mannosyltransferase | | |
| | ENST00000368400 /// | phosphate | polypeptide 3 | | |
| | AK293625 /// | mannosyltransferase | | | |
| | NM_153741 /// | polypeptide 3, transcript variant | | | |
| | NM_018973 | 1, mRNA | | | |
| | | gene:ENSG00000179085 /// | | | |
| | | Isoform 1 of Dolichol-phosphate | | | |
| | | mannosyltransferase subunit 3 | | | |
| | | gene:ENSG00000179085 /// | | | |
| | | Isoform 1 of Dolichol-phosphate | | | |
| | | mannosyltransferase subunit 3 | | | |
| | | gene:ENSG00000179085 /// | | | |
| | | Homo sapiens cDNA FLJ60436 | | | |
| | | complete cds, highly similar to | | | |
| | | Homo sapiens dolichyl- | | | |
| | | phosphate | | | |
| | | mannosyltransferase | | | |
| | | polypeptide 3, transcript variant | | | |
| | | 1, mRNA. /// Homo sapiens | | | |
| | | dolichyl-phosphate | | | |
| | | mannosyltransferase | | | |
| | | polypeptide 3 (DPM3), | | | |
| | | transcript variant 2, mRNA. /// | | | |
| | | Homo sapiens dolichyl- | | | |
| | | phosphate | | | |
| | | mannosyltransferase | | | |
| | | polypeptide 3 (DPM3), | | | |
| | | transcript variant 1, mRNA. | | | |
| 8110916 | ENST00000382550 /// | cDNA FLJ42124 fis, clone | similar to hypothetical | LOC442132 | 218 |
| | AK124118 | TESTI2009477, weakly similar | protein FLJ36144 | | |
| | | to TRICHOHYALIN | | | |
| | | gene:ENSG00000205976 /// | | | |
| | | Homo sapiens cDNA FLJ42124 | | | |
| | | fis, clone TESTI2009477, | | | |
| | | weakly similar to | | | |
| | | TRICHOHYALIN. | | | |
| 7943051 | ENST00000321955 /// | N-acetylated-alpha-linked acidic | N-acetylated alpha- | NAALAD2 | 219 |
| | ENST00000375944 /// | dipeptidase 2 | linked acidic | | |
| | BC096316 /// | gene:ENSG00000077616 /// | dipeptidase 2 | | |
| | NM_005467 | NAALAD2 protein | | | |
| | | gene:ENSG00000077616 /// | | | |
| | | Homo sapiens N-acetylated | | | |
| | | alpha-linked acidic dipeptidase | | | |
| | | 2, mRNA (cDNA clone | | | |
| | | MGC:116994 | | | |
| | | IMAGE:40007638), complete | | | |
| | | cds. /// Homo sapiens N- | | | |
| | | acetylated alpha-linked acidic | | | |
| | | dipeptidase 2 (NAALAD2), | | | |
| | | mRNA. | | | |
| 7938951 | ENST00000324559 /// | Anoctamin-5 | anoctamin 5 | ANO5 | 220 |
| | AL833271 /// | gene:ENSG00000171714 /// | | | |
| | NM_213599 | Homo sapiens mRNA; cDNA | | | |
| | | DKFZp451A148 (from clone | | | |
| | | DKFZp451A148). /// Homo sapiens | | | |
| | | anoctamin 5 (ANO5), | | | |
| | | mRNA. | | | |
| 8083839 | ENST00000402305 /// | Probable G-protein coupled | G protein-coupled | GPR160 | 221 |
| | ENST00000355897 /// | receptor 160 | receptor 160 | | |
| | BC000181 /// | gene:ENSG00000173890 /// | | | |
| | NM_014373 | Probable G-protein coupled | | | |
| | | receptor 160 | | | |
| | | gene:ENSG00000173890 /// | | | |
| | | Homo sapiens G protein- | | | |
| | | coupled receptor 160, mRNA | | | |
| | | (cDNA clone MGC:5003 | | | |
| | | IMAGE:3048193), complete | | | |
| | | cds. /// Homo sapiens G | | | |
| | | protein-coupled receptor 160 | | | |
| | | (GPR160), mRNA. | | | |
| 8046020 | ENST00000375437 /// | Isoform 1 of Sodium channel | sodium channel, | SCN2A | 222 |
| | ENST00000357398 /// | protein type 2 subunit alpha | voltage-gated, type II, | | |
| | ENST00000283256 /// | gene:ENSG00000136531 /// | alpha subunit | | |
| | ENST00000375427 /// | Isoform 2 of Sodium channel | | | |
| | AB208888 /// | protein type 2 subunit alpha | | | |
| | NM_021007 /// | gene:ENSG00000136531 /// | | | |
| | NM_001040142 /// | Isoform 1 of Sodium channel | | | |
| | NM_001040143 | protein type 2 subunit alpha | | | |
| | | gene:ENSG00000136531 /// | | | |
| | | Isoform 2 of Sodium channel | | | |
| | | protein type 2 subunit alpha | | | |
| | | gene:ENSG00000136531 /// | | | |
| | | Homo sapiens mRNA for | | | |
| | | Sodium channel protein type II | | | |
| | | alpha subunit variant protein. /// | | | |
| | | Homo sapiens sodium channel, | | | |
| | | voltage-gated, type II, alpha | | | |
| | | subunit (SCN2A), transcript | | | |
| | | variant 1, mRNA. /// Homo sapiens | | | |
| | | sodium channel, | | | |
| | | voltage-gated, type II, alpha | | | |
| | | subunit (SCN2A), transcript | | | |
| | | variant 2, mRNA. /// Homo sapiens | | | |
| | | sodium channel, | | | |
| | | voltage-gated, type II, alpha | | | |
| | | subunit (SCN2A), transcript | | | |
| | | variant 3, mRNA. | | | |
| 8127658 | ENST00000364421 | ncrna:snRNA | — | — | 223 |
| | | chromosome:NCBI36:6:76240174:76240330:−1 | | | |
| | | gene:ENSG00000201291 | | | |
| 8129097 | AK096882 | Homo sapiens cDNA FLJ39563 | TSPY-like 1 | TSPYL1 | 224 |
| | | fis, clone SKMUS2001164. | | | |
| 7903079 | ENST00000370272 /// | Protein Dr1 | down-regulator of | DR1 | 225 |
| | ENST00000370267 /// | gene:ENSG00000117505 /// | transcription 1, TBP- | | |
| | BC035507 /// | Protein Dr1 | binding (negative | | |
| | NM_001938 | gene:ENSG00000117505 /// | cofactor 2) | | |
| | | Homo sapiens down-regulator | | | |
| | | of transcription 1, TBP-binding | | | |
| | | (negative cofactor 2), mRNA | | | |
| | | (cDNA clone MGC:29766 | | | |
| | | IMAGE:4555131), complete | | | |
| | | cds. /// Homo sapiens down- | | | |
| | | regulator of transcription 1, | | | |
| | | TBP-binding (negative cofactor | | | |
| | | 2) (DR1), mRNA. | | | |
| 8110104 | ENST00000410179 | ncrna:misc_RNA | — | — | 226 |
| | | chromosome:NCBI36:5:174987797:174988108:1 | | | |
| | | gene:ENSG00000222111 | | | |
| 8127145 | ENST00000304434 /// | Elongation of very long chain | ELOVL family | ELOVL5 | 227 |
| | ENST00000370918 /// | fatty acids protein 5 | member 5, elongation | | |
| | AF338241 /// | gene:ENSG00000012660 /// 35 | of long chain fatty | | |
| | NM_021814 | kDa protein | acids (FEN1/Elo2, | | |
| | | gene:ENSG00000012660 /// | SUR4/Elo3-like, | | |
| | | Homo sapiens elongation of | yeast) | | |
| | | very long chain fatty acids | | | |
| | | protein-like protein 2 (ELOVL2) | | | |
| | | mRNA, complete cds. /// Homo sapiens | | | |
| | | ELOVL family member | | | |
| | | 5, elongation of long chain fatty | | | |
| | | acids (FEN1/Elo2, SUR4/Elo3- | | | |
| | | like, yeast) (ELOVL5), mRNA. | | | |
| 8175299 | ENST00000391440 /// | Protein FAM127C | family with sequence | FAM127C | 228 |
| | BC086860 /// | gene:ENSG00000212747 /// | similarity 127, | | |
| | NM_001078173 | Homo sapiens cDNA clone | member C | | |
| | | IMAGE:6153002. /// Homo sapiens | | | |
| | | family with sequence | | | |
| | | similarity 127, member C | | | |
| | | (FAM127C), mRNA. | | | |
| 8033248 | ENST00000245912 /// | tumor necrosis factor ligand | tumor necrosis factor | TNFSF14 | 229 |
| | AF064090 /// | superfamily, member 14 | (ligand) superfamily, | | |
| | NM_172014 /// | isoform 1 precursor | member 14 | | |
| | NM_003807 | gene:ENSG00000125735 /// | | | |
| | | Homo sapiens ligand for | | | |
| | | herpesvirus entry mediator | | | |
| | | (HVEM-L) mRNA, complete | | | |
| | | cds. /// Homo sapiens tumor | | | |
| | | necrosis factor (ligand) | | | |
| | | superfamily, member 14 | | | |
| | | (TNFSF14), transcript variant 2, | | | |
| | | mRNA. /// Homo sapiens tumor | | | |
| | | necrosis factor (ligand) | | | |
| | | superfamily, member 14 | | | |
| | | (TNFSF14), transcript variant 1, | | | |
| | | mRNA. | | | |
| 7933075 | ENST00000374694 /// | Frizzled-8 | frizzled homolog 8 | FZD8 | 230 |
| | AB043703 /// | gene:ENSG00000177283 /// | (Drosophila) | | |
| | NM_031866 | Homo sapiens FZD8 mRNA for | | | |
| | | seven-transmembrane receptor | | | |
| | | Frizzled-8, complete cds. /// | | | |
| | | Homo sapiens frizzled homolog | | | |
| | | 8 (Drosophila) (FZD8), mRNA. | | | |
| 7915870 | ENST00000371937 /// | ATP synthase mitochondrial F1 | ATP synthase | ATPAF1 | 231 |
| | ENST00000329231 /// | complex assembly factor 1 | mitochondrial F1 | | |
| | BC008498 /// | gene:ENSG00000123472 /// | complex assembly | | |
| | NM_022745 /// | ATP synthase mitochondrial F1 | factor 1 | | |
| | NM_001042546 | complex assembly factor 1 | | | |
| | | isoform 2 precursor | | | |
| | | gene:ENSG00000123472 /// | | | |
| | | Homo sapiens ATP synthase | | | |
| | | mitochondrial F1 complex | | | |
| | | assembly factor 1, mRNA | | | |
| | | (cDNA clone MGC:14830 | | | |
| | | IMAGE:4281102), complete | | | |
| | | cds. /// Homo sapiens ATP | | | |
| | | synthase mitochondrial F1 | | | |
| | | complex assembly factor 1 | | | |
| | | (ATPAF1), nuclear gene | | | |
| | | encoding mitochondrial protein, | | | |
| | | transcript variant 1, mRNA. /// | | | |
| | | Homo sapiens ATP synthase | | | |
| | | mitochondrial F1 complex | | | |
| | | assembly factor 1 (ATPAF1), | | | |
| | | nuclear gene encoding | | | |
| | | mitochondrial protein, transcript | | | |
| | | variant 2, mRNA. | | | |
| 8121064 | ENST00000388700 /// | ncrna:scRNA_pseudogene | — | — | 232 |
| | ENST00000411294 | chromosome:NCBI36:6:88612087:88612384:1 | | | |
| | | gene:ENSG00000211435 /// | | | |
| | | ncrna:misc_RNA | | | |
| | | chromosome:NCBI36:6:88612087:88612382:1 | | | |
| | | gene:ENSG00000223226 | | | |
| 8161437 | GENSCAN00000015556 /// | cdna:Genscan | similar to | LOC100132357 | 233 |
| | ENST00000407551 /// | chromosome:NCBI36:9:46007163:46060142:−1 /// | hCG1656091 | | |
| | XM_001714380 | cdna:pseudogene | | | |
| | | chromosome:NCBI36:9:46277008:46277218:1 | | | |
| | | gene:ENSG00000216653 /// | | | |
| | | PREDICTED: Homo sapiens | | | |
| | | similar to hCG1656091 | | | |
| | | (LOC100132357), mRNA. | | | |
| 8008980 | ENST00000335108 /// | Putative uncharacterized | chromosome 17 open | C17orf82 | 234 |
| | BC046200 | protein C17orf82 | reading frame 82 | | |
| | | gene:ENSG00000187013 /// | | | |
| | | Homo sapiens chromosome 17 | | | |
| | | open reading frame 82, mRNA | | | |
| | | (cDNA clone MGC:57831 | | | |
| | | IMAGE:6152618), complete | | | |
| | | cds. | | | |
| 8154563 | ENST00000340967 /// | Isoform 1 of Alkaline | alkaline ceramidase 2 | ACER2 | 235 |
| | ENST00000380376 /// | ceramidase 2 | | | |
| | BC092487 /// | gene:ENSG00000177076 /// | | | |
| | NM_001010887 | Isoform 3 of Alkaline | | | |
| | | ceramidase 2 | | | |
| | | gene:ENSG00000177076 /// | | | |
| | | Homo sapiens N- | | | |
| | | acylsphingosine | | | |
| | | amidohydrolase 3-like, mRNA | | | |
| | | (cDNA clone MGC:104688 | | | |
| | | IMAGE:30528463), complete | | | |
| | | cds. /// Homo sapiens N- | | | |
| | | acylsphingosine | | | |
| | | amidohydrolase 3-like | | | |
| | | (ASAH3L), mRNA. | | | |
| 8161426 | GENSCAN00000015556 /// | cdna:Genscan | similar to | LOC100132357 | 236 |
| | ENST00000407551 /// | chromosome:NCBI36:9:46007163:46060142:−1 /// | hCG1656091 | | |
| | XM_001714380 | cdna:pseudogene | | | |
| | | chromosome:NCBI36:9:46277008:46277218:1 | | | |
| | | gene:ENSG00000216653 /// | | | |
| | | PREDICTED: Homo sapiens | | | |
| | | similar to hCG1656091 | | | |
| | | (LOC100132357), mRNA. | | | |
| 8083223 | ENST00000315691 /// | UPF0672 protein C3orf58 | chromosome 3 open | C3orf58 | 237 |
| | BC037293 /// | gene:ENSG00000181744 /// | reading frame 58 | | |
| | NM_173552 /// | Homo sapiens chromosome 3 | | | |
| | NM_001134470 | open reading frame 58, mRNA | | | |
| | | (cDNA clone MGC:33365 | | | |
| | | IMAGE:5267770), complete | | | |
| | | cds. /// Homo sapiens | | | |
| | | chromosome 3 open reading | | | |
| | | frame 58 (C3orf58), transcript | | | |
| | | variant 1, mRNA. /// Homo sapiens | | | |
| | | chromosome 3 open | | | |
| | | reading frame 58 (C3orf58), | | | |
| | | transcript variant 2, mRNA. | | | |
| 8001329 | ENST00000219197 /// | Cerebellin-1 | cerebellin 1 precursor | CBLN1 | 238 |
| | M58583 /// | gene:ENSG00000102924 /// | | | |
| | NM_004352 | Human precerebellin and | | | |
| | | cerebellin mRNA, complete | | | |
| | | cds. /// Homo sapiens cerebellin | | | |
| | | 1 precursor (CBLN1), mRNA. | | | |
| 8165575 | ENST00000371457 /// | cDNA FLJ45411 fis, clone | patatin-like | PNPLA7 | 239 |
| | ENST00000277531 /// | BRHIP3032374, moderately | phospholipase | | |
| | ENST00000406427 /// | similar to Homo sapiens | domain containing 7 | | |
| | ENST00000371451 /// | neuropathy target esterase | | | |
| | ENST00000371450 /// | gene:ENSG00000130653 /// | | | |
| | ENST00000371446 /// | Isoform 1 of Patatin-like | | | |
| | AK297623 /// | phospholipase domain- | | | |
| | NM_152286 /// | containing protein 7 | | | |
| | NM_001098537 | gene:ENSG00000130653 /// | | | |
| | | patatin-like phospholipase | | | |
| | | domain containing 7 isoform a | | | |
| | | gene:ENSG00000130653 /// | | | |
| | | Putative uncharacterized | | | |
| | | protein PNPLA7 | | | |
| | | gene:ENSG00000130653 /// | | | |
| | | Isoform 2 of Patatin-like | | | |
| | | phospholipase domain- | | | |
| | | containing protein 7 | | | |
| | | gene:ENSG00000130653 /// 23 | | | |
| | | kDa protein | | | |
| | | gene:ENSG00000130653 /// | | | |
| | | Homo sapiens cDNA FLJ55553 | | | |
| | | complete cds. /// Homo sapiens | | | |
| | | patatin-like phospholipase | | | |
| | | domain containing 7 (PNPLA7), | | | |
| | | transcript variant 2, mRNA. /// | | | |
| | | Homo sapiens patatin-like | | | |
| | | phospholipase domain | | | |
| | | containing 7 (PNPLA7), | | | |
| | | transcript variant 1, mRNA. | | | |
| 7971731 | ENST00000400366 /// | Isoform 3 of Copper- | ATPase, Cu++ | ATP7B | 240 |
| | ENST00000344297 /// | transporting ATPase 2 | transporting, beta | | |
| | ENST00000242839 /// | gene:ENSG00000123191 /// | polypeptide | | |
| | ENST00000400370 /// | Isoform 2 of Copper- | | | |
| | U11700 /// | transporting ATPase 2 | | | |
| | NM_000053 /// | gene:ENSG00000123191 /// | | | |
| | NM_001005918 | Isoform 1 of Copper- | | | |
| | | transporting ATPase 2 | | | |
| | | gene:ENSG00000123191 /// | | | |
| | | ATP7B protein | | | |
| | | gene:ENSG00000123191 /// | | | |
| | | Human copper transporting | | | |
| | | ATPase mRNA, complete cds. /// | | | |
| | | Homo sapiens ATPase, | | | |
| | | Cu++ transporting, beta | | | |
| | | polypeptide (ATP7B), transcript | | | |
| | | variant 1, mRNA. /// Homo sapiens | | | |
| | | ATPase, Cu++ | | | |
| | | transporting, beta polypeptide | | | |
| | | (ATP7B), transcript variant 2, | | | |
| | | mRNA. | | | |
| 8028600 | ENST00000339852 /// | FBA domain-containing protein | non-specific cytotoxic | NCCRP1 | 241 |
| | BC092493 /// | LOC342897 | cell receptor protein 1 | | |
| | NM_001001414 | gene:ENSG00000188505 /// | homolog (zebrafish) | | |
| | | Homo sapiens similar to F-box | | | |
| | | only protein 2, mRNA (cDNA | | | |
| | | clone MGC:104713 | | | |
| | | IMAGE:30337042), complete | | | |
| | | cds. /// Homo sapiens | | | |
| | | nonspecific cytotoxic cell | | | |
| | | receptor protein 1 homolog | | | |
| | | (zebrafish) (NCCRP1), mRNA. | | | |
| 8155569 | ENST00000407551 /// | cdna:pseudogene | similar to | LOC100132357 | 242 |
| | XM_001714380 | chromosome:NCBI36:9:46277008:46277218:1 | hCG1656091 | | |
| | | gene:ENSG00000216653 /// | | | |
| | | PREDICTED: Homo sapiens | | | |
| | | similar to hCG1656091 | | | |
| | | (LOC100132357), mRNA. | | | |
| 8039605 | ENST00000342088 /// | Zinc finger protein 835 | zinc finger protein 835 | ZNF835 | 243 |
| | AK023017 /// | gene:ENSG00000127903 /// | | | |
| | NM_001005850 | Homo sapiens cDNA FLJ12955 | | | |
| | | fis, clone NT2RP2005496, | | | |
| | | moderately similar to ZINC | | | |
| | | FINGER PROTEIN 135. /// | | | |
| | | Homo sapiens zinc finger | | | |
| | | protein 835 (ZNF835), mRNA. | | | |
| 7937971 | ENST00000322493 | cdna:pseudogene | — | — | 244 |
| | | chromosome:NCBI36:11:4764560:4765496:1 | | | |
| | | gene:ENSG00000176951 | | | |
| 8137925 | ENST00000384168 | ncrna:misc_RNA | — | — | 245 |
| | | chromosome:NCBI36:7:4817345:4817446:−1 | | | |
| | | gene:ENSG00000206895 | | | |
| 7915408 | ENST00000372572 /// | Isoform 1 of Forkhead box | forkhead box J3 | FOXJ3 | 246 |
| | ENST00000372571 /// | protein J3 | | | |
| | ENST00000372573 /// | gene:ENSG00000198815 /// 15 | | | |
| | ENST00000361346 /// | kDa protein | | | |
| | ENST00000361776 /// | gene:ENSG00000198815 /// | | | |
| | BC152441 /// | Isoform 1 of Forkhead box | | | |
| | NM_014947 | protein J3 | | | |
| | | gene:ENSG00000198815 /// | | | |
| | | Isoform 1 of Forkhead box | | | |
| | | protein J3 | | | |
| | | gene:ENSG00000198815 /// | | | |
| | | Isoform 2 of Forkhead box | | | |
| | | protein J3 | | | |
| | | gene:ENSG00000198815 /// | | | |
| | | Homo sapiens forkhead box J3, | | | |
| | | mRNA (cDNA clone | | | |
| | | MGC:176686 | | | |
| | | IMAGE:8862565), complete | | | |
| | | cds. /// Homo sapiens forkhead | | | |
| | | box J3 (FOXJ3), mRNA. | | | |
| 8014891 | ENST00000394189 /// | Aiolos isoform hAio-del | IKAROS family zinc | IKZF3 | 247 |
| | ENST00000377944 /// | gene:ENSG00000161405 /// | finger 3 (Aiolos) | | |
| | ENST00000348427 /// | Aiolos isoform hAio-del | | | |
| | ENST00000346872 /// | gene:ENSG00000161405 /// | | | |
| | ENST00000377958 /// | Isoform 2 of Zinc finger protein | | | |
| | ENST00000293068 /// | Aiolos | | | |
| | ENST00000351680 /// | gene:ENSG00000161405 /// | | | |
| | ENST00000350532 /// | Isoform 5 of Zinc finger protein | | | |
| | ENST00000377945 /// | Aiolos | | | |
| | ENST00000346243 /// | gene:ENSG00000161405 /// | | | |
| | ENST00000377952 /// | Aiolos isoform hAio-del | | | |
| | AY377981 /// | gene:ENSG00000161405 /// | | | |
| | NM_183230 /// | Isoform 1 of Zinc finger protein | | | |
| | NM_183231 /// | Aiolos | | | |
| | NM_183232 /// | gene:ENSG00000161405 /// | | | |
| | NM_012481 /// | Isoform 3 of Zinc finger protein | | | |
| | NM_183228 /// | Aiolos | | | |
| | NM_183229 | gene:ENSG00000161405 /// | | | |
| | | Isoform 4 of Zinc finger protein | | | |
| | | Aiolos | | | |
| | | gene:ENSG00000161405 /// | | | |
| | | Aiolos isoform hAio-del | | | |
| | | gene:ENSG00000161405 /// | | | |
| | | Isoform 6 of Zinc finger protein | | | |
| | | Aiolos | | | |
| | | gene:ENSG00000161405 /// | | | |
| | | Aiolos isoform hAio-del | | | |
| | | gene:ENSG00000161405 /// | | | |
| | | Homo sapiens aiolos isoform | | | |
| | | hAio-ALT (ZNFN1A3) mRNA, | | | |
| | | complete cds, alternatively | | | |
| | | spliced. /// Homo sapiens | | | |
| | | IKAROS family zinc finger 3 | | | |
| | | (Aiolos) (IKZF3), transcript | | | |
| | | variant 4, mRNA. /// Homo sapiens | | | |
| | | IKAROS family zinc | | | |
| | | finger 3 (Aiolos) (IKZF3), | | | |
| | | transcript variant 5, mRNA. /// | | | |
| | | Homo sapiens IKAROS family | | | |
| | | zinc finger 3 (Aiolos) (IKZF3), | | | |
| | | transcript variant 6, mRNA. /// | | | |
| | | Homo sapiens IKAROS family | | | |
| | | zinc finger 3 (Aiolos) (IKZF3), | | | |
| | | transcript variant 1, mRNA. /// | | | |
| | | Homo sapiens IKAROS family | | | |
| | | zinc finger 3 (Aiolos) (IKZF3), | | | |
| | | transcript variant 2, mRNA. /// | | | |
| | | Homo sapiens IKAROS family | | | |
| | | zinc finger 3 (Aiolos) (IKZF3), | | | |
| | | transcript variant 3, mRNA. | | | |
| 7999406 | ENST00000386866 | ncrna:Mt_tRNA_pseudogene | — | — | 248 |
| | | chromosome:NCBI36:16:10722924:10722982:−1 | | | |
| | | gene:ENSG00000209601 | | | |
| 8128087 | ENST00000369451 /// | gamma-aminobutyric acid | gamma-aminobutyric | GABRR1 | 249 |
| | BC130344 /// | (GABA) receptor, rho 1 | acid (GABA) receptor, | | |
| | NM_002042 | gene:ENSG00000146276 /// | rho 1 | | |
| | | Homo sapiens gamma- | | | |
| | | aminobutyric acid (GABA) | | | |
| | | receptor, rho 1, mRNA (cDNA | | | |
| | | clone MGC:163216 | | | |
| | | IMAGE:40146375), complete | | | |
| | | cds. /// Homo sapiens gamma- | | | |
| | | aminobutyric acid (GABA) | | | |
| | | receptor, rho 1 (GABRR1), | | | |
| | | mRNA. | | | |
| 8011968 | ENST00000225728 /// | Mediator of RNA polymerase II | mediator complex | MED31 | 250 |
| | AF151883 /// | transcription subunit 31 | subunit 31 | | |
| | NM_016060 | gene:ENSG00000108590 /// | | | |
| | | Homo sapiens CGI-125 protein | | | |
| | | mRNA, complete cds. /// Homo sapiens | | | |
| | | mediator complex | | | |
| | | subunit 31 (MED31), mRNA. | | | |
| 7950555 | ENST00000404995 /// | Leucine-rich repeat-containing | leucine rich repeat | LRRC32 | 251 |
| | ENST00000407242 /// | protein 32 | containing 32 | | |
| | ENST00000260061 /// | gene:ENSG00000137507 /// | | | |
| | BC070079 /// | Leucine-rich repeat-containing | | | |
| | NM_005512 /// | protein 32 | | | |
| | NM_001128922 | gene:ENSG00000137507 /// | | | |
| | | Leucine-rich repeat-containing | | | |
| | | protein 32 | | | |
| | | gene:ENSG00000137507 /// | | | |
| | | Homo sapiens leucine rich | | | |
| | | repeat containing 32, mRNA | | | |
| | | (cDNA clone MGC:87399 | | | |
| | | IMAGE:30344529), complete | | | |
| | | cds. /// Homo sapiens leucine | | | |
| | | rich repeat containing 32 | | | |
| | | (LRRC32), transcript variant 1, | | | |
| | | mRNA. /// Homo sapiens | | | |
| | | leucine rich repeat containing | | | |
| | | 32 (LRRC32), transcript variant | | | |
| | | 2, mRNA. | | | |
| 8012535 | ENST00000329805 /// | UPF0537 transmembrane | major facilitator | MFSD6L | 252 |
| | AY129026 /// | protein | superfamily domain | | |
| | NM_152599 | gene:ENSG00000185156 /// | containing 6-like | | |
| | | Homo sapiens clone FP7072 | | | |
| | | unknown mRNA. /// Homo sapiens | | | |
| | | major facilitator | | | |
| | | superfamily domain containing | | | |
| | | 6-like (MFSD6L), mRNA. | | | |
| 7988767 | ENST00000396402 /// | Cytochrome P450 19A1 | cytochrome P450, | CYP19A1 | 253 |
| | ENST00000396404 /// | gene:ENSG00000137869 /// | family 19, subfamily | | |
| | ENST00000260433 /// | Cytochrome P450 19A1 | A, polypeptide 1 | | |
| | ENST00000405913 /// | gene:ENSG00000137869 /// | | | |
| | AK291778 /// | Cytochrome P450 19A1 | | | |
| | NM_031226 /// | gene:ENSG00000137869 /// | | | |
| | NM_000103 | CYP19A1 protein | | | |
| | | gene:ENSG00000137869 /// | | | |
| | | Homo sapiens cDNA FLJ75846 | | | |
| | | complete cds, highly similar to | | | |
| | | Homo sapiens cytochrome | | | |
| | | P450, family 19, subfamily A, | | | |
| | | polypeptide 1 (CYP19A1), | | | |
| | | transcript variant 1, mRNA. /// | | | |
| | | Homo sapiens cytochrome | | | |
| | | P450, family 19, subfamily A, | | | |
| | | polypeptide 1 (CYP19A1), | | | |
| | | transcript variant 2, mRNA. /// | | | |
| | | Homo sapiens cytochrome | | | |
| | | P450, family 19, subfamily A, | | | |
| | | polypeptide 1 (CYP19A1), | | | |
| | | transcript variant 1, mRNA. | | | |
| 8036291 | ENST00000355114 /// | cDNA FLJ32728 fis, clone | zinc finger protein 565 | ZNF565 | 254 |
| | ENST00000392173 /// | TESTI2001049, highly similar to | | | |
| | ENST00000304116 /// | Zinc finger protein 565 | | | |
| | BC068453 /// | gene:ENSG00000196357 /// | | | |
| | NM_001042474 /// | Zinc finger protein 565 | | | |
| | NM_152477 | gene:ENSG00000196357 /// | | | |
| | | Zinc finger protein 565 | | | |
| | | gene:ENSG00000196357 /// | | | |
| | | Homo sapiens zinc finger | | | |
| | | protein 565, mRNA (cDNA | | | |
| | | clone IMAGE:30343899). /// | | | |
| | | Homo sapiens zinc finger | | | |
| | | protein 565 (ZNF565), | | | |
| | | transcript variant 1, mRNA. /// | | | |
| | | Homo sapiens zinc finger | | | |
| | | protein 565 (ZNF565), | | | |
| | | transcript variant 2, mRNA. | | | |
| 8064351 | ENST00000400227 /// | Casein kinase 2 alpha isoform | casein kinase 2, | CSNK2A1P /// | 255 |
| | ENST00000400217 /// | gene:ENSG00000101266 /// | alpha 1 polypeptide | CSNK2A1 | |
| | ENST00000361797 /// | casein kinase II alpha 1 subunit | pseudogene /// casein | | |
| | ENST00000217244 /// | isoform b | kinase 2, alpha 1 | | |
| | ENST00000349736 /// | gene:ENSG00000101266 /// | polypeptide | | |
| | ENST00000381973 /// | casein kinase II alpha 1 subunit | | | |
| | AY112721 /// | isoform b | | | |
| | BC053532 /// | gene:ENSG00000101266 /// | | | |
| | NM_001895 /// | Casein kinase II subunit alpha | | | |
| | NR_002207 /// | gene:ENSG00000101266 /// | | | |
| | NM_177559 /// | Casein kinase II subunit alpha | | | |
| | NM_177560 | gene:ENSG00000101266 /// | | | |
| | | CSNK2A1 protein | | | |
| | | gene:ENSG00000101266 /// | | | |
| | | Homo sapiens casein kinase II | | | |
| | | alpha subunit mRNA, complete | | | |
| | | cds. /// Homo sapiens casein | | | |
| | | kinase 2, alpha 1 polypeptide, | | | |
| | | mRNA (cDNA clone | | | |
| | | MGC:61540 IMAGE:3908058), | | | |
| | | complete cds. /// Homo sapiens | | | |
| | | casein kinase 2, alpha 1 | | | |
| | | polypeptide (CSNK2A1), | | | |
| | | transcript variant 2, mRNA. /// | | | |
| | | Homo sapiens casein kinase 2, | | | |
| | | alpha 1 polypeptide | | | |
| | | pseudogene (CSNK2A1P), | | | |
| | | non-coding RNA. /// Homo sapiens | | | |
| | | casein kinase 2, alpha | | | |
| | | 1 polypeptide (CSNK2A1), | | | |
| | | transcript variant 1, mRNA. /// | | | |
| | | Homo sapiens casein kinase 2, | | | |
| | | alpha 1 polypeptide | | | |
| | | (CSNK2A1), transcript variant | | | |
| | | 3, mRNA. | | | |
| 8131719 | ENST00000409508 /// | cdna:known | dynein, axonemal, | DNAH11 | 256 |
| | ENST00000328843 /// | chromosome:NCBI36:7:21549358:21907711:1 | heavy chain 11 | | |
| | AK095018 /// | gene:ENSG00000105877 /// | | | |
| | NM_003777 | Dynein heavy chain 11, | | | |
| | | axonemal | | | |
| | | gene:ENSG00000105877 /// | | | |
| | | Homo sapiens cDNA FLJ37699 | | | |
| | | fis, clone BRHIP2016788. /// | | | |
| | | Homo sapiens dynein, | | | |
| | | axonemal, heavy chain 11 | | | |
| | | (DNAH11), mRNA. | | | |
| 8095331 | ENST00000365299 | ncrna:misc_RNA | — | — | 257 |
| | | chromosome:NCBI36:4:62454626:62454721:1 | | | |
| | | gene:ENSG00000202169 | | | |
| 8180232 | — | — | — | — | 258 |
| 8105040 | ENST00000274276 /// | Isoform 1 of Oncostatin-M | oncostatin M receptor | OSMR | 259 |
| | U60805 /// | specific receptor subunit beta | | | |
| | NM_003999 | gene:ENSG00000145623 /// | | | |
| | | Human oncostatin-M specific | | | |
| | | receptor beta subunit (OSMRB) | | | |
| | | mRNA, complete cds. /// Homo sapiens | | | |
| | | oncostatin M receptor | | | |
| | | (OSMR), mRNA. | | | |
| 7924495 | ENST00000391137 | ncrna:rRNA | — | — | 260 |
| | | chromosome:NCBI36:1:220936485:220936584:−1 | | | |
| | | gene:ENSG00000212439 | | | |
| 8122684 | ENST00000326669 /// | SMT3 suppressor of mif two 3 | SMT3 suppressor of | SUMO4 | 261 |
| | AY340238 /// | homolog 4 | mif two 3 homolog 4 | | |
| | NM_001002255 | gene:ENSG00000177688 /// | (S. cerevisiae) | | |
| | | Homo sapiens small ubiquitin- | | | |
| | | like protein 4 mRNA, complete | | | |
| | | cds. /// Homo sapiens SMT3 | | | |
| | | suppressor of mif two 3 | | | |
| | | homolog 4 (S. cerevisiae) | | | |
| | | (SUMO4), mRNA. | | | |
| 7940622 | ENST00000306238 /// | Secretoglobin family 1D | secretoglobin, family | SCGB1D1 | 262 |
| | BC062693 /// | member 1 | 1D, member 1 | | |
| | NM_006552 | gene:ENSG00000168515 /// | | | |
| | | Homo sapiens secretoglobin, | | | |
| | | family 1D, member 1, mRNA | | | |
| | | (cDNA clone MGC:71958 | | | |
| | | IMAGE:30327780), complete | | | |
| | | cds. /// Homo sapiens | | | |
| | | secretoglobin, family 1D, | | | |
| | | member 1 (SCGB1D1), mRNA. | | | |
| 8092654 | ENST00000296277 /// | 60S ribosomal protein L39-like | ribosomal protein | RPL39L | 263 |
| | BC012328 /// | gene:ENSG00000163923 /// | L39-like | | |
| | NM_052969 | Homo sapiens ribosomal | | | |
| | | protein L39-like, mRNA (cDNA | | | |
| | | clone MGC:20168 | | | |
| | | IMAGE:4555759), complete | | | |
| | | cds. /// Homo sapiens | | | |
| | | ribosomal protein L39-like | | | |
| | | (RPL39L), mRNA. | | | |
| 7944867 | ENST00000363408 | ncrna:rRNA | — | — | 264 |
| | | chromosome:NCBI36:11:124011565:124011685:1 | | | |
| | | gene:ENSG00000200278 | | | |
| 7925087 | ENST00000384108 | ncrna:snRNA | — | — | 265 |
| | | chromosome:NCBI36:1:231034386:231034556:−1 | | | |
| | | gene:ENSG00000206835 | | | |
| 7985920 | ENST00000341735 /// | mesoderm posterior 2 homolog | mesoderm posterior 2 | MESP2 | 266 |
| | BC111413 /// | gene:ENSG00000188095 /// | homolog (mouse) | | |
| | NM_001039958 | Homo sapiens mesoderm | | | |
| | | posterior 2 homolog (mouse), | | | |
| | | mRNA (cDNA clone | | | |
| | | MGC:133018 | | | |
| | | IMAGE:40004357), complete | | | |
| | | cds. /// Homo sapiens | | | |
| | | mesoderm posterior 2 homolog | | | |
| | | (mouse) (MESP2), mRNA. | | | |
| 7939314 | ENST00000257831 /// | Isoform 1 of ETS homologous | ets homologous factor | EHF | 267 |
| | AF203977 /// | factor | | | |
| | NM_012153 | gene:ENSG00000135373 /// | | | |
| | | Homo sapiens ETS-family | | | |
| | | transcription factor EHF (EHF) | | | |
| | | mRNA, complete cds. /// Homo sapiens | | | |
| | | ets homologous factor | | | |
| | | (EHF), mRNA. | | | |
| 7979179 | ENST00000395686 /// | Putative uncharacterized | ERO1-like | ERO1L | 268 |
| | ENST00000359133 /// | protein ERO1L | (S. cerevisiae) | | |
| | AF081886 /// | gene:ENSG00000197930 /// | | | |
| | NM_014584 | ERO1-like protein alpha | | | |
| | | gene:ENSG00000197930 /// | | | |
| | | Homo sapiens ERO1-like | | | |
| | | protein (ERO1-L) mRNA, | | | |
| | | complete cds. /// Homo sapiens | | | |
| | | ERO1-like (S. cerevisiae) | | | |
| | | (ERO1L), mRNA. | | | |
| 8123819 | ENST00000379715 /// | Eukaryotic translation | eukaryotic translation | EEF1E1 | 269 |
| | BC005291 /// | elongation factor 1 epsilon-1 | elongation factor 1 | | |
| | NM_004280 /// | gene:ENSG00000124802 /// | epsilon 1 | | |
| | NM_001135650 | Homo sapiens eukaryotic | | | |
| | | translation elongation factor 1 | | | |
| | | epsilon 1, mRNA (cDNA clone | | | |
| | | MGC:12352 IMAGE:3685030), | | | |
| | | complete cds. /// Homo sapiens | | | |
| | | eukaryotic translation | | | |
| | | elongation factor 1 epsilon 1 | | | |
| | | (EEF1E1), transcript variant 1, | | | |
| | | mRNA. /// Homo sapiens | | | |
| | | eukaryotic translation | | | |
| | | elongation factor 1 epsilon 1 | | | |
| | | (EEF1E1), transcript variant 2, | | | |
| | | mRNA. | | | |
| 8173414 | ENST00000298085 /// | Cationic amino acid transporter | solute carrier family 7 | SLC7A3 | 270 |
| | ENST00000374299 /// | 3 gene:ENSG00000165349 /// | (cationic amino acid | | |
| | BC033816 /// | Cationic amino acid transporter | transporter, y+ | | |
| | NM_032803 /// | 3 gene:ENSG00000165349 /// | system), member 3 | | |
| | NM_001048164 | Homo sapiens solute carrier | | | |
| | | family 7 (cationic amino acid | | | |
| | | transporter, y+ system), | | | |
| | | member 3, mRNA (cDNA clone | | | |
| | | MGC:44839 IMAGE:5206252), | | | |
| | | complete cds. /// Homo sapiens | | | |
| | | solute carrier family 7 (cationic | | | |
| | | amino acid transporter, y+ | | | |
| | | system), member 3 (SLC7A3), | | | |
| | | transcript variant 1, mRNA. /// | | | |
| | | Homo sapiens solute carrier | | | |
| | | family 7 (cationic amino acid | | | |
| | | transporter, y+ system), | | | |
| | | member 3 (SLC7A3), transcript | | | |
| | | variant 2, mRNA. | | | |
| 7952797 | ENST00000299140 /// | Spermatogenesis-associated | spermatogenesis | SPATA19 | 271 |
| | BC058039 /// | protein 19, mitochondrial | associated 19 | | |
| | NM_174927 | gene:ENSG00000166118 /// | | | |
| | | Homo sapiens | | | |
| | | spermatogenesis associated | | | |
| | | 19, mRNA (cDNA clone | | | |
| | | MGC:62071 IMAGE:6619434), | | | |
| | | complete cds. /// Homo sapiens | | | |
| | | spermatogenesis associated 19 | | | |
| | | (SPATA19), mRNA. | | | |
| 8031387 | ENST00000291890 /// | Isoform 1 of Natural cytotoxicity | natural cytotoxicity | NCR1 | 272 |
| | ENST00000338835 /// | triggering receptor 1 | triggering receptor 1 | | |
| | ENST00000350790 /// | gene:ENSG00000189430 /// | | | |
| | ENST00000357397 /// | Isoform 2 of Natural cytotoxicity | | | |
| | BC064806 /// | triggering receptor 1 | | | |
| | NM_004829 | gene:ENSG00000189430 /// | | | |
| | | Isoform 3 of Natural cytotoxicity | | | |
| | | triggering receptor 1 | | | |
| | | gene:ENSG00000189430 /// | | | |
| | | Isoform 5 of Natural cytotoxicity | | | |
| | | triggering receptor 1 | | | |
| | | gene:ENSG00000189430 /// | | | |
| | | Homo sapiens natural | | | |
| | | cytotoxicity triggering receptor | | | |
| | | 1, mRNA (cDNA clone | | | |
| | | MGC:65100 IMAGE:5218848), | | | |
| | | complete cds. /// Homo sapiens | | | |
| | | natural cytotoxicity triggering | | | |
| | | receptor 1 (NCR1), mRNA. | | | |
| 8038655 | ENST00000324041 /// | Kallikrein-4 | kallikrein-related | KLK4 | 273 |
| | AF113140 /// | gene:ENSG00000167749 /// | peptidase 4 | | |
| | NM_004917 | Homo sapiens serine protease | | | |
| | | prostase mRNA, complete cds. /// | | | |
| | | Homo sapiens kallikrein- | | | |
| | | related peptidase 4 (KLK4), | | | |
| | | mRNA. | | | |
| 8074106 | ENST00000252783 /// | Kelch domain-containing | kelch domain | KLHDC7B | 274 |
| | ENST00000395676 /// | protein 7B | containing 7B | | |
| | BC009980 /// | gene:ENSG00000130487 /// | | | |
| | NM_138433 | kelch domain containing 7B | | | |
| | | gene:ENSG00000130487 /// | | | |
| | | Homo sapiens kelch domain | | | |
| | | containing 7B, mRNA (cDNA | | | |
| | | clone MGC:16635 | | | |
| | | IMAGE:4121528), complete | | | |
| | | cds. /// Homo sapiens kelch | | | |
| | | domain containing 7B | | | |
| | | (KLHDC7B), mRNA. | | | |
| 8166382 | ENST00000379484 /// | Membrane-bound transcription | membrane-bound | MBTPS2 | 275 |
| | ENST00000365779 /// | factor site-2 protease | transcription factor | | |
| | AK292933 /// | gene:ENSG00000012174 /// 36 | peptidase, site 2 | | |
| | NM_015884 | kDa protein | | | |
| | | gene:ENSG00000012174 /// | | | |
| | | Homo sapiens cDNA FLJ75833 | | | |
| | | complete cds, highly similar to | | | |
| | | Homo sapiens membrane- | | | |
| | | bound transcription factor | | | |
| | | peptidase, site 2 (MBTPS2), | | | |
| | | mRNA. /// Homo sapiens | | | |
| | | membrane-bound transcription | | | |
| | | factor peptidase, site 2 | | | |
| | | (MBTPS2), mRNA. | | | |
| 7965884 | ENST00000307000 /// | Phenylalanine-4-hydroxylase | phenylalanine | PAH | 276 |
| | U49897 /// | gene:ENSG00000171759 /// | hydroxylase | | |
| | NM_000277 | Homo sapiens phenylalanine | | | |
| | | hydroxylase (PAH) mRNA, | | | |
| | | complete cds. /// Homo sapiens | | | |
| | | phenylalanine hydroxylase | | | |
| | | (PAH), mRNA. | | | |
| 8103684 | ENST00000261511 /// | UPF0609 protein C4orf27 | chromosome 4 open | C4orf27 | 277 |
| | BC010367 /// | gene:ENSG00000056050 /// | reading frame 27 | | |
| | NM_017867 | Homo sapiens chromosome 4 | | | |
| | | open reading frame 27, mRNA | | | |
| | | (cDNA clone MGC:13432 | | | |
| | | IMAGE:4334172), complete | | | |
| | | cds. /// Homo sapiens | | | |
| | | chromosome 4 open reading | | | |
| | | frame 27 (C4orf27), mRNA. | | | |
| 8139592 | ENST00000258774 /// | Checkpoint protein HUS1 | HUS1 checkpoint | HUS1 | 278 |
| | AF076844 /// | gene:ENSG00000136273 /// | homolog (S. pombe) | | |
| | NM_004507 | Homo sapiens Hus1-like protein | | | |
| | | (HUS1) mRNA, complete cds. /// | | | |
| | | Homo sapiens HUS1 | | | |
| | | checkpoint homolog (S. pombe) | | | |
| | | (HUS1), mRNA. | | | |
| 8004957 | ENST00000361801 /// | dynein, axonemal, heavy chain | dynein, axonemal, | DNAH9 | 279 |
| | ENST00000262442 /// | 9 isoform 1 | heavy chain 9 | | |
| | ENST00000396001 /// | gene:ENSG00000007174 /// | | | |
| | AJ404468 /// | Isoform 1 of Dynein heavy | | | |
| | NM_001372 /// | chain 9, axonemal | | | |
| | NM_004662 | gene:ENSG00000007174 /// | | | |
| | | dynein, axonemal, heavy chain | | | |
| | | 9 isoform 1 | | | |
| | | gene:ENSG00000007174 /// | | | |
| | | Homo sapiens mRNA for | | | |
| | | dynein heavy chain 9 (DNAH9 | | | |
| | | gene). /// Homo sapiens dynein, | | | |
| | | axonemal, heavy chain 9 | | | |
| | | (DNAH9), transcript variant 2, | | | |
| | | mRNA. /// Homo sapiens | | | |
| | | dynein, axonemal, heavy chain | | | |
| | | 9 (DNAH9), transcript variant 1, | | | |
| | | mRNA. | | | |
| 8130553 | AK130765 | Homo sapiens cDNA FLJ27255 | hypothetical | FLJ27255 | 280 |
| | | fis, clone SYN09519. | LOC401281 | | |
| 8094830 | ENST00000264452 /// | Transmembrane protein 33 | transmembrane | TMEM33 | 281 |
| | AY659966 /// | gene:ENSG00000109133 /// | protein 33 | | |
| | NM_018126 | Homo sapiens SHINC3 | | | |
| | | (SHINC3) mRNA, complete | | | |
| | | cds. /// Homo sapiens | | | |
| | | transmembrane protein 33 | | | |
| | | (TMEM33), mRNA. | | | |
| 8149438 | ENST00000329135 /// | Zeta-sarcoglycan | sarcoglycan zeta | SGCZ | 282 |
| | ENST00000382080 /// | gene:ENSG00000185053 /// | | | |
| | AY028700 /// | sarcoglycan zeta | | | |
| | NM_139167 | gene:ENSG00000185053 /// | | | |
| | | Homo sapiens zeta- | | | |
| | | sarcoglycan mRNA, complete | | | |
| | | cds. /// Homo sapiens | | | |
| | | sarcoglycan zeta (SGCZ), | | | |
| | | mRNA. | | | |
| 8180029 | ENST00000399426 /// | Major histocompatibility | major | HLA- | 283 |
| | ENST00000399424 /// | complex, class II, DQ beta 2 | histocompatibility | DQB2 | |
| | ENST00000399427 /// | gene:ENSG00000215008 /// | complex, class II, DQ | | |
| | ENST00000383099 /// | Major histocompatibility | beta 2 | | |
| | ENST00000383245 /// | complex, class II, DQ beta 2 | | | |
| | ENST00000323109 /// | gene:ENSG00000215008 /// | | | |
| | ENST00000399658 /// | Major histocompatibility | | | |
| | ENST00000399661 /// | complex, class II, DQ beta 2 | | | |
| | ENST00000323143 /// | gene:ENSG00000215008 /// | | | |
| | ENST00000399053 /// | Major histocompatibility | | | |
| | ENST00000374931 /// | complex, class II, DQ beta 2 | | | |
| | ENST00000374934 /// | gene:ENSG00000215008 /// | | | |
| | AK098007 /// | Major histocompatibility | | | |
| | NR_003937 | complex, class II, DQ beta 2 | | | |
| | | gene:ENSG00000196610 /// | | | |
| | | Major histocompatibility | | | |
| | | complex, class II, DQ beta 2 | | | |
| | | gene:ENSG00000196610 /// | | | |
| | | Major histocompatibility | | | |
| | | complex, class II, DQ beta 2 | | | |
| | | gene:ENSG00000196610 /// | | | |
| | | cdna:known | | | |
| | | chromosome:NCBI36:c6_QBL:32795451:32802909:−1 | | | |
| | | gene:ENSG00000196610 /// | | | |
| | | Major histocompatibility | | | |
| | | complex, class II, DQ beta 2 | | | |
| | | gene:ENSG00000204275 /// | | | |
| | | Major histocompatibility | | | |
| | | complex, class II, DQ beta 2 | | | |
| | | gene:ENSG00000204275 /// | | | |
| | | Major histocompatibility | | | |
| | | complex, class II, DQ beta 2 | | | |
| | | gene:ENSG00000204275 /// | | | |
| | | Major histocompatibility | | | |
| | | complex, class II, DQ beta 2 | | | |
| | | gene:ENSG00000204275 /// | | | |
| | | Homo sapiens cDNA FLJ40688 | | | |
| | | fis, clone THYMU2024185, | | | |
| | | highly similar to HLA class II | | | |
| | | histocompatibility antigen, DX | | | |
| | | beta chain precursor. /// Homo sapiens | | | |
| | | major histocompatibility | | | |
| | | complex, class II, DQ beta 2 | | | |
| | | (HLA-DQB2), non-coding RNA. | | | |
| 8015060 | ENST00000264651 /// | Keratin, type I cytoskeletal 24 | keratin 24 | KRT24 | 284 |
| | AK000268 /// | gene:ENSG00000167916 /// | | | |
| | NM_019016 | Homo sapiens cDNA FLJ20261 | | | |
| | | fis, clone COLF7630. /// Homo sapiens | | | |
| | | keratin 24 (KRT24), | | | |
| | | mRNA. | | | |
| 8066384 | ENST00000373005 /// | gametocyte specific factor 1- | gametocyte specific | GTSF1L | 285 |
| | ENST00000373003 /// | like isoform 2 | factor 1-like | | |
| | BC040049 /// | gene:ENSG00000124196 /// | | | |
| | NM_176791 /// | Gametocyte-specific factor 1- | | | |
| | NM_001008901 | like gene:ENSG00000124196 /// | | | |
| | | Homo sapiens gametocyte | | | |
| | | specific factor 1-like, mRNA | | | |
| | | (cDNA clone MGC:50820 | | | |
| | | IMAGE:5744556), complete | | | |
| | | cds. /// Homo sapiens | | | |
| | | gametocyte specific factor 1- | | | |
| | | like (GTSF1L), transcript variant | | | |
| | | 1, mRNA. /// Homo sapiens | | | |
| | | gametocyte specific factor 1- | | | |
| | | like (GTSF1L), transcript variant | | | |
| | | 2, mRNA. | | | |
| 8001197 | ENST00000303155 /// | Isoform 1 of Neuropilin and | neuropilin (NRP) and | NETO2 | 286 |
| | AY358718 /// | tolloid-like protein 2 | tolloid (TLL)-like 2 | | |
| | NM_018092 | gene:ENSG00000171208 /// | | | |
| | | Homo sapiens clone | | | |
| | | DNA84912 Neto2 (UNQ1926) | | | |
| | | mRNA, complete cds. /// Homo sapiens | | | |
| | | neuropilin (NRP) and | | | |
| | | tolloid (TLL)-like 2 (NETO2), | | | |
| | | mRNA. | | | |
| 8177195 | ENST00000253323 /// | Putative transcript Y 9 protein | testis-specific | TTTY9A /// | 287 |
| | ENST00000253325 /// | gene:ENSG00000131007 /// | transcript, Y-linked 9A /// | TTTY9B | |
| | AF332238 /// | Putative transcript Y 9 protein | testis-specific | | |
| | NR_001530 /// | gene:ENSG00000131009 /// | transcript, Y-linked 9B | | |
| | NR_002159 | Homo sapiens testis transcript | | | |
| | | Y 9 (TTY9) mRNA, complete | | | |
| | | cds. /// Homo sapiens testis- | | | |
| | | specific transcript, Y-linked 9A | | | |
| | | (TTTY9A), non-coding RNA. /// | | | |
| | | Homo sapiens testis-specific | | | |
| | | transcript, Y-linked 9B | | | |
| | | (TTTY9B), non-coding RNA. | | | |
| 8008885 | hsa-mir-21 /// | MI0000077 Homo sapiens miR- | microRNA 21 | MIR21 | 288 |
| | hsa-mir-21 /// | 21 stem-loop /// MI0000077 | | | |
| | AY699265 | Homo sapiens miR-21 stem- | | | |
| | | loop /// Homo sapiens | | | |
| | | microRNA pri-miR-21, complete | | | |
| | | sequence. | | | |
| 8176692 | ENST00000253323 /// | Putative transcript Y 9 protein | testis-specific | TTTY9A /// | 289 |
| | ENST00000253325 /// | gene:ENSG00000131007 /// | transcript, Y-linked 9A /// | TTTY9B | |
| | AF332238 /// | Putative transcript Y 9 protein | testis-specific | | |
| | NR_001530 /// | gene:ENSG00000131009 /// | transcript, Y-linked 9B | | |
| | NR_002159 | Homo sapiens testis transcript | | | |
| | | Y 9 (TTY9) mRNA, complete | | | |
| | | cds. /// Homo sapiens testis- | | | |
| | | specific transcript, Y-linked 9A | | | |
| | | (TTTY9A), non-coding RNA. /// | | | |
| | | Homo sapiens testis-specific | | | |
| | | transcript, Y-linked 9B | | | |
| | | (TTTY9B), non-coding RNA. | | | |
| 8056959 | ENST00000308618 /// | Homeobox even-skipped | even-skipped | EVX2 | 290 |
| | NM_001080458 | homolog protein 2 | homeobox 2 | | |
| | | gene:ENSG00000174279 /// | | | |
| | | Homo sapiens even-skipped | | | |
| | | homeobox 2 (EVX2), mRNA. | | | |
| 7974562 | ENST00000363948 | ncrna:snRNA | — | — | 291 |
| | | chromosome:NCBI36:14:56361457:56361563:1 | | | |
| | | gene:ENSG00000200818 | | | |
| 8136557 | ENST00000336425 /// | thromboxane A synthase 1 | thromboxane A | TBXAS1 | 292 |
| | ENST00000263552 /// | isoform TXS-I | synthase 1 (platelet) | | |
| | BC014117 /// | gene:ENSG00000059377 /// | | | |
| | NM_001130966 /// | thromboxane A synthase 1 | | | |
| | NM_001061 /// | isoform TXS-II gene:ENSG00000059377 /// | | | |
| | NM_030984 | Homo sapiens thromboxane A | | | |
| | | synthase 1 (platelet), mRNA | | | |
| | | (cDNA clone MGC:20885 | | | |
| | | IMAGE:4548935), complete | | | |
| | | cds. /// Homo sapiens | | | |
| | | thromboxane A synthase 1 | | | |
| | | (platelet) (TBXAS1), transcript | | | |
| | | variant TXS-III, mRNA. /// | | | |
| | | Homo sapiens thromboxane A | | | |
| | | synthase 1 (platelet) (TBXAS1), | | | |
| | | transcript variant TXS-I, mRNA. /// | | | |
| | | Homo sapiens thromboxane | | | |
| | | A synthase 1 (platelet) | | | |
| | | (TBXAS1), transcript variant | | | |
| | | TXS-II, mRNA. | | | |
| 7936968 | ENST00000368679 /// | Isoform 1 of ADAM 12 | ADAM | ADAM12 | 293 |
| | ENST00000368683 /// | gene:ENSG00000148848 /// | metallopeptidase | | |
| | ENST00000368676 /// | Isoform 4 of ADAM 12 | domain 12 | | |
| | AF023476 /// | gene:ENSG00000148848 /// | | | |
| | NM_003474 /// | Isoform 2 of ADAM 12 | | | |
| | NM_021641 | gene:ENSG00000148848 /// | | | |
| | | Homo sapiens meltrin-L | | | |
| | | precursor (ADAM12) mRNA, | | | |
| | | complete cds, alternatively | | | |
| | | spliced. /// Homo sapiens | | | |
| | | ADAM metallopeptidase | | | |
| | | domain 12 (ADAM12), | | | |
| | | transcript variant 1, mRNA. /// | | | |
| | | Homo sapiens ADAM | | | |
| | | metallopeptidase domain 12 | | | |
| | | (ADAM12), transcript variant 2, | | | |
| | | mRNA. | | | |
| 8019478 | ENST00000312648 /// | T-cell antigen CD7 | CD7 molecule | CD7 | 294 |
| | AY935535 /// | gene:ENSG00000173762 /// | | | |
| | NM_006137 | Homo sapiens clone 14 CD7 | | | |
| | | antigen mRNA, complete cds. /// | | | |
| | | Homo sapiens CD7 molecule | | | |
| | | (CD7), mRNA. | | | |
| 8073799 | ENST00000252934 /// | Ataxin-10 | ataxin 10 | ATXN10 | 295 |
| | ENST00000396011 /// | gene:ENSG00000130638 /// | | | |
| | ENST00000381061 /// | HUMEEP | | | |
| | ENST00000402380 /// | gene:ENSG00000130638 /// | | | |
| | BC007508 /// | Putative uncharacterized | | | |
| | NM_013236 | protein ATXN10 | | | |
| | | gene:ENSG00000130638 /// | | | |
| | | Ataxin 10 | | | |
| | | gene:ENSG00000130638 /// | | | |
| | | Homo sapiens ataxin 10, | | | |
| | | mRNA (cDNA clone MGC:4152 | | | |
| | | IMAGE:3030062), complete | | | |
| | | cds. /// Homo sapiens ataxin 10 | | | |
| | | (ATXN10), mRNA. | | | |
| 8035789 | ENST00000358224 /// | Zinc finger protein 826 | zinc finger protein 826 | ZNF826 | 296 |
| | BC016785 /// | gene:ENSG00000178604 /// | | | |
| | NM_001039884 | Homo sapiens zinc finger | | | |
| | | protein 826, mRNA (cDNA | | | |
| | | clone IMAGE:4096414), | | | |
| | | complete cds. /// Homo sapiens | | | |
| | | zinc finger protein 826 | | | |
| | | (ZNF826), mRNA. | | | |
| 7951485 | ENST00000265836 /// | Isoform 1 of Solute carrier | solute carrier family | SLC35F2 | 297 |
| | ENST00000375682 /// | family 35 member F2 | 35, member F2 | | |
| | AK128062 /// | gene:ENSG00000110660 /// | | | |
| | NM_017515 | Putative uncharacterized | | | |
| | | protein SLC35F2 (Fragment) | | | |
| | | gene:ENSG00000110660 /// | | | |
| | | Homo sapiens cDNA FLJ46182 | | | |
| | | fis, clone TESTI4004539. /// | | | |
| | | Homo sapiens solute carrier | | | |
| | | family 35, member F2 | | | |
| | | (SLC35F2), mRNA. | | | |
| 8114805 | ENST00000359370 /// | Heparin-binding growth factor 1 | fibroblast growth | FGF1 | 298 |
| | ENST00000378046 /// | gene:ENSG00000113578 /// | factor 1 (acidic) | | |
| | ENST00000337706 /// | Heparin-binding growth factor 1 | | | |
| | ENST00000360966 /// | gene:ENSG00000113578 /// | | | |
| | ENST00000407758 /// | Heparin-binding growth factor 1 | | | |
| | ENST00000405304 /// | gene:ENSG00000113578 /// | | | |
| | ENST00000403113 /// | fibroblast growth factor 1 | | | |
| | ENST00000378047 /// | (acidic) isoform 2 precursor | | | |
| | ENST00000394496 /// | gene:ENSG00000113578 /// | | | |
| | ENST00000394493 /// | Putative uncharacterized | | | |
| | BC032697 /// | protein FGF1 | | | |
| | NM_033136 /// | gene:ENSG00000113578 /// | | | |
| | NM_033137 /// | fibroblast growth factor 1 | | | |
| | NM_000800 | (acidic) isoform 2 precursor | | | |
| | | gene:ENSG00000113578 /// | | | |
| | | Heparin-binding growth factor 1 | | | |
| | | gene:ENSG00000113578 /// | | | |
| | | Heparin-binding growth factor 1 | | | |
| | | gene:ENSG00000113578 /// | | | |
| | | Heparin-binding growth factor 1 | | | |
| | | gene:ENSG00000113578 /// | | | |
| | | fibroblast growth factor 1 | | | |
| | | (acidic) isoform 3 precursor | | | |
| | | gene:ENSG00000113578 /// | | | |
| | | Homo sapiens fibroblast growth | | | |
| | | factor 1 (acidic), mRNA (cDNA | | | |
| | | clone MGC:44867 | | | |
| | | IMAGE:5403677), complete | | | |
| | | cds. /// Homo sapiens fibroblast | | | |
| | | growth factor 1 (acidic) (FGF1), | | | |
| | | transcript variant 2, mRNA. /// | | | |
| | | Homo sapiens fibroblast growth | | | |
| | | factor 1 (acidic) (FGF1), | | | |
| | | transcript variant 3, mRNA. /// | | | |
| | | Homo sapiens fibroblast growth | | | |
| | | factor 1 (acidic) (FGF1), | | | |
| | | transcript variant 1, mRNA. | | | |
| 8095680 | ENST00000401931 /// | 11 kDa protein | interleukin 8 | IL8 | 299 |
| | ENST00000307407 /// | gene:ENSG00000169429 /// | | | |
| | ENST00000395775 /// | Isoform 1 of Interleukin-8 | | | |
| | M17017 /// | gene:ENSG00000169429 /// 15 | | | |
| | NM_000584 | kDa protein | | | |
| | | gene:ENSG00000169429 /// | | | |
| | | Human beta-thromboglobulin- | | | |
| | | like protein mRNA, complete | | | |
| | | cds. /// Homo sapiens | | | |
| | | interleukin 8 (IL8), mRNA. | | | |
| 8017827 | ENST00000364677 | ncrna:misc_RNA | — | — | 300 |
| | | chromosome:NCBI36:17:62835352:62835459:−1 | | | |
| | | gene:ENSG00000201547 | | | |
| 8083034 | ENST00000332210 /// | Calsyntenin-2 | calsyntenin 2 | CLSTN2 | 301 |
| | AY753303 /// | gene:ENSG00000158258 /// | | | |
| | NM_022131 | Homo sapiens alcadein gamma | | | |
| | | mRNA, complete cds. /// Homo sapiens | | | |
| | | calsyntenin 2 | | | |
| | | (CLSTN2), mRNA. | | | |
| 8131140 | ENST00000313156 /// | FLJ00049 protein (Fragment) | FLJ00049 protein | FLJ00049 | 302 |
| | AK024457 | gene:ENSG00000175873 /// | | | |
| | | Homo sapiens mRNA for | | | |
| | | FLJ00049 protein, partial cds. | | | |
| 7977003 | ENST00000262241 /// | REST corepressor 1 | REST corepressor 1 | RCOR1 | 303 |
| | AF155595 /// | gene:ENSG00000089902 /// | | | |
| | NM_015156 | Homo sapiens CoREST protein | | | |
| | | (COREST) mRNA, complete | | | |
| | | cds. /// Homo sapiens REST | | | |
| | | corepressor 1 (RCOR1), | | | |
| | | mRNA. | | | |
| 8144774 | ENST00000324815 /// | Isoform 3 of Vacuolar protein | vacuolar protein | VPS37A | 304 |
| | ENST00000324849 /// | sorting-associated protein 37A | sorting 37 homolog A | | |
| | BC067754 /// | gene:ENSG00000155975 /// | (S. cerevisiae) | | |
| | NM_152415 | Isoform 1 of Vacuolar protein | | | |
| | | sorting-associated protein 37A | | | |
| | | gene:ENSG00000155975 /// | | | |
| | | Homo sapiens vacuolar protein | | | |
| | | sorting 37 homolog A | | | |
| | | (S. cerevisiae), mRNA (cDNA | | | |
| | | clone MGC:87029 | | | |
| | | IMAGE:5275060), complete | | | |
| | | cds. /// Homo sapiens vacuolar | | | |
| | | protein sorting 37 homolog A | | | |
| | | (S. cerevisiae) (VPS37A), | | | |
| | | mRNA. | | | |
| 8172266 | hsa-mir-221 /// | MI0000298 Homo sapiens miR- | — | — | 305 |
| | hsa-mir-221 | 221 stem-loop /// MI0000298 | | | |
| | | Homo sapiens miR-221 stem- | | | |
| | | loop | | | |
| 8091186 | — | — | — | — | 306 |
| 8076298 | ENST00000386767 | ncrna:scRNA_pseudogene | — | — | 307 |
| | | chromosome:NCBI36:22:39776868:39776953:−1 | | | |
| | | gene:ENSG00000209502 | | | |
| 8094704 | ENST00000295963 /// | Isoform 2 of Ubiquitin- | ubiquitin-conjugating | UBE2K | 308 |
| | ENST00000261427 /// | conjugating enzyme E2 K | enzyme E2K (UBC1 | | |
| | U58522 /// | gene:ENSG00000078140 /// | homolog, yeast) | | |
| | NM_001111112 /// | Isoform 1 of Ubiquitin- | | | |
| | NM_001111113 /// | conjugating enzyme E2 K | | | |
| | NM_005339 | gene:ENSG00000078140 /// | | | |
| | | Human huntingtin interacting | | | |
| | | protein (HIP2) mRNA, complete | | | |
| | | cds. /// Homo sapiens ubiquitin- | | | |
| | | conjugating enzyme E2K | | | |
| | | (UBC1 homolog, yeast) | | | |
| | | (UBE2K), transcript variant 2, | | | |
| | | mRNA. /// Homo sapiens | | | |
| | | ubiquitin-conjugating enzyme | | | |
| | | E2K (UBC1 homolog, yeast) | | | |
| | | (UBE2K), transcript variant 3, | | | |
| | | mRNA. /// Homo sapiens | | | |
| | | ubiquitin-conjugating enzyme | | | |
| | | E2K (UBC1 homolog, yeast) | | | |
| | | (UBE2K), transcript variant 1, | | | |
| | | mRNA. | | | |
| 8089329 | ENST00000273353 /// | Myosin-15 | myosin, heavy chain | MYH15 | 309 |
| | AK126801 /// | gene:ENSG00000144821 /// | 15 | | |
| | NM_014981 | Homo sapiens cDNA FLJ44851 | | | |
| | | fis, clone BRACE3051819, | | | |
| | | moderately similar to Myosin | | | |
| | | heavy chain, cardiac muscle | | | |
| | | alpha isoform. /// Homo sapiens | | | |
| | | myosin, heavy chain 15 | | | |
| | | (MYH15), mRNA. | | | |
| 8116548 | ENST00000344450 /// | Isoform 1 of Dual specificity | dual specificity | DUSP22 | 310 |
| | BC022847 /// | protein phosphatase 22 | phosphatase 22 | | |
| | NM_020185 | gene:ENSG00000112679 /// | | | |
| | | Homo sapiens dual specificity | | | |
| | | phosphatase 22, mRNA (cDNA | | | |
| | | clone MGC:15090 | | | |
| | | IMAGE:3942055), complete | | | |
| | | cds. /// Homo sapiens dual | | | |
| | | specificity phosphatase 22 | | | |
| | | (DUSP22), mRNA. | | | |
| 7903294 | ENST00000370152 /// | Hippocampus abundant | hippocampus | HIAT1 | 311 |
| | AK057172 /// | transcript 1 protein | abundant transcript 1 | | |
| | NM_033055 | gene:ENSG00000156875 /// | | | |
| | | Homo sapiens cDNA FLJ32610 | | | |
| | | fis, clone STOMA2000055, | | | |
| | | highly similar to Mouse mRNA | | | |
| | | for tetracycline transporter-like | | | |
| | | protein. /// Homo sapiens | | | |
| | | hippocampus abundant | | | |
| | | transcript 1 (HIAT1), mRNA. | | | |
| 7977567 | ENST00000344581 /// | similar to hCG2036672 | kelch-like 33 | KLHL33 | 312 |
| | BC146951 | gene:ENSG00000185271 /// | (Drosophila) | | |
| | | Homo sapiens kelch-like 33 | | | |
| | | (Drosophila), mRNA (cDNA | | | |
| | | clone MGC:182054 | | | |
| | | IMAGE:9056879), complete | | | |
| | | cds. | | | |
| 7902685 | ENST00000411322 | ncrna:misc_RNA | — | — | 313 |
| | | chromosome:NCBI36:1:85502567:85502673:1 | | | |
| | | gene:ENSG00000223254 | | | |
| 8137219 | ENST00000343855 /// | Uncharacterized protein | chromosome 7 open | C7orf29 | 314 |
| | AL832660 /// | C7orf29 | reading frame 29 | | |
| | NM_138434 | gene:ENSG00000188707 /// | | | |
| | | Homo sapiens mRNA; cDNA | | | |
| | | DKFZp313D2012 (from clone | | | |
| | | DKFZp313D2012). /// Homo sapiens | | | |
| | | chromosome 7 open | | | |
| | | reading frame 29 (C7orf29), | | | |
| | | mRNA. | | | |
| 7927153 | ENST00000387115 /// | ncrna:snRNA_pseudogene | — | — | 315 |
| | ENST00000408541 | chromosome:NCBI36:10:43157240:43157363:1 | | | |
| | | gene:ENSG00000209850 /// | | | |
| | | ncrna:snRNA | | | |
| | | chromosome:NCBI36:10:43157240:43157364:1 | | | |
| | | gene:ENSG00000221468 | | | |
| 7919572 | ENST00000386002 | ncrna:tRNA_pseudogene | — | — | 316 |
| | | chromosome:NCBI36:1:147478572:147478645:−1 | | | |
| | | gene:ENSG00000208737 | | | |
| 8073544 | hsa-mir-33a /// | MI0000091 Homo sapiens miR- | — | — | 317 |
| | hsa-mir-33a | 33a stem-loop /// MI0000091 | | | |
| | | Homo sapiens miR-33a stem- | | | |
| | | loop | | | |
| 8090091 | ENST00000383657 /// | Protein-tyrosine phosphatase- | protein tyrosine | PTPLB | 318 |
| | BC049369 /// | like member B | phosphatase-like | | |
| | NM_198402 | gene:ENSG00000206527 /// | (proline instead of | | |
| | | Homo sapiens protein tyrosine | catalytic arginine), | | |
| | | phosphatase-like (proline | member b | | |
| | | instead of catalytic arginine), | | | |
| | | member b, mRNA (cDNA clone | | | |
| | | MGC:57203 IMAGE:5286864), | | | |
| | | complete cds. /// Homo sapiens | | | |
| | | protein tyrosine phosphatase- | | | |
| | | like (proline instead of catalytic | | | |
| | | arginine), member b (PTPLB), | | | |
| | | mRNA. | | | |
| 8045889 | ENST00000263635 /// | Isoform 1 of Protein TANC1 | tetratricopeptide | TANC1 | 319 |
| | AK128859 /// | gene:ENSG00000115183 /// | repeat, ankyrin repeat | | |
| | NM_033394 | Homo sapiens cDNA FLJ46667 | and coiled-coil | | |
| | | fis, clone TRACH3007689. /// | containing 1 | | |
| | | Homo sapiens tetratricopeptide | | | |
| | | repeat, ankyrin repeat and | | | |
| | | coiled-coil containing 1 | | | |
| | | (TANC1), mRNA. | | | |
| 7974689 | ENST00000395151 /// | 60 kDa protein | dapper, antagonist of | DACT1 | 320 |
| | ENST00000335867 /// | gene:ENSG00000165617 /// | beta-catenin, | | |
| | ENST00000395153 /// | Dapper homolog 1 | homolog 1 | | |
| | AF251079 /// | gene:ENSG00000165617 /// | (Xenopus laevis) | | |
| | NM_001079520 /// | dapper 1 isoform 2 | | | |
| | NM_016651 | gene:ENSG00000165617 /// | | | |
| | | Homo sapiens heptacellular | | | |
| | | carcinoma novel gene-3 protein | | | |
| | | mRNA, complete cds. /// Homo sapiens | | | |
| | | dapper, antagonist of | | | |
| | | beta-catenin, homolog 1 | | | |
| | | (Xenopus laevis) (DACT1), | | | |
| | | transcript variant 2, mRNA. /// | | | |
| | | Homo sapiens dapper, | | | |
| | | antagonist of beta-catenin, | | | |
| | | homolog 1 (Xenopus laevis) | | | |
| | | (DACT1), transcript variant 1, | | | |
| | | mRNA. | | | |
| 8160040 | ENST00000346816 /// | Isoform 3 of Receptor-type | protein tyrosine | PTPRD | 321 |
| | ENST00000381196 /// | tyrosine-protein phosphatase | phosphatase, | | |
| | ENST00000356435 /// | delta gene:ENSG00000153707 /// | receptor type, D | | |
| | ENST00000358503 /// | Isoform 1 of Receptor-type | | | |
| | ENST00000360074 /// | tyrosine-protein phosphatase | | | |
| | ENST00000397617 /// | delta gene:ENSG00000153707 /// | | | |
| | ENST00000397611 /// | Isoform 1 of Receptor-type | | | |
| | ENST00000355233 /// | tyrosine-protein phosphatase | | | |
| | ENST00000397606 /// | delta gene:ENSG00000153707 /// | | | |
| | AB211400 /// | 214 kDa protein | | | |
| | NM_130393 /// | gene:ENSG00000153707 /// | | | |
| | NM_130392 /// | Protein tyrosine phosphatase | | | |
| | NM_130391 /// | receptor type D | | | |
| | NM_002839 /// | gene:ENSG00000153707 /// | | | |
| | NM_001040712 | 215 kDa protein | | | |
| | | gene:ENSG00000153707 /// | | | |
| | | 170 kDa protein | | | |
| | | gene:ENSG00000153707 /// | | | |
| | | PTPRD protein | | | |
| | | gene:ENSG00000153707 /// | | | |
| | | PTPRD protein | | | |
| | | gene:ENSG00000153707 /// | | | |
| | | Homo sapiens PTPRD mRNA | | | |
| | | for protein tyrosine | | | |
| | | phosphatase receptor type D, | | | |
| | | complete cds. /// Homo sapiens | | | |
| | | protein tyrosine phosphatase, | | | |
| | | receptor type, D (PTPRD), | | | |
| | | transcript variant 4, mRNA. /// | | | |
| | | Homo sapiens protein tyrosine | | | |
| | | phosphatase, receptor type, D | | | |
| | | (PTPRD), transcript variant 3, | | | |
| | | mRNA. /// Homo sapiens | | | |
| | | protein tyrosine phosphatase, | | | |
| | | receptor type, D (PTPRD), | | | |
| | | transcript variant 2, mRNA. /// | | | |
| | | Homo sapiens protein tyrosine | | | |
| | | phosphatase, receptor type, D | | | |
| | | (PTPRD), transcript variant 1, | | | |
| | | mRNA. /// Homo sapiens | | | |
| | | protein tyrosine phosphatase, | | | |
| | | receptor type, D (PTPRD), | | | |
| | | transcript variant 5, mRNA. | | | |
| 8142878 | AF503918 | Homo sapiens CDC26 subunit | cell division cycle 26 | CDC26 | 322 |
| | | of anaphase promoting | homolog | | |
| | | complex (CDC26) mRNA, | (S. cerevisiae) | | |
| | | complete cds. | | | |
| 8065603 | BC101556 /// | Homo sapiens TSPY-like 3 | TSPY-like 3 | TSPYL3 | 323 |
| | NR_002781 | (pseudogene), mRNA (cDNA | (pseudogene) | | |
| | | clone MGC:126605 | | | |
| | | IMAGE:8069062), complete | | | |
| | | cds. /// Homo sapiens TSPY- | | | |
| | | like 3 (pseudogene) (TSPYL3), | | | |
| | | non-coding RNA. | | | |
| 8104781 | ENST00000330120 /// | Relaxin-3 receptor 1 | relaxin/insulin-like | RXFP3 | 324 |
| | D88437 /// | gene:ENSG00000182631 /// | family peptide | | |
| | NM_016568 | Homo sapiens mRNA for G- | receptor 3 | | |
| | | protein coupled receptor | | | |
| | | SALPR, complete cds. /// Homo sapiens | | | |
| | | relaxin/insulin-like | | | |
| | | family peptide receptor 3 | | | |
| | | (RXFP3), mRNA. | | | |
| 7959012 | ENST00000410526 /// | ncrna:misc_RNA | — | — | 325 |
| | ENST00000386460 | chromosome:NCBI36:12:114639906:114640199:1 | | | |
| | | gene:ENSG00000222458 /// | | | |
| | | ncrna:scRNA_pseudogene | | | |
| | | chromosome:NCBI36:12:114639907:114640200:1 | | | |
| | | gene:ENSG00000209195 | | | |
| 7954692 | ENST00000313737 /// | Putative uncharacterized | hypothetical protein | FLJ13224 | 326 |
| | AK023286 | protein FLJ13224 | FLJ13224 | | |
| | | gene:ENSG00000177340 /// | | | |
| | | Homo sapiens cDNA FLJ13224 | | | |
| | | fis, clone OVARC1000008. | | | |
| 7951038 | AK128061 /// | Homo sapiens cDNA FLJ46181 | TATA box binding | TAF1D /// | 327 |
| | NR_002973 | fis, clone TESTI4004210. /// | protein (TBP)- | SNORA40 | |
| | | Homo sapiens small nucleolar | associated factor, | | |
| | | RNA, H/ACA box 40 | RNA polymerase I, D, | | |
| | | (SNORA40), non-coding RNA. | 41 kDa /// small | | |
| | | | nucleolar RNA, | | |
| | | | H/ACA box 40 | | |
| 8169984 | ENST00000370796 /// | Putative uncharacterized | hypoxanthine | HPRT1 | 328 |
| | ENST00000298556 /// | protein HPRT1 | phosphoribosyltransferase | | |
| | M31642 /// | gene:ENSG00000165704 /// | 1 | | |
| | NM_000194 | Hypoxanthine-guanine | | | |
| | | phosphoribosyltransferase | | | |
| | | gene:ENSG00000165704 /// | | | |
| | | Homo sapiens hypoxanthine | | | |
| | | phosphoribosyltransferase 1 | | | |
| | | (HPRT1) mRNA, complete cds. /// | | | |
| | | Homo sapiens hypoxanthine | | | |
| | | phosphoribosyltransferase 1 | | | |
| | | (HPRT1), mRNA. | | | |
| 7933204 | ENST00000298295 /// | Protein DEPP | chromosome 10 open | C10orf10 | 329 |
| | AB022718 /// | gene:ENSG00000165507 /// | reading frame 10 | | |
| | NM_007021 | Homo sapiens mRNA for DEPP | | | |
| | | (decidual protein induced by | | | |
| | | progesterone), complete cds. /// | | | |
| | | Homo sapiens chromosome 10 | | | |
| | | open reading frame 10 | | | |
| | | (C10orf10), mRNA. | | | |
| 7953747 | ENST00000364910 | ncrna:snRNA | — | — | 330 |
| | | chromosome:NCBI36:12:8528609:8528715:1 | | | |
| | | gene:ENSG00000201780 | | | |
| 8175256 | uc004exm.1 /// | /// cdna:Genscan | hypothetical protein | MGC16121 | 331 |
| | GENSCAN00000003290 /// | chromosome:NCBI36:X:133504408:133568038:−1 /// | MGC16121 | | |
| | BC007360 /// | Homo sapiens | | | |
| | XM_001715787 /// | hypothetical protein | | | |
| | XM_001128419 /// | MGC16121, mRNA (cDNA | | | |
| | XM_001715872 | clone IMAGE:3627113), | | | |
| | | complete cds. /// PREDICTED: | | | |
| | | Homo sapiens hypothetical | | | |
| | | protein MGC16121 | | | |
| | | (MGC16121), mRNA. /// | | | |
| | | PREDICTED: Homo sapiens | | | |
| | | hypothetical protein MGC16121 | | | |
| | | (MGC16121), mRNA. /// | | | |
| | | PREDICTED: Homo sapiens | | | |
| | | hypothetical protein MGC16121 | | | |
| | | (MGC16121), mRNA. | | | |
| 8180344 | — | — | — | — | 332 |
| 8013517 | ENST00000387217 | ncrna:Mt_tRNA_pseudogene | — | — | 333 |
| | | chromosome:NCBI36:17:21942958:21943026:−1 | | | |
| | | gene:ENSG00000209952 | | | |
| 8155802 | ENST00000376993 /// | Guanine deaminase | guanine deaminase | GDA | 334 |
| | ENST00000376989 /// | gene:ENSG00000119125 /// | | | |
| | ENST00000358399 /// | Guanine deaminase | | | |
| | ENST00000238018 /// | gene:ENSG00000119125 /// | | | |
| | AF019638 /// | Guanine aminohydrolase | | | |
| | NM_004293 | gene:ENSG00000119125 /// | | | |
| | | cDNA FLJ60569, highly similar | | | |
| | | to Guanine deaminase | | | |
| | | gene:ENSG00000119125 /// | | | |
| | | Homo sapiens nedasin s-form | | | |
| | | mRNA, complete cds. /// Homo sapiens | | | |
| | | guanine deaminase | | | |
| | | (GDA), mRNA. | | | |
| 8114468 | NR_002913 | Homo sapiens small nucleolar | small nucleolar RNA, | SNORD63 | 335 |
| | | RNA, C/D box 63 (SNORD63), | C/D box 63 | | |
| | | non-coding RNA. | | | |
| 8140398 | ENST00000307630 /// | 14-3-3 protein gamma | tyrosine 3- | YWHAG | 336 |
| | BC020963 /// | gene:ENSG00000170027 /// | monooxygenase/ | | |
| | NM_012479 | Homo sapiens tyrosine 3- | tryptophan 5- | | |
| | | monooxygenase/tryptophan 5- | monooxygenase | | |
| | | monooxygenase activation | activation protein, | | |
| | | protein, gamma polypeptide, | gamma polypeptide | | |
| | | mRNA (cDNA clone MGC:8908 | | | |
| | | IMAGE:3915246), complete | | | |
| | | cds. /// Homo sapiens tyrosine | | | |
| | | 3-monooxygenase/tryptophan | | | |
| | | 5-monooxygenase activation | | | |
| | | protein, gamma polypeptide | | | |
| | | (YWHAG), mRNA. | | | |
| 7982000 | NR_003340 | Homo sapiens small nucleolar | small nuclear | SNRPN /// | 337 |
| | | RNA, C/D box 116-26 | ribonucleoprotein | SNORD116-26 | |
| | | (SNORD116-26), non-coding | polypeptide N /// small | | |
| | | RNA. | nucleolar RNA, C/D | | |
| | | | box 116-26 | | |
| 8141150 | ENST00000394309 /// | Asparagine synthetase | asparagine | ASNS | 338 |
| | ENST00000175506 /// | [glutamine-hydrolyzing] | synthetase | | |
| | ENST00000394308 /// | gene:ENSG00000070669 /// | | | |
| | BC008723 /// | Asparagine synthetase | | | |
| | | [glutamine-hydrolyzing] | | | |
| | | gene:ENSG00000070669 /// | | | |
| | | Asparagine synthetase | | | |
| | NM_133436 /// | [glutamine-hydrolyzing] | — | — | |
| | NM_001673 /// | gene:ENSG00000070669 /// | | | |
| | NM_183356 | Homo sapiens asparagine | | | |
| | | synthetase, mRNA (cDNA | | | |
| | | clone MGC:8639 | | | |
| | | IMAGE:2961551), complete | | | |
| | | cds. /// Homo sapiens | | | |
| | | asparagine synthetase (ASNS), | | | |
| | | transcript variant 1, mRNA. /// | | | |
| | | Homo sapiens asparagine | | | |
| | | synthetase (ASNS), transcript | | | |
| | | variant 2, mRNA. /// Homo sapiens | | | |
| | | asparagine synthetase | | | |
| | | (ASNS), transcript variant 3, | | | |
| | | mRNA. | | | |
| 8055265 | ENST00000385636 | ncrna:Mt_tRNA_pseudogene | — | — | 339 |
| | | chromosome:NCBI36:2:131846208:131846265:−1 | | | |
| | | gene:ENSG00000208371 | | | |
| 8105607 | ENST00000389074 | UPF0514 membrane protein | — | — | 340 |
| | | FAM159B | | | |
| | | gene:ENSG00000145642 | | | |
| 8099130 | ENST00000363891 | ncrna:misc_RNA | — | — | 341 |
| | | chromosome:NCBI36:4:4973381:4973692:−1 | | | |
| | | gene:ENSG00000200761 | | | |
| 8015456 | ENST00000319121 /// | Kelch-like protein 11 | kelch-like 11 | KLHL11 | 342 |
| | BC034470 /// | gene:ENSG00000178502 /// | (Drosophila) | | |
| | NM_018143 | Homo sapiens kelch-like 11 | | | |
| | | (Drosophila), mRNA (cDNA | | | |
| | | clone MGC:26174 | | | |
| | | IMAGE:4822768), complete | | | |
| | | cds. /// Homo sapiens kelch-like | | | |
| | | 11 (Drosophila) (KLHL11), | | | |
| | | mRNA. | | | |
| 8028332 | ENST00000263372 /// | Isoform 1 of Potassium channel | potassium channel, | KCNK6 | 343 |
| | AF117708 /// | subfamily K member 6 | subfamily K, member | | |
| | NM_004823 | gene:ENSG00000099337 /// | 6 | | |
| | | Homo sapiens tandem pore | | | |
| | | domain potassium channel | | | |
| | | TWIK-2 (KCNK6) mRNA, | | | |
| | | complete cds. /// Homo sapiens | | | |
| | | potassium channel, subfamily | | | |
| | | K, member 6 (KCNK6), mRNA. | | | |
| 7947147 | ENST00000354193 /// | Small VCP/p97-interacting | small VCP/p97- | SVIP | 344 |
| | AL833119 /// | protein | interacting protein | | |
| | NM_148893 | gene:ENSG00000198168 /// | | | |
| | | Homo sapiens mRNA; cDNA | | | |
| | | DKFZp313A2432 (from clone | | | |
| | | DKFZp313A2432). /// Homo sapiens | | | |
| | | small VCP/p97- | | | |
| | | interacting protein (SVIP), | | | |
| | | mRNA. | | | |
| 7961198 | ENST00000023165 /// | killer cell lectin-like receptor | killer cell lectin-like | KLRA1 | 345 |
| | ENST00000341141 /// | subfamily A, member 1 | receptor subfamily A, | | |
| | AK296103 /// | gene:ENSG00000021602 /// | member 1 | | |
| | NM_006611 | KLRA1 | | | |
| | | gene:ENSG00000021602 /// | | | |
| | | Homo sapiens cDNA FLJ59270 | | | |
| | | complete cds, highly similar to | | | |
| | | Homo sapiens killer cell lectin- | | | |
| | | like receptor subfamily A, | | | |
| | | member 1 (KLRA1), mRNA. /// | | | |
| | | Homo sapiens killer cell lectin- | | | |
| | | like receptor subfamily A, | | | |
| | | member 1 (KLRA1), mRNA. | | | |
| 7957008 | ENST00000266679 /// | Isoform 2 of Cleavage and | cleavage and | CPSF6 | 346 |
| | ENST00000351671 /// | polyadenylation specificity | polyadenylation | | |
| | BC000714 /// | factor subunit 6 | specific factor 6, | | |
| | NM_007007 | gene:ENSG00000111605 /// | 68 kDa | | |
| | | Isoform 1 of Cleavage and | | | |
| | | polyadenylation specificity | | | |
| | | factor subunit 6 | | | |
| | | gene:ENSG00000111605 /// | | | |
| | | Homo sapiens cleavage and | | | |
| | | polyadenylation specific factor | | | |
| | | 6, 68 kDa, mRNA (cDNA clone | | | |
| | | MGC:1242 IMAGE:3506481), | | | |
| | | complete cds. /// Homo sapiens | | | |
| | | cleavage and polyadenylation | | | |
| | | specific factor 6, 68 kDa | | | |
| | | (CPSF6), mRNA. | | | |
| 8046790 | ENST00000384449 | ncrna:snRNA | — | — | 347 |
| | | chromosome:NCBI36:2:183446814:183446920:1 | | | |
| | | gene:ENSG00000207178 | | | |
| 8049375 | ENST00000389758 /// | similar to hCG2012694 | — | — | 348 |
| | ENST00000396517 /// | gene:ENSG00000185038 /// | | | |
| | ENST00000327506 | similar to hCG2012694 | | | |
| | | gene:ENSG00000185038 /// | | | |
| | | similar to hCG2012694 | | | |
| | | gene:ENSG00000185038 | | | |
| 8122732 | GENSCAN00000041083 /// | cdna:Genscan | — | — | 349 |
| | ENST00000309074 | chromosome:NCBI36:6:150340754:150341242:1 /// | | | |
| | | cdna:pseudogene | | | |
| | | chromosome:NCBI36:6:150340754:150341242:1 | | | |
| | | gene:ENSG00000173909 | | | |
| 7907024 | ENST00000367876 /// | Pogo transposable element | pogo transposable | POGK | 350 |
| | ENST00000367875 /// | with KRAB domain | element with KRAB | | |
| | AB040946 /// | gene:ENSG00000143157 /// | domain | | |
| | NM_017542 | Pogo transposable element | | | |
| | | with KRAB domain | | | |
| | | gene:ENSG00000143157 /// | | | |
| | | Homo sapiens mRNA for | | | |
| | | KIAA1513 protein, partial cds. /// | | | |
| | | Homo sapiens pogo | | | |
| | | transposable element with | | | |
| | | KRAB domain (POGK), mRNA. | | | |
| 8161774 | ENST00000376870 /// | Isoform M6-kinase 3 of | transient receptor | TRPM6 | 351 |
| | ENST00000360774 /// | Transient receptor potential | potential cation | | |
| | ENST00000361255 /// | cation channel subfamily M | channel, subfamily M, | | |
| | ENST00000376872 /// | member 6 | member 6 | | |
| | ENST00000376871 /// | gene:ENSG00000119121 /// | | | |
| | ENST00000376864 /// | Isoform TRPM6a of Transient | | | |
| | ENST00000312449 /// | receptor potential cation | | | |
| | ENST00000359047 /// | channel subfamily M member 6 | | | |
| | AF350881 /// | gene:ENSG00000119121 /// | | | |
| | NM_017662 | Putative uncharacterized | | | |
| | | protein TRPM6 | | | |
| | | gene:ENSG00000119121 /// | | | |
| | | Isoform M6-kinase 1 of | | | |
| | | Transient receptor potential | | | |
| | | cation channel subfamily M | | | |
| | | member 6 | | | |
| | | gene:ENSG00000119121 /// | | | |
| | | Isoform M6-kinase 2 of | | | |
| | | Transient receptor potential | | | |
| | | cation channel subfamily M | | | |
| | | member 6 | | | |
| | | gene:ENSG00000119121 /// | | | |
| | | Isoform TRPM6t of Transient | | | |
| | | receptor potential cation | | | |
| | | channel subfamily M member 6 | | | |
| | | gene:ENSG00000119121 /// | | | |
| | | Isoform TRPM6t of Transient | | | |
| | | receptor potential cation | | | |
| | | channel subfamily M member 6 | | | |
| | | gene:ENSG00000119121 /// | | | |
| | | Transient receptor potential | | | |
| | | cation channel, subfamily M, | | | |
| | | member 6, isoform CRA_j | | | |
| | | gene:ENSG00000119121 /// | | | |
| | | Homo sapiens channel kinase 2 | | | |
| | | (CHAK2) mRNA, complete cds. /// | | | |
| | | Homo sapiens transient | | | |
| | | receptor potential cation | | | |
| | | channel, subfamily M, member | | | |
| | | 6 (TRPM6), mRNA. | | | |
| 8157144 | ENST00000322940 /// | UPF0436 protein C9orf6 | chromosome 9 open | C9orf6 | 352 |
| | ENST00000374621 /// | gene:ENSG00000119328 /// | reading frame 6 | | |
| | ENST00000374624 /// | Putative uncharacterized | | | |
| | BC015795 /// | protein C9orf6 (Fragment) | | | |
| | NM_017832 | gene:ENSG00000119328 /// 13 | | | |
| | | kDa protein | | | |
| | | gene:ENSG00000119328 /// | | | |
| | | Homo sapiens chromosome 9 | | | |
| | | open reading frame 6, mRNA | | | |
| | | (cDNA clone MGC:8859 | | | |
| | | IMAGE:3910513), complete | | | |
| | | cds. /// Homo sapiens | | | |
| | | chromosome 9 open reading | | | |
| | | frame 6 (C9orf6), mRNA. | | | |
| 8018377 | ENST00000411285 /// | ncrna:snRNA | — | — | 353 |
| | ENST00000388598 | chromosome:NCBI36:17:70916762:70916860:−1 | | | |
| | | gene:ENSG00000223217 /// | | | |
| | | ncrna:snRNA_pseudogene | | | |
| | | chromosome:NCBI36:17:70916768:70916864:−1 | | | |
| | | gene:ENSG00000211333 | | | |
| 8146564 | ENST00000262646 /// | Ras-related protein Rab-2A | RAB2A, member RAS | RAB2A | 354 |
| | ENST00000396697 /// | gene:ENSG00000104388 /// 24 | oncogene family | | |
| | ENST00000396696 /// | kDa protein | | | |
| | BC008929 /// | gene:ENSG00000104388 /// | | | |
| | NM_002865 | Putative uncharacterized | | | |
| | | protein RAB2A | | | |
| | | gene:ENSG00000104388 /// | | | |
| | | Homo sapiens RAB2A, member | | | |
| | | RAS oncogene family, mRNA | | | |
| | | (cDNA clone MGC:1656 | | | |
| | | IMAGE:2966694), complete | | | |
| | | cds. /// Homo sapiens RAB2A, | | | |
| | | member RAS oncogene family | | | |
| | | (RAB2A), mRNA. | | | |
| 7957298 | ENST00000266692 /// | Isoform 3 of Neuron navigator 3 | neuron navigator 3 | NAV3 | 355 |
| | ENST00000228327 /// | gene:ENSG00000067798 /// | | | |
| | ENST00000397909 /// | Isoform 1 of Neuron navigator 3 | | | |
| | ENST00000378640 /// | gene:ENSG00000067798 /// | | | |
| | BC017667 /// | Isoform 2 of Neuron navigator 3 | | | |
| | NM_014903 | gene:ENSG00000067798 /// | | | |
| | | 253 kDa protein | | | |
| | | gene:ENSG00000067798 /// | | | |
| | | Homo sapiens neuron navigator | | | |
| | | 3, mRNA (cDNA clone | | | |
| | | IMAGE:3914378), partial cds. /// | | | |
| | | Homo sapiens neuron | | | |
| | | navigator 3 (NAV3), mRNA. | | | |
| 8091118 | — | — | — | — | 356 |
| 8107909 | ENST00000200652 /// | Solute carrier family 22 member | solute carrier family | SLC22A4 | 357 |
| | BC028313 /// | 4 gene:ENSG00000197208 /// | 22 (organic | | |
| | NM_003059 | Homo sapiens solute carrier | cation/ergothioneine | | |
| | | family 22 (organic | transporter), member | | |
| | | cation/ergothioneine | 4 | | |
| | | transporter), member 4, mRNA | | | |
| | | (cDNA clone MGC:34546 | | | |
| | | IMAGE:5186192), complete | | | |
| | | cds. /// Homo sapiens solute | | | |
| | | carrier family 22 (organic | | | |
| | | cation/ergothioneine | | | |
| | | transporter), member 4 | | | |
| | | (SLC22A4), mRNA. | | | |
| 8101723 | ENST00000323061 /// | Nucleosome assembly protein | nucleosome | NAP1L5 | 358 |
| | BC104883 /// | 1-like 5 | assembly protein 1- | | |
| | NM_153757 | gene:ENSG00000177432 /// | like 5 | | |
| | | Homo sapiens nucleosome | | | |
| | | assembly protein 1-like 5, | | | |
| | | mRNA (cDNA clone | | | |
| | | MGC:132543 | | | |
| | | IMAGE:8143886), complete | | | |
| | | cds. /// Homo sapiens | | | |
| | | nucleosome assembly protein | | | |
| | | 1-like 5 (NAP1L5), mRNA. | | | |
| 8053311 | ENST00000363618 | ncrna:misc_RNA | — | — | 359 |
| | | chromosome:NCBI36:2:76525713:76526044:−1 | | | |
| | | gene:ENSG00000200488 | | | |
| 8054364 | ENST00000393359 /// | Transforming growth factor- | transforming growth | TGFBRAP1 | 360 |
| | ENST00000258449 /// | beta receptor-associated | factor, beta receptor | | |
| | BC020548 /// | protein 1 | associated protein 1 | | |
| | NM_004257 | gene:ENSG00000135966 /// | | | |
| | | Transforming growth factor- | | | |
| | | beta receptor-associated | | | |
| | | protein 1 | | | |
| | | gene:ENSG00000135966 /// | | | |
| | | Homo sapiens transforming | | | |
| | | growth factor, beta receptor | | | |
| | | associated protein 1, mRNA | | | |
| | | (cDNA clone MGC:21319 | | | |
| | | IMAGE:4420120), complete | | | |
| | | cds. /// Homo sapiens | | | |
| | | transforming growth factor, beta | | | |
| | | receptor associated protein 1 | | | |
| | | (TGFBRAP1), mRNA. | | | |
| 7938295 | ENST00000314138 /// | 60S ribosomal protein L27a | ribosomal protein | RPL27A | 361 |
| | NM_000990 | gene:ENSG00000166441 /// | L27a | | |
| | | Homo sapiens ribosomal | | | |
| | | protein L27a (RPL27A), mRNA. | | | |
| 7995267 | ENST00000315486 /// | Isoform 1 of TP53-target gene 3 | TP53 target 3 /// | TP53TG3 /// | 362 |
| | ENST00000341305 /// | protein | similar to TP53TG3 | LOC729355 | |
| | ENST00000398682 /// | gene:ENSG00000180598 /// | protein | | |
| | ENST00000354614 /// | Isoform 2 of TP53-target gene 3 | | | |
| | ENST00000398680 /// | protein | | | |
| | ENST00000398667 /// | gene:ENSG00000180598 /// | | | |
| | ENST00000398666 /// | Isoform 2 of TP53-target gene 3 | | | |
| | ENST00000360260 /// | protein | | | |
| | ENST00000398664 /// | gene:ENSG00000183632 /// | | | |
| | ENST00000380147 /// | Isoform 3 of TP53-target gene 3 | | | |
| | ENST00000380148 /// | protein | | | |
| | AB023508 /// | gene:ENSG00000183632 /// | | | |
| | NM_016212 /// | Isoform 1 of TP53-target gene 3 | | | |
| | NM_001099687 | protein | | | |
| | | gene:ENSG00000183632 /// | | | |
| | | Isoform 2 of TP53-target gene 3 | | | |
| | | protein | | | |
| | | gene:ENSG00000205457 /// | | | |
| | | Isoform 1 of TP53-target gene 3 | | | |
| | | protein | | | |
| | | gene:ENSG00000205457 /// | | | |
| | | Isoform 3 of TP53-target gene 3 | | | |
| | | protein | | | |
| | | gene:ENSG00000205457 /// | | | |
| | | Isoform 2 of TP53-target gene 3 | | | |
| | | protein | | | |
| | | gene:ENSG00000205456 /// | | | |
| | | Isoform 1 of TP53-target gene 3 | | | |
| | | protein | | | |
| | | gene:ENSG00000205456 /// | | | |
| | | Isoform 3 of TP53-target gene 3 | | | |
| | | protein | | | |
| | | gene:ENSG00000205456 /// | | | |
| | | Homo sapiens mRNA for | | | |
| | | TP53TG3b, complete cds. /// | | | |
| | | Homo sapiens TP53 target 3 | | | |
| | | (TP53TG3), mRNA. /// Homo sapiens | | | |
| | | similar to TP53TG3 | | | |
| | | protein (LOC729355), mRNA. | | | |
| 8032465 | ENST00000300961 /// | Junctional sarcoplasmic | junctional | JSRP1 | 363 |
| | BC021201 /// | reticulum protein 1 | sarcoplasmic | | |
| | NM_144616 | gene:ENSG00000167476 /// | reticulum protein 1 | | |
| | | Homo sapiens junctional | | | |
| | | sarcoplasmic reticulum protein | | | |
| | | 1, mRNA (cDNA clone | | | |
| | | MGC:13120 IMAGE:4106867), | | | |
| | | complete cds. /// Homo sapiens | | | |
| | | junctional sarcoplasmic | | | |
| | | reticulum protein 1 (JSRP1), | | | |
| | | mRNA. | | | |
| 8167953 | ENST00000411174 /// | ncrna:misc_RNA | — | — | 364 |
| | ENST00000388411 | chromosome:NCBI36:X:63347296:63347591:1 | | | |
| | | gene:ENSG00000223106 /// | | | |
| | | ncrna:scRNA_pseudogene | | | |
| | | chromosome:NCBI36:X:63347296:63347593:1 | | | |
| | | gene:ENSG00000211146 | | | |
| 8102643 | ENST00000274026 /// | Cyclin-A2 | cyclin A2 | CCNA2 | 365 |
| | AK291931 /// | gene:ENSG00000145386 /// | | | |
| | NM_001237 | Homo sapiens cDNA FLJ77347 | | | |
| | | complete cds, highly similar to | | | |
| | | Homo sapiens cyclin A2 | | | |
| | | (CCNA2), mRNA. /// Homo sapiens | | | |
| | | cyclin A2 (CCNA2), | | | |
| | | mRNA. | | | |
| 7897960 | ENST00000332530 /// | arylacetamide deacetylase-like | arylacetamide | AADACL3 | 366 |
| | ENST00000359318 /// | 3 isoform 2 | deacetylase-like 3 | | |
| | NM_001103169 /// | gene:ENSG00000188984 /// | | | |
| | NM_001103170 | Arylacetamide deacetylase-like | | | |
| | | 3 gene:ENSG00000188984 /// | | | |
| | | Homo sapiens arylacetamide | | | |
| | | deacetylase-like 3 (AADACL3), | | | |
| | | transcript variant 2, mRNA. /// | | | |
| | | Homo sapiens arylacetamide | | | |
| | | deacetylase-like 3 (AADACL3), | | | |
| | | transcript variant 1, mRNA. | | | |
| 8055424 | ENST00000363794 | ncrna:misc_RNA | — | — | 367 |
| | | chromosome:NCBI36:2:136284209:136284319:−1 | | | |
| | | gene:ENSG00000200664 | | | |
| 8097704 | ENST00000296582 /// | Isoform 1 of Transmembrane | transmembrane | TMEM184C | 368 |
| | BC046128 /// | protein 184C | protein 184C | | |
| | NM_018241 | gene:ENSG00000164168 /// | | | |
| | | Homo sapiens transmembrane | | | |
| | | protein 184C, mRNA (cDNA | | | |
| | | clone MGC:57601 | | | |
| | | IMAGE:5750613), complete | | | |
| | | cds. /// Homo sapiens | | | |
| | | transmembrane protein 184C | | | |
| | | (TMEM184C), mRNA. | | | |
| 8106820 | ENST00000399107 /// | DNA-directed RNA polymerase III | polymerase (RNA) III | POLR3G | 369 |
| | AK295434 /// | subunit G | (DNA directed) | | |
| | NM_006467 | gene:ENSG00000113356 /// | polypeptide G (32 kD) | | |
| | | Homo sapiens cDNA FLJ60555 | | | |
| | | complete cds, highly similar to | | | |
| | | DNA-directed RNA polymerase III | | | |
| | | subunit G (EC2.7.7.6). /// | | | |
| | | Homo sapiens polymerase | | | |
| | | (RNA) III (DNA directed) | | | |
| | | polypeptide G (32 kD) | | | |
| | | (POLR3G), mRNA. | | | |
| 7992782 | ENST00000396927 /// | Claudin-9 | claudin 9 /// HCTP4- | CLDN9 /// | 370 |
| | AK091002 /// | gene:ENSG00000213937 /// | binding protein | LOC100134406 | |
| | AY390431 /// | Homo sapiens cDNA FLJ33683 | | | |
| | NM_020982 | fis, clone BRAWH2002623, | | | |
| | | highly similar to CLAUDIN-9. /// | | | |
| | | Homo sapiens HCTP4-binding | | | |
| | | protein mRNA, complete cds. /// | | | |
| | | Homo sapiens claudin 9 | | | |
| | | (CLDN9), mRNA. | | | |
| 8089830 | ENST00000295628 /// | Leucine-rich repeat-containing | leucine rich repeat | LRRC58 | 371 |
| | NM_001099678 | protein 58 | containing 58 | | |
| | | gene:ENSG00000163428 /// | | | |
| | | Homo sapiens leucine rich | | | |
| | | repeat containing 58 (LRRC58), | | | |
| | | mRNA. | | | |
| 7998629 | GENSCAN00000001581 | cdna:Genscan | — | — | 372 |
| | | chromosome:NCBI36:16:1831818:1893847:−1 | | | |
| 7943736 | hsa-mir-34b /// | MI0000742 Homo sapiens miR- | — | — | 373 |
| | hsa-mir-34b | 34b stem-loop /// MI0000742 | | | |
| | | Homo sapiens miR-34b stem- | | | |
| | | loop | | | |
| 7921356 | ENST00000392265 /// | Seven transmembrane helix | olfactory receptor, | OR10K2 | 374 |
| | ENST00000314902 /// | receptor | family 10, subfamily | | |
| | NM_001004476 | gene:ENSG00000180708 /// | K, member 2 | | |
| | | Olfactory receptor 10K2 | | | |
| | | gene:ENSG00000180708 /// | | | |
| | | Homo sapiens olfactory | | | |
| | | receptor, family 10, subfamily | | | |
| | | K, member 2 (OR10K2), | | | |
| | | mRNA. | | | |
| 8052413 | GENSCAN00000048378 /// | cdna:Genscan | — | — | 375 |
| | ENST00000404638 | chromosome:NCBI36:2:60815529:60816357:−1 /// | | | |
| | | cdna:pseudogene | | | |
| | | chromosome:NCBI36:2:60815529:60816366:−1 | | | |
| | | gene:ENSG00000217407 | | | |
| 7961865 | ENST00000256078 /// | Isoform 2A of GTPase KRas | v-Ki-ras2 Kirsten rat | KRAS | 376 |
| | ENST00000311936 /// | gene:ENSG00000133703 /// | sarcoma viral | | |
| | ENST00000395977 /// | Isoform 2B of GTPase KRas | oncogene homolog | | |
| | M54968 /// | gene:ENSG00000133703 /// | | | |
| | NM_004985 /// | Isoform 2A of GTPase KRas | | | |
| | NM_033360 | gene:ENSG00000133703 /// | | | |
| | | Homo sapiens K-ras oncogene | | | |
| | | protein (KRAS) mRNA, | | | |
| | | complete cds. /// Homo sapiens | | | |
| | | v-Ki-ras2 Kirsten rat sarcoma | | | |
| | | viral oncogene homolog | | | |
| | | (KRAS), transcript variant b, | | | |
| | | mRNA. /// Homo sapiens v-Ki- | | | |
| | | ras2 Kirsten rat sarcoma viral | | | |
| | | oncogene homolog (KRAS), | | | |
| | | transcript variant a, mRNA. | | | |
| 8142019 | ENST00000297431 /// | Origin recognition complex | origin recognition | ORC5L | 377 |
| | AK302122 /// | subunit 5 | complex, subunit 5- | | |
| | NM_002553 /// | gene:ENSG00000164815 /// | like (yeast) | | |
| | NM_181747 | Homo sapiens cDNA FLJ53851 | | | |
| | | complete cds, highly similar to | | | |
| | | Origin recognition complex | | | |
| | | subunit 5. /// Homo sapiens | | | |
| | | origin recognition complex, | | | |
| | | subunit 5-like (yeast) (ORC5L), | | | |
| | | transcript variant 1, mRNA. /// | | | |
| | | Homo sapiens origin | | | |
| | | recognition complex, subunit 5- | | | |
| | | like (yeast) (ORC5L), transcript | | | |
| | | variant 2, mRNA. | | | |
| 8009873 | ENST00000325720 /// | cdna:known | myosin XVB | MYO15B | 378 |
| | AF418286 /// | chromosome:NCBI36:17:71097148:71101765:1 | pseudogene | | |
| | NR_003587 | gene:ENSG00000204326 /// | | | |
| | | Homo sapiens clone 1 myosin | | | |
| | | XVBP (MYO15B) pseudogene, | | | |
| | | partial sequence. /// Homo sapiens | | | |
| | | myosin XVB | | | |
| | | pseudogene (MYO15B) on | | | |
| | | chromosome 17. | | | |
| 8149749 | ENST00000312584 /// | Tumor necrosis factor receptor | tumor necrosis factor | TNFRSF10D | 379 |
| | AY358285 /// | superfamily member 10D | receptor superfamily, | | |
| | NM_003840 | gene:ENSG00000173530 /// | member 10d, decoy | | |
| | | Homo sapiens clone | with truncated death | | |
| | | DNA35663 DcR2-TNFR | domain | | |
| | | (UNQ251) mRNA, complete | | | |
| | | cds. /// Homo sapiens tumor | | | |
| | | necrosis factor receptor | | | |
| | | superfamily, member 10d, | | | |
| | | decoy with truncated death | | | |
| | | domain (TNFRSF10D), mRNA. | | | |
| 7986665 | ENST00000337451 /// | Non-imprinted in Prader- | non imprinted in | NIPA2 | 380 |
| | ENST00000359727 /// | Willi/Angelman syndrome | Prader- | | |
| | ENST00000398014 /// | region protein 2 | Willi/Angelman | | |
| | ENST00000398013 /// | gene:ENSG00000140157 /// | syndrome 2 | | |
| | BC011775 /// | non imprinted in Prader- | | | |
| | NM_030922 /// | Willi/Angelman syndrome 2 | | | |
| | NM_001008892 /// | isoform b | | | |
| | NM_001008894 /// | gene:ENSG00000140157 /// | | | |
| | NM_001008860 | Non-imprinted in Prader- | | | |
| | | Willi/Angelman syndrome | | | |
| | | region protein 2 | | | |
| | | gene:ENSG00000140157 /// | | | |
| | | Non-imprinted in Prader- | | | |
| | | Willi/Angelman syndrome | | | |
| | | region protein 2 | | | |
| | | gene:ENSG00000140157 /// | | | |
| | | Homo sapiens non imprinted in | | | |
| | | Prader-Willi/Angelman | | | |
| | | syndrome 2, mRNA (cDNA | | | |
| | | clone MGC:19609 | | | |
| | | IMAGE:3640970), complete | | | |
| | | cds. /// Homo sapiens non | | | |
| | | imprinted in Prader- | | | |
| | | Willi/Angelman syndrome 2 | | | |
| | | (NIPA2), transcript variant 1, | | | |
| | | mRNA. /// Homo sapiens non | | | |
| | | imprinted in Prader- | | | |
| | | Willi/Angelman syndrome 2 | | | |
| | | (NIPA2), transcript variant 3, | | | |
| | | mRNA. /// Homo sapiens non | | | |
| | | imprinted in Prader- | | | |
| | | Willi/Angelman syndrome 2 | | | |
| | | (NIPA2), transcript variant 4, | | | |
| | | mRNA. /// Homo sapiens non | | | |
| | | imprinted in Prader- | | | |
| | | Willi/Angelman syndrome 2 | | | |
| | | (NIPA2), transcript variant 2, | | | |
| | | mRNA. | | | |
| 8163015 | ENST00000386231 /// | ncrna:scRNA_pseudogene | — | — | 381 |
| | ENST00000411190 | chromosome:NCBI36:9:109613716:109614016:−1 | | | |
| | | gene:ENSG00000208966 /// | | | |
| | | ncrna:misc_RNA | | | |
| | | chromosome:NCBI36:9:109613719:109614016:−1 | | | |
| | | gene:ENSG00000223122 | | | |
| 8007548 | AF143236 | Homo sapiens apoptosis | chromosome 17 open | C17orf88 | 382 |
| | | related protein APR-2 mRNA, | reading frame 88 | | |
| | | complete cds. | | | |
| 7923659 | ENST00000367188 /// | Protein phosphatase 1 | protein phosphatase | PPP1R15B | 383 |
| | BC065280 /// | regulatory subunit 15B | 1, regulatory | | |
| | NM_032833 | gene:ENSG00000158615 /// | (inhibitor) subunit 15B | | |
| | | Homo sapiens protein | | | |
| | | phosphatase 1, regulatory | | | |
| | | (inhibitor) subunit 15B, mRNA | | | |
| | | (cDNA clone MGC:74824 | | | |
| | | IMAGE:6172811), complete | | | |
| | | cds. /// Homo sapiens protein | | | |
| | | phosphatase 1, regulatory | | | |
| | | (inhibitor) subunit 15B | | | |
| | | (PPP1R15B), mRNA. | | | |
| 8001666 | ENST00000262506 /// | Casein kinase II subunit alpha' | casein kinase 2, | CSNK2A2 | 384 |
| | M55268 /// | gene:ENSG00000070770 /// | alpha prime | | |
| | NM_001896 | Human casein kinase II alpha' | polypeptide | | |
| | | subunit mRNA, complete cds. /// | | | |
| | | Homo sapiens casein kinase | | | |
| | | 2, alpha prime polypeptide | | | |
| | | (CSNK2A2), mRNA. | | | |
| 8155418 | ENST00000316269 /// | hypothetical protein | hypothetical protein | LOC100133036 /// | 385 |
| | AK125850 /// | gene:ENSG00000204831 /// | LOC100133036 /// | FAM95B1 | |
| | AL833349 | Homo sapiens cDNA FLJ43862 | family with sequence | | |
| | | fis, clone TESTI4007775. /// | similarity 95, member | | |
| | | Homo sapiens mRNA; cDNA | B1 | | |
| | | DKFZp686P0734 (from clone | | | |
| | | DKFZp686P0734). | | | |
| 7952046 | ENST00000278937 /// | Myelin protein zero-like protein | myelin protein zero- | MPZL2 | 386 |
| | BC017774 /// | 2 gene:ENSG00000149573 /// | like 2 | | |
| | NM_144765 /// | Homo sapiens myelin protein | | | |
| | NM_005797 | zero-like 2, mRNA (cDNA clone | | | |
| | | MGC:22243 IMAGE:4692569), | | | |
| | | complete cds. /// Homo sapiens | | | |
| | | myelin protein zero-like 2 | | | |
| | | (MPZL2), transcript variant 2, | | | |
| | | mRNA. /// Homo sapiens myelin | | | |
| | | protein zero-like 2 (MPZL2), | | | |
| | | transcript variant 1, mRNA. | | | |
| 8141843 | uc003vad.1 /// | /// PREDICTED: Homo sapiens | similar to HSPC047 | LOC100134722 /// | 387 |
| | XM_001725218 /// | similar to HSPC047 protein | protein /// similar to | LOC100133005 | |
| | XM_001718200 | (LOC100134722), mRNA. /// | RAS p21 protein | | |
| | | PREDICTED: Homo sapiens | activator 4 | | |
| | | similar to RAS p21 protein | | | |
| | | activator 4 (LOC100133005), | | | |
| | | mRNA. | | | |
| 8023175 | ENST00000256433 /// | Immediate early response 3- | immediate early | IER3IP1 | 388 |
| | | interacting protein 1 | response 3 interacting | | |
| | | gene:ENSG00000134049 /// | protein 1 | | |
| | AK027108 /// | Homo sapiens cDNA: | | | |
| | NM_016097 | FLJ23455 fis, clone HSI07063, | | | |
| | | highly similar to AF125100 | | | |
| | | Homo sapiens HSPC039 | | | |
| | | protein mRNA. /// Homo sapiens | | | |
| | | immediate early | | | |
| | | response 3 interacting protein 1 | | | |
| | | (IER3IP1), mRNA. | | | |
| 8072153 | ENST00000249064 /// | Isoform 1 of Coiled-coil domain- | coiled-coil domain | CCDC117 | 389 |
| | BC053874 /// | containing protein 117 | containing 117 | | |
| | NM_173510 | gene:ENSG00000159873 /// | | | |
| | | Homo sapiens coiled-coil | | | |
| | | domain containing 117, mRNA | | | |
| | | (cDNA clone MGC:61737 | | | |
| | | IMAGE:5531689), complete | | | |
| | | cds. /// Homo sapiens coiled- | | | |
| | | coil domain containing 117 | | | |
| | | (CCDC117), mRNA. | | | |
| 7928308 | ENST00000307365 /// | DNA-damage-inducible | DNA-damage- | DDIT4 | 390 |
| | BC007714 /// | transcript 4 protein | inducible transcript 4 | | |
| | NM_019058 | gene:ENSG00000168209 /// | | | |
| | | Homo sapiens DNA-damage- | | | |
| | | inducible transcript 4, mRNA | | | |
| | | (cDNA clone MGC:12610 | | | |
| | | IMAGE:4302878), complete | | | |
| | | cds. /// Homo sapiens DNA- | | | |
| | | damage-inducible transcript 4 | | | |
| | | (DDIT4), mRNA. | | | |
| 8108099 | ENST00000322887 /// | Isoform 2 of Protein transport | SEC24 family, | SEC24A | 391 |
| | ENST00000398844 /// | protein Sec24A | member A | | |
| | ENST00000265341 /// | gene:ENSG00000113615 /// | (S. cerevisiae) | | |
| | AK304060 /// | Isoform 1 of Protein transport | | | |
| | NM_021982 | protein Sec24A | | | |
| | | gene:ENSG00000113615 /// | | | |
| | | cDNA FLJ61651, highly similar | | | |
| | | to Protein transport protein | | | |
| | | Sec24A | | | |
| | | gene:ENSG00000113615 /// | | | |
| | | Homo sapiens cDNA FLJ61651 | | | |
| | | complete cds, highly similar to | | | |
| | | Protein transport protein | | | |
| | | Sec24A. /// Homo sapiens | | | |
| | | SEC24 family, member A | | | |
| | | (S. cerevisiae) (SEC24A), mRNA. | | | |

### REFERENCES

1. DeGroot, A., Patch Testing. Test Concentrations and Vehicles for 4350 Chemicals. 3rd edition ed. 2008: acdegroot publishing.
2. Luechtefeld, T., et al., Analysis of publically available skin sensitization data from REACH registrations 2008-2014. ALTEX, 2016. 33(2): p. 135-48.
3. Boverhof, D. R., et al., Respiratory sensitization and allergy: current research approaches and needs. Toxicol Appl Pharmacol, 2008. 226(1): p. 1-13.
4. Thomas, W. R., et al., Characterization and immunobiology of house dust mite allergens. Int Arch Allergy Immunol, 2002. 129(1): p. 1-18.
5. Baur, X., Enzymes as occupational and environmental respiratory sensitisers. Int Arch Occup Environ Health, 2005. 78(4): p. 279-86.
6. Baur, X., L. T. Budnik, and G. von Kirchbach, Allergic asthma caused by exposure to bacterial alpha-amylase Termamyl®. Am J Ind Med, 2013. 56(3): p. 378-80.
7. Budnik, L. T., et al., Sensitising effects of genetically modified enzymes used in flavour, fragrance, detergence and pharmaceutical production: cross-sectional study. Occup Environ Med, 2016.
8. http://imparas.eu/, accessed 161117.
9. McClain, S., et al., Allergic sensitization: food- and protein-related factors. Clin Transl Allergy, 2014. 4(1): p. 11.
10. North, C. M., et al., Developing a framework for assessing chemical respiratory sensitization: A workshop report. Regul Toxicol Pharmacol, 2016. 80: p. 295-309.
11. OECD, E. C. s. J. R. C., the United States Environmental Protection Agency, the US Army Engineer Research & Development Center, User's handbook supplement to the guidance document for developing and assessing AOPs. 2014.
12. Roggen, E. L., In vitro approaches for detection of chemical sensitization. Basic Clin Pharmacol Toxicol, 2014. 115(1): p. 32-40.
13. Johansson, H., et al., Genomic allergen rapid detection in-house validation—a proof of concept. Toxicol Sci, 2014. 139(2): p. 362-70.
14. Schuurhuis, D. H., et al., Ins and outs of dendritic cells. Int Arch Allergy Immunol, 2006. 140(1): p. 53-72.
15. OECD, The Adverse Outcome Pathway for Skin Sensitisation Initiated by Covalent

Binding to Proteins. Part 1: Scientific Evidence. 2012: p. 1-59.
16. Forreryd, A., et al., Prediction of chemical respiratory sensitizers using GARD, a novel in vitro assay based on a genomic biomarker signature. PLoS One, 2015. 10(3): p. e0118808.
17. Johansson, H., et al., A genomic biomarker signature can predict skin sensitizers using a cell-based in vitro alternative to animal tests. BMC Genomics, 2011. 12: p. 399.
18. Johansson, H., et al., The GARD assay for assessment of chemical skin sensitizers. Toxicol In Vitro, 2013. 27(3): p. 1163-9.
19. Piccolo, S. R., et al., A single-sample microarray normalization method to facilitate personalized-medicine workflows. Genomics, 2012. 100(6): p. 337-44.
20. Piccolo, S. R., et al., Multiplatform single-sample estimates of transcriptional activation. Proc Natl Acad Sci USA, 2013. 110(44): p. 17778-83.
21. Benjamini, Y. and Y. Hochberg, Controlling the False Discovery Rate: A Practical and Powerful Approach to Multiple Testing. Journal of the Royal Statistical Society. Series B (Methodological), 1995. 57(1): p. 289-300.
22. Noble, W. S., What is a support vector machine? Nat Biotech, 2006. 24(12): p. 1565-1567.
23. Carlsson, A., et al., Molecular serum portraits in patients with primary breast cancer predict the development of distant metastases. Proc Natl Acad Sci USA, 2011. 108(34): p. 14252-7.
24. Key Pathway Advisor by Clarivate Analytics (Formerly the IP & Science business of Thomson Reuters). http://ipscience.thomsonreuters.com/product/metacore/. 2016.
25. Cortes, C. and V. Vapnik, Support-Vector Networks. Machine Learning, 1995. 20(3): p. 273-297.
26. Konig, K., et al., Cytokine profiles in nasal fluid of patients with seasonal or persistent allergic rhinitis. Allergy Asthma Clin Immunol, 2015. 11(1): p. 26.
27. King, C., et al., Dust mite proteolytic allergens induce cytokine release from cultured airway epithelium. J Immunol, 1998. 161(7): p. 3645-51.
28. http://www.serumsourceintl.com/questions_answers.html. 2015 [cited 2016 Sep. 5, 2016].
29. Gough, L, et al., Proteolytic activity of the house dust mite allergen Der p 1 enhances allergenicity in a mouse inhalation model. Clin Exp Allergy, 2003. 33(8): p. 1159-63.
30. Gough, L., et al., The cysteine protease activity of the major dust mite allergen Der p 1 selectively enhances the immunoglobulin E antibody response. J Exp Med, 1999. 190(12): p. 1897-902.
31. Porter, P., et al., Link between allergic asthma and airway mucosal infection suggested by proteinase-secreting household fungi. Mucosal Immunol, 2009. 2(6): p. 504-17.
32. Corsini, E., et al., Role of oxidative stress in chemical allergens induced skin cells activation. Food Chem Toxicol, 2013. 61: p. 74-81.
33. Takai, T., et al., TSLP expression induced via Toll-like receptor pathways in human keratinocytes. Methods Enzymol, 2014. 535: p. 371-87.
34. Roggen, E. L., Breakdown of the Molecular Processes Driving the Adverse Outcome Pathways (AOPs) of Skin and Respiratory Sensitization Induction in Humans Exposed to Xenobiotics and Proteins, in Molecular Immunotoxicology, E. C. a. H. v. Loveren, Editor. 2014, Wiley-VCH Verlag GmbH & Co. KGaA: Weinheim, Germany.
35. Ather, J. L., et al., Airway epithelial NF-kappaB activation promotes allergic sensitization to an innocuous inhaled antigen. Am J Respir Cell Mol Biol, 2011. 44(5): p. 631-8.
36. Chiou, Y. L. and C. Y. Lin, Der p2 activates airway smooth muscle cells in a TLR2/MyD88-dependent manner to induce an inflammatory response. J Cell Physiol, 2009. 220(2): p. 311-8.
37. Yazdi, A S., K. Ghoreschi, and M. Rocken, Inflammasome activation in delayed-type hypersensitivity reactions. J Invest Dermatol, 2007. 127(8): p. 1853-5.
38. Kool, M., et al., An unexpected role for uric acid as an inducer of T helper 2 cell immunity to inhaled antigens and inflammatory mediator of allergic asthma. Immunity, 2011. 34(4): p. 527-40.
39. Basketter, D. A., et al., Defining occupational and consumer exposure limits for enzyme protein respiratory allergens under REACH. Toxicology, 2010. 268(3): p. 165-70.
40. Sarlo, K., et al., Proteolytic detergent enzymes enhance the allergic antibody responses of guinea pigs to nonproteolytic detergent enzymes in a mixture: implications for occupational exposure. J Allergy Clin Immunol, 1997. 100(4): p. 480-7.
41. Scheurer, S., M. Toda, and S. Vieths, What makes an allergen? Clin Exp Allergy, 2015. 45(7): p. 1150-61.
42. Schweigert, M. K., D. P. Mackenzie, and K. Sarlo, Occupational asthma and allergy associated with the use of enzymes in the detergent industry—a review of the epidemiology, toxicology and methods of prevention. Clin Exp Allergy, 2000. 30(11): p. 1511-8.
43. Chapman, M. D., S. Wunschmann, and A. Pomes, Proteases as Th2 adjuvants. Curr Allergy Asthma Rep, 2007. 7(5): p. 363-7.
44. Straube, F., et al., Contact allergens and irritants show discrete differences in the activation of human monocyte-derived dendritic cells: consequences for in vitro detection of contact allergens. Arch Toxicol, 2005. 79(1): p. 37-46.
45. Pedersen, L. K., et al., Augmentation of skin response by exposure to a combination of allergens and irritants—a review. Contact Dermatitis, 2004. 50(5): p. 265-73.
46. Grabbe, S., et al., Dissection of antigenic and irritative effects of epicutaneously applied haptens in mice. Evidence that not the antigenic component but nonspecific proinflammatory effects of haptens determine the concentration-dependent elicitation of allergic contact dermatitis. J Clin Invest, 1996. 98(5): p. 1158-64.
47. Martin, S. F., Adaptation in the innate immune system and heterologous innate immunity. Cell Mol Life Sci, 2014. 71(21): p. 4115-30.
48. Piccolo, S. R., et al., A single-sample microarray normalization method to facilitate personalized-medicine workflows. Genomics, 2012. 100(6): p. 337-44.
49. Piccolo, S. R., et al., Multiplatform single-sample estimates of transcriptional activation. Proc Natl Acad Sci USA, 2013. 110(44): p. 17778-83.

1. A method for identifying proteins which are allergenic in a mammal comprising or consisting of the steps of:
(a) providing a population of dendritic cells or a population of dendritic-like cells;
(b) exposing the cells provided in step (a) to a test protein; and
(c) measuring in the cells of step (b) the expression of two or more biomarkers selected from the group defined in Table A;
wherein the expression of the two or more biomarkers measured in step (c) is indicative of the allergenicity of the test protein of step (b).

2. The method according to claim 1 wherein step (c) comprises or consists of measuring the expression of one or more biomarker listed in Table A(i), for example, 2, 3, 4, 5, 6, 7, 8, 9, 10, 11, 12, or 13 of the biomarkers listed in Table A(i).

3. The method according to any one of the preceding claims wherein step (c) comprises or consists of measuring the expression of all of the biomarkers listed in Table A(i).

4. The method according to any one of the preceding claims wherein step (c) comprises or consists of measuring the expression of one or more biomarkers listed in Table A(ii), for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, or 378 of the biomarkers listed in Table A(ii).

5. The method according to any one of the preceding claims wherein step (c) comprises or consists of measuring the expression of all of the biomarkers listed in Table A(ii).

6. The method according to any one of the preceding claims wherein step (c) comprises or consists of measuring the expression of three or more of the biomarkers listed in Table A, for example, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or 391 of the biomarkers listed in Table A.

7. The method according to any one of the preceding claims wherein step (c) comprises or consists of measuring the expression of all of the biomarkers listed in Table A.

8. The method according to any previous claim further comprising:
d) exposing a separate population of the dendritic cells or dendritic-like cells to one or more negative control agent that is not allergenic in a mammal; and
e) measuring in the cells of step (d) the expression of the two or more biomarkers measured in step (c)
wherein the test protein is identified as allergenic in the event that the expression of the two or more biomarkers measured in step (e) differs from the expression of the two or more biomarkers measured in step (c).

9. The method any previous claim further comprising:
f) exposing a separate population of the dendritic cells or dendritic-like cells to one or more positive control agent that is allergenic in a mammal; and
g) measuring in the cells of step (f) the expression of the two or more biomarkers measured in step (c)
wherein the test protein is identified as allergenic in the event that the expression of the two or more biomarkers measured in step (f) corresponds to the expression of the two or more biomarkers measured in step (c).

10. The method according to any one of the preceding claims wherein step (c) comprises measuring the expression of a nucleic acid molecule of one or more of the biomarkers.

11. The method according to claim 10 wherein the nucleic acid molecule is a cDNA molecule or an mRNA molecule.

12. The method according to claim 11 wherein the nucleic acid molecule is an mRNA molecule.

13. The method according to claim 11 wherein the nucleic acid molecule is a cDNA molecule.

14. The method according to any one of claims 10 to 13 wherein measuring the expression of one or more of the biomarkers in step (c) is performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation.

15. The method according to any one of claims 10 to 14 wherein measuring the expression of one or more of the biomarkers in step (c) is determined using a DNA microarray.

16. The method according to any one of the preceding claims wherein measuring the expression of one or more of the biomarkers in step (c) is performed using one or more binding moieties, each capable of binding selectively to a nucleic acid molecule encoding one of the biomarkers identified in Table A.

17. The method according to claim 16 wherein the one or more binding moieties each comprise or consist of a nucleic acid molecule.

18. The method according to claim 16 wherein the one or more binding moieties each comprise or consist of DNA, RNA, PNA, LNA, GNA, TNA or PMO.

19. The method according to claim 17 or 18 wherein the one or more binding moieties each comprise or consist of DNA.

20. The method according to any one of claims 16 to 19 wherein the one or more binding moieties are 5 to 100 nucleotides in length.

21. The method according to any one of claims 16 to 20 wherein the one or more binding moieties are 15 to 35 nucleotides in length.

22. The method according to any one of claims 16 to 21 wherein the binding moiety comprises a detectable moiety.

23. The method according to claim 22 wherein the detectable moiety is selected from the group consisting of: a fluorescent moiety; a luminescent moiety; a chemiluminescent moiety; a radioactive moiety (for example, a radioactive atom); or an enzymatic moiety.

24. The method according to claim 23 wherein the detectable moiety comprises or consists of a radioactive atom.

25. The method according to claim 24 wherein the radioactive atom is selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

26. The method according to claim 23 wherein the detectable moiety of the binding moiety is a fluorescent moiety.

27. The method according to any one of claims 1 to 9 wherein step (c) comprises or consists of measuring the expression of the protein of one or more of the biomarkers.

28. The method according to claim 27 wherein measuring the expression of one or more of the biomarkers in step (c) is performed using one or more binding moieties each capable of binding selectively to one of the biomarkers identified in Table A.

29. The method according to claim 28 wherein the one or more binding moieties comprise or consist of an antibody or an antigen-binding fragment thereof.

30. The method according to any one of claims 28 to 29 wherein the one or more binding moieties comprise a detectable moiety.

31. The method according to claim 30 wherein the detectable moiety is selected from the group consisting of a fluorescent moiety, a luminescent moiety, a chemiluminescent moiety, a radioactive moiety and an enzymatic moiety.

32. The method according to any one of the preceding claims wherein step (c) is performed using an array.

33. The method according to claim 32 wherein the array is a bead-based array.

34. The method according to claim 33 wherein the array is a surface-based array.

35. The method according to any one of claims 32 to 34 wherein the array is selected from the group consisting of: macroarray; microarray; nanoarray.

36. The method according to any one of the preceding claims wherein the method is performed in vitro, in vivo, ex vivo or in silico.

37. The method according to claim 36 wherein the method is performed in vitro.

38. The method according to any one of the preceding claims wherein the population of dendritic cells or population of dendritic-like cells comprises or consists of immortal and/or non-naturally occurring cells.

39. The method according to any one of the preceding claims wherein the population of dendritic cells or population of dendritic-like cells is a population of dendritic-like cells.

40. The method according to claim 39 wherein the dendritic-like cells are myeloid dendritic-like cells.

41. The method according to claim 40 wherein the myeloid dendritic-like cells are derived from myeloid dendritic cells.

42. The method according to claim 41 wherein the cells derived from myeloid dendritic cells are myeloid leukaemia-derived cells such as those selected from the group consisting of KG-1, THP-1, U-937, HL-60, Monomac-6, AML-193 and MUTZ-3.

43. The method according to any one of the preceding claims for identifying proteins capable of inducing a type I hypersensitivity response in a mammal.

44. The method according to any one of the preceding claims for identifying proteins capable of inducing respiratory sensitization in a mammal.

45. The method according to any one of the preceding claims for identifying proteins capable of inducing a respiratory hypersensitivity response.

46. The method according to any one of the preceding claims wherein the hypersensitivity response is a humoral hypersensitivity response.

47. The method according to any one of the preceding claims for identifying allergenic food proteins.

48. The method according to any one of the claims 8 to 47 wherein the one or more negative control agent provided in step (d) is selected from the group consisting of: unstimulated cells; cell media; vehicle control; DMSO; LPS.

49. The method according to any one of claims 9 to 48 wherein the one or more positive control agent provided in step (f) comprises or consists of one or more agent selected from the group consisting of: Der p 1; and Der p 7.

50. The method according to any one of the preceding claims wherein the method is indicative of the allergenic potency of the sample to be tested.

51. The method according to any one of the preceding claims wherein the method comprises one or more of the following steps:
(i) cultivating dendritic or dendritic-like cells;
(ii) seeding cells of (i) in one or more well(s), e.g. wells of one or more multi-well assay plates;
(iii) adding to a one or more well(s) of (ii) the protein(s) to be tested;
(iv) adding to one or more separate well(s) of (ii) one or more positive control(s), e.g. Der p 1 and/or Der p 7;
(v) adding to one or more separate well(s) of (ii) one or more negative control(s), e.g. DMSO, and/or leaving one or more separate wells(s) of (ii) unstimulated to obtain a medium control;
(vi) incubating cells in wells of (iii)-(v), preferably for about 24 hours;
(vii) isolating purified total RNA from cells of (vi) and, optionally, convert mRNA into cDNA;
(viii) quantifying expression levels of individual mRNA transcripts from (vii), e.g. using an array, such as an Affymetrix Human Gene 1.0 ST array;
(ix) exporting and normalizing expression data from (viii);
(x) isolating data from (ix) originating from biomarkers of the GARD Protein Allergen Prediction Signature (i.e. the biomarkers of Table A);
(xi) applying a prediction model to data from (x), e.g. a frozen SVM model previously established and trained on historical data, e.g. data obtained in Example 1, to predict the allergenicity of tested protein(s) and negative/positive control(s).

52. An array for use in the method according to any one of claims 1-51, the array comprising one or more binding moieties as defined in any one of claims 16-26 and 28-31.

53. The array according to claim 52 wherein the array comprises one or more binding moiety for each of the biomarkers defined in any one of the preceding claims.

54. Use of two or more biomarkers selected from the group defined in Table A for determining the allergenicity of a protein, preferably wherein one or more of the biomarkers is selected from the group defined in Table A(i).

55. Use of two or more binding moieties each with specificity for a biomarker selected from the group defined in Table A for determining the allergenicity of a protein, preferably wherein one or more of the binding moieties has specificity for a biomarker selected from the group defined in Table A(i).

56. An analytical kit for use in a method according any one of claims 1-55 comprising:
(a) an array according to any one of claims 52-53; and
(b) (optionally) one or more control agent.
(c) (optionally) instructions for performing the method as defined in any one of claims 1-51.

57. A method use, array or kit substantially as described herein.
